(19) United States
(12) Patent Application Publication
(10) Pub. No.: US 2025/0034283 A1
(43) Pub. Date: Jan. 30, 2025

(54) ANTI-PHOSPHOCHOLINE ANTIBODIES AND METHODS OF USE THEREOF

(71) Applicant: Oxitope Pharma B.V., Naarden (NL)

(72) Inventors: Jean-Philippe Burckert, South San Francisco, CA (US); Katherine Vousden, Saffron Walden (GB)

(21) Appl. No.: 18/920,412

(22) Filed: Oct. 18, 2024

Related U.S. Application Data
(63) Continuation of application No. PCT/IB2024/000127, filed on Mar. 18, 2023.
(60) Provisional application No. 63/452,798, filed on Mar. 17, 2023.

(51) Int. Cl. C07K 16/44

(52) U.S. Cl. CPC C07K 16/44; C07K 2317/52; C07K 2317/565; C07K 2317/622

(57) ABSTRACT

The present disclosure provides polypeptides that specifically bind to phosphocholine (PC). Also provided are pharmaceutical compositions comprising these polypeptides, nucleic acids encoding these polypeptides, expression vectors and host cells for making these polypeptides, and methods of treating a subject using these polypeptides.

### CROSS-REFERENCE TO RELATED APPLICATIONS

This application is a continuation of International Application Serial No. PCT/IB2024/000127, filed Mar. 18, 2024, which claims priority to U.S. Provisional Patent Application Ser. No. 63/452,798, filed Mar. 17, 2023, the entire disclosures of which are hereby incorporated herein by reference.

### REFERENCE TO SEQUENCE LISTING

This application contains a sequence listing which has been submitted electronically in ST.26 format and is hereby incorporated by reference in its entirety. Said ST.26 copy, created on Oct. 7, 2024, is named “212225_seqlist.xml” and is 644,762 bytes in size.

### FIELD

The present disclosure relates to antibodies that are specific for phosphocholine (PC), and methods of use thereof.

### BACKGROUND

Phospholipids that contain polyunsaturated fatty acids are highly susceptible to modification by reactive oxygen species (ROS) and non-radical oxidizing agents. Oxidized lipids in cell membranes and in circulation can have both advantageous and harmful effects on the human body. Endogenous oxidized phospholipids (OxPL) have been shown to function as stress signals, causing phagocytes such as macrophages to respond by triggering inflammatory programs and eliminating the source of stress. However, excessive amounts of oxidized phospholipid products have been linked to chronic inflammation and the pathogenesis of various cardiopulmonary disorders, such as atherosclerosis and thrombosis, acute lung injury, and neurodegenerative processes.

Oxidized low-density lipoprotein (OxLDL) contains a number of OxPL species and is found in atherosclerotic lesions and detected at high concentration in sera from patients with hyperlipidemia, diabetes mellitus, and liver disease. The phosphocholine (PC) headgroup of OxPLs has been shown to be important for phospholipid binding to integral membrane proteins such as CD36 and the innate defense molecule C-reactive protein (CRP) (Boullier et al., J Lipid Res. 2005 May; 46 (5): 969-76; Gershov et al., J Exp Med. 2000 Nov. 6; 192 (9): 1353-64).

Thus, there is a need for therapies, including antibodies, that bind PC for targeting OxPL across a wide range of diseases.

### SUMMARY

The present disclosure provides polypeptides (e.g., antibodies) that specifically bind to PC. Also provided are pharmaceutical compositions comprising these antibodies, nucleic acids encoding these antibodies, expression vectors and host cells for making these antibodies, and methods of treating a subject using these antibodies. The polypeptides provided herein are particularly advantageous because they bind PC with high affinity, display favorable stability, stress resistance, and immunogenicity profiles, and can be produced at high yields. The polypeptides provided herein are particularly useful for treating an inflammatory disorder or degenerative disease in a subject.

Accordingly, in one aspect, the present disclosure provides an antibody that specifically binds phosphocholine (PC), the antibody comprising: a VH comprising the CDRH1, CDRH2, and CDRH3 amino acid sequences of any one of the VH amino acid sequences set forth in SEQ ID NOs: 1-180; and a VL comprising the CDRL1, CDRL2, and CDRL3 amino acid sequences of any one of the VL amino acid sequences set forth in SEQ ID NOs: 181-368.

In an embodiment, the antibody comprises the CDRH1, CDRH2, CDRH3, CDRL1, CDRL2, and CDRL3 amino acid sequences of the VH and VL amino acid sequences, respectively, set forth in SEQ ID NOs: 1 and 181; 2 and 182; 3 and 183; 4 and 184; 5 and 185; 1 and 184; 6 and 184; 1 and 186; 7 and 187; 8 and 188; 9 and 189; 10 and 190; 4 and 191; 11 and 192; 12 and 193; 13 and 194; 14 and 195; 15 and 196; 16 and 184; 17 and 197; 18 and 198; 1 and 199; 19 and 200; 20 and 201; 21 and 202; 22 and 203; 23 and 204; 24 and 205; 25 and 184; 4 and 206; 26 and 207; 27 and 208; 28 and 209; 9 and 210; 29 and 211; 30 and 212; 31 and 213; 32 and 214; 33 and 215; 34 and 216; 17 and 217; 1 and 218; 35 and 184; 36 and 184; 35 and 219; 35 and 220; 37 and 184; 38 and 221; 39 and 222; 9 and 223; 9 and 224; 40 and 184; 41 and 225; 8 and 226; 42 and 227; 35 and 228; 43 and 229; 4 and 230; 9 and 231; 8 and 232; 1 and 233; 8 and 234; 44 and 184; 1 and 235; 45 and 236; 30 and 237; 46 and 184; 47 and 238; 48 and 239; 1 and 230; 49 and 190; 44 and 240; 50 and 184; 51 and 241; 52 and 242; 53 and 243; 54 and 184; 55 and 244; 56 and 245; 57 and 246; 58 and 247; 59 and 248; 60 and 249; 8 and 250; 61 and 184; 35 and 251; 62 and 252; 1 and 253; 63 and 254; 64 and 181; 65 and 255; 66 and 256; 67 and 257; 68 and 198; 35 and 258; 69 and 259; 70 and 260; 71 and 261; 72 and 262; 73 and 263; 74 and 264; 75 and 265; 76 and 266; 77 and 267; 4 and 205; 78 and 268; 1 and 269; 27 and 269; 79 and 269; 80 and 270; 81 and 269; 22 and 269; 82 and 269; 83 and 271; 84 and 272; 1 and 273; 8 and 274; 8 and 275; 22 and 276; 8 and 277; 85 and 278; 86 and 279; 87 and 269; 88 and 280; 89 and 269; 60 and 281; 90 and 269; 91 and 282; 22 and 283; 92 and 269; 93 and 284; 94 and 285; 95 and 286; 96 and 269; 73 and 287; 97 and 288; 68 and 289; 98 and 269; 99 and 290; 100 and 291; 4 and 292; 4 and 289; 101 and 284; 66 and 289; 89 and 293; 102 and 294; 103 and 269; 22 and 295; 104 and 296; 105 and 297; 106 and 270; 107 and 269; 67 and 269; 62 and 298; 66 and 269; 108 and 299; 109 and 300; 110 and 301; 111 and 302; 112 and 269; 113 and 303; 1 and 304; 72 and 305; 114 and 306; 115 and 307; 116 and 269; 117 and 308; 118 and 269; 110 and 309; 119 and 310; 120 and 311; 121 and 269; 122 and 312; 123 and 289; 9 and 313; 124 and 314; 120 and 289; 125 and 315; 110 and 316; 126 and 317; 127 and 318; 49 and 319; 128 and 320; 9 and 321; 22 and 322; 129 and 323; 130 and 269; 131 and 324; 132 and 325; 133 and 326; 134 and 327; 53 and 328; 135 and 272; 136 and 329; 4 and 301; 137 and 330; 138 and 269; 1 and 284; 22 and 331; 139 and 269; 140 and 332; 141 and 269; 142 and 333; 143 and 334; 144 and 335; 101 and 298; 1 and 336; 145 and 269; 146 and 337; 147 and 269; 17 and 338; 22 and 320; 4 and 339; 148 and 340; 149 and 341; 150 and 342; 151 and 343; 152 and 344; 94 and 289; 153 and 345; 154 and 346; 155 and 347; 22 and 348; 89 and 282; 156 and 349; 157 and 296; 158 and 350; 159 and 329; 24 and 351; 160 and 352; 161 and 269; 162 and 353; 163 and 269; 1 and 354; 164 and 355; 165 and 269; 1 and 356; 166 and 357; 167 and 358; 110 and 334; 87 and 359; 168 and 360; 169 and 269; 170 and 269; 171 and 269; 172 and 361; 8 and 362; 173 and 304; 174 and 269; 1 and 363; 175 and 364; 176 and 365; 177 and 269; 178 and 366; 179 and 269; 180 and 367; or 89 and 368.

In an embodiment, the antibody comprises the CDRH1, CDRH2, and CDRH3 amino acid sequences, respectively, set forth in SEQ ID NOs: 369, 428, and 484; 370, 429, and 485; 371, 428, and 486; 372, 428, and 484; 373, 429, and 484; 374, 430, and 484; 375, 428, and 487; 376, 428, and 484; 377, 428, and 484; 378, 428, and 484; 379, 428, and 484; 372, 428, and 488; 376, 431, and 484; 380, 432, and 484; 372, 428, and 489; 381, 428, and 490; 382, 428, and 484; 381, 428, and 491; 377, 428, and 492; 376, 433, and 484; 383, 434, and 484; 383, 428, and 484; 384, 435, and 484; 369, 436, and 484; 372, 437, and 487; 385, 428, and 493; 386, 428, and 484; 372, 437, and 484; 387, 428, and 484; 385, 428, and 484; 372, 438, and 484; 388, 428, and 494; 369, 439, and 484; 389, 428, and 484; 381, 428, and 484; 376, 440, and 495; 390, 441, and 484; 370, 442, and 484; 391, 428, and 484; 392, 432, and 484; 377, 443, and 484; 393, 428, and 484; 383, 444, and 489; 394, 428, and 484; 383, 444, and 496; 376, 434, and 484; 395, 445, and 497; 396, 446, and 487; 392, 428, and 484; 393, 428, and 498; 376, 428, and 495; 369, 440, and 484; 397, 428, and 484; 389, 447, and 484; 398, 448, and 492; 373, 449, and 490; 399, 428, and 484; 400, 450, and 484; 401, 428, and 484; 390, 428, and 484; 400, 428, and 499; 384, 429, and 484; 402, 436, and 484; 381, 451, and 484; 403, 428, and 484; 369, 429, and 484; 404, 429, and 484; 372, 429, and 484; 388, 428, and 484; 405, 451, and 484; 406, 428, and 484; 400, 428, and 484; 404, 428, and 484; 384, 432, and 500; 376, 452, and 501; 407, 428, and 484; 375, 428, and 484; 408, 428, and 484; 369, 428, and 497; 403, 453, and 484; 402, 454, and 484; 383, 434, and 500; 376, 455, and 502; 383, 435, and 484; 376, 428, and 490; 369, 428, and 503; 409, 428, and 484; 383, 428, and 504; 369, 434, and 484; 369, 456, and 484; 370, 457, and 484; 404, 458, and 499; 410, 428, and 484; 383, 459, and 484; 407, 428, and 489; 374, 434, and 484; 411, 460, and 505; 383, 428, and 485; 386, 460, and 490; 412, 455, and 493; 383, 461, and 484; 376, 437, and 484; 383, 462, and 500; 400, 463, and 484; 376, 464, and 484; 400, 434, and 506; 369, 436, and 507; 377, 465, and 484; 413, 459, and 484; 369, 452, and 484; 414, 466, and 484; 373, 428, and 484; 415, 428, and 489; 369, 428, and 491; 403, 458, and 508; 392, 467, and 484; 369, 452, and 509; 388, 468, and 490; 377, 440, and 484; 370, 428, and 484; 384, 467, and 487; 392, 469, and 510; 378, 464, and 484; 416, 434, and 484; 370, 428, and 511; 376, 428, and 502; 417, 428, and 484; 376, 428, and 512; 381, 428, and 493; 383, 470, and 501; 404, 471, and 484; 418, 428, and 484; 383, 472, and 513; 419, 428, and 484; 372, 473, and 484; 383, 474, and 484; 420, 434, and 484; 370, 428, and 514; 369, 475, and 484; 421, 436, and 484; 378, 452, and 484; 376, 476, and 515; 376, 428, and 504; 422, 469, and 484; 369, 428, and 516; 405, 460, and 484; 413, 431, and 484; 406, 429, and 484; 383, 428, and 517; 377, 437, and 518; 369, 477, and 487; 403, 464, and 484; 369, 454, and 484; 372, 478, and 519; 423, 429, and 484; 382, 434, and 484; 424, 479, and 484; 376, 429, and 484; 425, 428, and 484; 403, 428, and 520; 417, 460, and 484; 409, 460, and 484; 404, 452, and 484; 383, 428, and 521; 372, 480, and 493; 369, 477, and 484; 369, 481, and 484; 384, 452, and 484; 377, 482, and 484; 410, 483, and 484; 384, 428, and 489; 426, 429, and 484; 427, 428, and 484; 373, 452, and 506; 369, 429, and 521; 405, 428, and 522; 376, 428, and 510; 384, 428, and 484; 410, 429, and 484; or 400, 452, and 484.

In an embodiment, the antibody comprises the CDRL1, CDRL2, and CDRL3 amino acid sequences, respectively, set forth in SEQ ID NOs: 523, 591, and 656; 523, 592, and 657; 523, 593, and 658; 523, 591, and 659; 523, 594, and 659; 523, 595, and 660; 523, 596, and 659; 524, 591, and 659; 523, 591, and 661; 523, 591, and 662; 523, 597, and 658; 523, 598, and 662; 525, 599, and 659; 523, 600, and 663; 523, 591, and 664; 523, 601, and 659; 526, 597, and 665; 523, 602, and 659; 527, 591, and 661; 528, 591, and 662; 529, 603, and 662; 523, 604, and 666; 523, 605, and 667; 530, 606, and 659; 523, 607, and 659; 523, 608, and 659; 523, 609, and 662; 531, 591, and 663; 523, 608, and 661; 523, 610, and 659; 523, 611, and 668; 523, 611, and 658; 523, 596, and 662; 523, 612, and 667; 523, 613, and 668; 523, 591, and 669; 523, 591, and 663; 523, 614, and 659; 532, 615, and 662; 523, 616, and 670; 523, 591, and 668; 523, 617, and 671; 533, 595, and 659; 523, 618, and 659; 534, 619, and 662; 535, 620, and 659; 536, 621, and 659; 537, 603, and 671; 523, 622, and 659; 523, 617, and 659; 523, 623, and 659; 538, 607, and 672; 523, 616, and 671; 523, 624, and 659; 523, 620, and 659; 523, 625, and 660; 523, 602, and 671; 523, 611, and 663; 523, 626, and 670; 523, 606, and 673; 523, 627, and 671; 523, 628, and 671; 539, 591, and 662; 523, 591, and 670; 523, 611, and 659; 537, 613, and 671; 540, 598, and 659; 541, 591, and 659; 542, 629, and 659; 523, 591, and 671; 523, 630, and 667; 543, 631, and 659; 523, 632, and 664; 523, 597, and 661; 523, 633, and 659; 544, 607, and 659; 523, 630, and 659; 523, 591, and 665; 523, 610, and 674; 523, 634, and 671; 545, 591, and 661; 523, 635, and 667; 546, 591, and 659; 523, 636, and 664; 547, 591, and 663; 548, 619, and 659; 523, 637, and 659; 523, 638, and 659; 549, 591, and 659; 550, 591, and 675; 551, 591, and 659; 552, 591, and 659; 553, 591, and 676; 554, 591, and 659; 523, 591, and 676; 555, 606, and 659; 556, 591, and 659; 523, 637, and 677; 557, 591, and 659; 558, 591, and 659; 523, 591, and 677; 553, 639, and 659; 549, 591, and 667; 559, 617, and 666; 523, 591, and 672; 523, 606, and 659; 523, 592, and 670; 560, 591, and 667; 530, 591, and 659; 561, 603, and 666; 562, 591, and 659; 523, 616, and 660; 523, 602, and 677; 523, 616, and 659; 523, 630, and 660; 563, 591, and 659; 523, 640, and 659; 564, 591, and 659; 523, 641, and 677; 565, 591, and 662; 523, 642, and 664; 566, 643, and 659; 523, 644, and 667; 567, 606, and 659; 568, 591, and 659; 569, 645, and 659; 523, 603, and 659; 570, 628, and 678; 571, 630, and 663; 572, 591, and 664; 573, 591, and 670; 523, 646, and 659; 574, 591, and 670; 523, 647, and 667; 575, 591, and 659; 537, 591, and 659; 523, 635, and 670; 576, 591, and 659; 577, 606, and 659; 523, 648, and 679; 523, 592, and 659; 523, 595, and 659; 523, 600, and 659; 523, 649, and 664; 578, 641, and 659; 523, 591, and 667; 523, 606, and 660; 523, 650, and 656; 579, 621, and 671; 580, 591, and 660; 527, 591, and 659; 581, 595, and 659; 582, 640, and 664; 583, 651, and 659; 534, 591, and 659; 584, 591, and 671; 523, 591, and 680; 523, 591, and 681; 585, 591, and 659; 523, 649, and 659; 586, 591, and 666; 587, 652, and 659; 588, 638, and 659; 523, 623, and 664; 523, 591, and 682; 553, 653, and 659; 523, 654, and 659; 523, 648, and 659; 560, 591, and 659; 580, 606, and 659; 589, 593, and 661; 523, 637, and 676; 590, 622, and 659; 555, 591, and 661; 523, 615, and 672; 523, 626, and 668; 523, 655, and 659; or 523, 606, and 680.

In an embodiment, the antibody comprises the CDRH1, CDRH2, CDRH3, CDRL1, CDRL2, and CDRL3 amino acid sequences, set forth in SEQ ID NOs: 369, 428, 484, 523, 591, and 656; 370, 429, 485, 523, 592, and 657; 371, 428, 486, 523, 593, and 658; 372, 428, 484, 523, 591, and 659; 373, 429, 484, 523, 594, and 659; 369, 428, 484, 523, 591, and 659; 374, 430, 484, 523, 591, and 659; 369, 428, 484, 523, 595, and 660; 375, 428, 487, 523, 596, and 659; 376, 428, 484, 524, 591, and 659; 377, 428, 484, 523, 591, and 661; 378, 428, 484, 523, 591, and 662; 372, 428, 484, 523, 597, and 658; 379, 428, 484, 523, 598, and 662; 372, 428, 488, 525, 599, and 659; 376, 431, 484, 523, 600, and 663; 380, 432, 484, 523, 591, and 664; 372, 428, 489, 523, 601, and 659; 381, 428, 490, 523, 591, and 659; 382, 428, 484, 526, 597, and 665; 381, 428, 491, 523, 602, and 659; 369, 428, 484, 527, 591, and 661; 377, 428, 492, 528, 591, and 662; 376, 433, 484, 529, 603, and 662; 383, 434, 484, 523, 604, and 666; 383, 428, 484, 523, 605, and 667; 384, 435, 484, 530, 606, and 659; 369, 436, 484, 523, 607, and 659; 372, 437, 487, 523, 591, and 659; 372, 428, 484, 523, 608, and 659; 385, 428, 493, 523, 609, and 662; 386, 428, 484, 531, 591, and 663; 372, 437, 484, 523, 608, and 661; 377, 428, 484, 523, 610, and 659; 387, 428, 484, 523, 611, and 668; 385, 428, 484, 523, 611, and 658; 372, 438, 484, 523, 596, and 662; 388, 428, 494, 523, 612, and 667; 369, 439, 484, 523, 613, and 668; 389, 428, 484, 523, 591, and 669; 382, 428, 484, 523, 591, and 663; 369, 428, 484, 523, 614, and 659; 381, 428, 484, 523, 591, and 659; 376, 440, 495, 523, 591, and 659; 381, 428, 484, 532, 615, and 662; 381, 428, 484, 523, 616, and 670; 390, 441, 484, 523, 591, and 659; 370, 442, 484, 523, 591, and 668; 391, 428, 484, 523, 617, and 671; 377, 428, 484, 533, 595, and 659; 377, 428, 484, 523, 618, and 659; 392, 432, 484, 523, 591, and 659; 377, 443, 484, 534, 619, and 662; 376, 428, 484, 535, 620, and 659; 393, 428, 484, 536, 621, and 659; 381, 428, 484, 537, 603, and 671; 383, 444, 489, 523, 622, and 59; 372, 428, 484, 523, 617, and 659; 377, 428, 484, 523, 623, and 659; 376, 428, 484, 538, 607, and 672; 369, 428, 484, 523, 616, and 671; 376, 428, 484, 523, 624, and 659; 394, 428, 484, 523, 591, and 659; 369, 428, 484, 523, 620, and 659; 383, 444, 496, 523, 625, and 660; 385, 428, 484, 523, 602, and 671; 376, 434, 484, 523, 591, and 659; 395, 445, 497, 523, 611, and 663; 396, 446, 487, 523, 626, and 670; 369, 428, 484, 523, 617, and 659; 392, 428, 484, 523, 591, and 662; 394, 428, 484, 523, 606, and 673; 393, 428, 498, 523, 591, and 659; 376, 428, 495, 523, 627, and 671; 369, 440, 484, 523, 628, and 671; 397, 428, 484, 539, 591, and 662; 389, 447, 484, 523, 591, and 659; 398, 448, 492, 523, 591, and 670; 373, 449, 490, 523, 611, and 659; 399, 428, 484, 537, 613, and 671; 400, 450, 484, 540, 598, and 659; 401, 428, 484, 541, 591, and 659; 390, 428, 484, 542, 629, and 659; 376, 428, 484, 523, 591, and 671; 400, 428, 499, 523, 591, and 659; 381, 428, 484, 523, 630, and 667; 384, 429, 484, 543, 631, and 659; 369, 428, 484, 523, 632, and 664; 402, 436, 484, 523, 597, and 661; 381, 451, 484, 523, 591, and 656; 403, 428, 484, 523, 633, and 659; 369, 429, 484, 544, 607, and 659; 404, 429, 484, 523, 630, and 659; 372, 429, 484, 523, 602, and 659; 381, 428, 484, 523, 591, and 665; 388, 428, 484, 523, 610, and 674; 405, 451, 484, 523, 634, and 671; 406, 428, 484, 545, 591, and 661; 400, 428, 484, 523, 635, and 667; 404, 428, 484, 546, 591, and 659; 384, 432, 500, 523, 636, and 664; 376, 452, 501, 547, 591, and 663; 407, 428, 484, 548, 619, and 659; 375, 428, 484, 523, 637, and 659; 372, 428, 484, 523, 607, and 659; 408, 428, 484, 523, 638, and 659; 386, 428, 484, 523, 591, and 659; 369, 428, 497, 523, 591, and 659; 403, 453, 484, 549, 591, and 659; 402, 454, 484, 523, 591, and 659; 383, 428, 484, 523, 591, and 659; 383, 434, 500, 523, 591, and 659; 376, 455, 502, 550, 591, and 675; 383, 435, 484, 523, 591, and 664; 369, 428, 484, 523, 602, and 659; 376, 428, 484, 551, 591, and 659; 376, 428, 484, 552, 591, and 659; 383, 428, 484, 553, 591, and 676; 376, 428, 484, 554, 591, and 659; 376, 428, 490, 523, 591, and 676; 369, 428, 503, 555, 606, and 659; 409, 428, 484, 523, 591, and 659; 383, 428, 504, 556, 591, and 659; 369, 434, 484, 523, 591, and 659; 390, 428, 484, 523, 637, and 677; 369, 456, 484, 523, 591, and 659; 370, 457, 484, 557, 591, and 659; 383, 428, 484, 558, 591, and 659; 404, 458, 499, 523, 591, and 659; 410, 428, 484, 523, 591, and 677; 383, 459, 484, 553, 639, and 659; 407, 428, 489, 549, 591, and 667; 374, 434, 484, 523, 591, and 659; 404, 428, 484, 559, 617, and 666; 411, 460, 505, 523, 591, and 672; 372, 429, 484, 523, 606, and 659; 383, 428, 485, 523, 591, and 659; 386, 460, 490, 523, 601, and 659; 412, 455, 493, 523, 592, and 670; 372, 428, 484, 560, 591, and 667; 372, 428, 484, 523, 606, and 659; 383, 461, 484, 523, 591, and 677; 369, 429, 484, 523, 606, and 659; 369, 434, 484, 530, 591, and 659; 376, 437, 484, 561, 603, and 666; 383, 462, 500, 523, 591, and 659; 383, 428, 484, 562, 591, and 659; 400, 463, 484, 523, 616, and 660; 376, 464, 484, 523, 602, and 677; 400, 434, 506, 549, 591, and 659; 369, 436, 507, 523, 591, and 659; 404, 429, 484, 523, 591, and 659; 384, 429, 484, 523, 616, and 659; 369, 429, 484, 523, 591, and 659; 377, 465, 484, 523, 630, and 660; 413, 459, 484, 563, 591, and 659; 369, 452, 484, 523, 591, and 663; 414, 466, 484, 523, 640, and 659; 373, 428, 484, 523, 591, and 659; 415, 428, 489, 564, 591, and 659; 369, 428, 484, 523, 591, and 671; 400, 428, 484, 523, 641, and 677; 369, 428, 491, 565, 591, and 662; 403, 458, 508, 523, 642, and 664; 392, 467, 484, 523, 591, and 659; 369, 452, 509, 566, 643, and 659; 388, 468, 490, 523, 591, and 659; 369, 452, 484, 523, 644, and 667; 377, 440, 484, 567, 606, and 659; 370, 428, 484, 523, 591, and 656; 384, 467, 487, 523, 591, and 659; 392, 469, 510, 568, 591, and 659; 378, 464, 484, 523, 606, and 659; 377, 428, 484, 569, 645, and 659; 416, 434, 484, 523, 603, and 659; 370, 428, 484, 523, 606, and 659; 370, 428, 511, 570, 628, and 678; 369, 452, 484, 571, 630, and 663; 376, 428, 502, 572, 591, and 664; 417, 428, 484, 523, 591, and 665; 392, 428, 484, 573, 591, and 670; 376, 428, 512, 523, 646, and 659; 377, 428, 484, 574, 591, and 670; 383, 428, 484, 523, 647, and 667; 381, 428, 493, 575, 591, and 659; 383, 470, 501, 523, 591, and 659; 404, 471, 484, 537, 591, and 659; 418, 428, 484, 523, 635, and 670; 383, 472, 513, 576, 591, and 659; 419, 428, 484, 577, 606, and 659; 397, 428, 484, 523, 648, and 679; 372, 473, 484, 523, 591, and 664; 383, 474, 484, 523, 592, and 659; 372, 428, 484, 523, 591, and 663; 420, 434, 484, 523, 595, and 659; 370, 428, 514, 523, 591, and 659; 369, 428, 484, 523, 591, and 677; 383, 428, 484, 523, 600, and 659; 369, 475, 484, 523, 591, and 659; 421, 436, 484, 523, 649, and 664; 378, 452, 484, 523, 591, and 659; 376, 476, 515, 578, 641, and 659; 376, 428, 504, 523, 591, and 667; 422, 469, 484, 523, 606, and 660; 383, 461, 484, 523, 616, and 659; 369, 428, 484, 523, 650, and 656; 369, 428, 516, 523, 591, and 659; 405, 460, 484, 523, 618, and 659; 413, 431, 484, 523, 591, and 659; 382, 428, 484, 579, 621, and 671; 383, 428, 484, 523, 646, and 659; 372, 428, 484, 580, 591, and 660; 406, 429, 484, 527, 591, and 659; 383, 428, 517, 581, 595, and 659; 377, 437, 518, 582, 640, and 664; 369, 477, 487, 583, 651, and 659; 403, 464, 484, 534, 591, and 659; 383, 459, 484, 523, 606, and 659; 369, 454, 484, 584, 591, and 671; 372, 478, 519, 523, 591, and 680; 423, 429, 484, 523, 591, and 681; 383, 428, 484, 585, 591, and 659; 369, 434, 484, 557, 591, and 659; 382, 434, 484, 523, 649, and 659; 424, 479, 484, 523, 616, and 660; 376, 429, 484, 586, 591, and 666; 425, 428, 484, 523, 592, and 659; 369, 436, 484, 587, 652, and 659; 403, 428, 520, 588, 638, and 659; 417, 460, 484, 523, 591, and 659; 409, 460, 484, 523, 623, and 664; 404, 452, 484, 523, 591, and 659; 369, 428, 484, 523, 591, and 682; 383, 428, 521, 553, 653, and 659; 372, 480, 493, 523, 591, and 659; 369, 428, 484, 523, 654, and 659; 369, 477, 484, 523, 648, and 659; 369, 481, 484, 560, 591, and 659; 369, 452, 484, 523, 591, and 667; 409, 428, 484, 580, 606, and 659; 384, 452, 484, 589, 593, and 661; 377, 482, 484, 523, 591, and 659; 410, 483, 484, 523, 591, and 659; 384, 428, 489, 523, 591, and 659; 426, 429, 484, 523, 622, and 659; 376, 428, 484, 523, 637, and 676; 427, 428, 484, 523, 591, and 671; 373, 452, 506, 523, 591, and 659; 369, 428, 484, 590, 622, and 659; 369, 429, 521, 555, 591, and 661; 405, 428, 522, 523, 615, and 672; 376, 428, 510, 523, 591, and 659; 384, 428, 484, 523, 626, and 668; 410, 429, 484, 523, 591, and 659; 400, 452, 484, 523, 655, and 659; or 369, 434, 484, 523, 606, and 680.

In an embodiment, the antibody comprises the VH amino acid sequence of any one of SEQ ID NOs: 1-180.

In an embodiment, the antibody comprises the VL amino acid sequence of any one of SEQ ID NOs: 181-368.

In an embodiment, the VH and VL comprise the amino acid sequences, respectively, set forth in SEQ ID NOs: 1 and 181; 2 and 182; 3 and 183; 4 and 184; 5 and 185; 1 and 184; 6 and 184; 1 and 186; 7 and 187; 8 and 188; 9 and 189; 10 and 190; 4 and 191; 11 and 192; 12 and 193; 13 and 194; 14 and 195; 15 and 196; 16 and 184; 17 and 197; 18 and 198; 1 and 199; 19 and 200; 20 and 201; 21 and 202; 22 and 203; 23 and 204; 24 and 205; 25 and 184; 4 and 206; 26 and 207; 27 and 208; 28 and 209; 9 and 210; 29 and 211; 30 and 212; 31 and 213; 32 and 214; 33 and 215; 34 and 216; 17 and 217; 1 and 218; 35 and 184; 36 and 184; 35 and 219; 35 and 220; 37 and 184; 38 and 221; 39 and 222; 9 and 223; 9 and 224; 40 and 184; 41 and 225; 8 and 226; 42 and 227; 35 and 228; 43 and 229; 4 and 230; 9 and 231; 8 and 232; 1 and 233; 8 and 234; 44 and 184; 1 and 235; 45 and 236; 30 and 237; 46 and 184; 47 and 238; 48 and 239; 1 and 230; 49 and 190; 44 and 240; 50 and 184; 51 and 241; 52 and 242; 53 and 243; 54 and 184; 55 and 244; 56 and 245; 57 and 246; 58 and 247; 59 and 248; 60 and 249; 8 and 250; 61 and 184; 35 and 251; 62 and 252; 1 and 253; 63 and 254; 64 and 181; 65 and 255; 66 and 256; 67 and 257; 68 and 198; 35 and 258; 69 and 259; 70 and 260; 71 and 261; 72 and 262; 73 and 263; 74 and 264; 75 and 265; 76 and 266; 77 and 267; 4 and 205; 78 and 268; 1 and 269; 27 and 269; 79 and 269; 80 and 270; 81 and 269; 22 and 269; 82 and 269; 83 and 271; 84 and 272; 1 and 273; 8 and 274; 8 and 275; 22 and 276; 8 and 277; 85 and 278; 86 and 279; 87 and 269; 88 and 280; 89 and 269; 60 and 281; 90 and 269; 91 and 282; 22 and 283; 92 and 269; 93 and 284; 94 and 285; 95 and 286; 96 and 269; 73 and 287; 97 and 288; 68 and 289; 98 and 269; 99 and 290; 100 and 291; 4 and 292; 4 and 289; 101 and 284; 66 and 289; 89 and 293; 102 and 294; 103 and 269; 22 and 295; 104 and 296; 105 and 297; 106 and 270; 107 and 269; 67 and 269; 62 and 298; 66 and 269; 108 and 299; 109 and 300; 110 and 301; 111 and 302; 112 and 269; 113 and 303; 1 and 304; 72 and 305; 114 and 306; 115 and 307; 116 and 269; 117 and 308; 118 and 269; 110 and 309; 119 and 310; 120 and 311; 121 and 269; 122 and 312; 123 and 289; 9 and 313; 124 and 314; 120 and 289; 125 and 315; 110 and 316; 126 and 317; 127 and 318; 49 and 319; 128 and 320; 9 and 321; 22 and 322; 129 and 323; 130 and 269; 131 and 324; 132 and 325; 133 and 326; 134 and 327; 53 and 328; 135 and 272; 136 and 329; 4 and 301; 137 and 330; 138 and 269; 1 and 284; 22 and 331; 139 and 269; 140 and 332; 141 and 269; 142 and 333; 143 and 334; 144 and 335; 101 and 298; 1 and 336; 145 and 269; 146 and 337; 147 and 269; 17 and 338; 22 and 320; 4 and 339; 148 and 340; 149 and 341; 150 and 342; 151 and 343; 152 and 344; 94 and 289; 153 and 345; 154 and 346; 155 and 347; 22 and 348; 89 and 282; 156 and 349; 157 and 296; 158 and 350; 159 and 329; 24 and 351; 160 and 352; 161 and 269; 162 and 353; 163 and 269; 1 and 354; 164 and 355; 165 and 269; 1 and 356; 166 and 357; 167 and 358; 110 and 334; 87 and 359; 168 and 360; 169 and 269; 170 and 269; 171 and 269; 172 and 361; 8 and 362; 173 and 304; 174 and 269; 1 and 363; 175 and 364; 176 and 365; 177 and 269; 178 and 366; 179 and 269; 180 and 367; or 89 and 368.

In an embodiment, the antibody is a single-chain variable fragment (scFv).

In an embodiment, the antibody comprises a heavy chain constant region, or an Fc region thereof, optionally selected from the group consisting of a human IgG₁, IgG₂, IgG₃, IgG₄, IgA₁, and IgA₂.

In an embodiment, the heavy chain constant region is a human IgG₁.

In an embodiment, the heavy chain constant region is a human IgG₄.

In an embodiment, the amino acid sequence of the human IgG₄ heavy chain constant region comprises a P at position 228, numbered according to the EU numbering system.

In an embodiment, the heavy chain constant region is a variant of a wild-type heavy chain constant region, wherein the variant heavy chain constant region binds to an Fc gamma receptor (FcγR) with lower affinity than the wild-type heavy chain constant region binds to the FcγR.

In an embodiment, the amino acid sequence of the heavy chain constant region comprises: A at position 234; A at position 235; A, Q or G at position 297; or A or G at position 329, in each case numbered according to the EU numbering system.

In an embodiment, the amino acid sequence of the heavy chain constant region comprises: A at positions 234 and 235; A at positions 234, 235, and 329; or A at positions 234, 235, and G at position 329, in each case numbered according to the EU numbering system.

In an embodiment, the heavy chain constant region is a variant of a wild-type heavy chain constant region, and wherein the variant heavy chain constant region has an increased affinity for human neonatal Fc receptor (FcRn) at pH 6 relative to the affinity of the wild-type heavy chain constant region for human FcRn at pH 6.

In an embodiment, the amino acid sequence of the heavy chain constant region comprises: L and S at positions 428 and 434, respectively; K, F, and Y at positions 433, 434, and 436, respectively; or Y, T, and E at positions 252, 254, and 256, respectively, in each case numbered according to the EU numbering system.

In an embodiment, the antibody comprises a light chain constant region, optionally a human kappa or lambda constant region.

In one aspect, the present disclosure provides a polypeptide comprising a VH comprising the CDRH1, CDRH2, and CDRH3 amino acid sequences of any one of the VH amino acid sequences set forth in SEQ ID NOs: 1-180.

In an embodiment, the polypeptide comprises the CDRH1, CDRH2, and CDRH3 amino acid sequences, respectively, set forth in SEQ ID NOs: 369, 428, and 484; 370, 429, and 485; 371, 428, and 486; 372, 428, and 484; 373, 429, and 484; 374, 430, and 484; 375, 428, and 487; 376, 428, and 484; 377, 428, and 484; 378, 428, and 484; 379, 428, and 484; 372, 428, and 488; 376, 431, and 484; 380, 432, and 484; 372, 428, and 489; 381, 428, and 490; 382, 428, and 484; 381, 428, and 491; 377, 428, and 492; 376, 433, and 484; 383, 434, and 484; 383, 428, and 484; 384, 435, and 484; 369, 436, and 484; 372, 437, and 487; 385, 428, and 493; 386, 428, and 484; 372, 437, and 484; 387, 428, and 484; 385, 428, and 484; 372, 438, and 484; 388, 428, and 494; 369, 439, and 484; 389, 428, and 484; 381, 428, and 484; 376, 440, and 495; 390, 441, and 484; 370, 442, and 484; 391, 428, and 484; 392, 432, and 484; 377, 443, and 484; 393, 428, and 484; 383, 444, and 489; 394, 428, and 484; 383, 444, and 496; 376, 434, and 484; 395, 445, and 497; 396, 446, and 487; 392, 428, and 484; 393, 428, and 498; 376, 428, and 495; 369, 440, and 484; 397, 428, and 484; 389, 447, and 484; 398, 448, and 492; 373, 449, and 490; 399, 428, and 484; 400, 450, and 484; 401, 428, and 484; 390, 428, and 484; 400, 428, and 499; 384, 429, and 484; 402, 436, and 484; 381, 451, and 484; 403, 428, and 484; 369, 429, and 484; 404, 429, and 484; 372, 429, and 484; 388, 428, and 484; 405, 451, and 484; 406, 428, and 484; 400, 428, and 484; 404, 428, and 484; 384, 432, and 500; 376, 452, and 501; 407, 428, and 484; 375, 428, and 484; 408, 428, and 484; 369, 428, and 497; 403, 453, and 484; 402, 454, and 484; 383, 434, and 500; 376, 455, and 502; 383, 435, and 484; 376, 428, and 490; 369, 428, and 503; 409, 428, and 484; 383, 428, and 504; 369, 434, and 484; 369, 456, and 484; 370, 457, and 484; 404, 458, and 499; 410, 428, and 484; 383, 459, and 484; 407, 428, and 489; 374, 434, and 484; 411, 460, and 505; 383, 428, and 485; 386, 460, and 490; 412, 455, and 493; 383, 461, and 484; 376, 437, and 484; 383, 462, and 500; 400, 463, and 484; 376, 464, and 484; 400, 434, and 506; 369, 436, and 507; 377, 465, and 484; 413, 459, and 484; 369, 452, and 484; 414, 466, and 484; 373, 428, and 484; 415, 428, and 489; 369, 428, and 491; 403, 458, and 508; 392, 467, and 484; 369, 452, and 509; 388, 468, and 490; 377, 440, and 484; 370, 428, and 484; 384, 467, and 487; 392, 469, and 510; 378, 464, and 484; 416, 434, and 484; 370, 428, and 511; 376, 428, and 502; 417, 428, and 484; 376, 428, and 512; 381, 428, and 493; 383, 470, and 501; 404, 471, and 484; 418, 428, and 484; 383, 472, and 513; 419, 428, and 484; 372, 473, and 484; 383, 474, and 484; 420, 434, and 484; 370, 428, and 514; 369, 475, and 484; 421, 436, and 484; 378, 452, and 484; 376, 476, and 515; 376, 428, and 504; 422, 469, and 484; 369, 428, and 516; 405, 460, and 484; 413, 431, and 484; 406, 429, and 484; 383, 428, and 517; 377, 437, and 518; 369, 477, and 487; 403, 464, and 484; 369, 454, and 484; 372, 478, and 519; 423, 429, and 484; 382, 434, and 484; 424, 479, and 484; 376, 429, and 484; 425, 428, and 484; 403, 428, and 520; 417, 460, and 484; 409, 460, and 484; 404, 452, and 484; 383, 428, and 521; 372, 480, and 493; 369, 477, and 484; 369, 481, and 484; 384, 452, and 484; 377, 482, and 484; 410, 483, and 484; 384, 428, and 489; 426, 429, and 484; 427, 428, and 484; 373, 452, and 506; 369, 429, and 521; 405, 428, and 522; 376, 428, and 510; 384, 428, and 484; 410, 429, and 484; or 400, 452, and 484.

In an embodiment, the VH comprises the CDRH1, CDRH2, and CDRH3 amino acid sequences, respectively, set forth in SEQ ID NOs: 369, 428, and 484; 370, 429, and 485; 371, 428, and 486; 372, 428, and 484; 373, 429, and 484; 374, 430, and 484; 375, 428, and 487; 376, 428, and 484; 377, 428, and 484; 378, 428, and 484; 379, 428, and 484; 372, 428, and 488; 376, 431, and 484; 380, 432, and 484; 372, 428, and 489; 381, 428, and 490; 382, 428, and 484; 381, 428, and 491; 377, 428, and 492; 376, 433, and 484; 383, 434, and 484; 383, 428, and 484; 384, 435, and 484; 369, 436, and 484; 372, 437, and 487; 385, 428, and 493; 386, 428, and 484; 372, 437, and 484; 387, 428, and 484; 385, 428, and 484; 372, 438, and 484; 388, 428, and 494; 369, 439, and 484; 389, 428, and 484; 381, 428, and 484; 376, 440, and 495; 390, 441, and 484; 370, 442, and 484; 391, 428, and 484; 392, 432, and 484; 377, 443, and 484; 393, 428, and 484; 383, 444, and 489; 394, 428, and 484; 383, 444, and 496; 376, 434, and 484; 395, 445, and 497; 396, 446, and 487; 392, 428, and 484; 393, 428, and 498; 376, 428, and 495; 369, 440, and 484; 397, 428, and 484; 389, 447, and 484; 398, 448, and 492; 373, 449, and 490; 399, 428, and 484; 400, 450, and 484; 401, 428, and 484; 390, 428, and 484; 400, 428, and 499; 384, 429, and 484; 402, 436, and 484; 381, 451, and 484; 403, 428, and 484; 369, 429, and 484; 404, 429, and 484; 372, 429, and 484; 388, 428, and 484; 405, 451, and 484; 406, 428, and 484; 400, 428, and 484; 404, 428, and 484; 384, 432, and 500; 376, 452, and 501; 407, 428, and 484; 375, 428, and 484; 408, 428, and 484; 369, 428, and 497; 403, 453, and 484; 402, 454, and 484; 383, 434, and 500; 376, 455, and 502; 383, 435, and 484; 376, 428, and 490; 369, 428, and 503; 409, 428, and 484; 383, 428, and 504; 369, 434, and 484; 369, 456, and 484; 370, 457, and 484; 404, 458, and 499; 410, 428, and 484; 383, 459, and 484; 407, 428, and 489; 374, 434, and 484; 411, 460, and 505; 383, 428, and 485; 386, 460, and 490; 412, 455, and 493; 383, 461, and 484; 376, 437, and 484; 383, 462, and 500; 400, 463, and 484; 376, 464, and 484; 400, 434, and 506; 369, 436, and 507; 377, 465, and 484; 413, 459, and 484; 369, 452, and 484; 414, 466, and 484; 373, 428, and 484; 415, 428, and 489; 369, 428, and 491; 403, 458, and 508; 392, 467, and 484; 369, 452, and 509; 388, 468, and 490; 377, 440, and 484; 370, 428, and 484; 384, 467, and 487; 392, 469, and 510; 378, 464, and 484; 416, 434, and 484; 370, 428, and 511; 376, 428, and 502; 417, 428, and 484; 376, 428, and 512; 381, 428, and 493; 383, 470, and 501; 404, 471, and 484; 418, 428, and 484; 383, 472, and 513; 419, 428, and 484; 372, 473, and 484; 383, 474, and 484; 420, 434, and 484; 370, 428, and 514; 369, 475, and 484; 421, 436, and 484; 378, 452, and 484; 376, 476, and 515; 376, 428, and 504; 422, 469, and 484; 369, 428, and 516; 405, 460, and 484; 413, 431, and 484; 406, 429, and 484; 383, 428, and 517; 377, 437, and 518; 369, 477, and 487; 403, 464, and 484; 369, 454, and 484; 372, 478, and 519; 423, 429, and 484; 382, 434, and 484; 424, 479, and 484; 376, 429, and 484; 425, 428, and 484; 403, 428, and 520; 417, 460, and 484; 409, 460, and 484; 404, 452, and 484; 383, 428, and 521; 372, 480, and 493; 369, 477, and 484; 369, 481, and 484; 384, 452, and 484; 377, 482, and 484; 410, 483, and 484; 384, 428, and 489; 426, 429, and 484; 427, 428, and 484; 373, 452, and 506; 369, 429, and 521; 405, 428, and 522; 376, 428, and 510; 384, 428, and 484; 410, 429, and 484; or 400, 452, and 484.

In an embodiment, the VH comprises any one of the amino acid sequences set forth in SEQ ID NOs: 1-180.

In one aspect, the present disclosure provides a polypeptide comprising a VL comprising the CDRL1, CDRL2, and CDRL3 amino acid sequences of any one of the VL amino acid sequences set forth in SEQ ID NOs: 181-368.

In an embodiment, the polypeptide comprises the CDRL1, CDRL2, and CDRL3 amino acid sequences, respectively, set forth in SEQ ID NOs: 523, 591, and 656; 523, 592, and 657; 523, 593, and 658; 523, 591, and 659; 523, 594, and 659; 523, 595, and 660; 523, 596, and 659; 524, 591, and 659; 523, 591, and 661; 523, 591, and 662; 523, 597, and 658; 523, 598, and 662; 525, 599, and 659; 523, 600, and 663; 523, 591, and 664; 523, 601, and 659; 526, 597, and 665; 523, 602, and 659; 527, 591, and 661; 528, 591, and 662; 529, 603, and 662; 523, 604, and 666; 523, 605, and 667; 530, 606, and 659; 523, 607, and 659; 523, 608, and 659; 523, 609, and 662; 531, 591, and 663; 523, 608, and 661; 523, 610, and 659; 523, 611, and 668; 523, 611, and 658; 523, 596, and 662; 523, 612, and 667; 523, 613, and 668; 523, 591, and 669; 523, 591, and 663; 523, 614, and 659; 532, 615, and 662; 523, 616, and 670; 523, 591, and 668; 523, 617, and 671; 533, 595, and 659; 523, 618, and 659; 534, 619, and 662; 535, 620, and 659; 536, 621, and 659; 537, 603, and 671; 523, 622, and 659; 523, 617, and 659; 523, 623, and 659; 538, 607, and 672; 523, 616, and 671; 523, 624, and 659; 523, 620, and 659; 523, 625, and 660; 523, 602, and 671; 523, 611, and 663; 523, 626, and 670; 523, 606, and 673; 523, 627, and 671; 523, 628, and 671; 539, 591, and 662; 523, 591, and 670; 523, 611, and 659; 537, 613, and 671; 540, 598, and 659; 541, 591, and 659; 542, 629, and 659; 523, 591, and 671; 523, 630, and 667; 543, 631, and 659; 523, 632, and 664; 523, 597, and 661; 523, 633, and 659; 544, 607, and 659; 523, 630, and 659; 523, 591, and 665; 523, 610, and 674; 523, 634, and 671; 545, 591, and 661; 523, 635, and 667; 546, 591, and 659; 523, 636, and 664; 547, 591, and 663; 548, 619, and 659; 523, 637, and 659; 523, 638, and 659; 549, 591, and 659; 550, 591, and 675; 551, 591, and 659; 552, 591, and 659; 553, 591, and 676; 554, 591, and 659; 523, 591, and 676; 555, 606, and 659; 556, 591, and 659; 523, 637, and 677; 557, 591, and 659; 558, 591, and 659; 523, 591, and 677; 553, 639, and 659; 549, 591, and 667; 559, 617, and 666; 523, 591, and 672; 523, 606, and 659; 523, 592, and 670; 560, 591, and 667; 530, 591, and 659; 561, 603, and 666; 562, 591, and 659; 523, 616, and 660; 523, 602, and 677; 523, 616, and 659; 523, 630, and 660; 563, 591, and 659; 523, 640, and 659; 564, 591, and 659; 523, 641, and 677; 565, 591, and 662; 523, 642, and 664; 566, 643, and 659; 523, 644, and 667; 567, 606, and 659; 568, 591, and 659; 569, 645, and 659; 523, 603, and 659; 570, 628, and 678; 571, 630, and 663; 572, 591, and 664; 573, 591, and 670; 523, 646, and 659; 574, 591, and 670; 523, 647, and 667; 575, 591, and 659; 537, 591, and 659; 523, 635, and 670; 576, 591, and 659; 577, 606, and 659; 523, 648, and 679; 523, 592, and 659; 523, 595, and 659; 523, 600, and 659; 523, 649, and 664; 578, 641, and 659; 523, 591, and 667; 523, 606, and 660; 523, 650, and 656; 579, 621, and 671; 580, 591, and 660; 527, 591, and 659; 581, 595, and 659; 582, 640, and 664; 583, 651, and 659; 534, 591, and 659; 584, 591, and 671; 523, 591, and 680; 523, 591, and 681; 585, 591, and 659; 523, 649, and 659; 586, 591, and 666; 587, 652, and 659; 588, 638, and 659; 523, 623, and 664; 523, 591, and 682; 553, 653, and 659; 523, 654, and 659; 523, 648, and 659; 560, 591, and 659; 580, 606, and 659; 589, 593, and 661; 523, 637, and 676; 590, 622, and 659; 555, 591, and 661; 523, 615, and 672; 523, 626, and 668; 523, 655, and 659; or 523, 606, and 680.

In an embodiment, the VL comprises the CDRL1, CDRL2, and CDRL3 amino acid sequences, respectively, set forth in SEQ ID NOs: 523, 591, and 656; 523, 592, and 657; 523, 593, and 658; 523, 591, and 659; 523, 594, and 659; 523, 595, and 660; 523, 596, and 659; 524, 591, and 659; 523, 591, and 661; 523, 591, and 662; 523, 597, and 658; 523, 598, and 662; 525, 599, and 659; 523, 600, and 663; 523, 591, and 664; 523, 601, and 659; 526, 597, and 665; 523, 602, and 659; 527, 591, and 661; 528, 591, and 662; 529, 603, and 662; 523, 604, and 666; 523, 605, and 667; 530, 606, and 659; 523, 607, and 659; 523, 608, and 659; 523, 609, and 662; 531, 591, and 663; 523, 608, and 661; 523, 610, and 659; 523, 611, and 668; 523, 611, and 658; 523, 596, and 662; 523, 612, and 667; 523, 613, and 668; 523, 591, and 669; 523, 591, and 663; 523, 614, and 659; 532, 615, and 662; 523, 616, and 670; 523, 591, and 668; 523, 617, and 671; 533, 595, and 659; 523, 618, and 659; 534, 619, and 662; 535, 620, and 659; 536, 621, and 659; 537, 603, and 671; 523, 622, and 659; 523, 617, and 659; 523, 623, and 659; 538, 607, and 672; 523, 616, and 671; 523, 624, and 659; 523, 620, and 659; 523, 625, and 660; 523, 602, and 671; 523, 611, and 663; 523, 626, and 670; 523, 606, and 673; 523, 627, and 671; 523, 628, and 671; 539, 591, and 662; 523, 591, and 670; 523, 611, and 659; 537, 613, and 671; 540, 598, and 659; 541, 591, and 659; 542, 629, and 659; 523, 591, and 671; 523, 630, and 667; 543, 631, and 659; 523, 632, and 664; 523, 597, and 661; 523, 633, and 659; 544, 607, and 659; 523, 630, and 659; 523, 591, and 665; 523, 610, and 674; 523, 634, and 671; 545, 591, and 661; 523, 635, and 667; 546, 591, and 659; 523, 636, and 664; 547, 591, and 663; 548, 619, and 659; 523, 637, and 659; 523, 638, and 659; 549, 591, and 659; 550, 591, and 675; 551, 591, and 659; 552, 591, and 659; 553, 591, and 676; 554, 591, and 659; 523, 591, and 676; 555, 606, and 659; 556, 591, and 659; 523, 637, and 677; 557, 591, and 659; 558, 591, and 659; 523, 591, and 677; 553, 639, and 659; 549, 591, and 667; 559, 617, and 666; 523, 591, and 672; 523, 606, and 659; 523, 592, and 670; 560, 591, and 667; 530, 591, and 659; 561, 603, and 666; 562, 591, and 659; 523, 616, and 660; 523, 602, and 677; 523, 616, and 659; 523, 630, and 660; 563, 591, and 659; 523, 640, and 659; 564, 591, and 659; 523, 641, and 677; 565, 591, and 662; 523, 642, and 664; 566, 643, and 659; 523, 644, and 667; 567, 606, and 659; 568, 591, and 659; 569, 645, and 659; 523, 603, and 659; 570, 628, and 678; 571, 630, and 663; 572, 591, and 664; 573, 591, and 670; 523, 646, and 659; 574, 591, and 670; 523, 647, and 667; 575, 591, and 659; 537, 591, and 659; 523, 635, and 670; 576, 591, and 659; 577, 606, and 659; 523, 648, and 679; 523, 592, and 659; 523, 595, and 659; 523, 600, and 659; 523, 649, and 664; 578, 641, and 659; 523, 591, and 667; 523, 606, and 660; 523, 650, and 656; 579, 621, and 671; 580, 591, and 660; 527, 591, and 659; 581, 595, and 659; 582, 640, and 664; 583, 651, and 659; 534, 591, and 659; 584, 591, and 671; 523, 591, and 680; 523, 591, and 681; 585, 591, and 659; 523, 649, and 659; 586, 591, and 666; 587, 652, and 659; 588, 638, and 659; 523, 623, and 664; 523, 591, and 682; 553, 653, and 659; 523, 654, and 659; 523, 648, and 659; 560, 591, and 659; 580, 606, and 659; 589, 593, and 661; 523, 637, and 676; 590, 622, and 659; 555, 591, and 661; 523, 615, and 672; 523, 626, and 668; 523, 655, and 659; or 523, 606, and 680.

In an embodiment, the VL comprises any one of the amino acid sequences set forth in SEQ ID NOs: 181-368.

In an embodiment, an antibody or polypeptide disclosed herein is conjugated to a cytotoxic agent, cytostatic agent, toxin, radionuclide, or detectable label.

In one aspect, the present disclosure provides a polynucleotide encoding a VH and/or a VL of an antibody disclosed herein, or a polypeptide disclosed herein.

In one aspect, the present disclosure provides a vector comprising the polynucleotide disclosed herein.

In an embodiment, the vector is an adeno-associated virus (AAV) vector.

In one aspect, the present disclosure provides a recombinant host cell comprising:
(a) a polynucleotide disclosed herein;
(b) a vector disclosed herein;
(c) a first polynucleotide encoding a VH or heavy chain of an antibody disclosed herein, and a second polynucleotide encoding a VL or light chain of an antibody disclosed herein; or
(d) a first vector comprising a first polynucleotide encoding a VH or heavy chain of an antibody disclosed herein, and a second vector comprising a second polynucleotide encoding a VL or light chain of an antibody disclosed herein.

In one aspect, the present disclosure provides a pharmaceutical composition comprising an antibody or polypeptide disclosed herein, a polynucleotide disclosed herein, a vector disclosed herein, or a host cell disclosed herein, and a pharmaceutically acceptable carrier or excipient.

In one aspect, the present disclosure provides a method of producing an antibody or polypeptide, the method comprising culturing a host cell disclosed herein under suitable conditions such that the polynucleotide is expressed, and the antibody or polypeptide is produced.

In one aspect, the present disclosure provides a method of inhibiting an activity of PC in a subject, the method comprising administering to the subject an effective amount of an antibody or polypeptide disclosed herein, a polynucleotide disclosed herein, a vector disclosed herein, a host cell disclosed herein, or a pharmaceutical composition disclosed herein.

In one aspect, the present disclosure provides a method of treating an inflammatory disorder or degenerative disease in a subject, the method comprising administering to the subject an effective amount of an antibody or polypeptide disclosed herein, a polynucleotide disclosed herein, a vector disclosed herein, a host cell disclosed herein, or a pharmaceutical composition disclosed herein.

In one aspect, the present disclosure provides use of an antibody or polypeptide disclosed herein, a polynucleotide disclosed herein, a vector disclosed herein, a host cell disclosed herein, or a pharmaceutical composition disclosed herein, for the manufacture of a medicament for the treatment of an inflammatory disorder or degenerative disease in a subject in need thereof.

In one aspect, the present disclosure provides an antibody or polypeptide disclosed herein, a polynucleotide disclosed herein, a vector disclosed herein, a host cell disclosed herein, or a pharmaceutical composition disclosed herein, for use in medicine.

In one aspect, the present disclosure provides an antibody or polypeptide disclosed herein, a polynucleotide disclosed herein, a vector disclosed herein, a host cell disclosed herein, or a pharmaceutical composition disclosed herein, for use in the treatment of an inflammatory disorder or degenerative disease in a subject in need thereof.

In an embodiment, the inflammatory disorder or degenerative disease is selected from the group consisting of organ reperfusion injury such as myocardial infarction induced reperfusion injury, Kawasaki disease, non-alcoholic steatohepatitis (NASH), organ transplant rejection, atherosclerosis, Type I or Type 2 Diabetes, rheumatoid arthritis, osteoporosis, acute lung injury, asthma, chronic obstructive pulmonary disease (COPD), lung fibrosis, pain, Alzheimer's disease, amyotrophic lateral sclerosis (ALS), age-related macular degeneration (AMD), stroke, Huntington's disease, frontotemporal dementia (FTD), multiple sclerosis (MS), isolated demyelinating diseases of the central nervous system, osteoarthritis, Crohn's Disease, and ulcerative colitis.

### DETAILED DESCRIPTION

The instant disclosure provides polypeptides (e.g., antibodies) that specifically bind to PC. Also provided are pharmaceutical compositions comprising these polypeptides, nucleic acids encoding these polypeptides, expression vectors and host cells for making these polypeptides, and methods of treating a subject using these polypeptides. The polypeptides provided herein are particularly advantageous because they bind PC with high affinity, display favorable stability, stress resistance, and immunogenicity profiles, and can be produced at high yields. The polypeptides provided herein are particularly useful for treating an inflammatory disorder or degenerative disease in a subject.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

“Multispecific antibodies” are antibodies (e.g., bispecific antibodies) that specifically bind to two or more different antigens or two or more different regions of the same antigen. Multispecific antibodies include bispecific antibodies that contain two different antigen-binding sites (exclusive of the Fc region). Multispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single-chain antibodies or linked-single-chain Fvs (scFv), camelized antibodies, affybodies, linked Fab fragments, F(ab′)₂ fragments, chemically-linked Fvs, and disulfide-linked Fvs (sdFv). Multispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, multispecific antibodies described herein are IgG antibodies, or a class (e.g., human IgG₁, IgG₂, or IgG₄) or subclass thereof.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., PC). CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In certain embodiments, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that specifically bind to an antigen bind to the antigen with a K_{A} that is at least 2 logs (e.g., factors of 10), 2.5 logs, 3 logs, 4 logs, or greater than the K_{A} when the molecules bind non-specifically to another antigen.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody or polynucleotide, the term “isolated” refers to an antibody or polynucleotide that is separated from one or more contaminants (e.g., polypeptides, polynucleotides, lipids, or carbohydrates, etc.) which are present in a natural source of the antibody or polynucleotide. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### Anti-PC Antibodies

In one aspect, the instant disclosure provides polypeptides (e.g., antibodies) that specifically bind to PC. The amino acid sequences of exemplary antibodies are set forth in Table 1.

TABLE 1
Amino Acid Sequences of Exemplary Antibodies.

| | SEQ ID NO: | | | | | | | |
|---|---|---|---|---|---|---|---|---|
| Antibody | VH | VL | CDRH1 | CDRH2 | CDRH3 | CDRL1 | CDRL2 | CDRL3 |
| PcOxP_DB01_A06 | 1 | 181 | 369 | 428 | 484 | 523 | 591 | 656 |
| PcOxP_DB01_A12 | 2 | 182 | 370 | 429 | 485 | 523 | 592 | 657 |
| PcOxP_DB01_B01 | 3 | 183 | 371 | 428 | 486 | 523 | 593 | 658 |
| PcOxP_DB01_B04 | 4 | 184 | 372 | 428 | 484 | 523 | 591 | 659 |
| PcOxP_DB01_B05 | 5 | 185 | 373 | 429 | 484 | 523 | 594 | 659 |
| PcOxP_DB01_B07 | 1 | 184 | 369 | 428 | 484 | 523 | 591 | 659 |
| PcOxP_DB01_B08 | 6 | 184 | 374 | 430 | 484 | 523 | 591 | 659 |
| PcOxP_DB01_B09 | 1 | 186 | 369 | 428 | 484 | 523 | 595 | 660 |
| PcOxP_DB01_B11 | 7 | 187 | 375 | 428 | 487 | 523 | 596 | 659 |
| PcOxP_DB01_C01 | 8 | 188 | 376 | 428 | 484 | 524 | 591 | 659 |
| PcOxP_DB01_C03 | 9 | 189 | 377 | 428 | 484 | 523 | 591 | 661 |
| PcOxP_DB01_C04 | 10 | 190 | 378 | 428 | 484 | 523 | 591 | 662 |
| PcOxP_DB01_C05 | 4 | 191 | 372 | 428 | 484 | 523 | 597 | 658 |
| PcOxP_DB01_C06 | 11 | 192 | 379 | 428 | 484 | 523 | 598 | 662 |
| PcOxP_DB01_C08 | 12 | 193 | 372 | 428 | 488 | 525 | 599 | 659 |
| PcOxP_DB01_C12 | 13 | 194 | 376 | 431 | 484 | 523 | 600 | 663 |
| PcOxP_DB01_D02 | 14 | 195 | 380 | 432 | 484 | 523 | 591 | 664 |
| PcOxP_DB01_D05 | 15 | 196 | 372 | 428 | 489 | 523 | 601 | 659 |
| PcOxP_DB01_D08 | 16 | 184 | 381 | 428 | 490 | 523 | 591 | 659 |
| PcOxP_DB01_D09 | 17 | 197 | 382 | 428 | 484 | 526 | 597 | 665 |
| PcOxP_DB01_E04 | 18 | 198 | 381 | 428 | 491 | 523 | 602 | 659 |
| PcOxP_DB01_E05 | 1 | 199 | 369 | 428 | 484 | 527 | 591 | 661 |
| PcOxP_DB01_E06 | 19 | 200 | 377 | 428 | 492 | 528 | 591 | 662 |
| PcOxP_DB01_E11 | 20 | 201 | 376 | 433 | 484 | 529 | 603 | 662 |
| PcOxP_DB01_F04 | 21 | 202 | 383 | 434 | 484 | 523 | 604 | 666 |
| PcOxP_DB01_F10 | 22 | 203 | 383 | 428 | 484 | 523 | 605 | 667 |
| PcOxP_DB01_G01 | 23 | 204 | 384 | 435 | 484 | 530 | 606 | 659 |
| PcOxP_DB01_G09 | 24 | 205 | 369 | 436 | 484 | 523 | 607 | 659 |
| PcOxP_DB01_G11 | 25 | 184 | 372 | 437 | 487 | 523 | 591 | 659 |
| PcOxP_DB01_G12 | 4 | 206 | 372 | 428 | 484 | 523 | 608 | 659 |
| PcOxP_DB01_H04 | 26 | 207 | 385 | 428 | 493 | 523 | 609 | 662 |
| PcOxP_DB01_H08 | 27 | 208 | 386 | 428 | 484 | 531 | 591 | 663 |
| PcOxP_DB01_H10 | 28 | 209 | 372 | 437 | 484 | 523 | 608 | 661 |
| PcOxP_DB02_A06 | 9 | 210 | 377 | 428 | 484 | 523 | 610 | 659 |
| PcOxP_DB02_A09 | 29 | 211 | 387 | 428 | 484 | 523 | 611 | 668 |
| PcOxP_DB02_A10 | 30 | 212 | 385 | 428 | 484 | 523 | 611 | 658 |
| PcOxP_DB02_A11 | 31 | 213 | 372 | 438 | 484 | 523 | 596 | 662 |
| PcOxP_DB02_A12 | 32 | 214 | 388 | 428 | 494 | 523 | 612 | 667 |
| PcOxP_DB02_B02 | 33 | 215 | 369 | 439 | 484 | 523 | 613 | 668 |
| PcOxP_DB02_B06 | 34 | 216 | 389 | 428 | 484 | 523 | 591 | 669 |
| PcOxP_DB02_B10 | 17 | 217 | 382 | 428 | 484 | 523 | 591 | 663 |
| PcOxP_DB02_B12 | 1 | 218 | 369 | 428 | 484 | 523 | 614 | 659 |
| PcOxP_DB02_C01 | 35 | 184 | 381 | 428 | 484 | 523 | 591 | 659 |
| PcOxP_DB02_C02 | 36 | 184 | 376 | 440 | 495 | 523 | 591 | 659 |
| PcOxP_DB02_C06 | 35 | 219 | 381 | 428 | 484 | 532 | 615 | 662 |
| PcOxP_DB02_C09 | 35 | 220 | 381 | 428 | 484 | 523 | 616 | 670 |
| PcOxP_DB02_C11 | 37 | 184 | 390 | 441 | 484 | 523 | 591 | 659 |
| PcOxP_DB02_D01 | 38 | 221 | 370 | 442 | 484 | 523 | 591 | 668 |
| PcOxP_DB02_D09 | 39 | 222 | 391 | 428 | 484 | 523 | 617 | 671 |
| PcOxP_DB02_D10 | 9 | 223 | 377 | 428 | 484 | 533 | 595 | 659 |
| PcOxP_DB02_E01 | 9 | 224 | 377 | 428 | 484 | 523 | 618 | 659 |
| PcOxP_DB02_E06 | 40 | 184 | 392 | 432 | 484 | 523 | 591 | 659 |
| PcOxP_DB02_E10 | 41 | 225 | 377 | 443 | 484 | 534 | 619 | 662 |
| PcOxP_DB02_F02 | 8 | 226 | 376 | 428 | 484 | 535 | 620 | 659 |
| PcOxP_DB02_F07 | 42 | 227 | 393 | 428 | 484 | 536 | 621 | 659 |
| PcOxP_DB02_F12 | 35 | 228 | 381 | 428 | 484 | 537 | 603 | 671 |
| PcOxP_DB02_G06 | 43 | 229 | 383 | 444 | 489 | 523 | 622 | 659 |
| PcOxP_DB02_G07 | 4 | 230 | 372 | 428 | 484 | 523 | 617 | 659 |
| PcOxP_DB02_G09 | 9 | 231 | 377 | 428 | 484 | 523 | 623 | 659 |
| PcOxP_DB02_H03 | 8 | 232 | 376 | 428 | 484 | 538 | 607 | 672 |
| PcOxP_DB02_H10 | 1 | 233 | 369 | 428 | 484 | 523 | 616 | 671 |
| PcOxP_DB03_A03 | 8 | 234 | 376 | 428 | 484 | 523 | 624 | 659 |
| PcOxP_DB03_A08 | 44 | 184 | 394 | 428 | 484 | 523 | 591 | 659 |
| PcOxP_DB03_A09 | 1 | 235 | 369 | 428 | 484 | 523 | 620 | 659 |
| PcOxP_DB03_A11 | 45 | 236 | 383 | 444 | 496 | 523 | 625 | 660 |
| PcOxP_DB03_B02 | 30 | 237 | 385 | 428 | 484 | 523 | 602 | 671 |
| PcOxP_DB03_C03 | 46 | 184 | 376 | 434 | 484 | 523 | 591 | 659 |
| PcOxP_DB03_C06 | 47 | 238 | 395 | 445 | 497 | 523 | 611 | 663 |
| PcOxP_DB03_C11 | 48 | 239 | 396 | 446 | 487 | 523 | 626 | 670 |
| PcOxP_DB03_C12 | 1 | 230 | 369 | 428 | 484 | 523 | 617 | 659 |
| PcOxP_DB03_D01 | 49 | 190 | 392 | 428 | 484 | 523 | 591 | 662 |
| PcOxP_DB03_D07 | 44 | 240 | 394 | 428 | 484 | 523 | 606 | 673 |
| PcOxP_DB03_D08 | 50 | 184 | 393 | 428 | 498 | 523 | 591 | 659 |
| PcOxP_DB03_D11 | 51 | 241 | 376 | 428 | 495 | 523 | 627 | 671 |
| PcOxP_DB03_E01 | 52 | 242 | 369 | 440 | 484 | 523 | 628 | 671 |
| PcOxP_DB03_E02 | 53 | 243 | 397 | 428 | 484 | 539 | 591 | 662 |
| PcOxP_DB03_E07 | 54 | 184 | 389 | 447 | 484 | 523 | 591 | 659 |
| PcOxP_DB03_E08 | 55 | 244 | 398 | 448 | 492 | 523 | 591 | 670 |
| PcOxP_DB03_E09 | 56 | 245 | 373 | 449 | 490 | 523 | 611 | 659 |
| PcOxP_DB03_F01 | 57 | 246 | 399 | 428 | 484 | 537 | 613 | 671 |
| PcOxP_DB03_F10 | 58 | 247 | 400 | 450 | 484 | 540 | 598 | 659 |
| PcOxP_DB03_G02 | 59 | 248 | 401 | 428 | 484 | 541 | 591 | 659 |
| PcOxP_DB03_G03 | 60 | 249 | 390 | 428 | 484 | 542 | 629 | 659 |
| PcOxP_DB03_G06 | 8 | 250 | 376 | 428 | 484 | 523 | 591 | 671 |
| PcOxP_DB03_G07 | 61 | 184 | 400 | 428 | 499 | 523 | 591 | 659 |
| PcOxP_DB03_G10 | 35 | 251 | 381 | 428 | 484 | 523 | 630 | 667 |
| PcOxP_DB03_H03 | 62 | 252 | 384 | 429 | 484 | 543 | 631 | 659 |
| PcOxP_DB03_H07 | 1 | 253 | 369 | 428 | 484 | 523 | 632 | 664 |
| PcOxP_DB03_H08 | 63 | 254 | 402 | 436 | 484 | 523 | 597 | 661 |
| PcOxP_DB03_H09 | 64 | 181 | 381 | 451 | 484 | 523 | 591 | 656 |
| PcOxP_DB04_A07 | 65 | 255 | 403 | 428 | 484 | 523 | 633 | 659 |
| PcOxP_DB04_A09 | 66 | 256 | 369 | 429 | 484 | 544 | 607 | 659 |
| PcOxP_DB04_A10 | 67 | 257 | 404 | 429 | 484 | 523 | 630 | 659 |
| PcOxP_DB04_A12 | 68 | 198 | 372 | 429 | 484 | 523 | 602 | 659 |
| PcOxP_DB04_B06 | 35 | 258 | 381 | 428 | 484 | 523 | 591 | 665 |
| PcOxP_DB04_B08 | 69 | 259 | 388 | 428 | 484 | 523 | 610 | 674 |
| PcOxP_DB04_C03 | 70 | 260 | 405 | 451 | 484 | 523 | 634 | 671 |
| PcOxP_DB04_C08 | 71 | 261 | 406 | 428 | 484 | 545 | 591 | 661 |
| PcOxP_DB04_D08 | 72 | 262 | 400 | 428 | 484 | 523 | 635 | 667 |
| PcOxP_DB04_F06 | 73 | 263 | 404 | 428 | 484 | 546 | 591 | 659 |
| PcOxP_DB04_F11 | 74 | 264 | 384 | 432 | 500 | 523 | 636 | 664 |
| PcOxP_DB04_G06 | 75 | 265 | 376 | 452 | 501 | 547 | 591 | 663 |
| PcOxP_DB04_H03 | 76 | 266 | 407 | 428 | 484 | 548 | 619 | 659 |
| PcOxP_DB04_H07 | 77 | 267 | 375 | 428 | 484 | 523 | 637 | 659 |
| PcOxP_DB04_H08 | 4 | 205 | 372 | 428 | 484 | 523 | 607 | 659 |
| PcOxP_DB04_H10 | 78 | 268 | 408 | 428 | 484 | 523 | 638 | 659 |
| PcOxP_DB01_A03 | 1 | 269 | 369 | 428 | 484 | 523 | 591 | 659 |
| PcOxP_DB01_A04 | 27 | 269 | 386 | 428 | 484 | 523 | 591 | 659 |
| PcOxP_DB01_A05 | 79 | 269 | 369 | 428 | 497 | 523 | 591 | 659 |
| PcOxP_DB01_A08 | 80 | 270 | 403 | 453 | 484 | 549 | 591 | 659 |
| PcOxP_DB01_B02 | 81 | 269 | 402 | 454 | 484 | 523 | 591 | 659 |
| PcOxP_DB01_B03 | 22 | 269 | 383 | 428 | 484 | 523 | 591 | 659 |
| PcOxP_DB01_B06 | 82 | 269 | 383 | 434 | 500 | 523 | 591 | 659 |
| PcOxP_DB01_B10 | 83 | 271 | 376 | 455 | 502 | 550 | 591 | 675 |
| PcOxP_DB01_B12 | 84 | 272 | 383 | 435 | 484 | 523 | 591 | 664 |
| PcOxP_DB01_C07 | 1 | 273 | 369 | 428 | 484 | 523 | 602 | 659 |
| PcOxP_DB01_C09 | 8 | 274 | 376 | 428 | 484 | 551 | 591 | 659 |
| PcOxP_DB01_C10 | 8 | 275 | 376 | 428 | 484 | 552 | 591 | 659 |
| PcOxP_DB01_D01 | 22 | 276 | 383 | 428 | 484 | 553 | 591 | 676 |
| PcOxP_DB01_D04 | 8 | 277 | 376 | 428 | 484 | 554 | 591 | 659 |
| PcOxP_DB01_D06 | 85 | 278 | 376 | 428 | 490 | 523 | 591 | 676 |
| PcOxP_DB01_D07 | 86 | 279 | 369 | 428 | 503 | 555 | 606 | 659 |
| PcOxP_DB01_D10 | 87 | 269 | 409 | 428 | 484 | 523 | 591 | 659 |
| PcOxP_DB01_D11 | 88 | 280 | 383 | 428 | 504 | 556 | 591 | 659 |
| PcOxP_DB01_D12 | 89 | 269 | 369 | 434 | 484 | 523 | 591 | 659 |
| PcOxP_DB01_E01 | 60 | 281 | 390 | 428 | 484 | 523 | 637 | 677 |
| PcOxP_DB01_E02 | 90 | 269 | 369 | 456 | 484 | 523 | 591 | 659 |
| PcOxP_DB01_E03 | 91 | 282 | 370 | 457 | 484 | 557 | 591 | 659 |
| PcOxP_DB01_E07 | 22 | 283 | 383 | 428 | 484 | 558 | 591 | 659 |
| PcOxP_DB01_E08 | 92 | 269 | 404 | 458 | 499 | 523 | 591 | 659 |
| PcOxP_DB01_E09 | 93 | 284 | 410 | 428 | 484 | 523 | 591 | 677 |
| PcOxP_DB01_E10 | 94 | 285 | 383 | 459 | 484 | 553 | 639 | 659 |
| PcOxP_DB01_F01 | 95 | 286 | 407 | 428 | 489 | 549 | 591 | 667 |
| PcOxP_DB01_F03 | 96 | 269 | 374 | 434 | 484 | 523 | 591 | 659 |
| PcOxP_DB01_F06 | 73 | 287 | 404 | 428 | 484 | 559 | 617 | 666 |
| PcOxP_DB01_F07 | 97 | 288 | 411 | 460 | 505 | 523 | 591 | 672 |
| PcOxP_DB01_F08 | 68 | 289 | 372 | 429 | 484 | 523 | 606 | 659 |
| PcOxP_DB01_F11 | 98 | 269 | 383 | 428 | 485 | 523 | 591 | 659 |
| PcOxP_DB01_F12 | 99 | 290 | 386 | 460 | 490 | 523 | 601 | 659 |
| PcOxP_DB01_G02 | 100 | 291 | 412 | 455 | 493 | 523 | 592 | 670 |
| PcOxP_DB01_G03 | 4 | 292 | 372 | 428 | 484 | 560 | 591 | 667 |
| PcOxP_DB01_G04 | 4 | 289 | 372 | 428 | 484 | 523 | 606 | 659 |
| PcOxP_DB01_G05 | 101 | 284 | 383 | 461 | 484 | 523 | 591 | 677 |
| PcOxP_DB01_G06 | 66 | 289 | 369 | 429 | 484 | 523 | 606 | 659 |
| PcOxP_DB01_G07 | 89 | 293 | 369 | 434 | 484 | 530 | 591 | 659 |
| PcOxP_DB01_G10 | 102 | 294 | 376 | 437 | 484 | 561 | 603 | 666 |
| PcOxP_DB01_H01 | 103 | 269 | 383 | 462 | 500 | 523 | 591 | 659 |
| PcOxP_DB01_H02 | 22 | 295 | 383 | 428 | 484 | 562 | 591 | 659 |
| PcOxP_DB01_H03 | 104 | 296 | 400 | 463 | 484 | 523 | 616 | 660 |
| PcOxP_DB01_H06 | 105 | 297 | 376 | 464 | 484 | 523 | 602 | 677 |
| PcOxP_DB01_H07 | 106 | 270 | 400 | 434 | 506 | 549 | 591 | 659 |
| PcOxP_DB01_H09 | 107 | 269 | 369 | 436 | 507 | 523 | 591 | 659 |
| PcOxP_DB02_A04 | 67 | 269 | 404 | 429 | 484 | 523 | 591 | 659 |
| PcOxP_DB02_A05 | 62 | 298 | 384 | 429 | 484 | 523 | 616 | 659 |
| PcOxP_DB02_B04 | 66 | 269 | 369 | 429 | 484 | 523 | 591 | 659 |
| PcOxP_DB02_B07 | 108 | 299 | 377 | 465 | 484 | 523 | 630 | 660 |
| PcOxP_DB02_B09 | 109 | 300 | 413 | 459 | 484 | 563 | 591 | 659 |
| PcOxP_DB02_C03 | 110 | 301 | 369 | 452 | 484 | 523 | 591 | 663 |
| PcOxP_DB02_C04 | 111 | 302 | 414 | 466 | 484 | 523 | 640 | 659 |
| PcOxP_DB02_C05 | 112 | 269 | 373 | 428 | 484 | 523 | 591 | 659 |
| PcOxP_DB02_C08 | 113 | 303 | 415 | 428 | 489 | 564 | 591 | 659 |
| PcOxP_DB02_C10 | 1 | 304 | 369 | 428 | 484 | 523 | 591 | 671 |
| PcOxP_DB02_C12 | 72 | 305 | 400 | 428 | 484 | 523 | 641 | 677 |
| PcOxP_DB02_D02 | 114 | 306 | 369 | 428 | 491 | 565 | 591 | 662 |
| PcOxP_DB02_D03 | 115 | 307 | 403 | 458 | 508 | 523 | 642 | 664 |
| PcOxP_DB02_E02 | 116 | 269 | 392 | 467 | 484 | 523 | 591 | 659 |
| PcOxP_DB02_E03 | 117 | 308 | 369 | 452 | 509 | 566 | 643 | 659 |
| PcOxP_DB02_E08 | 118 | 269 | 388 | 468 | 490 | 523 | 591 | 659 |
| PcOxP_DB02_E09 | 110 | 309 | 369 | 452 | 484 | 523 | 644 | 667 |
| PcOxP_DB02_E11 | 119 | 310 | 377 | 440 | 484 | 567 | 606 | 659 |
| PcOxP_DB02_E12 | 120 | 311 | 370 | 428 | 484 | 523 | 591 | 656 |
| PcOxP_DB02_F04 | 121 | 269 | 384 | 467 | 487 | 523 | 591 | 659 |
| PcOxP_DB02_F06 | 122 | 312 | 392 | 469 | 510 | 568 | 591 | 659 |
| PcOxP_DB02_F09 | 123 | 289 | 378 | 464 | 484 | 523 | 606 | 659 |
| PcOxP_DB02_F10 | 9 | 313 | 377 | 428 | 484 | 569 | 645 | 659 |
| PcOxP_DB02_F11 | 124 | 314 | 416 | 434 | 484 | 523 | 603 | 659 |
| PcOxP_DB02_G02 | 120 | 289 | 370 | 428 | 484 | 523 | 606 | 659 |
| PcOxP_DB02_G03 | 125 | 315 | 370 | 428 | 511 | 570 | 628 | 678 |
| PcOxP_DB02_G04 | 110 | 316 | 369 | 452 | 484 | 571 | 630 | 663 |
| PcOxP_DB02_G05 | 126 | 317 | 376 | 428 | 502 | 572 | 591 | 664 |
| PcOxP_DB02_H02 | 127 | 318 | 417 | 428 | 484 | 523 | 591 | 665 |
| PcOxP_DB02_H04 | 49 | 319 | 392 | 428 | 484 | 573 | 591 | 670 |
| PcOxP_DB02_H05 | 128 | 320 | 376 | 428 | 512 | 523 | 646 | 659 |
| PcOxP_DB02_H06 | 9 | 321 | 377 | 428 | 484 | 574 | 591 | 670 |
| PcOxP_DB02_H07 | 22 | 322 | 383 | 428 | 484 | 523 | 647 | 667 |
| PcOxP_DB02_H08 | 129 | 323 | 381 | 428 | 493 | 575 | 591 | 659 |
| PcOxP_DB02_H09 | 130 | 269 | 383 | 470 | 501 | 523 | 591 | 659 |
| PcOxP_DB03_A04 | 131 | 324 | 404 | 471 | 484 | 537 | 591 | 659 |
| PcOxP_DB03_A05 | 132 | 325 | 418 | 428 | 484 | 523 | 635 | 670 |
| PcOxP_DB03_A06 | 133 | 326 | 383 | 472 | 513 | 576 | 591 | 659 |
| PcOxP_DB03_A07 | 134 | 327 | 419 | 428 | 484 | 577 | 606 | 659 |
| PcOxP_DB03_A10 | 53 | 328 | 397 | 428 | 484 | 523 | 648 | 679 |
| PcOxP_DB03_A12 | 135 | 272 | 372 | 473 | 484 | 523 | 591 | 664 |
| PcOxP_DB03_B01 | 136 | 329 | 383 | 474 | 484 | 523 | 592 | 659 |
| PcOxP_DB03_B03 | 4 | 301 | 372 | 428 | 484 | 523 | 591 | 663 |
| PcOxP_DB03_B06 | 137 | 330 | 420 | 434 | 484 | 523 | 595 | 659 |
| PcOxP_DB03_B07 | 138 | 269 | 370 | 428 | 514 | 523 | 591 | 659 |
| PcOxP_DB03_B08 | 1 | 284 | 369 | 428 | 484 | 523 | 591 | 677 |
| PcOxP_DB03_B09 | 22 | 331 | 383 | 428 | 484 | 523 | 600 | 659 |
| PcOxP_DB03_B10 | 139 | 269 | 369 | 475 | 484 | 523 | 591 | 659 |
| PcOxP_DB03_B11 | 140 | 332 | 421 | 436 | 484 | 523 | 649 | 664 |
| PcOxP_DB03_B12 | 141 | 269 | 378 | 452 | 484 | 523 | 591 | 659 |
| PcOxP_DB03_C01 | 142 | 333 | 376 | 476 | 515 | 578 | 641 | 659 |
| PcOxP_DB03_C04 | 143 | 334 | 376 | 428 | 504 | 523 | 591 | 667 |
| PcOxP_DB03_C08 | 144 | 335 | 422 | 469 | 484 | 523 | 606 | 660 |
| PcOxP_DB03_C10 | 101 | 298 | 383 | 461 | 484 | 523 | 616 | 659 |
| PcOxP_DB03_D02 | 1 | 336 | 369 | 428 | 484 | 523 | 650 | 656 |
| PcOxP_DB03_D03 | 145 | 269 | 369 | 428 | 516 | 523 | 591 | 659 |
| PcOxP_DB03_D10 | 146 | 337 | 405 | 460 | 484 | 523 | 618 | 659 |
| PcOxP_DB03_D12 | 147 | 269 | 413 | 431 | 484 | 523 | 591 | 659 |
| PcOxP_DB03_E03 | 17 | 338 | 382 | 428 | 484 | 579 | 621 | 671 |
| PcOxP_DB03_E04 | 22 | 320 | 383 | 428 | 484 | 523 | 646 | 659 |
| PcOxP_DB03_E11 | 4 | 339 | 372 | 428 | 484 | 580 | 591 | 660 |
| PcOxP_DB03_E12 | 148 | 340 | 406 | 429 | 484 | 527 | 591 | 659 |
| PcOxP_DB03_F02 | 149 | 341 | 383 | 428 | 517 | 581 | 595 | 659 |
| PcOxP_DB03_F03 | 150 | 342 | 377 | 437 | 518 | 582 | 640 | 664 |
| PcOxP_DB03_F05 | 151 | 343 | 369 | 477 | 487 | 583 | 651 | 659 |
| PcOxP_DB03_F07 | 152 | 344 | 403 | 464 | 484 | 534 | 591 | 659 |
| PcOxP_DB03_F09 | 94 | 289 | 383 | 459 | 484 | 523 | 606 | 659 |
| PcOxP_DB03_F11 | 153 | 345 | 369 | 454 | 484 | 584 | 591 | 671 |
| PcOxP_DB03_G01 | 154 | 346 | 372 | 478 | 519 | 523 | 591 | 680 |
| PcOxP_DB03_G09 | 155 | 347 | 423 | 429 | 484 | 523 | 591 | 681 |
| PcOxP_DB03_G11 | 22 | 348 | 383 | 428 | 484 | 585 | 591 | 659 |
| PcOxP_DB03_G12 | 89 | 282 | 369 | 434 | 484 | 557 | 591 | 659 |
| PcOxP_DB03_H02 | 156 | 349 | 382 | 434 | 484 | 523 | 649 | 659 |
| PcOxP_DB03_H10 | 157 | 296 | 424 | 479 | 484 | 523 | 616 | 660 |
| PcOxP_DB04_A04 | 158 | 350 | 376 | 429 | 484 | 586 | 591 | 666 |
| PcOxP_DB04_A05 | 159 | 329 | 425 | 428 | 484 | 523 | 592 | 659 |
| PcOxP_DB04_A11 | 24 | 351 | 369 | 436 | 484 | 587 | 652 | 659 |
| PcOxP_DB04_B01 | 160 | 352 | 403 | 428 | 520 | 588 | 638 | 659 |
| PcOxP_DB04_B07 | 161 | 269 | 417 | 460 | 484 | 523 | 591 | 659 |
| PcOxP_DB04_B10 | 162 | 353 | 409 | 460 | 484 | 523 | 623 | 664 |
| PcOxP_DB04_B11 | 163 | 269 | 404 | 452 | 484 | 523 | 591 | 659 |
| PcOxP_DB04_B12 | 1 | 354 | 369 | 428 | 484 | 523 | 591 | 682 |
| PcOxP_DB04_C09 | 164 | 355 | 383 | 428 | 521 | 553 | 653 | 659 |
| PcOxP_DB04_C10 | 165 | 269 | 372 | 480 | 493 | 523 | 591 | 659 |
| PcOxP_DB04_C11 | 1 | 356 | 369 | 428 | 484 | 523 | 654 | 659 |
| PcOxP_DB04_D01 | 166 | 357 | 369 | 477 | 484 | 523 | 648 | 659 |
| PcOxP_DB04_D05 | 167 | 358 | 369 | 481 | 484 | 560 | 591 | 659 |
| PcOxP_DB04_D07 | 110 | 334 | 369 | 452 | 484 | 523 | 591 | 667 |
| PcOxP_DB04_D09 | 87 | 359 | 409 | 428 | 484 | 580 | 606 | 659 |
| PcOxP_DB04_D10 | 168 | 360 | 384 | 452 | 484 | 589 | 593 | 661 |
| PcOxP_DB04_D12 | 169 | 269 | 377 | 482 | 484 | 523 | 591 | 659 |
| PcOxP_DB04_E05 | 170 | 269 | 410 | 483 | 484 | 523 | 591 | 659 |
| PcOxP_DB04_E06 | 171 | 269 | 384 | 428 | 489 | 523 | 591 | 659 |
| PcOxP_DB04_E08 | 172 | 361 | 426 | 429 | 484 | 523 | 622 | 659 |
| PcOxP_DB04_E12 | 8 | 362 | 376 | 428 | 484 | 523 | 637 | 676 |
| PcOxP_DB04_F01 | 173 | 304 | 427 | 428 | 484 | 523 | 591 | 671 |
| PcOxP_DB04_F03 | 174 | 269 | 373 | 452 | 506 | 523 | 591 | 659 |
| PcOxP_DB04_F05 | 1 | 363 | 369 | 428 | 484 | 590 | 622 | 659 |
| PcOxP_DB04_G01 | 175 | 364 | 369 | 429 | 521 | 555 | 591 | 661 |
| PcOxP_DB04_G03 | 176 | 365 | 405 | 428 | 522 | 523 | 615 | 672 |
| PcOxP_DB04_G05 | 177 | 269 | 376 | 428 | 510 | 523 | 591 | 659 |
| PcOxP_DB04_G08 | 178 | 366 | 384 | 428 | 484 | 523 | 626 | 668 |
| PcOxP_DB04_G09 | 179 | 269 | 410 | 429 | 484 | 523 | 591 | 659 |
| PcOxP_DB04_G11 | 180 | 367 | 400 | 452 | 484 | 523 | 655 | 659 |
| PcOxP_DB04_H01 | 89 | 368 | 369 | 434 | 484 | 523 | 606 | 680 |

In certain embodiments, the instant disclosure provides an antibody that specifically binds to PC, the antibody comprising a VH domain comprising one, two, or all three of the CDRs of a VH domain set forth in Table 1. In certain embodiments, the antibody comprises the CDRH1 of a VH domain set forth in Table 1. In certain embodiments, the antibody comprises the CDRH2 of a VH domain set forth in Table 1. In certain embodiments, the antibody comprises the CDRH3 of a VH domain set forth in Table 1.

In certain embodiments, the instant disclosure provides an antibody that specifically binds to PC, the antibody comprising a VL domain comprising one, two, or all three of the CDRs of a VL domain disclosed in Table 1. In certain embodiments, the antibody comprises the CDRL1 of a VL domain set forth in Table 1. In certain embodiments, the antibody comprises the CDRL2 of a VL domain set forth in Table 1. In certain embodiments, the antibody comprises the CDRL3 of a VL domain set forth in Table 1.

The individual CDRs of an antibody disclosed herein can be determined according to any CDR numbering scheme known in the art.

In certain embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In certain embodiments, the instant disclosure provides antibodies that specifically bind to PC and comprise CDRs of an antibody disclosed in Table 1 herein as determined by the Kabat numbering scheme.

In certain embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226, all of which are herein incorporated by reference in their entireties).

In certain embodiments, the instant disclosure provides antibodies that specifically bind to PC and comprise CDRs of an antibody disclosed in Table 1 herein, as determined by the Chothia numbering system.

In certain embodiments, one or more of the CDRs of an antibody disclosed herein can be determined according to MacCallum R M et al., (1996) J Mol Biol 262:732-745, herein incorporated by reference in its entirety. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001), herein incorporated by reference in its entirety.

In certain embodiments, the instant disclosure provides antibodies that specifically bind to PC and comprise CDRs of an antibody disclosed in Table 1 herein, as determined by the MacCallum numbering system.

In certain embodiments, the CDRs of an antibody disclosed herein can be determined according to the IMGT numbering system as described in: Lefranc M-P, (1999) The Immunologist 7:132-136; Lefranc M-P et al., (1999) Nucleic Acids Res 27:209-212, each of which is herein incorporated by reference in its entirety; and Lefranc M-P et al., (2009) Nucleic Acids Res 37: D1006-D1012.

In certain embodiments, the instant disclosure provides antibodies that specifically bind to PC and comprise CDRs of an antibody disclosed in Table 1 herein, as determined by the IMGT numbering system.

In certain embodiments, the CDRs of an antibody disclosed herein can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

In certain embodiments, the instant disclosure provides antibodies that specifically bind to PC and comprise CDRs of an antibody disclosed in Table 1 herein as determined by the AbM numbering scheme.

In certain embodiments, the CDRs of an antibody disclosed herein can be determined according to the AHo numbering system, as described in Honegger and Plückthun, A., J. Mol. Biol. 309:657-670 (2001), herein incorporated by reference in its entirety.

In certain embodiments, the instant disclosure provides antibodies that specifically bind to PC and comprise CDRs of an antibody disclosed in Table 1 herein, as determined by the AHo numbering system.

In certain embodiments, the individual CDRs of an antibody disclosed herein are each independently determined according to one of the Kabat, Chothia, MacCallum, IMGT, AHo, or AbM numbering schemes, or by structural analysis of the molecule, wherein the structural analysis identifies residues in the variable region(s) predicted to make contact with an epitope region of PC.

In certain embodiments, the instant disclosure provides an antibody or polypeptide that specifically binds PC, comprising a VH comprising the CDRH1, CDRH2, and CDRH3 amino acid sequences of any one of the VH amino acid sequences set forth in SEQ ID NOs: 1-180, and a VL comprising the CDRL1, CDRL2, and CDRL3 amino acid sequences of any one of the VL amino acid sequences set forth in SEQ ID NOs: 181-368, wherein each CDR is independently determined according to one of the Kabat, Chothia, MacCallum, IMGT, AHo, or AbM numbering schemes, or by structural analysis of the molecule, wherein the structural analysis identifies residues in the variable region(s) predicted to make contact with an epitope region of PC.

In certain embodiments, the instant disclosure provides an antibody or polypeptide that specifically binds to PC, wherein the antibody or polypeptide comprises the CDRH1, CDRH2, CDRH3, CDRL1, CDRL2, and CDRL3 amino acid sequences of the VH and VL amino acid sequences set forth in SEQ ID NOs: 1 and 181; 2 and 182; 3 and 183; 4 and 184; 5 and 185; 1 and 184; 6 and 184; 1 and 186; 7 and 187; 8 and 188; 9 and 189; 10 and 190; 4 and 191; 11 and 192; 12 and 193; 13 and 194; 14 and 195; 15 and 196; 16 and 184; 17 and 197; 18 and 198; 1 and 199; 19 and 200; 20 and 201; 21 and 202; 22 and 203; 23 and 204; 24 and 205; 25 and 184; 4 and 206; 26 and 207; 27 and 208; 28 and 209; 9 and 210; 29 and 211; 30 and 212; 31 and 213; 32 and 214; 33 and 215; 34 and 216; 17 and 217; 1 and 218; 35 and 184; 36 and 184; 35 and 219; 35 and 220; 37 and 184; 38 and 221; 39 and 222; 9 and 223; 9 and 224; 40 and 184; 41 and 225; 8 and 226; 42 and 227; 35 and 228; 43 and 229; 4 and 230; 9 and 231; 8 and 232; 1 and 233; 8 and 234; 44 and 184; 1 and 235; 45 and 236; 30 and 237; 46 and 184; 47 and 238; 48 and 239; 1 and 230; 49 and 190; 44 and 240; 50 and 184; 51 and 241; 52 and 242; 53 and 243; 54 and 184; 55 and 244; 56 and 245; 57 and 246; 58 and 247; 59 and 248; 60 and 249; 8 and 250; 61 and 184; 35 and 251; 62 and 252; 1 and 253; 63 and 254; 64 and 181; 65 and 255; 66 and 256; 67 and 257; 68 and 198; 35 and 258; 69 and 259; 70 and 260; 71 and 261; 72 and 262; 73 and 263; 74 and 264; 75 and 265; 76 and 266; 77 and 267; 4 and 205; 78 and 268; 1 and 269; 27 and 269; 79 and 269; 80 and 270; 81 and 269; 22 and 269; 82 and 269; 83 and 271; 84 and 272; 1 and 273; 8 and 274; 8 and 275; 22 and 276; 8 and 277; 85 and 278; 86 and 279; 87 and 269; 88 and 280; 89 and 269; 60 and 281; 90 and 269; 91 and 282; 22 and 283; 92 and 269; 93 and 284; 94 and 285; 95 and 286; 96 and 269; 73 and 287; 97 and 288; 68 and 289; 98 and 269; 99 and 290; 100 and 291; 4 and 292; 4 and 289; 101 and 284; 66 and 289; 89 and 293; 102 and 294; 103 and 269; 22 and 295; 104 and 296; 105 and 297; 106 and 270; 107 and 269; 67 and 269; 62 and 298; 66 and 269; 108 and 299; 109 and 300; 110 and 301; 111 and 302; 112 and 269; 113 and 303; 1 and 304; 72 and 305; 114 and 306; 115 and 307; 116 and 269; 117 and 308; 118 and 269; 110 and 309; 119 and 310; 120 and 311; 121 and 269; 122 and 312; 123 and 289; 9 and 313; 124 and 314; 120 and 289; 125 and 315; 110 and 316; 126 and 317; 127 and 318; 49 and 319; 128 and 320; 9 and 321; 22 and 322; 129 and 323; 130 and 269; 131 and 324; 132 and 325; 133 and 326; 134 and 327; 53 and 328; 135 and 272; 136 and 329; 4 and 301; 137 and 330; 138 and 269; 1 and 284; 22 and 331; 139 and 269; 140 and 332; 141 and 269; 142 and 333; 143 and 334; 144 and 335; 101 and 298; 1 and 336; 145 and 269; 146 and 337; 147 and 269; 17 and 338; 22 and 320; 4 and 339; 148 and 340; 149 and 341; 150 and 342; 151 and 343; 152 and 344; 94 and 289; 153 and 345; 154 and 346; 155 and 347; 22 and 348; 89 and 282; 156 and 349; 157 and 296; 158 and 350; 159 and 329; 24 and 351; 160 and 352; 161 and 269; 162 and 353; 163 and 269; 1 and 354; 164 and 355; 165 and 269; 1 and 356; 166 and 357; 167 and 358; 110 and 334; 87 and 359; 168 and 360; 169 and 269; 170 and 269; 171 and 269; 172 and 361; 8 and 362; 173 and 304; 174 and 269; 1 and 363; 175 and 364; 176 and 365; 177 and 269; 178 and 366; 179 and 269; 180 and 367; or 89 and 368, respectively.

In certain embodiments, the instant disclosure provides an antibody or polypeptide that specifically binds to PC, wherein the antibody or polypeptide comprises a VH comprising the CDRH1, CDRH2 and CDRH3 amino acid sequences set forth in SEQ ID NOs: 369, 428, and 484; 370, 429, and 485; 371, 428, and 486; 372, 428, and 484; 373, 429, and 484; 374, 430, and 484; 375, 428, and 487; 376, 428, and 484; 377, 428, and 484; 378, 428, and 484; 379, 428, and 484; 372, 428, and 488; 376, 431, and 484; 380, 432, and 484; 372, 428, and 489; 381, 428, and 490; 382, 428, and 484; 381, 428, and 491; 377, 428, and 492; 376, 433, and 484; 383, 434, and 484; 383, 428, and 484; 384, 435, and 484; 369, 436, and 484; 372, 437, and 487; 385, 428, and 493; 386, 428, and 484; 372, 437, and 484; 387, 428, and 484; 385, 428, and 484; 372, 438, and 484; 388, 428, and 494; 369, 439, and 484; 389, 428, and 484; 381, 428, and 484; 376, 440, and 495; 390, 441, and 484; 370, 442, and 484; 391, 428, and 484; 392, 432, and 484; 377, 443, and 484; 393, 428, and 484; 383, 444, and 489; 394, 428, and 484; 383, 444, and 496; 376, 434, and 484; 395, 445, and 497; 396, 446, and 487; 392, 428, and 484; 393, 428, and 498; 376, 428, and 495; 369, 440, and 484; 397, 428, and 484; 389, 447, and 484; 398, 448, and 492; 373, 449, and 490; 399, 428, and 484; 400, 450, and 484; 401, 428, and 484; 390, 428, and 484; 400, 428, and 499; 384, 429, and 484; 402, 436, and 484; 381, 451, and 484; 403, 428, and 484; 369, 429, and 484; 404, 429, and 484; 372, 429, and 484; 388, 428, and 484; 405, 451, and 484; 406, 428, and 484; 400, 428, and 484; 404, 428, and 484; 384, 432, and 500; 376, 452, and 501; 407, 428, and 484; 375, 428, and 484; 408, 428, and 484; 369, 428, and 497; 403, 453, and 484; 402, 454, and 484; 383, 434, and 500; 376, 455, and 502; 383, 435, and 484; 376, 428, and 490; 369, 428, and 503; 409, 428, and 484; 383, 428, and 504; 369, 434, and 484; 369, 456, and 484; 370, 457, and 484; 404, 458, and 499; 410, 428, and 484; 383, 459, and 484; 407, 428, and 489; 374, 434, and 484; 411, 460, and 505; 383, 428, and 485; 386, 460, and 490; 412, 455, and 493; 383, 461, and 484; 376, 437, and 484; 383, 462, and 500; 400, 463, and 484; 376, 464, and 484; 400, 434, and 506; 369, 436, and 507; 377, 465, and 484; 413, 459, and 484; 369, 452, and 484; 414, 466, and 484; 373, 428, and 484; 415, 428, and 489; 369, 428, and 491; 403, 458, and 508; 392, 467, and 484; 369, 452, and 509; 388, 468, and 490; 377, 440, and 484; 370, 428, and 484; 384, 467, and 487; 392, 469, and 510; 378, 464, and 484; 416, 434, and 484; 370, 428, and 511; 376, 428, and 502; 417, 428, and 484; 376, 428, and 512; 381, 428, and 493; 383, 470, and 501; 404, 471, and 484; 418, 428, and 484; 383, 472, and 513; 419, 428, and 484; 372, 473, and 484; 383, 474, and 484; 420, 434, and 484; 370, 428, and 514; 369, 475, and 484; 421, 436, and 484; 378, 452, and 484; 376, 476, and 515; 376, 428, and 504; 422, 469, and 484; 369, 428, and 516; 405, 460, and 484; 413, 431, and 484; 406, 429, and 484; 383, 428, and 517; 377, 437, and 518; 369, 477, and 487; 403, 464, and 484; 369, 454, and 484; 372, 478, and 519; 423, 429, and 484; 382, 434, and 484; 424, 479, and 484; 376, 429, and 484; 425, 428, and 484; 403, 428, and 520; 417, 460, and 484; 409, 460, and 484; 404, 452, and 484; 383, 428, and 521; 372, 480, and 493; 369, 477, and 484; 369, 481, and 484; 384, 452, and 484; 377, 482, and 484; 410, 483, and 484; 384, 428, and 489; 426, 429, and 484; 427, 428, and 484; 373, 452, and 506; 369, 429, and 521; 405, 428, and 522; 376, 428, and 510; 384, 428, and 484; 410, 429, and 484; or 400, 452, and 484, respectively.

In certain embodiments, the instant disclosure provides an antibody or polypeptide that specifically binds to PC, wherein the antibody or polypeptide comprises a VL comprising the CDRL1, CDRL2, and CDRL3 amino acid sequences set forth in SEQ ID NOs: 523, 591, and 656; 523, 592, and 657; 523, 593, and 658; 523, 591, and 659; 523, 594, and 659; 523, 595, and 660; 523, 596, and 659; 524, 591, and 659; 523, 591, and 661; 523, 591, and 662; 523, 597, and 658; 523, 598, and 662; 525, 599, and 659; 523, 600, and 663; 523, 591, and 664; 523, 601, and 659; 526, 597, and 665; 523, 602, and 659; 527, 591, and 661; 528, 591, and 662; 529, 603, and 662; 523, 604, and 666; 523, 605, and 667; 530, 606, and 659; 523, 607, and 659; 523, 608, and 659; 523, 609, and 662; 531, 591, and 663; 523, 608, and 661; 523, 610, and 659; 523, 611, and 668; 523, 611, and 658; 523, 596, and 662; 523, 612, and 667; 523, 613, and 668; 523, 591, and 669; 523, 591, and 663; 523, 614, and 659; 532, 615, and 662; 523, 616, and 670; 523, 591, and 668; 523, 617, and 671; 533, 595, and 659; 523, 618, and 659; 534, 619, and 662; 535, 620, and 659; 536, 621, and 659; 537, 603, and 671; 523, 622, and 659; 523, 617, and 659; 523, 623, and 659; 538, 607, and 672; 523, 616, and 671; 523, 624, and 659; 523, 620, and 659; 523, 625, and 660; 523, 602, and 671; 523, 611, and 663; 523, 626, and 670; 523, 606, and 673; 523, 627, and 671; 523, 628, and 671; 539, 591, and 662; 523, 591, and 670; 523, 611, and 659; 537, 613, and 671; 540, 598, and 659; 541, 591, and 659; 542, 629, and 659; 523, 591, and 671; 523, 630, and 667; 543, 631, and 659; 523, 632, and 664; 523, 597, and 661; 523, 633, and 659; 544, 607, and 659; 523, 630, and 659; 523, 591, and 665; 523, 610, and 674; 523, 634, and 671; 545, 591, and 661; 523, 635, and 667; 546, 591, and 659; 523, 636, and 664; 547, 591, and 663; 548, 619, and 659; 523, 637, and 659; 523, 638, and 659; 549, 591, and 659; 550, 591, and 675; 551, 591, and 659; 552, 591, and 659; 553, 591, and 676; 554, 591, and 659; 523, 591, and 676; 555, 606, and 659; 556, 591, and 659; 523, 637, and 677; 557, 591, and 659; 558, 591, and 659; 523, 591, and 677; 553, 639, and 659; 549, 591, and 667; 559, 617, and 666; 523, 591, and 672; 523, 606, and 659; 523, 592, and 670; 560, 591, and 667; 530, 591, and 659; 561, 603, and 666; 562, 591, and 659; 523, 616, and 660; 523, 602, and 677; 523, 616, and 659; 523, 630, and 660; 563, 591, and 659; 523, 640, and 659; 564, 591, and 659; 523, 641, and 677; 565, 591, and 662; 523, 642, and 664; 566, 643, and 659; 523, 644, and 667; 567, 606, and 659; 568, 591, and 659; 569, 645, and 659; 523, 603, and 659; 570, 628, and 678; 571, 630, and 663; 572, 591, and 664; 573, 591, and 670; 523, 646, and 659; 574, 591, and 670; 523, 647, and 667; 575, 591, and 659; 537, 591, and 659; 523, 635, and 670; 576, 591, and 659; 577, 606, and 659; 523, 648, and 679; 523, 592, and 659; 523, 595, and 659; 523, 600, and 659; 523, 649, and 664; 578, 641, and 659; 523, 591, and 667; 523, 606, and 660; 523, 650, and 656; 579, 621, and 671; 580, 591, and 660; 527, 591, and 659; 581, 595, and 659; 582, 640, and 664; 583, 651, and 659; 534, 591, and 659; 584, 591, and 671; 523, 591, and 680; 523, 591, and 681; 585, 591, and 659; 523, 649, and 659; 586, 591, and 666; 587, 652, and 659; 588, 638, and 659; 523, 623, and 664; 523, 591, and 682; 553, 653, and 659; 523, 654, and 659; 523, 648, and 659; 560, 591, and 659; 580, 606, and 659; 589, 593, and 661; 523, 637, and 676; 590, 622, and 659; 555, 591, and 661; 523, 615, and 672; 523, 626, and 668; 523, 655, and 659; or 523, 606, and 680, respectively.

In certain embodiments, the instant disclosure provides an antibody or polypeptide that specifically binds to PC, wherein the antibody or polypeptide comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 369, 428, 484, 523, 591, and 656; 370, 429, 485, 523, 592, and 657; 371, 428, 486, 523, 593, and 658; 372, 428, 484, 523, 591, and 659; 373, 429, 484, 523, 594, and 659; 369, 428, 484, 523, 591, and 659; 374, 430, 484, 523, 591, and 659; 369, 428, 484, 523, 595, and 660; 375, 428, 487, 523, 596, and 659; 376, 428, 484, 524, 591, and 659; 377, 428, 484, 523, 591, and 661; 378, 428, 484, 523, 591, and 662; 372, 428, 484, 523, 597, and 658; 379, 428, 484, 523, 598, and 662; 372, 428, 488, 525, 599, and 659; 376, 431, 484, 523, 600, and 663; 380, 432, 484, 523, 591, and 664; 372, 428, 489, 523, 601, and 659; 381, 428, 490, 523, 591, and 659; 382, 428, 484, 526, 597, and 665; 381, 428, 491, 523, 602, and 659; 369, 428, 484, 527, 591, and 661; 377, 428, 492, 528, 591, and 662; 376, 433, 484, 529, 603, and 662; 383, 434, 484, 523, 604, and 666; 383, 428, 484, 523, 605, and 667; 384, 435, 484, 530, 606, and 659; 369, 436, 484, 523, 607, and 659; 372, 437, 487, 523, 591, and 659; 372, 428, 484, 523, 608, and 659; 385, 428, 493, 523, 609, and 662; 386, 428, 484, 531, 591, and 663; 372, 437, 484, 523, 608, and 661; 377, 428, 484, 523, 610, and 659; 387, 428, 484, 523, 611, and 668; 385, 428, 484, 523, 611, and 658; 372, 438, 484, 523, 596, and 662; 388, 428, 494, 523, 612, and 667; 369, 439, 484, 523, 613, and 668; 389, 428, 484, 523, 591, and 669; 382, 428, 484, 523, 591, and 663; 369, 428, 484, 523, 614, and 659; 381, 428, 484, 523, 591, and 659; 376, 440, 495, 523, 591, and 659; 381, 428, 484, 532, 615, and 662; 381, 428, 484, 523, 616, and 670; 390, 441, 484, 523, 591, and 659; 370, 442, 484, 523, 591, and 668; 391, 428, 484, 523, 617, and 671; 377, 428, 484, 533, 595, and 659; 377, 428, 484, 523, 618, and 659; 392, 432, 484, 523, 591, and 659; 377, 443, 484, 534, 619, and 662; 376, 428, 484, 535, 620, and 659; 393, 428, 484, 536, 621, and 659; 381, 428, 484, 537, 603, and 671; 383, 444, 489, 523, 622, and 59; 372, 428, 484, 523, 617, and 659; 377, 428, 484, 523, 623, and 659; 376, 428, 484, 538, 607, and 672; 369, 428, 484, 523, 616, and 671; 376, 428, 484, 523, 624, and 659; 394, 428, 484, 523, 591, and 659; 369, 428, 484, 523, 620, and 659; 383, 444, 496, 523, 625, and 660; 385, 428, 484, 523, 602, and 671; 376, 434, 484, 523, 591, and 659; 395, 445, 497, 523, 611, and 663; 396, 446, 487, 523, 626, and 670; 369, 428, 484, 523, 617, and 659; 392, 428, 484, 523, 591, and 662; 394, 428, 484, 523, 606, and 673; 393, 428, 498, 523, 591, and 659; 376, 428, 495, 523, 627, and 671; 369, 440, 484, 523, 628, and 671; 397, 428, 484, 539, 591, and 662; 389, 447, 484, 523, 591, and 659; 398, 448, 492, 523, 591, and 670; 373, 449, 490, 523, 611, and 659; 399, 428, 484, 537, 613, and 671; 400, 450, 484, 540, 598, and 659; 401, 428, 484, 541, 591, and 659; 390, 428, 484, 542, 629, and 659; 376, 428, 484, 523, 591, and 671; 400, 428, 499, 523, 591, and 659; 381, 428, 484, 523, 630, and 667; 384, 429, 484, 543, 631, and 659; 369, 428, 484, 523, 632, and 664; 402, 436, 484, 523, 597, and 661; 381, 451, 484, 523, 591, and 656; 403, 428, 484, 523, 633, and 659; 369, 429, 484, 544, 607, and 659; 404, 429, 484, 523, 630, and 659; 372, 429, 484, 523, 602, and 659; 381, 428, 484, 523, 591, and 665; 388, 428, 484, 523, 610, and 674; 405, 451, 484, 523, 634, and 671; 406, 428, 484, 545, 591, and 661; 400, 428, 484, 523, 635, and 667; 404, 428, 484, 546, 591, and 659; 384, 432, 500, 523, 636, and 664; 376, 452, 501, 547, 591, and 663; 407, 428, 484, 548, 619, and 659; 375, 428, 484, 523, 637, and 659; 372, 428, 484, 523, 607, and 659; 408, 428, 484, 523, 638, and 659; 386, 428, 484, 523, 591, and 659; 369, 428, 497, 523, 591, and 659; 403, 453, 484, 549, 591, and 659; 402, 454, 484, 523, 591, and 659; 383, 428, 484, 523, 591, and 659; 383, 434, 500, 523, 591, and 659; 376, 455, 502, 550, 591, and 675; 383, 435, 484, 523, 591, and 664; 369, 428, 484, 523, 602, and 659; 376, 428, 484, 551, 591, and 659; 376, 428, 484, 552, 591, and 659; 383, 428, 484, 553, 591, and 676; 376, 428, 484, 554, 591, and 659; 376, 428, 490, 523, 591, and 676; 369, 428, 503, 555, 606, and 659; 409, 428, 484, 523, 591, and 659; 383, 428, 504, 556, 591, and 659; 369, 434, 484, 523, 591, and 659; 390, 428, 484, 523, 637, and 677; 369, 456, 484, 523, 591, and 659; 370, 457, 484, 557, 591, and 659; 383, 428, 484, 558, 591, and 659; 404, 458, 499, 523, 591, and 659; 410, 428, 484, 523, 591, and 677; 383, 459, 484, 553, 639, and 659; 407, 428, 489, 549, 591, and 667; 374, 434, 484, 523, 591, and 659; 404, 428, 484, 559, 617, and 666; 411, 460, 505, 523, 591, and 672; 372, 429, 484, 523, 606, and 659; 383, 428, 485, 523, 591, and 659; 386, 460, 490, 523, 601, and 659; 412, 455, 493, 523, 592, and 670; 372, 428, 484, 560, 591, and 667; 372, 428, 484, 523, 606, and 659; 383, 461, 484, 523, 591, and 677; 369, 429, 484, 523, 606, and 659; 369, 434, 484, 530, 591, and 659; 376, 437, 484, 561, 603, and 666; 383, 462, 500, 523, 591, and 659; 383, 428, 484, 562, 591, and 659; 400, 463, 484, 523, 616, and 660; 376, 464, 484, 523, 602, and 677; 400, 434, 506, 549, 591, and 659; 369, 436, 507, 523, 591, and 659; 404, 429, 484, 523, 591, and 659; 384, 429, 484, 523, 616, and 659; 369, 429, 484, 523, 591, and 659; 377, 465, 484, 523, 630, and 660; 413, 459, 484, 563, 591, and 659; 369, 452, 484, 523, 591, and 663; 414, 466, 484, 523, 640, and 659; 373, 428, 484, 523, 591, and 659; 415, 428, 489, 564, 591, and 659; 369, 428, 484, 523, 591, and 671; 400, 428, 484, 523, 641, and 677; 369, 428, 491, 565, 591, and 662; 403, 458, 508, 523, 642, and 664; 392, 467, 484, 523, 591, and 659; 369, 452, 509, 566, 643, and 659; 388, 468, 490, 523, 591, and 659; 369, 452, 484, 523, 644, and 667; 377, 440, 484, 567, 606, and 659; 370, 428, 484, 523, 591, and 656; 384, 467, 487, 523, 591, and 659; 392, 469, 510, 568, 591, and 659; 378, 464, 484, 523, 606, and 659; 377, 428, 484, 569, 645, and 659; 416, 434, 484, 523, 603, and 659; 370, 428, 484, 523, 606, and 659; 370, 428, 511, 570, 628, and 678; 369, 452, 484, 571, 630, and 663; 376, 428, 502, 572, 591, and 664; 417, 428, 484, 523, 591, and 665; 392, 428, 484, 573, 591, and 670; 376, 428, 512, 523, 646, and 659; 377, 428, 484, 574, 591, and 670; 383, 428, 484, 523, 647, and 667; 381, 428, 493, 575, 591, and 659; 383, 470, 501, 523, 591, and 659; 404, 471, 484, 537, 591, and 659; 418, 428, 484, 523, 635, and 670; 383, 472, 513, 576, 591, and 659; 419, 428, 484, 577, 606, and 659; 397, 428, 484, 523, 648, and 679; 372, 473, 484, 523, 591, and 664; 383, 474, 484, 523, 592, and 659; 372, 428, 484, 523, 591, and 663; 420, 434, 484, 523, 595, and 659; 370, 428, 514, 523, 591, and 659; 369, 428, 484, 523, 591, and 677; 383, 428, 484, 523, 600, and 659; 369, 475, 484, 523, 591, and 659; 421, 436, 484, 523, 649, and 664; 378, 452, 484, 523, 591, and 659; 376, 476, 515, 578, 641, and 659; 376, 428, 504, 523, 591, and 667; 422, 469, 484, 523, 606, and 660; 383, 461, 484, 523, 616, and 659; 369, 428, 484, 523, 650, and 656; 369, 428, 516, 523, 591, and 659; 405, 460, 484, 523, 618, and 659; 413, 431, 484, 523, 591, and 659; 382, 428, 484, 579, 621, and 671; 383, 428, 484, 523, 646, and 659; 372, 428, 484, 580, 591, and 660; 406, 429, 484, 527, 591, and 659; 383, 428, 517, 581, 595, and 659; 377, 437, 518, 582, 640, and 664; 369, 477, 487, 583, 651, and 659; 403, 464, 484, 534, 591, and 659; 383, 459, 484, 523, 606, and 659; 369, 454, 484, 584, 591, and 671; 372, 478, 519, 523, 591, and 680; 423, 429, 484, 523, 591, and 681; 383, 428, 484, 585, 591, and 659; 369, 434, 484, 557, 591, and 659; 382, 434, 484, 523, 649, and 659; 424, 479, 484, 523, 616, and 660; 376, 429, 484, 586, 591, and 666; 425, 428, 484, 523, 592, and 659; 369, 436, 484, 587, 652, and 659; 403, 428, 520, 588, 638, and 659; 417, 460, 484, 523, 591, and 659; 409, 460, 484, 523, 623, and 664; 404, 452, 484, 523, 591, and 659; 369, 428, 484, 523, 591, and 682; 383, 428, 521, 553, 653, and 659; 372, 480, 493, 523, 591, and 659; 369, 428, 484, 523, 654, and 659; 369, 477, 484, 523, 648, and 659; 369, 481, 484, 560, 591, and 659; 369, 452, 484, 523, 591, and 667; 409, 428, 484, 580, 606, and 659; 384, 452, 484, 589, 593, and 661; 377, 482, 484, 523, 591, and 659; 410, 483, 484, 523, 591, and 659; 384, 428, 489, 523, 591, and 659; 426, 429, 484, 523, 622, and 659; 376, 428, 484, 523, 637, and 676; 427, 428, 484, 523, 591, and 671; 373, 452, 506, 523, 591, and 659; 369, 428, 484, 590, 622, and 659; 369, 429, 521, 555, 591, and 661; 405, 428, 522, 523, 615, and 672; 376, 428, 510, 523, 591, and 659; 384, 428, 484, 523, 626, and 668; 410, 429, 484, 523, 591, and 659; 400, 452, 484, 523, 655, and 659; or 369, 434, 484, 523, 606, and 680, respectively.

In certain embodiments, the instant disclosure provides an antibody or polypeptide that specifically binds to PC, comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to any one of the amino acid sequences set forth in SEQ ID NOs: 1-180. In certain embodiments, the instant disclosure provides an antibody or polypeptide that specifically binds to PC, comprising a VH comprising any one of the amino acid sequences set forth in SEQ ID NOS: 1-180. In certain embodiments, the amino acid sequence of the VH consists of any one of the amino acid sequences set forth in SEQ ID NOs: 1-180.

In certain embodiments, the instant disclosure provides an antibody or polypeptide that specifically binds to PC, comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to any one of the amino acid sequences set forth in SEQ ID NOs: 181-368. In certain embodiments, the instant disclosure provides an antibody or polypeptide that specifically binds to PC, comprising a VL comprising any one of the amino acid sequences set forth in SEQ ID NOs: 181-368. In certain embodiments, the amino acid sequence of the VL consists of any one of the amino acid sequences set forth in SEQ ID NOs: 181-368.

In certain embodiments, the instant disclosure provides an antibody or polypeptide that specifically binds to PC, comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to any one of the amino acid sequences set forth in SEQ ID NOs: 1-180, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to any one of the amino acid sequences set forth in SEQ ID NOs: 181-368. In certain embodiments, the instant disclosure provides an antibody or polypeptide that specifically binds to PC in, comprising a VH comprising any one of the amino acid sequences set forth in SEQ ID NOs: 1-180, and a VL comprising any one of the amino acid sequences set forth in SEQ ID NOs: 181-368. In certain embodiments, the amino acid sequence of the VH consists of any one of the amino acid sequences set forth in SEQ ID NOs: 1-180; and the amino acid sequence of the VL consists of any one of the amino acid sequences set forth in SEQ ID NOs: 181-368.

In certain embodiments, the instant disclosure provides an antibody or polypeptide that specifically binds to PC, comprising the VH and VL amino acid sequences set forth in SEQ ID NOs: 1 and 181; 2 and 182; 3 and 183; 4 and 184; 5 and 185; 1 and 184; 6 and 184; 1 and 186; 7 and 187; 8 and 188; 9 and 189; 10 and 190; 4 and 191; 11 and 192; 12 and 193; 13 and 194; 14 and 195; 15 and 196; 16 and 184; 17 and 197; 18 and 198; 1 and 199; 19 and 200; 20 and 201; 21 and 202; 22 and 203; 23 and 204; 24 and 205; 25 and 184; 4 and 206; 26 and 207; 27 and 208; 28 and 209; 9 and 210; 29 and 211; 30 and 212; 31 and 213; 32 and 214; 33 and 215; 34 and 216; 17 and 217; 1 and 218; 35 and 184; 36 and 184; 35 and 219; 35 and 220; 37 and 184; 38 and 221; 39 and 222; 9 and 223; 9 and 224; 40 and 184; 41 and 225; 8 and 226; 42 and 227; 35 and 228; 43 and 229; 4 and 230; 9 and 231; 8 and 232; 1 and 233; 8 and 234; 44 and 184; 1 and 235; 45 and 236; 30 and 237; 46 and 184; 47 and 238; 48 and 239; 1 and 230; 49 and 190; 44 and 240; 50 and 184; 51 and 241; 52 and 242; 53 and 243; 54 and 184; 55 and 244; 56 and 245; 57 and 246; 58 and 247; 59 and 248; 60 and 249; 8 and 250; 61 and 184; 35 and 251; 62 and 252; 1 and 253; 63 and 254; 64 and 181; 65 and 255; 66 and 256; 67 and 257; 68 and 198; 35 and 258; 69 and 259; 70 and 260; 71 and 261; 72 and 262; 73 and 263; 74 and 264; 75 and 265; 76 and 266; 77 and 267; 4 and 205; 78 and 268; 1 and 269; 27 and 269; 79 and 269; 80 and 270; 81 and 269; 22 and 269; 82 and 269; 83 and 271; 84 and 272; 1 and 273; 8 and 274; 8 and 275; 22 and 276; 8 and 277; 85 and 278; 86 and 279; 87 and 269; 88 and 280; 89 and 269; 60 and 281; 90 and 269; 91 and 282; 22 and 283; 92 and 269; 93 and 284; 94 and 285; 95 and 286; 96 and 269; 73 and 287; 97 and 288; 68 and 289; 98 and 269; 99 and 290; 100 and 291; 4 and 292; 4 and 289; 101 and 284; 66 and 289; 89 and 293; 102 and 294; 103 and 269; 22 and 295; 104 and 296; 105 and 297; 106 and 270; 107 and 269; 67 and 269; 62 and 298; 66 and 269; 108 and 299; 109 and 300; 110 and 301; 111 and 302; 112 and 269; 113 and 303; 1 and 304; 72 and 305; 114 and 306; 115 and 307; 116 and 269; 117 and 308; 118 and 269; 110 and 309; 119 and 310; 120 and 311; 121 and 269; 122 and 312; 123 and 289; 9 and 313; 124 and 314; 120 and 289; 125 and 315; 110 and 316; 126 and 317; 127 and 318; 49 and 319; 128 and 320; 9 and 321; 22 and 322; 129 and 323; 130 and 269; 131 and 324; 132 and 325; 133 and 326; 134 and 327; 53 and 328; 135 and 272; 136 and 329; 4 and 301; 137 and 330; 138 and 269; 1 and 284; 22 and 331; 139 and 269; 140 and 332; 141 and 269; 142 and 333; 143 and 334; 144 and 335; 101 and 298; 1 and 336; 145 and 269; 146 and 337; 147 and 269; 17 and 338; 22 and 320; 4 and 339; 148 and 340; 149 and 341; 150 and 342; 151 and 343; 152 and 344; 94 and 289; 153 and 345; 154 and 346; 155 and 347; 22 and 348; 89 and 282; 156 and 349; 157 and 296; 158 and 350; 159 and 329; 24 and 351; 160 and 352; 161 and 269; 162 and 353; 163 and 269; 1 and 354; 164 and 355; 165 and 269; 1 and 356; 166 and 357; 167 and 358; 110 and 334; 87 and 359; 168 and 360; 169 and 269; 170 and 269; 171 and 269; 172 and 361; 8 and 362; 173 and 304; 174 and 269; 1 and 363; 175 and 364; 176 and 365; 177 and 269; 178 and 366; 179 and 269; 180 and 367; or 89 and 368, respectively.

In certain embodiments, the amino acid sequences of VH and VL consist of the amino acid sequences set forth in SEQ ID NOs: 1 and 181; 2 and 182; 3 and 183; 4 and 184; 5 and 185; 1 and 184; 6 and 184; 1 and 186; 7 and 187; 8 and 188; 9 and 189; 10 and 190; 4 and 191; 11 and 192; 12 and 193; 13 and 194; 14 and 195; 15 and 196; 16 and 184; 17 and 197; 18 and 198; 1 and 199; 19 and 200; 20 and 201; 21 and 202; 22 and 203; 23 and 204; 24 and 205; 25 and 184; 4 and 206; 26 and 207; 27 and 208; 28 and 209; 9 and 210; 29 and 211; 30 and 212; 31 and 213; 32 and 214; 33 and 215; 34 and 216; 17 and 217; 1 and 218; 35 and 184; 36 and 184; 35 and 219; 35 and 220; 37 and 184; 38 and 221; 39 and 222; 9 and 223; 9 and 224; 40 and 184; 41 and 225; 8 and 226; 42 and 227; 35 and 228; 43 and 229; 4 and 230; 9 and 231; 8 and 232; 1 and 233; 8 and 234; 44 and 184; 1 and 235; 45 and 236; 30 and 237; 46 and 184; 47 and 238; 48 and 239; 1 and 230; 49 and 190; 44 and 240; 50 and 184; 51 and 241; 52 and 242; 53 and 243; 54 and 184; 55 and 244; 56 and 245; 57 and 246; 58 and 247; 59 and 248; 60 and 249; 8 and 250; 61 and 184; 35 and 251; 62 and 252; 1 and 253; 63 and 254; 64 and 181; 65 and 255; 66 and 256; 67 and 257; 68 and 198; 35 and 258; 69 and 259; 70 and 260; 71 and 261; 72 and 262; 73 and 263; 74 and 264; 75 and 265; 76 and 266; 77 and 267; 4 and 205; 78 and 268; 1 and 269; 27 and 269; 79 and 269; 80 and 270; 81 and 269; 22 and 269; 82 and 269; 83 and 271; 84 and 272; 1 and 273; 8 and 274; 8 and 275; 22 and 276; 8 and 277; 85 and 278; 86 and 279; 87 and 269; 88 and 280; 89 and 269; 60 and 281; 90 and 269; 91 and 282; 22 and 283; 92 and 269; 93 and 284; 94 and 285; 95 and 286; 96 and 269; 73 and 287; 97 and 288; 68 and 289; 98 and 269; 99 and 290; 100 and 291; 4 and 292; 4 and 289; 101 and 284; 66 and 289; 89 and 293; 102 and 294; 103 and 269; 22 and 295; 104 and 296; 105 and 297; 106 and 270; 107 and 269; 67 and 269; 62 and 298; 66 and 269; 108 and 299; 109 and 300; 110 and 301; 111 and 302; 112 and 269; 113 and 303; 1 and 304; 72 and 305; 114 and 306; 115 and 307; 116 and 269; 117 and 308; 118 and 269; 110 and 309; 119 and 310; 120 and 311; 121 and 269; 122 and 312; 123 and 289; 9 and 313; 124 and 314; 120 and 289; 125 and 315; 110 and 316; 126 and 317; 127 and 318; 49 and 319; 128 and 320; 9 and 321; 22 and 322; 129 and 323; 130 and 269; 131 and 324; 132 and 325; 133 and 326; 134 and 327; 53 and 328; 135 and 272; 136 and 329; 4 and 301; 137 and 330; 138 and 269; 1 and 284; 22 and 331; 139 and 269; 140 and 332; 141 and 269; 142 and 333; 143 and 334; 144 and 335; 101 and 298; 1 and 336; 145 and 269; 146 and 337; 147 and 269; 17 and 338; 22 and 320; 4 and 339; 148 and 340; 149 and 341; 150 and 342; 151 and 343; 152 and 344; 94 and 289; 153 and 345; 154 and 346; 155 and 347; 22 and 348; 89 and 282; 156 and 349; 157 and 296; 158 and 350; 159 and 329; 24 and 351; 160 and 352; 161 and 269; 162 and 353; 163 and 269; 1 and 354; 164 and 355; 165 and 269; 1 and 356; 166 and 357; 167 and 358; 110 and 334; 87 and 359; 168 and 360; 169 and 269; 170 and 269; 171 and 269; 172 and 361; 8 and 362; 173 and 304; 174 and 269; 1 and 363; 175 and 364; 176 and 365; 177 and 269; 178 and 366; 179 and 269; 180 and 367; or 89 and 368, respectively.

Any antibody format can be used in the antibodies disclosed herein. In certain embodiments, the antibody is a single chain antibody or single-chain Fv (scFv). In certain embodiments, the antibody is an scFv fused with an Fc region (scFv-Fc). In certain embodiments, the antibody is a Fab fragment. In certain embodiments, the antibody is a F(ab′)₂ fragment.

In certain embodiments, the antibody or polypeptide disclosed herein is a multispecific antibody (e.g., a bispecific antibody) which specifically binds to PC and a second antigen.

In certain embodiments, the antibody or polypeptide disclosed herein is conjugated to a cytotoxic agent, cytostatic agent, toxin, radionuclide, or detectable label. In certain embodiments, the cytotoxic agent is able to induce death or destruction of a cell in contact therewith. In certain embodiments, the cytostatic agent is able to prevent or substantially reduce proliferation and/or inhibits the activity or function of a cell in contact therewith. In certain embodiments, the cytotoxic agent or cytostatic agent is a chemotherapeutic agent. In certain embodiments, the radionuclide is selected from the group consisting of the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. In certain embodiments, the detectable label comprises a fluorescent moiety or a click chemistry handle.

Any immunoglobulin (Ig) constant region can be used in the polypeptides and antibodies disclosed herein. In certain embodiments, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In certain embodiments, the instant disclosure provides a polypeptide (e.g., an antibody) that specifically binds to PC, the polypeptide comprising a heavy chain constant region, optionally selected from the group consisting of human IgG₁, IgG₂, IgG₃, IgG₄, IgA₁, and IgA₂.

In certain embodiments, the instant disclosure provides a polypeptide (e.g., an antibody) that specifically binds to PC, the polypeptide comprising a heavy chain constant region that is a variant of a wild-type heavy chain constant region, wherein the variant heavy chain constant region binds to an FcγR with lower affinity than the wild-type heavy chain constant region binds to the FcγR.

In certain embodiments, the instant disclosure provides an antibody that specifically binds to PC, the antibody comprising a heavy chain constant region comprising the amino acid sequence of SEQ ID NO: 683 or 684. In certain embodiments, the instant disclosure provides an antibody that specifically binds to PC, the antibody comprising a heavy chain constant region consisting of the amino acid sequence of SEQ ID NO: 683 or 684. In certain embodiments, the instant disclosure provides an antibody that specifically binds to PC, the antibody comprising an IgG₁ heavy chain constant region of any allotype or isoallotype. In certain embodiments, the instant disclosure provides an antibody that specifically binds to PC, the antibody comprising an IgG₁ heavy chain constant region of an allotype selected from the group consisting of G1m1 (a), G1m2 (x), G1m3 (f), and G1m17 (z). See, e.g., Jefferis and Lefranc (2009) mAbs 1 (4): 332-338.

In certain embodiments, one, two, or more mutations (e.g., amino acid substitutions) are introduced into an Fc region (e.g., a CH2 domain (residues 231-340 of human IgG₁) and/or a CH3 domain (residues 341-447 of human IgG₁), numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In a specific embodiment, one, two, or more amino acid mutations (e.g., substitutions, insertions, or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions, or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions, or deletions) are introduced into an IgG constant region, or FcRn-binding fragment thereof (preferably an Fc or hinge-Fc fragment) to increase the half-life of the antibody in vivo. In a specific embodiment, the antibodies may have one or more amino acid mutations (e.g., substitutions) in the second constant (CH2) domain (residues 231-340 of human IgG₁) and/or the third constant (CH3) domain (residues 341-447 of human IgG₁), numbered according to the EU numbering system. In a specific embodiment, the constant region of the IgG₁ of antibody described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall'Acqua W F et al., (2006) J Biol Chem 281:23514-24, which is herein incorporated by reference in its entirety). In certain embodiments, an antibody comprises an IgG constant region comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into an Fc region (e.g., a CH2 domain (residues 231-340 of human IgG₁) and/or a CH3 domain (residues 341-447 of human IgG₁), numbered according to the EU numbering system) and/or a hinge region (residues 216-230, numbered according to the EU numbering system) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG₁, a variant human IgG₂, or a variant human IgG₄ heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276:6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; an A330L substitution; an I332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, I332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant region of an IgG₁ with an N297Q or N297A amino acid substitution, numbered according to the EU numbering system. In certain embodiments, an antibody described herein comprises the constant region of an IgG₁ with a mutation selected from the group consisting of D265A, P329A, and a combination thereof, numbered according to the EU numbering system. In another embodiment, an antibody described herein comprises the constant region of an IgG₁ with a mutation selected from the group consisting of L234A, L235A, and a combination thereof, numbered according to the EU numbering system. In another embodiment, an antibody described herein comprises the constant region of an IgG₁ with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG, heavy chain, numbered according to the EU numbering system, are not L, L, and D, respectively. This approach is described in detail in International Publication No. WO 14/108483, which is herein incorporated by reference in its entirety. In certain embodiments, the amino acids corresponding to positions L234, L235, and D265 in a human IgG₁ heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered Clq binding and/or reduced or abolished complement dependent cytotoxicity (CDC). This approach is described in further detail in U.S. Pat. No. 6,194,551 (Idusogic et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, an antibody described herein comprises a modified constant region of an IgG₁, wherein the modification increases the ability of the antibody to mediate antibody dependent cellular cytotoxicity (ADCC). In certain embodiments, 0.1, 1, or 10 μg/mL of the antibody is capable of inducing cell death of at least 20%, 25%, 30%, 35%, 40%, 45%, 50%, 55%, or 60% of PC-expressing cells within 1, 2, or 3 hours, as assessed by methods described herein and/or known to a person of skill in the art. In certain embodiments, the modified constant region of an IgG₁ comprises S239D and I332E substitutions, numbered according to the EU numbering system. In certain embodiments, the modified constant region of an IgG₁ comprises S239D, A330L, and I332E substitutions, numbered according to the EU numbering system. In certain embodiments, the modified constant region of an IgG₁ comprises L235V, F243L, R292P, Y300L, and P396L substitutions, numbered according to the EU numbering system. In certain embodiments, the antibody is capable of inducing cell death in effector T cells and Tregs, wherein the percentage of Tregs that undergo cell death is higher than the percentage of effector T cells that undergo cell death by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, or 5 fold.

In certain embodiments, an antibody described herein comprises the constant region of an IgG₄ antibody and the serine at amino acid residue 228 of the heavy chain, numbered according to the EU numbering system, is substituted for proline. In certain embodiments, the instant disclosure provides an antibody that specifically binds to PC, the antibody comprising a heavy chain constant region comprising the amino acid sequence of SEQ ID NO: 684.

In certain embodiments, any of the constant region mutations or modifications described herein can be introduced into one or both heavy chain constant regions of an antibody described herein having two heavy chain constant regions.

### Pharmaceutical Compositions

Provided herein are compositions comprising an anti-PC antibody disclosed herein having the desired degree of purity in a physiologically acceptable carrier, excipient, or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates, including glucose, mannose, or dextrins; chelating agents such as EDTA; sugars such as sucrose, mannitol, trehalose, or sorbitol; salt-forming counter-ions such as sodium; metal complexes (e.g., Zn-protein complexes); and/or non-ionic surfactants such as TWEEN™ PLURONIC™ or polyethylene glycol (PEG).

In a specific embodiment, pharmaceutical compositions comprise an anti-PC antibody disclosed herein, and optionally one or more additional prophylactic or therapeutic agents, in a pharmaceutically acceptable carrier. In a specific embodiment, pharmaceutical compositions comprise an anti-PC antibody disclosed herein, and optionally one or more additional prophylactic or therapeutic agents, in a pharmaceutically acceptable carrier. In certain embodiments, the antibody is the only active ingredient included in the pharmaceutical composition. Pharmaceutical compositions described herein can be useful in decreasing or inhibiting PC activity and treating a condition, such as an inflammatory disorder. In certain embodiments, the present disclosure relates to a pharmaceutical composition of the present disclosure comprising an anti-PC antibody of the present disclosure for use as a medicament. In another embodiment, the present disclosure relates to a pharmaceutical composition of the present disclosure for use in a method for the treatment of an inflammatory disorder.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering, or chelating agents and other pharmaceutically acceptable substances. Examples of aqueous vehicles include Sodium Chloride Injection, Ringer's Injection, Isotonic Dextrose Injection, Sterile Water Injection, Dextrose and Lactated Ringer's Injection. Nonaqueous parenteral vehicles include fixed oils of vegetable origin, cottonseed oil, corn oil, sesame oil, and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose, and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol, and propylene glycol for water miscible vehicles; and sodium hydroxide, hydrochloric acid, citric acid, or lactic acid for pH adjustment.

A pharmaceutical composition can be formulated for any route of administration to a subject. Specific examples of routes of administration include intranasal, oral, pulmonary, transdermal, intradermal, and parenteral. Parenteral administration, characterized by either subcutaneous, intramuscular, or intravenous injection, is also contemplated herein. Injectables can be prepared in conventional forms, either as liquid solutions or suspensions, solid forms suitable for solution or suspension in liquid prior to injection, or as emulsions. The injectables, solutions, and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol, and mixtures thereof.

Topical mixtures comprising an antibody are prepared as described for the local and systemic administration. The resulting mixture can be a solution, suspension, emulsion, or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

An anti-PC antibody disclosed herein can be formulated as an aerosol for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in certain embodiments, have diameters of less than 50 microns, in certain embodiments less than 10 microns.

An anti-PC antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising an antibody described herein is a lyophilized powder, which can be reconstituted for administration as solutions, emulsions, and other mixtures. It may also be reconstituted and formulated as solids or gels. The lyophilized powder is prepared by dissolving antibody described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or other suitable agent. The solvent may also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides the desired formulation. In certain embodiments, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The anti-PC antibodies disclosed herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874, all of which are herein incorporated by reference in their entireties.

The compositions to be used for in vivo administration can be sterile. This is readily accomplished by filtration through, e.g., sterile filtration membranes.

### Methods of Use and Uses

In another aspect, the instant disclosure provides a method of treating a subject using the anti-PC antibodies disclosed herein. Any disease or disorder in a subject that would benefit from decrease of oxidized phospholipid function can be treated using the anti-PC antibodies disclosed herein.

In certain embodiments, the anti-PC antibodies disclosed herein are particularly useful for inhibiting an activity of PC in a subject. In certain embodiments, the anti-PC antibodies disclosed herein are particularly useful for inhibiting the inflammatory activity of PC in a subject. In certain embodiments, the anti-PC antibodies disclosed herein are particularly useful for treating an inflammatory disorder or degenerative disease. In certain embodiments, the anti-PC antibodies disclosed herein can be used as an anti-inflammatory agent and/or as an anti-atherosclerotic agent. In certain embodiments, the anti-PC antibodies disclosed herein can be used to treat pain. In certain embodiments, the anti-PC antibodies disclosed herein can be used to treat osteoporosis. In certain embodiments, the anti-PC antibodies disclosed herein can be used to treat fibrotic disease, including, without limitation, lung fibrosis. In certain embodiments, the anti-PC antibodies disclosed herein can be used to treat NETosis-driven diseases, including, without limitation, anti-neutrophil cytoplasmic antibody (ANCA) vasculitis and Type I diabetes. In certain embodiments, the anti-PC antibodies disclosed herein can be used to treat chronic obstructive pulmonary disease (COPD). In certain embodiments, the anti-PC antibodies disclosed herein can be used to treat acute respiratory diseases, acute fulminating pneumonias, or other similar disorders. In certain embodiments, the anti-PC antibodies disclosed herein can be used to treat severe asthma. In certain embodiments, the anti-PC antibodies disclosed herein can be used to treat patients with acute hepatitis, or with non-alcoholic steatohepatitis (NASH) and metabolic syndrome. In certain embodiments, the anti-PC antibodies disclosed herein can be used as an anti-atherosclerotic agent to treat cardiovascular disease and calcific aortic stenosis (CAS). In certain embodiments, the anti-PC antibodies disclosed herein can be used to treat patients with acute coronary syndromes or with “crescendo angina.” In certain embodiments, the anti-PC antibodies disclosed herein can be used as a proactive measure in treating high risk patients, e.g., patients who have a propensity for stroke or developing atherosclerosis. In certain embodiments, the anti-PC antibodies disclosed herein can be used treat high risk patients with coronary artery disease (CAD). Thus, the anti-PC antibodies disclosed herein can be used to treat inflammatory diseases and disorders, cardiovascular diseases, and diseases associated with oxidized phospholipids, including, without limitation, atherosclerosis, acute coronary syndrome, acute myocardial infarction, myocardial infarction (heart attack), stable and unstable angina pectoris, aneurysms, coronary artery disease (CAD), ischemic heart disease, ischemic myocardium, cardiac and sudden cardiac death, cardiomyopathy, congestive heart failure, heart failure, stenosis, peripheral arterial disease (PAD), intermittent claudication, critical limb ischemia, and stroke. See, U.S. Pat. No. 11,008,381, which is incorporated herein by reference in its entirety.

In certain embodiments, the anti-PC antibodies disclosed herein can be used to treat cardiovascular disease, atherosclerosis, rheumatoid arthritis, osteoarthritis, lung tissue injury (e.g., caused by smoking), brain lesions, apoptosis, senescence, Crohn's disease, ulcerative colitis, fatty liver disease (e.g., NASH), and non-alcoholic fatty liver disease (NAFLD). Thus, the anti-PC antibodies disclosed herein can be used to treat inflammatory diseases and disorders, cardiovascular diseases, liver diseases and disorder (e.g., NASH, NAFLD) and diseases associated with oxidative stress and damage. In certain embodiments, the anti-PC antibodies disclosed herein can be used to treat non-alcoholic fatty liver disease. In certain embodiments, the anti-PC antibodies disclosed herein can be used to treat non-alcoholic steatohepatitis (NASH). In certain embodiments, the anti-PC antibodies disclosed herein can be used to inhibit the progression of NAFLD to NASH. In certain embodiments, the anti-PC antibodies disclosed herein can be used to treat or inhibit atherogenesis. See, U.S. Pat. No. 11,168,148, which is incorporated herein by reference in its entirety.

In certain embodiments, the anti-PC antibodies disclosed herein can be used to treat reperfusion injury. In certain embodiments, reperfusion injury comprises organ reperfusion injury. In certain embodiments, reperfusion injury comprises ischemic-reperfusion injury. In certain embodiments, organ reperfusion injury is induced by an ischemic event (e.g., myocardial infarction induced reperfusion injury). In certain embodiments, the anti-PC antibodies disclosed herein can be used to treat a subject that is at risk of suffering or has suffered an ischemic event, including, without limitation, an ischemic event associated with a condition selected from the group consisting of cerebral ischemia; intestinal ischemia; spinal cord ischemia; cardiovascular ischemia; myocardial ischemia associated with myocardial infarction; myocardial ischemia associated with congestive heart failure (CHF), ischemia associated with age-related macular degeneration (AME); liver ischemia; kidney/renal ischemia; dermal ischemia; vasoconstriction-induced tissue ischemia; penile ischemia as a consequence of priapism and erectile dysfunction; ischemia associated with thromboembolytic disease; ischemia associated with microvascular disease; ischemia associated with thrombosis; and ischemia associated with diabetic ulcers, gangrenous conditions, post-trauma syndrome, cardiac arrest resuscitation, hypothermia, peripheral nerve damage and neuropathies. In certain embodiments, the anti-PC antibodies disclosed herein can be used to treat a subject that is at risk of suffering or has suffered an ischemic event that is the result of an induced injury, including, without limitation, from surgery, transplantation, accidental trauma, and mechanical support devices. In certain embodiments, the anti-PC antibodies disclosed herein can be used to treat a subject that is at risk of suffering or has suffered an ischemic event that is the result of heart surgery, kidney surgery, brain surgery, liver surgery, and bypass surgery. In certain embodiments, the anti-PC antibodies disclosed herein can be used to treat acute ischemic stroke. In certain embodiments, the anti-PC antibodies disclosed herein can be used to treat reperfusion-driven indications including transplantation of organs such as the liver, heart, and kidney. In certain embodiments, the anti-PC antibodies disclosed herein can be used to treat a disease associated with thrombosis, including, without limitation sickle cell disease, deep vein thrombosis, pulmonary embolism, cardiac embolism, hypercoagulable state, thrombophilia, Factor V Leiden, Antithrombin III deficiency, Protein C deficiency, Protein S deficiency, Prothrombin gene mutation (G20210A), hyperhomocysteinemia, antiphospholipid antibody syndrome (APS), anticardiolipin antibody (ACLA) thrombosis syndrome, and lupus anticoagulant (LA) syndrome. In certain embodiments, the anti-PC antibodies disclosed herein can be used to treat or inhibit atherogenesis. See, U.S. patent application Ser. No. 16/759,331, which is incorporated herein by reference in its entirety.

In certain embodiments, the anti-PC antibodies disclosed herein can be used to treat a toll-like receptor 2 (TLR2)-mediated disease or disorder, including, without limitation, Kawasaki disease including IVIG-refractory Kawasaki Disease, type 2 diabetes, rheumatoid arthritis, dermatologic disease, multiple sclerosis, systemic lupus erythematosus, ulcerative colitis, Graves' Disease, Sjögren's syndrome, autoimmune thyroid diseases, or vasculitis. See, U.S. patent application Ser. No. 16/965,271, which is incorporated herein by reference in its entirety.

In certain embodiments, the anti-PC antibodies disclosed herein can be used to treat bacterial infection. In certain embodiments, the anti-PC antibodies disclosed herein can be used to treat neuroinflammatory diseases, including, without limitation, amyotrophic lateral sclerosis (ALS), Parkinson's disease (PD), and Alzheimer's disease (AD). In certain embodiments, the anti-PC antibodies disclosed herein can be used to treat Huntington's disease, frontotemporal dementia (FTD), multiple sclerosis (MS), clinically isolated syndrome (CIS), and other demyelinating diseases of the central nervous system. In certain embodiments, the anti-PC antibodies disclosed herein can be used to treat ocular disease, including, without limitation, age-related macular degeneration (AMD) and geographic atrophy (GA).

In certain embodiments, these methods further comprise administering an additional therapeutic agent to the subject. In certain embodiments, the additional therapeutic agent is a HMG-COA reductase inhibitor (statin), immunosuppressive agent, ezetimide, perixosome, nicotinic acid, squalene inhibitor, proliferative activated receptor (PPAR) agonist, cholesteryl ester transfer protein (CETP) inhibitor, ApoA-1 Milano, corticosteroid, non-steroidal anti-inflammatory drug, steroidal anti-inflammatory drug, analgesic, growth factor, anti-atherosclerosis drug, anti-proliferative agent, HSP, Beta-2-glycoprotein-I, and any derivative and analog thereof.

HMGCoA reductase inhibitors (statins) are well-known drugs that effectively reduce low-density lipoprotein (LDL)-cholesterol levels by inhibiting the enzyme that regulates the rate of cholesterol production and increasing the clearance of LDL-cholesterol (LDL-C) present in the blood by the liver. Non-limiting examples of commonly prescribed statins include Atorvastatin, Fluvastatin, Lovastatin, Pravastatin, and Simvastatin.

Non-limiting examples of immunosuppressive agents include biologics such as infliximab, abatacept, adalimumab, anakinra, certolizumab, etanercept, golimumab, ixekizumab, natalizumab, rituximab, secukinumab, tocilizumab, ustekinumab, canakinumab, and vedolizumab, calcineurin inhibitors such as cyclosporine and tacrolimus, mTOR inhibitors such as sirolimus and everolimus, inosine monophosphate dehydrogenase (IMDH) inhibitors such as azathioprine, leflunomide, and mycophenolate, methotrexate, monoclonal antibodies such as daclizumab and basiliximab, muromonab-CD3, and Janus kinase inhibitors such as tofacitinib.

Ezetimibe is the first of a new class of cholesterol absorption inhibitors that potently and selectively inhibits dietary and biliary cholesterol absorption at the brush border of the intestinal epithelium, without affecting the absorption of triglyceride or fat-soluble vitamins. Ezetimibe thus reduces overall cholesterol delivery to the liver, secondarily inducing increased expression of LDL receptors, resulting in an increased removal of LDL-C from the plasma.

The peroxisome is a single-membrane organelle present in nearly all eukaryotic cells. One of the most important metabolic processes of the peroxisome is the β-oxidation of long and very long chain fatty acids. The peroxisome is also involved in bile acid synthesis, cholesterol synthesis, plasmalogen synthesis, amino acid metabolism, and purine metabolism.

Nicotinic acid is a known agent that lowers total cholesterol, LDL-cholesterol, and triglyceride levels, while raising high-density lipoprotein (HDL)-cholesterol levels. There are three types of nicotinic acid drugs: immediate release, timed release, and extended release. Nicotinic acid or niacin, the water-soluble B vitamin, improves all lipoproteins when given in doses well above the vitamin requirement.

Squalene, an isoprenoid compound structurally similar to beta-carotene, is an intermediate metabolite in the synthesis of cholesterol. In humans, about 60 percent of dietary squalene is absorbed. It is transported in serum generally in association with very low-density lipoproteins and is distributed ubiquitously in human tissues, with the greatest concentration in the skin, where it is one of the major components of skin surface lipids. Squalene inhibitors (e.g., monooxygenase and synthase) serve as cholesterol biosynthesis inhibitors.

Proliferative Activated Receptor (PPAR) agonists, e.g., fibrates, are fatty acid-activated members of the nuclear receptor superfamily that play important roles in lipid and glucose metabolism, and have been implicated in obesity-related metabolic diseases such as hyperlipidemia, insulin resistance, and coronary artery disease. Fibrates are generally effective in lowering elevated plasma triglycerides and cholesterol and act as PPAR agonists. The most pronounced effect of fibrates includes a decrease in plasma triglyceride-rich lipoproteins (TRLs). Levels of LDL-C generally decrease in individuals with elevated baseline plasma concentrations, and HDL cholesterol (HDL-C) levels are usually increased when baseline plasma concentrations are low. Non-limiting examples of commonly prescribed fibrates include bezafibrate, gemfibrozil, and fenofibrate.

Cholesteryl Ester Transfer Protein (CETP) inhibitors play a major role in atherogenesis, by reducing cholesteryl ester accumulation within macrophages and the arterial wall, and thus reducing foam cell formation and affecting the cholesterol absorption. The most promising presently known CETP inhibitor is avisimibe.

ApoA-1 Milano is typically used as a recombinant complex with phospholipid (ETC-216) and produces significant regression of coronary atherosclerosis.

Non-limiting examples of non-steroidal anti-inflammatory drugs include oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

Non-limiting examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of analgesics (pain relievers) include aspirin and other salicylates (such as choline or magnesium salicylate), ibuprofen, ketoprofen, naproxen sodium, and acetaminophen.

Growth factors are hormones which have numerous functions, including regulation of adhesion molecule production, altering cellular proliferation, increasing vascularization, enhancing collagen synthesis, regulating bone metabolism, and altering migration of cells into given area. Non-limiting examples of growth factors include insulin-like growth factor-1 (IGF-1), transforming growth factor-β (TGF-β), a bone morphogenic protein (BMP), and the like.

Non-limiting examples of anti-proliferative agents include an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier, miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Specific examples of chemotherapeutic agents include, for example, a nitrogen mustard, an cpipodophyllotoxin, an antibiotic, a platinum coordination complex, bleomycin, doxorubicin, paclitaxel, ctoposide, 4-OH cyclophosphamide, and cisplatinum.

The HSP family consists of approximately 25 proteins discerned by their molecular weights with highly conserved structures. Almost all humans have cellular and humoral immune reactions against microbial heat-shock protein 60 (HSP60). Because a high degree of antigenic homology exists between microbial (bacterial and parasitic) and human HSP60, the ‘cost’ of immunity to microbes might be the danger of cross-reactivity with human HSP60 expressed by the endothelial cells of stressed arteries. Genuine autoimmunity against altered autologous HSP60 might trigger this process also (Wick et al., TRENDS in Immunology. 2001; 22 (12): 665-669). HSP has been implicated as a target autoantigen in several experimental autoimmune diseases (arthritis, type I diabetes). Anti-HSP65 as well as anti-HSP60 antibodies have been demonstrably associated with atheromatous lesions in humans. Studies conducted in rabbits and mice show that the generation of an HSP65-induced immune response by immunization with the recombinant protein or with an HSP65-rich preparation of Mycobacterium tuberculosis enhances atherogenesis. As autoimmune processes pointing to HSP65 as a possible antigenic candidate, creating a state of unresponsiveness by induction of mucosal “tolerization” has been employed in order to block these responses, our group reported that early atherosclerosis was attenuated in HSP65-fed mice, compared with either BSA or PBS fed mice (Harats et al., J Am Coll Cardiol. 2002; 40:1333-1338), this was further supported by Maron who demonstrated that nasal vaccination with HSP reduces the inflammatory process associated with atherosclerosis (Maron ct al., Circulation. 2002; 106:1708-1715).

Beta-2-glycoprotein I (beta2GPI) is a phospholipid binding protein shown to serve as a target for prothrombotic anti-phospholipid antibodies. It has recently been demonstrated to drive an immune mediated reaction and enhance murine atherosclerosis. β-Antibodies to beta-2-GPI have the ability to activate monocytes and endothelial cells and can induce an immune response to beta2GPI in atherosclerosis-prone mice accelerated atherosclerosis. When beta2GPI-reactive lymph node and spleen cells were transferred to LDL-receptor-deficient mice they promoted fatty streak formation, proving a direct proatherogenic role for beta2GPI-specific lymphocytes. Inducing immunological tolerance to beta2GPI by prior oral feeding with the antigen resulted in a significant reduction in the extent of atherosclerotic lesions. Thus, beta2GPI is a candidate player in the atherosclerotic plaque, and can possibly be employed as an immunomodulator of plaque progression. Oral feeding with of beta2GPI inhibited lymph node cell reactivity to beta2GPI in mice immunized against the human protein. IL-4 and IL-10 production was upregulated in lymph node cells of beta2GPI-tolerant mice immunized against beta2GPI, upon priming with the respective protein. Thus, oral administration of beta2GPI is an effective means of suppressing atherogenesis in mice (George et al., Cardiovasc Res. 2004; 62 (3): 603-9).

In certain embodiments, the anti-PC antibodies or pharmaceutical compositions disclosed herein synergize with the additional therapeutic agent.

In certain embodiments, the present disclosure relates to an antibody and/or pharmaceutical composition of the present disclosure for use in a method of the present disclosure, wherein the method further comprises administering an additional therapeutic agent to the subject. In certain embodiments, the present disclosure relates to (a) an antibody and/or pharmaceutical composition of the present disclosure, and (b) an additional therapeutic agent for use as a medicament. In certain embodiments, the present disclosure relates to (a) an antibody and/or pharmaceutical composition of the present disclosure, and (b) an additional therapeutic agent for use in a method for the treatment of an inflammatory disorder. In a further embodiment, the present disclosure relates to a pharmaceutical composition, kit or kit-of-parts comprising (a) an antibody and/or pharmaceutical composition of the present disclosure, and (b) an additional therapeutic agent. In certain embodiments, the additional therapeutic agent is an HMG-COA reductase inhibitor (statin), immunosuppressive agent, ezetimibe, peroxisome, nicotinic acid, squalene inhibitor, proliferative activated receptor (PPAR) agonist, cholesteryl ester transfer protein (CETP) inhibitor, ApoA-1 Milano, non-steroidal anti-inflammatory drug, steroidal anti-inflammatory drug, analgesic, growth factor, anti-atherosclerosis drug, anti-proliferative agent, HSP, or Beta-2-glycoprotein-I.

The anti-PC antibody and the additional therapeutic agent (e.g., HMG-COA reductase inhibitor (statin), immunosuppressive agent, ezetimibe, peroxisome, nicotinic acid, squalene inhibitor, proliferative activated receptor (PPAR) agonist, cholesteryl ester transfer protein (CETP) inhibitor, ApoA-1 Milano, non-steroidal anti-inflammatory drug, steroidal anti-inflammatory drug, analgesic, growth factor, anti-atherosclerosis drug, anti-proliferative agent, HSP, or Beta-2-glycoprotein-I) can be administered separately, sequentially, or concurrently as separate dosage forms.

An antibody or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intra-arterial, and subcutaneous routes. Pulmonary administration can also be employed, e.g., by use of an inhaler or nebulizer, and formulation with an aerosolizing agent for use as a spray. In certain embodiments, the antibody or pharmaceutical composition described herein is delivered subcutaneously or intravenously. In certain embodiments, the antibody or pharmaceutical composition described herein is delivered intra-arterially. In certain embodiments, the antibody or pharmaceutical composition described herein is delivered intratumorally. In certain embodiments, the antibody or pharmaceutical composition described herein is delivered into a tumor draining lymph node.

The amount of an antibody or composition which will be effective in the treatment and/or prevention of a condition will depend on the nature of the disease, and can be determined by standard clinical techniques.

The precise dose to be employed in a composition will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, and health), whether the patient is human or an animal, other medications administered, or whether treatment is prophylactic or therapeutic. Usually, the patient is a human, but non-human mammals, including transgenic mammals, can also be treated. Treatment dosages are optimally titrated to optimize safety and efficacy.

An anti-PC antibody described herein can also be used to assay OxPL levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody described herein. Alternatively, a second antibody that recognizes an anti-PC antibody described herein can be labeled and used in combination with an anti-PC antibody to detect OxPL levels. Therefore, in certain embodiments, the present disclosure relates to the use of an anti-PC antibody of the present disclosure for in vitro detection of OxPL in a biological sample. In a further embodiment, the present disclosure relates to the use of an anti-PC antibody of the disclosure, for assaying and/or detecting OxPL levels in a biological sample in vitro or ex vivo, optionally wherein the anti-PC antibody is conjugated to a radionuclide or detectable label, and/or carries a label described herein, and/or wherein an immunohistological method is used.

Assaying for the level of OxPL is intended to include qualitatively or quantitatively measuring or estimating the level of OxPL in a first biological sample either directly (e.g., by determining or estimating absolute OxPL level) or relatively (e.g., by comparing to the disease associated OxPL level in a second biological sample). OxPL level in the first biological sample can be measured or estimated and compared to a standard OxPL level, the standard being taken, for example, from a second biological sample obtained from an individual not having the disorder or being determined by averaging levels from a population of individuals not having the disorder. As will be appreciated in the art, once the “standard” OxPL level is known, it can be used repeatedly as a standard for comparison. Therefore, in a further embodiment, the present disclosure relates to an in vitro method for assaying and/or detecting OxPL levels, for example human OxPL levels, in a biological sample, comprising qualitatively or quantitatively measuring or estimating the level of OxPL, for example of human OxPL, in a biological sample, by an immunohistological method.

As used herein, the term “biological sample” refers to any biological sample obtained from a subject, cell line, tissue, or other source of cells potentially containing oxidized phospholipids. Methods for obtaining tissue biopsies and body fluids from animals (e.g., humans or cynomolgus monkeys) are well known in the art. Biological samples include peripheral blood mononuclear cells (PBMCs).

An anti-PC antibody described herein can be used for prognostic, diagnostic, monitoring, and screening applications, including in vitro and in vivo applications well known and standard to the skilled artisan and based on the present description. Prognostic, diagnostic, monitoring, and screening assays and kits for in vitro assessment and evaluation of immune system status and/or immune response may be utilized to predict, diagnose, and monitor to evaluate patient samples, including those known to have or suspected of having an immune system dysfunction or with regard to an anticipated or desired immune system response or antigen response. The assessment and evaluation of immune system status and/or immune response is also useful in determining the suitability of a patient for a clinical trial of a drug or for the administration of a particular therapeutic agent or antibody, including combinations thereof, versus a different agent or antibody. In vivo applications include directed cell therapy and immune system modulation and radio imaging of immune responses. Therefore, in certain embodiments, the present disclosure relates to an anti-PC antibody and/or pharmaceutical composition of the present disclosure for use as a diagnostic. In certain embodiments, the present disclosure relates to an anti-PC antibody and/or pharmaceutical composition of the present disclosure for use in a method for the prediction, diagnosis, and/or monitoring of a subject having or suspected to have an immune system dysfunction and/or with regard to an anticipated or desired immune system response or antigen response. In another embodiment, the present disclosure relates to the use of an anti-PC antibody of the disclosure, for predicting, diagnosing, and/or monitoring of a subject having or suspected to have an immune system dysfunction and/or with regard to an anticipated or desired immune system response or antigen response, by assaying and/or detecting human OxPL levels in a biological sample of the subject in vitro or in vivo.

In certain embodiments, an anti-PC antibody can be used in immunohistochemistry of biopsy samples. In certain embodiments, the method is an in vitro method. In another embodiment, an anti-PC antibody can be used to detect levels of OxPL, or levels of cells which contain OxPL, the levels of which can then be linked to certain disease symptoms. Anti-PC antibodies described herein may carry a detectable or functional label and/or may be conjugated to a radionuclide or detectable label. When fluorescence labels are used, currently available microscopy and fluorescence-activated cell sorter analysis (FACS) or combination of both methods procedures known in the art may be utilized to identify and to quantitate the specific binding members. Anti-PC antibodies described herein may carry or may be conjugated to a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-PC antibody may carry or may be conjugated to a radioactive label or radionuclide, such as the isotopes ³H, ¹⁴C, ³²P, ³⁵S, ³⁶Cl, ⁵¹Cr, ⁵⁷Co, ⁵⁸Co, ⁵⁹Fc, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. When radioactive labels are used, currently available counting procedures known in the art may be utilized to identify and quantitate the specific binding of an anti-PC antibody to PC. In the instance where the label is an enzyme, detection may be accomplished by any of the presently utilized colorimetric, spectrophotometric, fluorospectrophotometric, amperometric, or gasometric techniques as known in the art. This can be achieved by contacting a sample or a control sample with an anti-PC antibody under conditions that allow for the formation of a complex between the anti-PC antibody and PC. Any complexes formed between the anti-PC antibody and PC, are detected and compared in the sample and the control. In light of the specific binding of the anti-PC antibodies described herein for PC, the anti-PC antibodies can be used to specifically detect PC. The anti-PC antibodies described herein can also be used to purify PC via immunoaffinity purification. Also included herein is an assay system which may be prepared in the form of a test kit, kit, or kit-of-parts for the quantitative analysis of the extent of the presence of, for instance, PC/PC ligand complexes. The system, test kit, kit, or kit-of-parts may comprise a labeled component, e.g., a labeled antibody, and one or more additional immunochemical reagents.

### Polynucleotides, Vectors, and Methods of Producing Antibodies

In another aspect, provided herein are polynucleotides comprising a nucleotide sequence encoding an antibody, or a portion thereof, described herein or a fragment thereof (e.g., a VL and/or VH; and a light chain and/or heavy chain) that specifically binds to a PC antigen, and vectors, e.g., vectors comprising such polynucleotides for recombinant expression in host cells (e.g., E. coli and mammalian cells). Provided herein are polynucleotides comprising nucleotide sequences encoding a heavy and/or light chain of any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecules having less than about 15%, 10%, 5%, 2%, 1%, 0.5%, or 0.1% (in particular less than about 10%) of other material, e.g., cellular material, culture medium, other nucleic acid molecules, chemical precursors, and/or other chemicals. In a specific embodiment, a nucleic acid molecule(s) encoding an antibody described herein is isolated or purified.

In particular aspects, provided herein are polynucleotides comprising nucleotide sequences encoding antibodies, which specifically bind to PC and comprises an amino acid sequence as described herein, as well as antibodies which compete with such antibodies for binding to PC (e.g., in a dose-dependent manner), or which binds to the same epitope as that of such antibodies.

In certain aspects, provided herein are polynucleotides comprising a nucleotide sequence encoding the light chain or heavy chain of antibody described herein. The polynucleotides can comprise nucleotide sequences encoding a light chain comprising the VL FRs and CDRs of antibodies described herein (see, e.g., Table 1) or nucleotide sequences encoding a heavy chain comprising the VH FRs and CDRs of antibodies described herein (see, e.g., Table 1). In certain embodiments, a polynucleotide encodes a VH, VL, heavy chain, and/or light chain of a described herein. In another embodiment, a polynucleotide encodes the first VH and the first VL of a described herein. In another embodiment, a polynucleotide encodes the second VH and the second VL of a described herein. In another embodiment, a polynucleotide encodes the first heavy chain and the first light chain of a described herein. In another embodiment, a polynucleotide encodes the second heavy chain and the second light chain of a described herein. In another embodiment, a polynucleotide encodes the VH and/or the VL, or the heavy chain and/or the light chain, of an antibody described herein.

Also provided herein are polynucleotides encoding an anti-PC antibody that are optimized, e.g., by codon/RNA optimization, replacement with heterologous signal sequences, and elimination of mRNA instability elements. Methods to generate optimized nucleic acids encoding an anti-PC antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an anti-PC antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-PC antibody encoded by polynucleotides that have not been optimized.

In certain embodiments, an optimized polynucleotide sequence encoding an anti-PC antibody described herein or a fragment thereof (e.g., VL domain and/or VH domain) can hybridize to an antisense (e.g., complementary) polynucleotide of an unoptimized polynucleotide sequence encoding an anti-PC antibody described herein or a fragment thereof (e.g., VL domain and/or VH domain). In specific embodiments, an optimized nucleotide sequence encoding an anti-PC antibody described herein or a fragment hybridizes under high stringency conditions to antisense polynucleotide of an unoptimized polynucleotide sequence encoding an anti-PC antibody described herein or a fragment thereof. In a specific embodiment, an optimized nucleotide sequence encoding an anti-PC antibody described herein or a fragment thereof hybridizes under high stringency, intermediate, or lower stringency hybridization conditions, to an antisense polynucleotide of an unoptimized nucleotide sequence encoding an anti-PC antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Table 1, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17:242-6, herein incorporated by reference in its entirety), which, briefly, involves the synthesis of overlapping oligonucleotides containing portions of the sequence encoding the antibody, annealing, and ligating of those oligonucleotides, and then amplification of the ligated oligonucleotides by PCR.

Alternatively, a polynucleotide encoding an antigen-binding region of a described here or an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning.

If a clone containing a nucleic acid encoding a particular antigen-binding region or antibody is not available, but the sequence of the antigen-binding region or antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody described herein) by PCR amplification using synthetic primers hybridizable to the 3′ and 5′ ends of the sequence, or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-PC antibodies described herein can be readily isolated and sequenced using conventional procedures (e.g., by using oligonucleotide probes that are capable of binding specifically to genes encoding the heavy and light chains of the anti-PC antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-PC antibodies in the recombinant host cells.

To generate whole antibodies or antigen-binding regions, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate, or lower stringency hybridization conditions, to polynucleotides that encode an antibody described herein. In specific embodiments, polynucleotides described herein hybridize under high stringency, intermediate, or lower stringency hybridization conditions, to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain aspects, provided herein are cells (e.g., host cells) expressing (e.g., recombinantly) antibodies described herein which specifically bind to PC, and related polynucleotides and expression vectors. Provided herein are vectors (e.g., expression vectors) comprising polynucleotides comprising nucleotide sequences encoding anti-PC antibodies or a fragment for recombinant expression in host cells, preferably in mammalian cells (e.g., CHO cells). Also provided herein are host cells comprising such vectors for recombinantly expressing anti-PC antibodies described herein (e.g., human or humanized antibody). In a particular aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to PC generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties) and variable regions of the antibody can be cloned into such a vector for expression of the entire heavy, the entire light chain, or both the entire heavy and light chains.

In certain embodiments, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce containing an antibody described herein or a fragment thereof. Thus, provided herein are host cells containing a polynucleotide encoding containing an antibody described herein or fragments thereof, or a heavy or light chain thereof, or fragment thereof, or a single-chain antibody described herein, operably linked to a promoter for expression of such sequences in the host cell.

In another embodiment, a host cell comprises a polynucleotide encoding the VH and VL of an antibody described herein. In another embodiment, a host cell comprises a vector comprising a polynucleotide encoding the VH and VL of an antibody described herein. In another embodiment, a host cell comprises a first polynucleotide encoding the VH of an antibody described herein, and a second polynucleotide encoding the VL of an antibody described herein. In another embodiment, a host cell comprises a first vector comprising a first polynucleotide encoding the VH of an antibody described herein, and a second vector comprising a second polynucleotide encoding the VL of an antibody described herein.

In specific embodiments, a heavy chain/heavy chain variable region expressed by a first cell is associated with a light chain/light chain variable region of a second cell to form an anti-PC antibody described herein. In certain embodiments, provided herein is a population of host cells comprising such first host cell and such second host cell.

In certain embodiments, provided herein is a population of vectors comprising a first vector comprising a polynucleotide encoding a light chain/light chain variable region of an anti-PC antibody described herein, and a second vector comprising a polynucleotide encoding a heavy chain/heavy chain variable region of an anti-PC antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are Chinese hamster ovary (CHO) cells, for example CHO cells from the CHO GS System™ (Lonza). In certain embodiments, the heavy chain and/or light chain of an antibody produced by a CHO cell may have an N-terminal glutamine or glutamate residue replaced by pyroglutamate. In certain embodiments, cells for expressing antibodies described herein are human cells, e.g., human cell lines. In a specific embodiment, a mammalian expression vector is pOptiVEC™ or pcDNA3.3. In certain embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus, are an effective expression system for antibodies (Foccking M K & Hofstetter H (1986) Gene 45:101-5; and Cockett M I et al., (1990) Biotechnology 8 (7): 662-7, each of which is herein incorporated by reference in its entirety). In certain embodiments, antibodies described herein are produced by CHO cells or NS0 cells. In a specific embodiment, the expression of nucleotide sequences encoding antibodies described herein which specifically bind to PC is regulated by a constitutive promoter, inducible promoter, or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ructher U & Mueller-Hill B (1983) EMBO J 2:1791-1794), in which the coding sequence can be ligated individually into the vector in frame with the lac Z coding region so that a fusion protein is produced; pIN vectors (Inouye S & Inouye M (1985) Nuc Acids Res 13:3101-3109; Van Hecke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells. In certain embodiments, anti-PC antibodies described herein are produced in mammalian cells, such as CHO cells.

In a specific embodiment, the antibodies described herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-PC antibody described herein can be engineered. In specific embodiments, a cell provided herein stably expresses a light chain/light chain variable region and a heavy chain/heavy chain variable region which associate to form an antigen-binding region or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-PC described herein or a fragment thereof. Such engineered cell lines can be particularly useful in screening and evaluation of compositions that interact directly or indirectly with the antibody molecule.

A number of selection systems can be used, including but not limited to the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al., (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of an antibody molecule can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the protein will also increase (Crouse G F et al., (1983) Mol Cell Biol 3:257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322:562-565; and Köhler G (1980) PNAS 77:2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10, or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. In certain embodiments, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

Anti-PC antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In a specific embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-PC antibody comprising culturing a cell or host cell described herein. In certain embodiments, the method is performed in vitro. In a certain aspect, provided herein is a method of making an anti-PC antibody comprising expressing (e.g., recombinantly expressing) the antibody using a cell or host cell described herein (e.g., a cell or a host cell comprising polynucleotides encoding an antibody described herein). In certain embodiments, the cell is an isolated cell. In certain embodiments, the exogenous polynucleotides have been introduced into the cell. In certain embodiments, the method further comprises the step of purifying the antibody obtained from the cell or host cell.

In certain embodiments, an antibody is produced by expressing in a cell a polynucleotide encoding the VH and VL of an antibody described herein under suitable conditions so that the polynucleotides are expressed and the antibody is produced. In another embodiment, an antibody is produced by expressing in a cell a polynucleotide encoding the heavy chain and light chain of an antibody described herein under suitable conditions so that the polynucleotides are expressed and the antibody is produced. In certain embodiments, an antibody is produced by expressing in a cell a first polynucleotide encoding the VH of an antibody described herein, and a second polynucleotide encoding the VL of an antibody described herein, under suitable conditions so that the polynucleotides are expressed and the antibody is produced. In certain embodiments, an antibody is produced by expressing in a cell a first polynucleotide encoding the heavy chain of an antibody described herein, and a second polynucleotide encoding the light chain of an antibody described herein, under suitable conditions so that the polynucleotides are expressed and the antibody is produced.

Methods for producing polyclonal antibodies are known in the art (see, for example, Chapter 11 in: Short Protocols in Molecular Biology, (2002) 5th Ed., Ausubel F M et al., eds., John Wiley and Sons, New York, which is herein incorporated by reference in its entirety).

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In specific embodiments, a “monoclonal antibody,” as used herein, is an antibody produced by a single cell (e.g., hybridoma or host cell producing a recombinant antibody), wherein the antibody specifically binds to PC as determined, e.g., by ELISA or other antigen-binding or competitive binding assay known in the art, or in the examples provided herein. In certain embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody is a monospecific or multispecific antibody (e.g., bispecific antibody). Monoclonal antibodies described herein can, for example, be made by the hybridoma method as described in Kohler G & Milstein C (1975) Nature 256:495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce or are capable of producing antibodies that will specifically bind to the protein (e.g., PC) used for immunization. Alternatively, lymphocytes may be immunized in vitro. Lymphocytes then are fused with myeloma cells using a suitable fusing agent, such as polyethylene glycol, to form a hybridoma cell (Goding J W (ed.), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In certain embodiments, mice (or other animals, such as rats, monkeys, donkeys, pigs, sheep, hamster, or dogs) can be immunized with an antigen (e.g., PC) and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthinc guanine phosphoribosyl transferase (HGPRT or HPRT), the culture medium for the hybridomas typically will include hypoxanthinc, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as the NS0 cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133:3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against PC. The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (ed.), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize PC, and can be generated by any technique known to those of skill in the art. For example, Fab and F(ab′)₂ fragments described herein can be produced by protcolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli, and the E. coli is infected with helper phage. Phage used in these methods are typically filamentous phage, including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12 (6): 864-9; Sawai H et al., (1995) Am J Reprod Immunol 34:26-34; and Better M et al., (1988) Science 240:1041-1043, all of which are herein incorporated by reference in their entireties.

In certain embodiments, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. Human kappa and lambda constant region sequences are known to those of skill in the art. Examples of human kappa and lambda constant region sequences are set forth in SEQ ID NOS: 688-693. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229:1202-7; Oi V T & Morrison S L (1986) BioTechniques 4:214-221; Gillies S D et al., (1989) J Immunol Methods 125:191-202; and U.S. Pat. Nos. 5,807,715, 4,816,567, 4,816,397, and 6,331,415, all of which are herein incorporated by reference in their entireties.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG₁, IgG₂, IgG₃, and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28 (4/5): 489-498; Studnicka G M et al., (1994) Prot Engineering 7 (6): 805-814; and Roguska M A et al., (1994) PNAS 91:969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169:1119-25; Caldas C et al., (2000) Protein Eng. 13 (5): 353-60; Morea V et al., (2000) Methods 20 (3): 267-79; Baca M et al., (1997) J Biol Chem 272 (16): 10678-84; Roguska M A et al., (1996) Protein Eng 9 (10): 895 904; Couto J R et al., (1995) Cancer Res. 55 (23 Supp): 5973s-5977s; Couto J R et al., (1995) Cancer Res 55 (8): 1717-22; Sandhu J S (1994) Gene 150 (2): 409-10; and Pedersen J T et al., (1994) J Mol Biol 235 (3): 959-73, all of which are herein incorporated by reference in their entireties. See also, U.S. Application Publication No. US 2005/0042664 A1 (Feb. 24, 2005) and International Publication No. WO 2008/020079, which are herein incorporated by reference in their entireties.

Methods for making multispecific antibodies (e.g., bispecific antibodies) have been described, see, for example, U.S. Pat. Nos. 7,951,917; 7,183,076; 8,227,577; 5,837,242; 5,989,830; 5,869,620; 6,132,992; and 8,586,713, all of which are herein incorporated by reference in their entireties.

Bispecific, bivalent antibodies, and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See generally, Int. Appl. Publ. Nos. WO 93/17715, WO 92/08802, WO 91/00360, and WO 92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110 (13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some embodiments, compositions of the disclosure have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in some instances contain, IgG₄ and IgG₁, IgG₄ and IgG₂, IgG₄ and IgG₃, or IgG₁ and IgG₃ chain heterodimers. Such heterodimeric heavy chain antibodies can routinely be engineered by, for example, modifying selected amino acids forming the interface of the CH3 domains in human IgG₄ and the IgG₁ or IgG₃, so as to favor heterodimeric heavy chain formation.

In certain embodiments, an antibody described herein, which binds to the same epitope of PC as an anti-PC antibody described herein, is a human antibody. In certain embodiments, an antibody described herein, which competitively blocks (e.g., in a dose-dependent manner) any one of the antibodies described herein, from binding to PC, is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., PC). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13:65-93, herein incorporated by reference in its entirety. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, all of which are herein incorporated by reference in their entireties. Examples of mice capable of producing human antibodies include the XenoMouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the TransChromo Mouse™ (Kirin) and the KM Mouse™ (Medarex/Kirin), all of which are herein incorporated by reference in their entireties.

Human antibodies that specifically bind to PC can be made by a variety of methods known in the art, including the phage display methods described above using antibody libraries derived from human immunoglobulin sequences. See also, U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., PC). Such methods are known and are described in the art, see, e.g., Shinmoto H et al., (2004) Cytotechnology 46:19-23; Naganawa Y et al., (2005) Human Antibodies 14:27-31, each of which is herein incorporated by reference in its entirety.

### Kits

Also provided are kits comprising one or more antibodies described herein, or pharmaceutical compositions or conjugates thereof. In a specific embodiment, provided herein is a pharmaceutical pack or kit comprising one or more containers filled with one or more of the ingredients of the pharmaceutical compositions described herein, such as one or more antibodies provided herein. In certain embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In certain embodiments, a kit comprises an antibody described herein, preferably purified antibody, in one or more containers. In a specific embodiment, kits described herein contain a substantially isolated PC antigen as a control. In another specific embodiment, the kits described herein further comprise a control antibody which does not react with PC antigen. In another specific embodiment, kits described herein contain one or more elements for detecting the binding of an antibody to an PC antigen (e.g., the antibody can be conjugated to a detectable substrate such as a fluorescent compound, an enzymatic substrate, a radioactive compound, or a luminescent compound, or a second antibody which recognizes the first antibody can be conjugated to a detectable substrate). In specific embodiments, a kit provided herein can include a recombinantly produced or chemically synthesized PC antigen. The PC antigen provided in the kit can also be attached to a solid support. In a more specific embodiment, the detecting means of the above-described kit includes a solid support to which an PC antigen is attached. Such a kit can also include a non-attached reporter-labeled anti-human antibody or anti-mouse/rat antibody. In this embodiment, binding of the antibody to the PC antigen can be detected by binding of the said reporter-labeled antibody. In certain embodiments, the present disclosure relates to the use of a kit of the present disclosure for in vitro assaying and/or detecting PC antigen in a biological sample.

### EXAMPLES

The following examples are offered by way of illustration and not by way of limitation.

### Example 1: Screening Summary

A multi-factor optimization platform was used for antibody optimization. The platform was designed to optimize for affinity, removal of liabilities, thermostability, species cross-reactivity, and selective activation.

E06 variable heavy (VH) and variable kappa (VK) CDR sequences were grafted onto 4 fully human VH germlines (VH1-46, VH3-15, VH3-23, VH5-51) and 4 fully human VK germlines (VK1-39, VK2-28, VK3-15, VK4-1) respectively. Of the sixteen combinations made, two were selected for platform library construction: E06_VK 1-39/VH 3-23 and E06_VK 4-1/VH 3-23. The individual VH and VK sequences are set forth in Table 2.

TABLE 2
Grafted VH and VK Sequences.

| | | SEQ ID |
|---|---|---|
| | Sequence (CDR sequences underlined) | NO: |
| E06_VH3-23 | EVQLLESGGGLVQPGGSLRLSCAASGFTFSDFYMEWVRQAP | 8 |
| | GKGLEWVAASRNKANDYTTEYADSVKGRFTISRDNSKNTL | |
| | YLQMNSLRAEDTAVYYCARDYYGSSYWYFDVWGQGTLVT | |
| | VSS | |
| E06_VK1-39 | DIQMTQSPSSLSASVGDRVTITCTASESLYSSKHKVHYLAW | 685 |
| | YQQKPGKAPKLLIYGASNRYIGVPSRFSGSGSGTDFTLTISSL | |
| | QPEDFATYYCAQFYSYPLTEGQGTKVEIK | |
| E06_VK4-1 | DIVMTQSPDSLAVSLGERATINCTASESLYSSKHKVHYLAW | 269 |
| | YQQKPGQPPKLLIYGASNRYIGVPDRFSGSGSGTDFTLTISSL | |
| | QAEDVAVYYCAQFYSYPLTFGGGTKVEIK | |

Phage display libraries of scFv reformatted framework grafts were designed based on structural and sequence analyses to retain CDR residues identified to exhibit key target binding interactions. The remaining CDR residues were used for guided 3-way sequence space exploration to maximize tolerability of human residues while limiting the potential impact on target binding. First, CDRH1, CDRH2, CDRL1, CDRL2, and CDRL3 were modified from parental CDR towards human germline CDRs with up to 4 changes per CDR. Second, CDRH1, CDRH2, CDRL1, CDRL2, and CDRL3 were modified from germline towards parental CDR residues with up to 4 changes. Third, all CDRs were subjected to single amino acid NNK walks omitting residue changes that would generate biochemical liabilities. E06 VK 1-39/VH 3-23 and VK 4-1/VH 3-23 scFv libraries were combined, resulting in a total size of 4×10⁹ transformants (referred to as the “platform library”).

Phosphocholine (PC) conjugated peptides were used for phage panning and scFv primary screening by ELISA. A summary of PC conjugated peptides used is set forth in Table 3.

TABLE 3
Phosphocholine (PC) Conjugated Peptides Used for Screening.

| Peptide | Conju- | | |
|---|---|---|---|
| # | gation | Tag | Full Sequence |
| 1 | PC | Biotin | Biotin-AGGGGSGGGGSGGGGSAAA-[Tyr(PC)]-acid |
| | | | (SEQ ID NO: 686) |
| 2 | PC | — | AGGGGSGGGGSGGGGSAAA-[Tyr(PC)]-acid |
| | | | (SEQ ID NO: 686) |
| 4 | — | Biotin | Biotin-AGGGGSGGGGSGGGGSAAA-Tyr-acid |
| | | | (SEQ ID NO: 686) |
| 5 | PC | Biotin | Biotin-AEAAAKEAAAKEAAAKEAAAKAAA-[Tyr(PC)]-acid |
| | | | (SEQ ID NO: 687) |
| 6 | PC | — | AEAAAKEAAAKEAAAKEAAAKAAA-[Tyr(PC)]-acid |
| | | | (SEQ ID NO: 687) |
| 8 | — | Biotin | Biotin-AEAAAKEAAAKEAAAKEAAAKAAA-Tyr-acid |
| | | | (SEQ ID NO: 687) |

PC = p-diazophenyl phosphoryl choline

Phage-peptide complexes were captured on the biotin tag of the peptides with streptavidin coated beads. After washing, bound phage were eluted using triethylamine and titered to determine enrichment. After each round of panning, eluted phage were used to infected E. coli ER2738 for propagation of phage particles to be used in subsequent rounds of selections.

Single colonies from the Round 4 panning arms (380 colonies from each; 1140 colonies in total) were cultured and scFvs were Sanger sequenced. 736 unique sequences were re-arrayed onto 96-well plates and proceeded to primary screening by scFv ELISA on Peptides 1, 4, 5, and 8.

A total of 368 unique binders to Peptide 5 and/or Peptide 1 from the primary screen were re-arrayed and proceeded to secondary screening by ELISA (n=2) on Peptides 1, 4, 5, and 8, as well as BSA. A total of 257 unique scFvs were identified with good reproducibility between primary and secondary screening data, and between replicates. These 257 sequences bound specifically to at least one of Peptide 1 and Peptide 5, with no binding to Peptide 4, Peptide 8, or BSA (thresholds for positive OD450 were determined using average OD450 for negative controls (a PD-1 specific scFv)+3×SD: See Example 2 for method).

### Example 2: scFv ELISA Summary

Periplasmic extract (PPE) containing scFv was generated by osmotic shock. Starter cultures (2×YT with 2% glucose, tetracycline (15 μg/mL) and carbenicillin (100 μg/mL), 700 μl/well in deep well plate) were inoculated with 5-10 μl of glycerol stock and grown over night at 30° C. and 700 rpm. The following day, expression cultures (2×YT with 0.1% glucose and carbenicillin (100 μg/mL), 1 ml/well in deep well plates) were inoculated with 30 μl starter culture and allowed to grow to OD600 0.5 at 37° C. and 700 rpm. Cultures were induced with 1 mM IPTG and grown for 6 hours at 25° C. and 700 rpm.

After 6 hours, plates were centrifuged to pellet bacteria and stored overnight at −20° C. The next day, pellets were thawed and resuspended in 80 μl Periplasmic Extraction Buffer with protease inhibitors and put on a shaker for 10 minutes at 4° C. and 500 rpm. After 10 minutes, 240 μl water and protease inhibitor were added to each well and incubated for 1 hour at 4° C. shaking at 500 rpm. Plates were then centrifuged at 4300×g for 10 min at 4° C. Supernatants were passed through a filter plate and stored at −80° C.

For ELISA analysis of scFv binding, 96-well polystyrene plates were coated with Peptide 1, 4, 5, 8 or 100 μL BSA (10 μg/mL) in PBS at 4° C. overnight. Plates were washed three times with PBS-Tween and blocked with 100 μL 1% BSA-TBS for 1 hour at room temperature. 96-well streptavidin coated plates were coated with 100 μL biotinylated PC-peptides (1 μg/mL) in PBS for 1 hour at room temperature.

All plates were washed three times with PBS-Tween before 40 μL aliquots of PPE were added to assay plates along with 50 μL 10% BSA, and plates were incubated for 1 hour at room temperature. After washing, 100 μL HRP conjugated anti-V5 in 5% BSA was added, and plates were incubated at room temperature for 30 mins. Plates were washed and 100 μL TMB substrate was added. Color was left to develop at room temperature before the reaction was stopped with 100 μL 1N HCl. The amount of scFv bound to each well was quantitated from absorbance measurement at 450 nm (OD450). Binding data for the 257 unique scFvs identified in Example 1 are summarized in Table 4.

TABLE 4
scFv ELISA Data.

| | | | OD450 | | | | |
|---|---|---|---|---|---|---|---|
| | SEQ ID NO: | | Pep- | Pep- | Pep- | Pep- | |
| Binder | VH | VL | tide 1 | tide 4 | tide 5 | tide 8 | BSA |
| PcOxP_DB01_A06 | 1 | 181 | 0.74 | 0.06 | 1.18 | 0.06 | 0.10 |
| PcOxP_DB01_A12 | 2 | 182 | 0.29 | 0.06 | 1.04 | 0.05 | 0.09 |
| PcOxP_DB01_B01 | 3 | 183 | 0.87 | 0.07 | 0.90 | 0.05 | 0.10 |
| PcOxP_DB01_B04 | 4 | 184 | 0.57 | 0.06 | 1.02 | 0.05 | 0.08 |
| PcOxP_DB01_B05 | 5 | 185 | 0.23 | 0.05 | 0.74 | 0.05 | 0.10 |
| PcOxP_DB01_B07 | 1 | 184 | 1.07 | 0.06 | 1.36 | 0.05 | 0.11 |
| PcOxP_DB01_B08 | 6 | 184 | 0.41 | 0.06 | 0.89 | 0.05 | 0.10 |
| PcOxP_DB01_B09 | 1 | 186 | 1.00 | 0.05 | 1.42 | 0.05 | 0.10 |
| PcOxP_DB01_B11 | 7 | 187 | 1.11 | 0.05 | 1.32 | 0.04 | 0.09 |
| PcOxP_DB01_C01 | 8 | 188 | 0.71 | 0.06 | 1.13 | 0.05 | 0.11 |
| PcOxP_DB01_C03 | 9 | 189 | 0.98 | 0.06 | 1.37 | 0.05 | 0.10 |
| PcOxP_DB01_C04 | 10 | 190 | 1.38 | 0.05 | 1.42 | 0.05 | 0.08 |
| PcOxP_DB01_C05 | 4 | 191 | 0.79 | 0.06 | 1.11 | 0.06 | 0.11 |
| PcOxP_DB01_C06 | 11 | 192 | 1.18 | 0.05 | 1.31 | 0.05 | 0.08 |
| PcOxP_DB01_C08 | 12 | 193 | 0.38 | 0.06 | 0.79 | 0.05 | 0.10 |
| PcOxP_DB01_C12 | 13 | 194 | 0.88 | 0.05 | 1.18 | 0.05 | 0.08 |
| PcOxP_DB01_D02 | 14 | 195 | 0.56 | 0.05 | 1.27 | 0.05 | 0.09 |
| PcOxP_DB01_D05 | 15 | 196 | 0.87 | 0.06 | 1.56 | 0.05 | 0.10 |
| PcOxP_DB01_D08 | 16 | 184 | 0.58 | 0.05 | 1.08 | 0.05 | 0.08 |
| PcOxP_DB01_D09 | 17 | 197 | 0.27 | 0.05 | 0.66 | 0.05 | 0.07 |
| PcOxP_DB01_E04 | 18 | 198 | 0.55 | 0.05 | 1.12 | 0.04 | 0.07 |
| PcOxP_DB01_E05 | 1 | 199 | 0.95 | 0.06 | 1.39 | 0.06 | 0.11 |
| PcOxP_DB01_E06 | 19 | 200 | 0.68 | 0.05 | 1.05 | 0.05 | 0.09 |
| PcOxP_DB01_E11 | 20 | 201 | 0.62 | 0.05 | 0.73 | 0.06 | 0.09 |
| PcOxP_DB01_F04 | 21 | 202 | 0.36 | 0.05 | 0.57 | 0.05 | 0.09 |
| PcOxP_DB01_F10 | 22 | 203 | 0.68 | 0.05 | 0.98 | 0.05 | 0.09 |
| PcOxP_DB01_G01 | 23 | 204 | 0.58 | 0.06 | 1.33 | 0.05 | 0.09 |
| PcOxP_DB01_G09 | 24 | 205 | 0.92 | 0.06 | 1.40 | 0.05 | 0.11 |
| PcOxP_DB01_G11 | 25 | 184 | 0.99 | 0.06 | 1.32 | 0.06 | 0.09 |
| PcOxP_DB01_G12 | 4 | 206 | 0.75 | 0.07 | 1.33 | 0.06 | 0.09 |
| PcOxP_DB01_H04 | 26 | 207 | 2.01 | 0.07 | 1.92 | 0.07 | 0.11 |
| PcOxP_DB01_H08 | 27 | 208 | 0.56 | 0.06 | 0.85 | 0.06 | 0.09 |
| PcOxP_DB01_H10 | 28 | 209 | 0.60 | 0.06 | 1.39 | 0.06 | 0.08 |
| PcOxP_DB02_A06 | 9 | 210 | 1.12 | 0.05 | 1.07 | 0.05 | 0.10 |
| PcOxP_DB02_A09 | 29 | 211 | 0.98 | 0.05 | 1.30 | 0.04 | 0.11 |
| PcOxP_DB02_A10 | 30 | 212 | 0.42 | 0.05 | 0.90 | 0.04 | 0.11 |
| PcOxP_DB02_A11 | 31 | 213 | 0.64 | 0.04 | 0.89 | 0.04 | 0.09 |
| PcOxP_DB02_A12 | 32 | 214 | 0.20 | 0.05 | 0.27 | 0.06 | 0.06 |
| PcOxP_DB02_B02 | 33 | 215 | 0.49 | 0.05 | 0.98 | 0.05 | 0.11 |
| PcOxP_DB02_B06 | 34 | 216 | 0.35 | 0.07 | 0.79 | 0.04 | 0.11 |
| PcOxP_DB02_B10 | 17 | 217 | 0.67 | 0.04 | 1.03 | 0.04 | 0.08 |
| PcOxP_DB02_B12 | 1 | 218 | 2.33 | 0.05 | 1.34 | 0.04 | 0.10 |
| PcOxP_DB02_C01 | 35 | 184 | 1.03 | 0.06 | 1.47 | 0.05 | 0.10 |
| PcOxP_DB02_C02 | 36 | 184 | 0.35 | 0.05 | 0.62 | 0.05 | 0.10 |
| PcOxP_DB02_C06 | 35 | 219 | 0.54 | 0.05 | 1.03 | 0.05 | 0.11 |
| PcOxP_DB02_C09 | 35 | 220 | 0.69 | 0.05 | 1.01 | 0.04 | 0.10 |
| PcOxP_DB02_C11 | 37 | 184 | 0.12 | 0.05 | 0.24 | 0.04 | 0.10 |
| PcOxP_DB02_D01 | 38 | 221 | 0.31 | 0.05 | 0.35 | 0.05 | 0.09 |
| PcOxP_DB02_D09 | 39 | 222 | 0.16 | 0.05 | 0.44 | 0.04 | 0.11 |
| PcOxP_DB02_D10 | 9 | 223 | 0.69 | 0.05 | 1.01 | 0.04 | 0.10 |
| PcOxP_DB02_E01 | 9 | 224 | 0.43 | 0.05 | 0.63 | 0.10 | 0.11 |
| PcOxP_DB02_E06 | 40 | 184 | 0.06 | 0.04 | 0.08 | 0.04 | 0.10 |
| PcOxP_DB02_E10 | 41 | 225 | 0.52 | 0.04 | 0.79 | 0.04 | 0.09 |
| PcOxP_DB02_F02 | 8 | 226 | 0.37 | 0.05 | 0.60 | 0.05 | 0.11 |
| PcOxP_DB02_F07 | 42 | 227 | 0.43 | 0.05 | 0.91 | 0.05 | 0.10 |
| PcOxP_DB02_F12 | 35 | 228 | 0.50 | 0.04 | 0.81 | 0.04 | 0.07 |
| PcOxP_DB02_G06 | 43 | 229 | 0.43 | 0.06 | 0.87 | 0.05 | 0.08 |
| PcOxP_DB02_G07 | 4 | 230 | 0.52 | 0.05 | 0.99 | 0.09 | 0.10 |
| PcOxP_DB02_G09 | 9 | 231 | 0.35 | 0.05 | 0.71 | 0.04 | 0.10 |
| PcOxP_DB02_H03 | 8 | 232 | 0.27 | 0.05 | 0.82 | 0.05 | 0.08 |
| PcOxP_DB02_H10 | 1 | 233 | 1.43 | 0.05 | 1.39 | 0.05 | 0.08 |
| PcOxP_DB03_A03 | 8 | 234 | 0.54 | 0.06 | 0.81 | 0.06 | 0.09 |
| PcOxP_DB03_A08 | 44 | 184 | 0.67 | 0.06 | 0.71 | 0.05 | 0.11 |
| PcOxP_DB03_A09 | 1 | 235 | 1.02 | 0.05 | 1.09 | 0.05 | 0.09 |
| PcOxP_DB03_A11 | 45 | 236 | 1.12 | 0.05 | 1.08 | 0.05 | 0.08 |
| PcOxP_DB03_B02 | 30 | 237 | 2.02 | 0.05 | 1.64 | 0.05 | 0.07 |
| PcOxP_DB03_C03 | 46 | 184 | 1.36 | 0.05 | 1.66 | 0.05 | 0.09 |
| PcOxP_DB03_C06 | 47 | 238 | 0.64 | 0.05 | 0.61 | 0.05 | 0.07 |
| PcOxP_DB03_C11 | 48 | 239 | 1.14 | 0.05 | 0.75 | 0.04 | 0.07 |
| PcOxP_DB03_C12 | 1 | 230 | 2.74 | 0.05 | 1.77 | 0.05 | 0.09 |
| PcOxP_DB03_D01 | 49 | 190 | 2.51 | 0.05 | 1.60 | 0.05 | 0.08 |
| PcOxP_DB03_D07 | 44 | 240 | 0.75 | 0.06 | 0.95 | 0.05 | 0.12 |
| PcOxP_DB03_D08 | 50 | 184 | 0.54 | 0.05 | 0.57 | 0.05 | 0.10 |
| PcOxP_DB03_D11 | 51 | 241 | 0.50 | 0.06 | 0.66 | 0.05 | 0.08 |
| PcOxP_DB03_E01 | 52 | 242 | 2.50 | 0.06 | 2.11 | 0.06 | 0.09 |
| PcOxP_DB03_E02 | 53 | 243 | 2.00 | 0.06 | 1.49 | 0.05 | 0.11 |
| PcOxP_DB03_E07 | 54 | 184 | 0.78 | 0.05 | 0.86 | 0.05 | 0.08 |
| PcOxP_DB03_E08 | 55 | 244 | 0.87 | 0.05 | 0.92 | 0.05 | 0.09 |
| PcOxP_DB03_E09 | 56 | 245 | 0.39 | 0.05 | 0.72 | 0.05 | 0.08 |
| PcOxP_DB03_F01 | 57 | 246 | 0.58 | 0.05 | 0.80 | 0.05 | 0.09 |
| PcOxP_DB03_F10 | 58 | 247 | 0.65 | 0.05 | 0.85 | 0.07 | 0.09 |
| PcOxP_DB03_G02 | 59 | 248 | 1.33 | 0.04 | 1.59 | 0.05 | 0.07 |
| PcOxP_DB03_G03 | 60 | 249 | 1.57 | 0.05 | 1.80 | 0.08 | 0.08 |
| PcOxP_DB03_G06 | 8 | 250 | 2.48 | 0.06 | 2.01 | 0.06 | 0.10 |
| PcOxP_DB03_G07 | 61 | 184 | 2.18 | 0.06 | 1.67 | 0.05 | 0.10 |
| PcOxP_DB03_G10 | 35 | 251 | 1.80 | 0.05 | 1.21 | 0.05 | 0.11 |
| PcOxP_DB03_H03 | 62 | 252 | 0.27 | 0.07 | 1.32 | 0.07 | 0.12 |
| PcOxP_DB03_H07 | 1 | 253 | 1.91 | 0.05 | 1.64 | 0.05 | 0.08 |
| PcOxP_DB03_H08 | 63 | 254 | 0.91 | 0.06 | 1.18 | 0.06 | 0.12 |
| PcOxP_DB03_H09 | 64 | 181 | 0.93 | 0.06 | 1.25 | 0.06 | 0.11 |
| PcOxP_DB04_A07 | 65 | 255 | 1.54 | 0.05 | 2.11 | 0.05 | 0.09 |
| PcOxP_DB04_A09 | 66 | 256 | 1.16 | 0.06 | 1.96 | 0.06 | 0.12 |
| PcOxP_DB04_A10 | 67 | 257 | 0.74 | 0.06 | 1.61 | 0.06 | 0.11 |
| PcOxP_DB04_A12 | 68 | 198 | 0.71 | 0.07 | 1.38 | 0.06 | 0.12 |
| PcOxP_DB04_B06 | 35 | 258 | 0.66 | 0.06 | 1.06 | 0.06 | 0.10 |
| PcOxP_DB04_B08 | 69 | 259 | 1.33 | 0.06 | 1.56 | 0.05 | 0.11 |
| PcOxP_DB04_C03 | 70 | 260 | 1.34 | 0.06 | 1.87 | 0.06 | 0.10 |
| PcOxP_DB04_C08 | 71 | 261 | 0.69 | 0.05 | 1.07 | 0.06 | 0.08 |
| PcOxP_DB04_D08 | 72 | 262 | 1.60 | 0.07 | 1.96 | 0.07 | 0.12 |
| PcOxP_DB04_F06 | 73 | 263 | 0.75 | 0.06 | 1.30 | 0.06 | 0.10 |
| PcOxP_DB04_F11 | 74 | 264 | 0.58 | 0.07 | 1.27 | 0.06 | 0.10 |
| PcOxP_DB04_G06 | 75 | 265 | 1.03 | 0.07 | 1.12 | 0.07 | 0.11 |
| PcOxP_DB04_H03 | 76 | 266 | 0.80 | 0.07 | 1.05 | 0.08 | 0.10 |
| PcOxP_DB04_H07 | 77 | 267 | 0.58 | 0.06 | 1.02 | 0.05 | 0.09 |
| PcOxP_DB04_H08 | 4 | 205 | 0.85 | 0.07 | 1.42 | 0.06 | 0.09 |
| PcOxP_DB04_H10 | 78 | 268 | 0.54 | 0.08 | 1.01 | 0.07 | 0.10 |
| PcOxP_DB01_A03 | 1 | 269 | 0.86 | 0.05 | 1.38 | 0.04 | 0.07 |
| PcOxP_DB01_A04 | 27 | 269 | 0.31 | 0.04 | 0.75 | 0.04 | 0.07 |
| PcOxP_DB01_A05 | 79 | 269 | 0.83 | 0.04 | 1.28 | 0.04 | 0.06 |
| PcOxP_DB01_A08 | 80 | 270 | 0.16 | 0.05 | 1.12 | 0.05 | 0.09 |
| PcOxP_DB01_B02 | 81 | 269 | 0.68 | 0.06 | 1.41 | 0.05 | 0.09 |
| PcOxP_DB01_B03 | 22 | 269 | 0.82 | 0.05 | 1.25 | 0.05 | 0.08 |
| PcOxP_DB01_B06 | 82 | 269 | 0.79 | 0.05 | 1.45 | 0.05 | 0.08 |
| PcOxP_DB01_B10 | 83 | 271 | 0.52 | 0.05 | 1.11 | 0.04 | 0.08 |
| PcOxP_DB01_B12 | 84 | 272 | 0.56 | 0.06 | 1.19 | 0.05 | 0.09 |
| PcOxP_DB01_C07 | 1 | 273 | 0.53 | 0.04 | 1.10 | 0.04 | 0.05 |
| PcOxP_DB01_C09 | 8 | 274 | 0.27 | 0.05 | 0.94 | 0.05 | 0.07 |
| PcOxP_DB01_C10 | 8 | 275 | 0.13 | 0.04 | 0.53 | 0.04 | 0.07 |
| PcOxP_DB01_D01 | 22 | 276 | 0.85 | 0.05 | 1.12 | 0.04 | 0.06 |
| PcOxP_DB01_D04 | 8 | 277 | 0.45 | 0.08 | 0.87 | 0.06 | 0.07 |
| PcOxP_DB01_D06 | 85 | 278 | 0.41 | 0.04 | 0.69 | 0.04 | 0.06 |
| PcOxP_DB01_D07 | 86 | 279 | 0.14 | 0.04 | 0.60 | 0.04 | 0.05 |
| PcOxP_DB01_D10 | 87 | 269 | 0.15 | 0.05 | 0.44 | 0.04 | 0.06 |
| PcOxP_DB01_D11 | 88 | 280 | 1.37 | 0.06 | 2.09 | 0.05 | 0.08 |
| PcOxP_DB01_D12 | 89 | 269 | 0.85 | 0.05 | 1.48 | 0.05 | 0.06 |
| PcOxP_DB01_E01 | 60 | 281 | 0.75 | 0.05 | 1.48 | 0.04 | 0.05 |
| PcOxP_DB01_E02 | 90 | 269 | 0.46 | 0.05 | 1.02 | 0.05 | 0.07 |
| PcOxP_DB01_E03 | 91 | 282 | 0.86 | 0.05 | 1.54 | 0.05 | 0.08 |
| PcOxP_DB01_E07 | 22 | 283 | 0.87 | 0.05 | 1.51 | 0.05 | 0.07 |
| PcOxP_DB01_E08 | 92 | 269 | 0.47 | 0.05 | 0.82 | 0.04 | 0.06 |
| PcOxP_DB01_E09 | 93 | 284 | 0.77 | 0.04 | 1.26 | 0.04 | 0.06 |
| PcOxP_DB01_E10 | 94 | 285 | 0.60 | 0.07 | 1.11 | 0.07 | 0.11 |
| PcOxP_DB01_F01 | 95 | 286 | 0.60 | 0.05 | 1.78 | 0.05 | 0.08 |
| PcOxP_DB01_F03 | 96 | 269 | 0.80 | 0.07 | 1.41 | 0.08 | 0.08 |
| PcOxP_DB01_F06 | 73 | 287 | 0.12 | 0.04 | 0.53 | 0.04 | 0.07 |
| PcOxP_DB01_F07 | 97 | 288 | 0.31 | 0.04 | 0.81 | 0.04 | 0.06 |
| PcOxP_DB01_F08 | 68 | 289 | 0.52 | 0.04 | 1.22 | 0.04 | 0.06 |
| PcOxP_DB01_F11 | 98 | 269 | 1.55 | 0.07 | 1.92 | 0.05 | 0.07 |
| PcOxP_DB01_F12 | 99 | 290 | 0.76 | 0.05 | 1.43 | 0.05 | 0.06 |
| PcOxP_DB01_G02 | 100 | 291 | 0.82 | 0.05 | 1.20 | 0.04 | 0.06 |
| PcOxP_DB01_G03 | 4 | 292 | 0.43 | 0.05 | 0.94 | 0.05 | 0.07 |
| PcOxP_DB01_G04 | 4 | 289 | 0.56 | 0.05 | 1.01 | 0.07 | 0.07 |
| PcOxP_DB01_G05 | 101 | 284 | 0.69 | 0.05 | 1.24 | 0.05 | 0.08 |
| PcOxP_DB01_G06 | 66 | 289 | 0.73 | 0.05 | 1.32 | 0.04 | 0.06 |
| PcOxP_DB01_G07 | 89 | 293 | 0.46 | 0.05 | 1.20 | 0.04 | 0.07 |
| PcOxP_DB01_G10 | 102 | 294 | 0.71 | 0.05 | 1.52 | 0.05 | 0.10 |
| PcOxP_DB01_H01 | 103 | 269 | 0.82 | 0.07 | 1.23 | 0.06 | 0.09 |
| PcOxP_DB01_H02 | 22 | 295 | 0.74 | 0.07 | 1.10 | 0.06 | 0.10 |
| PcOxP_DB01_H03 | 104 | 296 | 0.46 | 0.07 | 1.29 | 0.06 | 0.10 |
| PcOxP_DB01_H06 | 105 | 297 | 0.96 | 0.05 | 1.79 | 0.07 | 0.11 |
| PcOxP_DB01_H07 | 106 | 270 | 0.10 | 0.05 | 0.92 | 0.05 | 0.07 |
| PcOxP_DB01_H09 | 107 | 269 | 0.06 | 0.09 | 0.09 | 0.04 | 0.09 |
| PcOxP_DB02_A04 | 67 | 269 | 0.33 | 0.04 | 0.82 | 0.04 | 0.09 |
| PcOxP_DB02_A05 | 62 | 298 | 0.99 | 0.05 | 1.24 | 0.04 | 0.07 |
| PcOxP_DB02_B04 | 66 | 269 | 0.75 | 0.05 | 1.32 | 0.04 | 0.08 |
| PcOxP_DB02_B07 | 108 | 299 | 0.40 | 0.04 | 0.77 | 0.04 | 0.06 |
| PcOxP_DB02_B09 | 109 | 300 | 0.27 | 0.04 | 0.83 | 0.04 | 0.07 |
| PcOxP_DB02_C03 | 110 | 301 | 0.54 | 0.05 | 0.61 | 0.05 | 0.10 |
| PcOxP_DB02_C04 | 111 | 302 | 0.37 | 0.05 | 0.75 | 0.05 | 0.08 |
| PcOxP_DB02_C05 | 112 | 269 | 0.64 | 0.04 | 0.92 | 0.04 | 0.06 |
| PcOxP_DB02_C08 | 113 | 303 | 0.22 | 0.06 | 0.39 | 0.04 | 0.06 |
| PcOxP_DB02_C10 | 1 | 304 | 0.44 | 0.04 | 0.92 | 0.04 | 0.06 |
| PcOxP_DB02_C12 | 72 | 305 | 0.97 | 0.04 | 1.58 | 0.04 | 0.07 |
| PcOxP_DB02_D02 | 114 | 306 | 0.16 | 0.04 | 0.27 | 0.04 | 0.05 |
| PcOxP_DB02_D03 | 115 | 307 | 0.42 | 0.04 | 0.71 | 0.04 | 0.08 |
| PcOxP_DB02_E02 | 116 | 269 | 0.09 | 0.04 | 0.34 | 0.05 | 0.06 |
| PcOxP_DB02_E03 | 117 | 308 | 0.39 | 0.04 | 0.75 | 0.05 | 0.08 |
| PcOxP_DB02_E08 | 118 | 269 | 0.24 | 0.04 | 0.49 | 0.04 | 0.07 |
| PcOxP_DB02_E09 | 110 | 309 | 0.56 | 0.04 | 0.74 | 0.04 | 0.07 |
| PcOxP_DB02_E11 | 119 | 310 | 0.23 | 0.04 | 0.76 | 0.04 | 0.06 |
| PcOxP_DB02_E12 | 120 | 311 | 0.26 | 0.05 | 0.58 | 0.06 | 0.06 |
| PcOxP_DB02_F04 | 121 | 269 | 0.47 | 0.04 | 0.74 | 0.05 | 0.09 |
| PcOxP_DB02_F06 | 122 | 312 | 0.16 | 0.04 | 0.45 | 0.04 | 0.06 |
| PcOxP_DB02_F09 | 123 | 289 | 0.39 | 0.04 | 0.93 | 0.04 | 0.08 |
| PcOxP_DB02_F10 | 9 | 313 | 0.51 | 0.05 | 1.20 | 0.04 | 0.09 |
| PcOxP_DB02_F11 | 124 | 314 | 0.23 | 0.04 | 0.66 | 0.04 | 0.08 |
| PcOxP_DB02_G02 | 120 | 289 | 1.01 | 0.04 | 1.13 | 0.04 | 0.06 |
| PcOxP_DB02_G03 | 125 | 315 | 0.06 | 0.05 | 0.10 | 0.05 | 0.06 |
| PcOxP_DB02_G04 | 110 | 316 | 0.32 | 0.05 | 0.70 | 0.05 | 0.11 |
| PcOxP_DB02_G05 | 126 | 317 | 1.49 | 0.05 | 1.80 | 0.07 | 0.11 |
| PcOxP_DB02_H02 | 127 | 318 | 0.07 | 0.04 | 0.19 | 0.06 | 0.06 |
| PcOxP_DB02_H04 | 49 | 319 | 0.22 | 0.05 | 0.20 | 0.04 | 0.06 |
| PcOxP_DB02_H05 | 128 | 320 | 0.20 | 0.04 | 0.44 | 0.04 | 0.09 |
| PcOxP_DB02_H06 | 9 | 321 | 0.92 | 0.05 | 1.04 | 0.05 | 0.07 |
| PcOxP_DB02_H07 | 22 | 322 | 0.56 | 0.04 | 0.88 | 0.05 | 0.07 |
| PcOxP_DB02_H08 | 129 | 323 | 0.40 | 0.04 | 1.34 | 0.06 | 0.07 |
| PcOxP_DB02_H09 | 130 | 269 | 0.25 | 0.04 | 0.58 | 0.04 | 0.07 |
| PcOxP_DB03_A04 | 131 | 324 | 0.19 | 0.04 | 0.47 | 0.04 | 0.06 |
| PcOxP_DB03_A05 | 132 | 325 | 0.93 | 0.04 | 0.78 | 0.04 | 0.06 |
| PcOxP_DB03_A06 | 133 | 326 | 0.99 | 0.05 | 1.07 | 0.05 | 0.08 |
| PcOxP_DB03_A07 | 134 | 327 | 0.94 | 0.04 | 1.01 | 0.04 | 0.06 |
| PcOxP_DB03_A10 | 53 | 328 | 0.60 | 0.04 | 0.54 | 0.04 | 0.06 |
| PcOxP_DB03_A12 | 135 | 272 | 2.28 | 0.06 | 1.67 | 0.05 | 0.07 |
| PcOxP_DB03_B01 | 136 | 329 | 0.21 | 0.04 | 0.64 | 0.04 | 0.06 |
| PcOxP_DB03_B03 | 4 | 301 | 1.12 | 0.04 | 1.10 | 0.04 | 0.07 |
| PcOxP_DB03_B06 | 137 | 330 | 0.68 | 0.04 | 1.01 | 0.05 | 0.06 |
| PcOxP_DB03_B07 | 138 | 269 | 0.66 | 0.06 | 0.81 | 0.05 | 0.12 |
| PcOxP_DB03_B08 | 1 | 284 | 2.63 | 0.05 | 1.95 | 0.04 | 0.07 |
| PcOxP_DB03_B09 | 22 | 331 | 1.09 | 0.05 | 0.89 | 0.05 | 0.08 |
| PcOxP_DB03_B10 | 139 | 269 | 0.41 | 0.05 | 0.71 | 0.05 | 0.08 |
| PcOxP_DB03_B11 | 140 | 332 | 0.50 | 0.05 | 0.72 | 0.05 | 0.07 |
| PcOxP_DB03_B12 | 141 | 269 | 1.21 | 0.05 | 0.85 | 0.05 | 0.06 |
| PcOxP_DB03_C01 | 142 | 333 | 0.84 | 0.05 | 1.47 | 0.05 | 0.08 |
| PcOxP_DB03_C04 | 143 | 334 | 2.11 | 0.04 | 1.72 | 0.04 | 0.07 |
| PcOxP_DB03_C08 | 144 | 335 | 0.98 | 0.05 | 0.90 | 0.05 | 0.06 |
| PcOxP_DB03_C10 | 101 | 298 | 1.10 | 0.05 | 1.10 | 0.04 | 0.07 |
| PcOxP_DB03_D02 | 1 | 336 | 0.67 | 0.05 | 1.37 | 0.04 | 0.09 |
| PcOxP_DB03_D03 | 145 | 269 | 0.73 | 0.04 | 1.11 | 0.04 | 0.10 |
| PcOxP_DB03_D10 | 146 | 337 | 1.19 | 0.05 | 1.41 | 0.06 | 0.06 |
| PcOxP_DB03_D12 | 147 | 269 | 0.20 | 0.04 | 0.30 | 0.04 | 0.06 |
| PcOxP_DB03_E03 | 17 | 338 | 0.27 | 0.04 | 0.79 | 0.04 | 0.05 |
| PcOxP_DB03_E04 | 22 | 320 | 0.96 | 0.06 | 1.20 | 0.04 | 0.08 |
| PcOxP_DB03_E11 | 4 | 339 | 1.40 | 0.05 | 1.58 | 0.04 | 0.07 |
| PcOxP_DB03_E12 | 148 | 340 | 0.57 | 0.06 | 0.88 | 0.06 | 0.06 |
| PcOxP_DB03_F02 | 149 | 341 | 1.56 | 0.05 | 1.68 | 0.05 | 0.07 |
| PcOxP_DB03_F03 | 150 | 342 | 1.13 | 0.05 | 1.93 | 0.05 | 0.08 |
| PcOxP_DB03_F05 | 151 | 343 | 0.73 | 0.05 | 1.04 | 0.04 | 0.06 |
| PcOxP_DB03_F07 | 152 | 344 | 0.53 | 0.05 | 1.19 | 0.05 | 0.08 |
| PcOxP_DB03_F09 | 94 | 289 | 0.72 | 0.05 | 1.12 | 0.05 | 0.08 |
| PcOxP_DB03_F11 | 153 | 345 | 1.47 | 0.06 | 1.43 | 0.06 | 0.12 |
| PcOxP_DB03_G01 | 154 | 346 | 0.66 | 0.05 | 1.19 | 0.05 | 0.09 |
| PcOxP_DB03_G09 | 155 | 347 | 0.74 | 0.05 | 1.07 | 0.05 | 0.07 |
| PcOxP_DB03_G11 | 22 | 348 | 0.26 | 0.05 | 0.72 | 0.05 | 0.10 |
| PcOxP_DB03_G12 | 89 | 282 | 1.57 | 0.06 | 1.91 | 0.05 | 0.08 |
| PcOxP_DB03_H02 | 156 | 349 | 1.07 | 0.05 | 1.69 | 0.04 | 0.05 |
| PcOxP_DB03_H10 | 157 | 296 | 0.24 | 0.04 | 0.39 | 0.07 | 0.05 |
| PcOxP_DB04_A04 | 158 | 350 | 1.09 | 0.08 | 1.90 | 0.08 | 0.11 |
| PcOxP_DB04_A05 | 159 | 329 | 0.72 | 0.04 | 1.29 | 0.04 | 0.06 |
| PcOxP_DB04_A11 | 24 | 351 | 0.64 | 0.07 | 1.60 | 0.08 | 0.10 |
| PcOxP_DB04_B01 | 160 | 352 | 0.45 | 0.05 | 0.78 | 0.05 | 0.06 |
| PcOxP_DB04_B07 | 161 | 269 | 0.93 | 0.06 | 1.33 | 0.05 | 0.09 |
| PcOxP_DB04_B10 | 162 | 353 | 1.05 | 0.05 | 1.56 | 0.04 | 0.06 |
| PcOxP_DB04_B11 | 163 | 269 | 0.26 | 0.05 | 0.31 | 0.05 | 0.08 |
| PcOxP_DB04_B12 | 1 | 354 | 0.13 | 0.04 | 0.34 | 0.04 | 0.09 |
| PcOxP_DB04_C09 | 164 | 355 | 0.55 | 0.07 | 1.21 | 0.06 | 0.11 |
| PcOxP_DB04_C10 | 165 | 269 | 0.54 | 0.06 | 1.03 | 0.05 | 0.08 |
| PcOxP_DB04_C11 | 1 | 356 | 0.81 | 0.05 | 1.34 | 0.04 | 0.07 |
| PcOxP_DB04_D01 | 166 | 357 | 1.23 | 0.06 | 1.66 | 0.06 | 0.07 |
| PcOxP_DB04_D05 | 167 | 358 | 0.87 | 0.05 | 1.27 | 0.06 | 0.08 |
| PcOxP_DB04_D07 | 110 | 334 | 1.02 | 0.05 | 1.05 | 0.05 | 0.08 |
| PcOxP_DB04_D09 | 87 | 359 | 0.41 | 0.05 | 0.83 | 0.08 | 0.06 |
| PcOxP_DB04_D10 | 168 | 360 | 1.14 | 0.05 | 1.35 | 0.05 | 0.07 |
| PcOxP_DB04_D12 | 169 | 269 | 0.64 | 0.06 | 1.03 | 0.05 | 0.08 |
| PcOxP_DB04_E05 | 170 | 269 | 0.67 | 0.06 | 1.03 | 0.05 | 0.09 |
| PcOxP_DB04_E06 | 171 | 269 | 1.04 | 0.04 | 1.59 | 0.05 | 0.07 |
| PcOxP_DB04_E08 | 172 | 361 | 1.39 | 0.06 | 2.01 | 0.05 | 0.09 |
| PcOxP_DB04_E12 | 8 | 362 | 1.03 | 0.05 | 1.39 | 0.05 | 0.07 |
| PcOxP_DB04_F01 | 173 | 304 | 0.56 | 0.07 | 0.85 | 0.07 | 0.09 |
| PcOxP_DB04_F03 | 174 | 269 | 0.58 | 0.06 | 0.87 | 0.05 | 0.10 |
| PcOxP_DB04_F05 | 1 | 363 | 0.76 | 0.06 | 0.97 | 0.05 | 0.07 |
| PcOxP_DB04_G01 | 175 | 364 | 0.63 | 0.07 | 1.39 | 0.09 | 0.09 |
| PcOxP_DB04_G03 | 176 | 365 | 0.19 | 0.04 | 0.40 | 0.05 | 0.07 |
| PcOxP_DB04_G05 | 177 | 269 | 1.20 | 0.05 | 1.67 | 0.05 | 0.07 |
| PcOxP_DB04_G08 | 178 | 366 | 1.39 | 0.05 | 1.67 | 0.05 | 0.07 |
| PcOxP_DB04_G09 | 179 | 269 | 0.48 | 0.05 | 1.08 | 0.06 | 0.08 |
| PcOxP_DB04_G11 | 180 | 367 | 0.68 | 0.05 | 0.79 | 0.05 | 0.07 |
| PcOxP_DB04_H01 | 89 | 368 | 1.22 | 0.06 | 1.80 | 0.07 | 0.07 |

### Example 3: IgG ELISA Summary

48 scFv were selected and reformatted along with the parental E06 antibody into human IgG1 using standard techniques. For ELISA analysis of antibody binding, 384-well polystyrene microtitre plates were coated with 20 μL of antigen (PC-BSA at 5 μg/mL) in PBS for 2 hours at room temperature. Plates were washed three times with DELFIA® wash buffer, then blocked with 20 μL/well 1% BSA-TBS for 1 hour at room temperature. After washing, 15 μL of the titrated antibody in 1% BSA-TBS was added to wells and incubated for 1 hour at room temperature. After further washing, 15 μL/well of DELFIA® Eu-N1 Anti-Human IgG secondary antibody in TBS was added to wells and incubated for 1 hour at room temperature. Plates were washed again, and the amount of antibody binding to each well was quantitated from TRF measurement following 5 min incubation at room temperature with DELFIA® enhancement buffer. Binding data of the parental E06 IgG1 antibody and various IgG1 binders are summarized in Table 5. No binding to BSA was detected for any of the antibodies shown in Table 5.

Physical properties (melting temperature, aggregation temperature, and polydispersity index) of the antibodies were determined using an Uncle instrument (Unchained Labs). Physical properties of the parental E06 IgG1 antibody and various IgG1 binders are summarized in Table 5.

TABLE 5
IgG ELISA Data.

| | EC50 | Physical Properties | | |
|---|---|---|---|---|
| | (nM) | Melting | Aggregation | Poly- |
| | PC | Temp. | Temp. | dispersity |
| Antibody | BSA | Tm (° C.) | Tagg (° C.) | Index (PDI) |
| E06 parental IgG1 | 0.390 | 69.5 | 66.3 | 0.07 |
| PcOxP_DB01_G05 | 0.263 | 71.5 | 71.65 | 0.246 |
| PcOxP_DB01_G09 | 0.308 | 72.5 | 73.44 | 0.058 |
| PcOxP_DB01_H01 | 0.275 | 71.58 | 74.57 | 0.093 |
| PcOxP_DB03_B11 | 0.299 | 71.24 | 74.52 | 0.104 |
| PcOxP_DB03_C10 | 0.285 | 72 | 73.29 | 0.167 |
| PcOxP_DB03_H08 | 0.445 | 68.7 | 69.56 | 0.182 |
| PcOxP_DB04_A11 | 0.413 | 71.5 | 72.03 | 0.097 |
| PcOxP_DB04_E05 | 0.282 | 70.53 | 72.22 | 0.126 |
| PcOxP_DB01_A06 | 0.43 | 71.21 | 72.53 | 0.197 |
| PcOxP_DB01_B09 | 0.317 | 71.5 | 74.55 | 0.036 |
| PcOxP_DB01_C04 | 0.310 | 33.5 | 72.81 | 0.11 |
| PcOxP_DB01_D01 | 0.274 | 70.5 | 70.73 | 0.029 |
| PcOxP_DB01_D11 | 0.242 | 69.14 | 69.67 | 0.124 |
| PcOxP_DB01_E05 | 0.417 | 71.5 | 73.26 | 0.106 |
| PcOxP_DB01_E10 | 0.316 | 72.2 | 74.66 | 0.172 |
| PcOxP_DB01_F01 | 0.672 | 73 | 72.68 | 0.347 |
| PcOxP_DB01_G10 | 0.407 | 71.5 | 72.96 | 0.027 |
| PcOxP_DB01_H02 | 0.43 | 72.12 | 74.88 | 0.304 |
| PcOxP_DB01_H04 | 0.456 | 72 | 72.97 | 0.436 |
| PcOxP_DB02_A06 | 0.643 | 70 | 70.54 | 0.235 |
| PcOxP_DB02_C01 | 0.64 | 71.5 | 72.51 | 0.207 |
| PcOxP_DB02_H07 | 0.423 | 71.74 | 74.2 | 0.192 |
| PcOxP_DB03_A09 | 0.664 | 70.5 | 72.92 | 0.095 |
| PcOxP_DB03_B09 | 0.426 | 71 | 73.2 | 0.047 |
| PcOxP_DB03_C03 | 0.339 | 70.22 | 73.18 | 0.231 |
| PcOxP_DB03_C12 | 0.436 | 70 | 73.79 | 0.098 |
| PcOxP_DB03_D08 | 0.401 | 70.23 | 71.27 | 0.149 |
| PcOxP_DB03_E01 | 0.857 | 72.01 | 73.07 | 0.166 |
| PcOxP_DB03_G07 | 0.636 | 73.06 | 74.2 | 0.223 |
| PcOxP_DB03_G12 | 0.557 | 72.74 | 73.67 | 0.06 |
| PcOxP_DB04_A09 | 0.269 | 72.5 | 72.15 | 0.023 |
| PcOxP_DB04_E08 | 0.251 | 72.73 | 76.24 | 0.167 |
| PcOxP_DB01_F06 | 0.216 | 72.18 | 71.28 | 0.219 |
| PcOxP_DB02_F10 | 0.659 | 72 | 73.59 | 0.12 |
| PcOxP_DB02_G04 | 0.231 | 70 | 67.12 | 0.143 |
| PcOxP_DB04_G01 | 0.257 | 72.5 | 74.34 | 0.288 |
| PcOxP_DB01_B06 | 0.219 | 72.6 | 74.28 | 0.437 |
| PcOxP_DB01_F04 | 0.344 | 71 | 70.95 | 0.184 |
| PcOxP_DB01_F11 | 0.244 | 71.18 | 76.16 | 0.074 |
| PcOxP_DB01_H08 | 0.244 | 70.51 | 70.08 | 0.092 |
| PcOxP_DB02_A05 | 0.264 | 71.5 | 73.63 | 0.25 |
| PcOxP_DB02_B04 | 0.278 | 71.58 | 76.93 | 0.078 |
| PcOxP_DB03_F09 | 0.28 | 72.62 | 73.62 | 0.129 |
| PcOxP_DB03_G03 | 0.216 | 58.52 | 73.65 | 0.22 |
| PcOxP_DB03_G11 | 0.244 | 73.06 | 74.11 | 0.217 |
| PcOxP_DB03_H03 | 0.229 | 70.5 | 70.1 | 0.156 |
| PcOxP_DB04_A04 | 0.294 | 74 | 76.96 | 0.134 |
| PcOxP_DB02_C09 | 0.394 | 72 | 68.48 | 0.265 |

### Example 4: Further IgG Characterization

Select human IgG1 antibodies (see Example 3) were further analyzed for antibody yield, stress testing, and immunogenicity characteristics. Yield was determined by transfection of HEK293 cells and subsequent expression in 25 mL cultures. For several of the antibodies, yield was determined in a scaled-up 40 L transient transfection culture. Antibody products were purified using protein A, and the resulting amount of antibody was measured via absorbance at 280 nm (OD280). The yield values for the parental E06 IgG1 antibody and select IgG1 binders are shown in Table 6.

TABLE 6
Yield Data.

| | Yield − 25 mL | Yield − 40 L |
|---|---|---|
| Antibody | culture (mg) | culture (mg/L) |
| E06 parental IgG1 | — | 15 |
| PcOxP_DB01_H08 | 3.3 | 649 |
| PcOxP_DB03_C03 | 3.4 | 392 |
| PcOxP_DB01_F11 | 8.5 | |
| PcOxP_DB01_B09 | 5.2 | |
| PcOxP_DB01_G09 | 4.8 | |
| PcOxP_DB03_C12 | 3.1 | |
| PcOxP_DB01_E05 | 5.1 | |

The parental E06 IgG1 antibody and the human IgG1 PcOxP_DB01_H08 antibody were analyzed in forced degradation studies to compare the relative stability and resistance to stress between the antibodies. Samples of each antibody were subjected to temperature stress, oxidation stress, and low and high pH stress, and the samples were analyzed and compared to non-stressed samples. Various analytical techniques were used to analyze the samples. In addition to concentration measurement and visual inspection, chemical stability was assessed via protein level RPC-MS at the intact and reduced antibody level, peptide mapping analysis under reducing conditions, iCIEF analysis, and CGE-SDS in non-reducing and reducing conditions, and physical stability was assessed using SE-HPLC.

For temperature stress, samples of each antibody were stored at 40° C. for 2 weeks, 3 weeks, or 4 weeks. For low pH stress, the pH in samples of each antibody was adjusted to 3.2 using 0.12M HCl, and the samples were incubated at 25° C. for 2 hours prior to buffer exchange back to formulation buffer. For high pH stress (forced deamidation), the pH in samples of each antibody was adjusted to 9.0 using 0.5 M Tris pH 11, and the samples were incubated for 3 days at 37° C. prior to buffer exchange back to formulation buffer. For oxidative stress, 3% H₂O₂ solution was added to samples of each antibody to reach a final concentration of 0.03%, and the samples were incubated for 1 day at 25° C. prior to buffer exchange back to formulation buffer. The stress testing and stability assay results are summarized qualitatively in Table 7 (“+”: moderately stable; “++”: highly stable).

TABLE 7
Stress Testing and Stability Comparison.

| | Storage | Low | Forced | Oxidative |
|---|---|---|---|---|
| Antibody | at 40° C. | pH stress | deamidation | stress |
| E06 parental IgG1 | + | ++ | + | ++ |
| PcOxP_DB01_H08 | ++ | ++ | ++ | ++ |

In vivo immunogenicity for the parental E06 IgG1 antibody and the human IgG1 PcOxP_DB01_H08 and PcOxP_DB03_C03 antibodies was predicted using the in vitro Epibase® immunogenicity assay (Lonza Biologics), the in silico Epibase® platform (Lonza Biologics) and the in silico ISPRI™ platform (EpiVax, Inc.). The immunogenicity profiles are summarized in Table 8. Overall, the immunogenicity assays show that PcOxP_DB01_H08 and PcOxP_DB03_C03 each have a low immunogenicity risk and a favorable profile when compared with several established antibody drugs.

TABLE 8
Immunogenicity Assay Data.

| | In vitro | In silico | In silico |
|---|---|---|---|
| | Epibase ® | Epibase ® | ISPRI ™ |
| Antibody | (% responders)* | score | score |
| E06 parental IgG1 | 17 | 2203 | +7.44 |
| PcOxP_DB01_H08 | 16 | 906 | −50.09 |
| PcOxP_DB03_C03 | 22 | 1048 | |

*32 donor samples, KLH positive control shows 100% effect
**a score of 0 indicates predicted immunogenic responses in approximately 5% of patients

SEQUENCE LISTING

| SEQ ID | |
|---|---|
| NO | SEQUENCE |
| 1 | EVQLLESGGGLVQPGGSLRLSCAASGFTFSDYYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 2 | EVQLLESGGGLVQPGGSLRLSCAASGFGFSDFYMEWVRQAPGKGLEWVAASRNKWNDYTTEY |
| | ADSVKGRFTISRDNSKNTLYLQMNSLRAEDTAVYYCARDYYGSSYWYYDVWGQGTLVTVSS |
| 3 | EVQLLESGGGLVQPGGSLRLSCAASGFTFSDQYIEWVRQAPGKGLEWVAASRNKANDYTTEY |
| | ADSVKGRFTISRDNSKNTLYLQMNSLRAEDTAVYYCARDYLGSSYWYFDVWGQGTLVTVSS |
| 4 | EVQLLESGGGLVQPGGSLRLSCAASGFTFSDGYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 5 | EVQLLESGGGLVQPGGSLRLSCAASGFTASDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 6 | EVQLLESGGGLVQPGGSLRLSCAASGFTFSDSYMEWVRQAPGKGLEWVAASRNKANDYTTSY |
| | ADSVKGRFTISRDNSKNTLYLQMNSLRAEDTAVYYCARDYYGSSYWYFDVWGQGTLVTVSS |
| 7 | EVQLLESGGGLVQPGGSLRLSCAASGFTFEDFYMEWVRQAPGKGLEWVAASRNKANDYTTEY |
| | ADSVKGRFTISRDNSKNTLYLQMNSLRAEDTAVYYCARDYFGSSYWYFDVWGQGTLVTVSS |
| 8 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 9 | EVQLLESGGGLVQPGGSLRLSCAASGFTFSDQYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 10 | EVQLLESGGGLVQPGGSLRLSCAASGFQFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 11 | EVQLLESGGGLVQPGGSLRLSCAASGFEESDNYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 12 | EVQLLESGGGLVQPGGSLRLSCAASGFTFSDGYMEWVRQAPGKGLEWVAASRNKANDYTTEY |
| | ADSVKGRFTISRDNSKNTLYLQMNSLRAEDTAVYYCARDYYASSYWYFDVWGQGTLVTVSS |
| 13 | EVQLLESGGGLVQPGGSLRLSCAASGFTFSDFYMEWVRQAPGKGLEWVAASRNKANDYTTEL |
| | ADSVKGRFTISRDNSKNTLYLQMNSLRAEDTAVYYCARDYYGSSYWYFDVWGQGTLVTVSS |
| 14 | EVQLLESGGGLVQPGGSLRLSCAASGFEFSDFYMEWVRQAPGKGLEWVAASRGKGNSYTTEY |
| | ADSVKGRFTISRDNSKNTLYLQMNSLRAEDTAVYYCARDYYGSSYWYFDVWGQGTLVTVSS |
| 15 | EVQLLESGGGLVQPGGSLRLSCAASGFTFSDGYMEWVRQAPGKGLEWVAASRNKANDYTTEY |
| | ADSVKGRFTISRDNSKNTLYLQMNSLRAEDTAVYYCARDYYGSPYWYFDVWGQGTLVTVSS |
| 16 | EVQLLESGGGLVQPGGSLRLSCAASGFTFSDAYMEWVRQAPGKGLEWVAASRNKANDYTTEY |
| | ADSVKGRFTISRDNSKNTLYLQMNSLRAEDTAVYYCARDYYGSSYWYFDSWGQGTLVTVSS |
| 17 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 18 | EVQLLESGGGLVQPGGSLRLSCAASGFTFSDAYMEWVRQAPGKGLEWVAASRNKANDYTTEY |
| | ADSVKGRFTISRDNSKNTLYLQMNSLRAEDTAVYYCARDYYGSSYWYFDTWGQGTLVTVSS |
| 19 | EVQLLESGGGLVQPGGSLRLSCAASGFTFSDQYMEWVRQAPGKGLEWVAASRNKANDYTTEY |
| | ADSVKGRFTISRDNSKNTLYLQMNSLRAEDTAVYYCARDYYGSSYWYFDLWGQGTLVTVSS |
| 20 | EVQLLESGGGLVQPGGSLRLSCAASGFTFSDFYMEWVRQAPGKGLEWVAASRNKANDYVTEY |
| | ADSVKGRFTISRDNSKNTLYLQMNSLRAEDTAVYYCARDYYGSSYWYFDVWGQGTLVTVSS |
| 21 | EVQLLESGGGLVQPGGSLRLSCAASGFTFSSYYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 22 | EVQLLESGGGLVQPGGSLRLSCAASGFTFSSYYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 23 | EVQLLESGGGLVQPGGSLRLSCAASGFTGSDFYMEWVRQAPGKGLEWVAASRNKGNSYTTEY |
| | ADSVKGRFTISRDNSKNTLYLQMNSLRAEDTAVYYCARDYYGSSYWYFDVWGQGTLVTVSS |
| 24 | EVQLLESGGGLVQPGGSLRLSCAASGFTFSDYYMEWVRQAPGKGLEWVAASRNKANTYTTEY |
| | ADSVKGRFTISRDNSKNTLYLQMNSLRAEDTAVYYCARDYYGSSYWYFDVWGQGTLVTVSS |
| 25 | EVQLLESGGGLVQPGGSLRLSCAASGFTFSDGYMEWVRQAPGKGLEWVAASRNKNNDYTTEY |
| | ADSVKGRFTISRDNSKNTLYLQMNSLRAEDTAVYYCARDYFGSSYWYFDVWGQGTLVTVSS |
| 26 | EVQLLESGGGLVQPGGSLRLSCAASGFAFSDFYMEWVRQAPGKGLEWVAASRNKANDYTTEY |
| | ADSVKGRFTISRDNSKNTLYLQMNSLRAEDTAVYYCARDYYGSHYWYFDVWGQGTLVTVSS |
| 27 | EVQLLESGGGLVQPGGSLRLSCAASGFTHSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 28 | EVQLLESGGGLVQPGGSLRLSCAASGFTFSDGYMEWVRQAPGKGLEWVAASRNKNNDYTTEY |
| | ADSVKGRFTISRDNSKNTLYLQMNSLRAEDTAVYYCARDYYGSSYWYFDVWGQGTLVTVSS |
| 29 | EVQLLESGGGLVQPGGSLRLSCAASGFTFSDTYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 30 | EVQLLESGGGLVQPGGSLRLSCAASGFAFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 31 | EVQLLESGGGLVQPGGSLRLSCAASGFTFSDGYMEWVRQAPGKGLEWVAASRNIANDYTTEY |
| | ADSVKGRFTISRDNSKNTLYLQMNSLRAEDTAVYYCARDYYGSSYWYFDVWGQGTLVTVSS |
| 32 | EVQLLESGGGLVQPGGSLRLSCAASGFTFSDDYMEWVRQAPGKGLEWVAASRNKANDYTTEY |
| | ADSVKGRFTISRDNSKNTLYLQMNSLRAEDTAVYYCARDYQGSSYWYFDVWGQGTLVTVSS |
| 33 | EVQLLESGGGLVQPGGSLRLSCAASGFTFSDYYMEWVRQAPGKGLEWVAASRNSGNDYTTEY |
| | ADSVKGRFTISRDNSKNTLYLQMNSLRAEDTAVYYCARDYYGSSYWYFDVWGQGTLVTVSS |
| 34 | EVQLLESGGGLVQPGGSLRLSCAASGFTFSDNYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 35 | EVQLLESGGGLVQPGGSLRLSCAASGFTFSDAYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 36 | EVQLLESGGGLVQPGGSLRLSCAASGFTFSDFYMEWVRQAPGKGLEWVAASRNKANDYTVEY |
| | ADSVKGRFTISRDNSKNTLYLQMNSLRAEDTAVYYCARDYYGHSYWYFDVWGQGTLVTVSS |
| 37 | EVQLLESGGGLVQPGGSLRLSCAASGFTSSDFYMEWVRQAPGKGLEWVAASRNKANDYYTEY |
| | ADSVKGRFTISRDNSKNTLYLQMNSLRAEDTAVYYCARDYYGSSYWYFDVWGQGTLVTVSS |
| 38 | EVQLLESGGGLVQPGGSLRLSCAASGFGFSDFYMEWVRQAPGKGLEWVAASRHKANDYTTEY |
| | ADSVKGRFTISRDNSKNTLYLQMNSLRAEDTAVYYCARDYYGSSYWYFDVWGQGTLVTVSS |
| 39 | EVQLLESGGGLVQPGGSLRLSCAASGFTFSTFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 40 | EVQLLESGGGLVQPGGSLRLSCAASGFTFSDEYMEWVRQAPGKGLEWVAASRGKGNSYTTEY |
| | ADSVKGRFTISRDNSKNTLYLQMNSLRAEDTAVYYCARDYYGSSYWYFDVWGQGTLVTVSS |
| 41 | EVQLLESGGGLVQPGGSLRLSCAASGFTFSDQYMEWVRQAPGKGLEWVAASRNKANDYQTEY |
| | ADSVKGRFTISRDNSKNTLYLQMNSLRAEDTAVYYCARDYYGSSYWYFDVWGQGTLVTVSS |
| 42 | EVQLLESGGGLVQPGGSLRLSCAASGFWFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 43 | EVQLLESGGGLVQPGGSLRLSCAASGFTFSSYYMEWVRQAPGKGLEWVAASRNQANDYTTEY |
| | ADSVKGRFTISRDNSKNTLYLQMNSLRAEDTAVYYCARDYYGSPYWYFDVWGQGTLVTVSS |
| 44 | EVQLLESGGGLVQPGGSLRLSCAASGFTFSDVYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 45 | EVQLLESGGGLVQPGGSLRLSCAASGFTFSSYYMEWVRQAPGKGLEWVAASRNQANDYTTEY |
| | ADSVKGRFTISRDNSKNTLYLQMNSLRAEDTAVYYCARDYYGSSYWYLDVWGQGTLVTVSS |
| 46 | EVQLLESGGGLVQPGGSLRLSCAASGFTFSDFYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 47 | EVQLLESGGGLVQPGGSLRLSCAASGATFSDFYMEWVRQAPGKGLEWVAASRNGANDYTTEY |
| | ADSVKGRFTISRDNSKNTLYLQMNSLRAEDTAVYYCARDYYGNSYWYFDVWGQGTLVTVSS |
| 48 | EVQLLESGGGLVQPGGSLRLSCAASGFDFSDFYMEWVRQAPGKGLEWVAASRNKANDYTTDY |
| | ADSVKGRETISRDNSKNTLYLQMNSLRAEDTAVYYCARDYFGSSYWYFDVWGQGTLVTVSS |
| 49 | EVQLLESGGGLVQPGGSLRLSCAASGFTFSDEYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 50 | EVQLLESGGGLVQPGGSLRLSCAASGFWFSDFYMEWVRQAPGKGLEWVAASRNKANDYTTEY |
| | ADSVKGRFTISRDNSKNTLYLQMNSLRAEDTAVYYCARDYYGSAYWYFDVWGQGTLVTVSS |
| 51 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGHSYWYFDVWGQGTLVTVSS |
| 52 | EVQLLESGGGLVQPGGSLRLSCAASGFTFSDYYMEWVRQAPGKGLEWVAASRNKANDYTVEY |
| | ADSVKGRFTISRDNSKNTLYLQMNSLRAEDTAVYYCARDYYGSSYWYFDVWGQGTLVTVSS |
| 53 | EVQLLESGGGLVQPGGSLRLSCAASGWTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 54 | EVQLLESGGGLVQPGGSLRLSCAASGFTFSDNYMEWVRQAPGKGLEWVAASRNKANDYTNEY |
| | ADSVKGRFTISRDNSKNTLYLQMNSLRAEDTAVYYCARDYYGSSYWYFDVWGQGTLVTVSS |
| 55 | EVQLLESGGGLVQPGGSLRLSCAASGFTFSSFYMEWVRQAPGKGLEWVAASRNAANDYTTEY |
| | ADSVKGRFTISRDNSKNTLYLQMNSLRAEDTAVYYCARDYYGSSYWYFDLWGQGTLVTVSS |
| 56 | EVQLLESGGGLVQPGGSLRLSCAASGFTASDFYMEWVRQAPGKGLEWVAASRNKANDQTTEY |
| | ADSVKGRFTISRDNSKNTLYLQMNSLRAEDTAVYYCARDYYGSSYWYFDSWGQGTLVTVSS |
| 57 | EVQLLESGGGLVQPGGSLRLSCAASGFTFNDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 58 | EVQLLESGGGLVQPGGSLRLSCAASGFTFSDHYMEWVRQAPGKGLEWVAASRNSGNSYTTEY |
| | ADSVKGRFTISRDNSKNTLYLQMNSLRAEDTAVYYCARDYYGSSYWYFDVWGQGTLVTVSS |
| 59 | EVQLLESGGGLVQPGGSLRLSCAASGFTFSDGYIEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 60 | EVQLLESGGGLVQPGGSLRLSCAASGFTSSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 61 | EVQLLESGGGLVQPGGSLRLSCAASGFTFSDHYMEWVRQAPGKGLEWVAASRNKANDYTTEY |
| | ADSVKGRFTISRDNSKNTLYLQMNSLRAEDTAVYYCARDYYGQSYWYFDVWGQGTLVTVSS |
| 62 | EVQLLESGGGLVQPGGSLRLSCAASGFTGSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 63 | EVQLLESGGGLVQPGGSLRLSCAASGGTFSDFYMEWVRQAPGKGLEWVAASRNKANTYTTEY |
| | ADSVKGRFTISRDNSKNTLYLQMNSLRAEDTAVYYCARDYYGSSYWYFDVWGQGTLVTVSS |
| 64 | EVQLLESGGGLVQPGGSLRLSCAASGFTFSDAYMEWVRQAPGKGLEWVAASRNKANSYTTEY |
| | ADSVKGRFTISRDNSKNTLYLQMNSLRAEDTAVYYCARDYYGSSYWYFDVWGQGTLVTVSS |
| 65 | EVQLLESGGGLVQPGGSLRLSCAASGFTQSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 66 | EVQLLESGGGLVQPGGSLRLSCAASGFTFSDYYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 67 | EVQLLESGGGLVQPGGSLRLSCAASGDTFSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 68 | EVQLLESGGGLVQPGGSLRLSCAASGFTFSDGYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 69 | EVQLLESGGGLVQPGGSLRLSCAASGFTFSDDYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 70 | EVQLLESGGGLVQPGGSLRLSCAASGFTTSDFYMEWVRQAPGKGLEWVAASRNKANSYTTEY |
| | ADSVKGRFTISRDNSKNTLYLQMNSLRAEDTAVYYCARDYYGSSYWYFDVWGQGTLVTVSS |
| 71 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 72 | EVQLLESGGGLVQPGGSLRLSCAASGFTFSDHYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 73 | EVQLLESGGGLVQPGGSLRLSCAASGDTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 74 | EVQLLESGGGLVQPGGSLRLSCAASGFTGSDFYMEWVRQAPGKGLEWVAASRGKGNSYTTEY |
| | ADSVKGRFTISRDNSKNTLYLQMNSLRAEDTAVYYCARDYYGSSTWYFDVWGQGTLVTVSS |
| 75 | EVQLLESGGGLVQPGGSLRLSCAASGFTFSDFYMEWVRQAPGKGLEWVAASRWKANDYTTEY |
| | ADSVKGRFTISRDNSKNTLYLQMNSLRAEDTAVYYCARDYYESSYWYFDVWGQGTLVTVSS |
| 76 | EVQLLESGGGLVQPGGSLRLSCAASGEPFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 77 | EVQLLESGGGLVQPGGSLRLSCAASGFTFEDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 78 | EVQLLESGGGLVQPGGSLRLSCAASGATFSDFYVEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 79 | EVQLLESGGGLVQPGGSLRLSCAASGFTFSDYYMEWVRQAPGKGLEWVAASRNKANDYTTEY |
| | ADSVKGRFTISRDNSKNTLYLQMNSLRAEDTAVYYCARDYYGNSYWYFDVWGQGTLVTVSS |
| 80 | EVQLLESGGGLVQPGGSLRLSCAASGFTQSDFYMEWVRQAPGKGLEWVAAIRNKANSYTTEY |
| | ADSVKGRFTISRDNSKNTLYLQMNSLRAEDTAVYYCARDYYGSSYWYFDVWGQGTLVTVSS |
| 81 | EVQLLESGGGLVQPGGSLRLSCAASGGTFSDFYMEWVRQAPGKGLEWVAASRNKANSYTTAY |
| | ADSVKGRFTISRDNSKNTLYLQMNSLRAEDTAVYYCARDYYGSSYWYFDVWGQGTLVTVSS |
| 82 | EVQLLESGGGLVQPGGSLRLSCAASGFTFSSYYMEWVRQAPGKGLEWVAASRNKYNDYTTEY |
| | ADSVKGRFTISRDNSKNTLYLQMNSLRAEDTAVYYCARDYYGSSTWYFDVWGQGTLVTVSS |
| 83 | EVQLLESGGGLVQPGGSLRLSCAASGFTFSDFYMEWVRQAPGKGLEWVAASRNKANDYTTTY |
| | ADSVKGRFTISRDNSKNTLYLQMNSLRAEDTAVYYCARDYYGSSHWYFDVWGQGTLVTVSS |
| 84 | EVQLLESGGGLVQPGGSLRLSCAASGFTFSSYYMEWVRQAPGKGLEWVAASRNKGNSYTTEY |
| | ADSVKGRFTISRDNSKNTLYLQMNSLRAEDTAVYYCARDYYGSSYWYFDVWGQGTLVTVSS |
| 85 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYFDSWGQGTLVTVSS |
| 86 | EVQLLESGGGLVQPGGSLRLSCAASGFTFSDYYMEWVRQAPGKGLEWVAASRNKANDYTTEY |
| | ADSVKGRFTISRDNSKNTLYLQMNSLRAEDTAVYYCARDYYGDSYWYFDVWGQGTLVTVSS |
| 87 | EVQLLESGGGLVQPGGSLRLSCAASGFTDSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 88 | EVQLLESGGGLVQPGGSLRLSCAASGFTFSSYYMEWVRQAPGKGLEWVAASRNKANDYTTEY |
| | ADSVKGRFTISRDNSKNTLYLQMNSLRAEDTAVYYCARDYYGSSYWYNDVWGQGTLVTVSS |
| 89 | EVQLLESGGGLVQPGGSLRLSCAASGFTFSDYYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 90 | EVQLLESGGGLVQPGGSLRLSCAASGFTFSDYYMEWVRQAPGKGLEWVAASRNKGNDYTTEY |
| | ADSVKGRFTISRDNSKNTLYLQMNSLRAEDTAVYYCARDYYGSSYWYFDVWGQGTLVTVSS |
| 91 | EVQLLESGGGLVQPGGSLRLSCAASGFGFSDFYMEWVRQAPGKGLEWVGASRNKHNDYTTEY |
| | ADSVKGRFTISRDNSKNTLYLQMNSLRAEDTAVYYCARDYYGSSYWYFDVWGQGTLVTVSS |
| 92 | EVQLLESGGGLVQPGGSLRLSCAASGDTFSDFYMEWVRQAPGKGLEWVAASRNKANAYTTEY |
| | ADSVKGRFTISRDNSKNTLYLQMNSLRAEDTAVYYCARDYYGQSYWYFDVWGQGTLVTVSS |
| 93 | EVQLLESGGGLVQPGGSLRLSCAASGNTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 94 | EVQLLESGGGLVQPGGSLRLSCAASGFTFSSYYMEWVRQAPGKGLEWVAASRNKENDYTTEY |
| | ADSVKGRFTISRDNSKNTLYLQMNSLRAEDTAVYYCARDYYGSSYWYFDVWGQGTLVTVSS |
| 95 | EVQLLESGGGLVQPGGSLRLSCAASGFPFSDFYMEWVRQAPGKGLEWVAASRNKANDYTTEY |
| | ADSVKGRFTISRDNSKNTLYLQMNSLRAEDTAVYYCARDYYGSPYWYFDVWGQGTLVTVSS |
| 96 | EVQLLESGGGLVQPGGSLRLSCAASGFTFSDSYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 97 | EVQLLESGGGLVQPGGSLRLSCAASGDTFSDFYLEWVRQAPGKGLEWVAASRNKANGYTTEY |
| | ADSVKGRFTISRDNSKNTLYLQMNSLRAEDTAVYYCARDYYHSSYWYFDVWGQGTLVTVSS |
| 98 | EVQLLESGGGLVQPGGSLRLSCAASGFTFSSYYMEWVRQAPGKGLEWVAASRNKANDYTTEY |
| | ADSVKGRFTISRDNSKNTLYLQMNSLRAEDTAVYYCARDYYGSSYWYYDVWGQGTLVTVSS |
| 99 | EVQLLESGGGLVQPGGSLRLSCAASGFTHSDFYMEWVRQAPGKGLEWVAASRNKANGYTTEY |
| | ADSVKGRFTISRDNSKNTLYLQMNSLRAEDTAVYYCARDYYGSSYWYFDSWGQGTLVTVSS |
| 100 | EVQLLESGGGLVQPGGSLRLSCAASGFLFSDFYMEWVRQAPGKGLEWVAASRNKANDYTTTY |
| | ADSVKGRFTISRDNSKNTLYLQMNSLRAEDTAVYYCARDYYGSHYWYFDVWGQGTLVTVSS |
| 101 | EVQLLESGGGLVQPGGSLRLSCAASGFTFSSYYMEWVRQAPGKGLEWVAASRNKANEYTTEY |
| | ADSVKGRFTISRDNSKNTLYLQMNSLRAEDTAVYYCARDYYGSSYWYFDVWGQGTLVTVSS |
| 102 | EVQLLESGGGLVQPGGSLRLSCAASGFTFSDFYMEWVRQAPGKGLEWVAASRNKNNDYTTEY |
| | ADSVKGRFTISRDNSKNTLYLQMNSLRAEDTAVYYCARDYYGSSYWYFDVWGQGTLVTVSS |
| 103 | EVQLLESGGGLVQPGGSLRLSCAASGFTFSSYYMEWVRQAPGKGLEWVAASRNKANLYTTEY |
| | ADSVKGRFTISRDNSKNTLYLQMNSLRAEDTAVYYCARDYYGSSTWYFDVWGQGTLVTVSS |
| 104 | EVQLLESGGGLVQPGGSLRLSCAASGFTFSDHYMEWVRQAPGKGLEWVAASRGKGNSYTTAY |
| | ADSVKGRFTISRDNSKNTLYLQMNSLRAEDTAVYYCARDYYGSSYWYFDVWGQGTLVTVSS |
| 105 | EVQLLESGGGLVQPGGSLRLSCAASGFTFSDFYMEWVRQAPGKGLEWVAASRNKGNSYTTAY |
| | ADSVKGRFTISRDNSKNTLYLQMNSLRAEDTAVYYCARDYYGSSYWYFDVWGQGTLVTVSS |
| 106 | EVQLLESGGGLVQPGGSLRLSCAASGFTFSDHYMEWVRQAPGKGLEWVAASRNKYNDYTTEY |
| | ADSVKGRFTISRDNSKNTLYLQMNSLRAEDTAVYYCARDIYGSSYWYFDVWGQGTLVTVSS |
| 107 | EVQLLESGGGLVQPGGSLRLSCAASGFTFSDYYMEWVRQAPGKGLEWVAASRNKANTYTTEY |
| | ADSVKGRFTISRDNSKNTLYLQMNSLRAEDTAVYYCARDYYGSSYWYDDVWGQGTLVTVSS |
| 108 | EVQLLESGGGLVQPGGSLRLSCAASGFTFSDQYMEWVRQAPGKGLEWVAASRNNANDYTTEY |
| | ADSVKGRFTISRDNSKNTLYLQMNSLRAEDTAVYYCARDYYGSSYWYFDVWGQGTLVTVSS |
| 109 | EVQLLESGGGLVQPGGSLRLSCAASGFTNSDFYMEWVRQAPGKGLEWVAASRNKENDYTTEY |
| | ADSVKGRFTISRDNSKNTLYLQMNSLRAEDTAVYYCARDYYGSSYWYFDVWGQGTLVTVSS |
| 110 | EVQLLESGGGLVQPGGSLRLSCAASGFTFSDYYMEWVRQAPGKGLEWVAASRWKANDYTTEY |
| | ADSVKGRFTISRDNSKNTLYLQMNSLRAEDTAVYYCARDYYGSSYWYFDVWGQGTLVTVSS |
| 111 | EVQLLESGGGLVQPGGSLRLSCAASGQTFSDFYMEWVRQAPGKGLEWVAASRNKANGYTTEY |
| | TDSVKGRFTISRDNSKNTLYLQMNSLRAEDTAVYYCARDYYGSSYWYFDVWGQGTLVTVSS |
| 112 | EVQLLESGGGLVQPGGSLRLSCAASGFTASDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 113 | EVQLLESGGGLVQPGGSLRLSCAASGFTFSDPYMEWVRQAPGKGLEWVAASRNKANDYTTEY |
| | ADSVKGRFTISRDNSKNTLYLQMNSLRAEDTAVYYCARDYYGSPYWYFDVWGQGTLVTVSS |
| 114 | EVQLLESGGGLVQPGGSLRLSCAASGFTFSDYYMEWVRQAPGKGLEWVAASRNKANDYTTEY |
| | ADSVKGRFTISRDNSKNTLYLQMNSLRAEDTAVYYCARDYYGSSYWYFDTWGQGTLVTVSS |
| 115 | EVQLLESGGGLVQPGGSLRLSCAASGFTQSDFYMEWVRQAPGKGLEWVAASRNKANAYTTEY |
| | ADSVKGRFTISRDNSKNTLYLQMNSLRAEDTAVYYCARDYYGYSYWYFDVWGQGTLVTVSS |
| 116 | EVQLLESGGGLVQPGGSLRLSCAASGFTFSDEYMEWVRQAPGKGLEWVAASRNSGNSYTTAY |
| | ADSVKGRFTISRDNSKNTLYLQMNSLRAEDTAVYYCARDYYGSSYWYFDVWGQGTLVTVSS |
| 117 | EVQLLESGGGLVQPGGSLRLSCAASGFTFSDYYMEWVRQAPGKGLEWVAASRWKANDYTTEY |
| | ADSVKGRFTISRDNSKNTLYLQMNSLRAEDTAVYYCARDYYGSSQWYFDVWGQGTLVTVSS |
| 118 | EVQLLESGGGLVQPGGSLRLSCAASGFTFSDDYMEWVRQAPGKGLEWVAATRNKANDYTTEY |
| | ADSVKGRFTISRDNSKNTLYLQMNSLRAEDTAVYYCARDYYGSSYWYFDSWGQGTLVTVSS |
| 119 | EVQLLESGGGLVQPGGSLRLSCAASGFTFSDQYMEWVRQAPGKGLEWVAASRNKANDYTVEY |
| | ADSVKGRFTISRDNSKNTLYLQMNSLRAEDTAVYYCARDYYGSSYWYFDVWGQGTLVTVSS |
| 120 | EVQLLESGGGLVQPGGSLRLSCAASGFGFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 121 | EVQLLESGGGLVQPGGSLRLSCAASGFTGSDFYMEWVRQAPGKGLEWVAASRNSGNSYTTAY |
| | ADSVKGRFTISRDNSKNTLYLQMNSLRAEDTAVYYCARDYFGSSYWYFDVWGQGTLVTVSS |
| 122 | EVQLLESGGGLVQPGGSLRLSCAASGFTFSDEYMEWVRQAPGKGLEWVAASRNKANDFTTEY |
| | ADSVKGRFTISRDNSKNTLYLQMNSLRAEDTAVYYCARDFYGSSYWYFDVWGQGTLVTVSS |
| 123 | EVQLLESGGGLVQPGGSLRLSCAASGFQFSDFYMEWVRQAPGKGLEWVAASRNKGNSYTTAY |
| | ADSVKGRFTISRDNSKNTLYLQMNSLRAEDTAVYYCARDYYGSSYWYFDVWGQGTLVTVSS |
| 124 | EVQLLESGGGLVQPGGSLRLSCAASGTTFSDFYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 125 | EVQLLESGGGLVQPGGSLRLSCAASGFGFSDFYMEWVRQAPGKGLEWVAASRNKANDYTTEY |
| | ADSVKGRFTISRDNSKNTLYLQMNSLRAEDTAVYYCARDYYGSSYWYHDVWGQGTLVTVSS |
| 126 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSHWYFDVWGQGTLVTVSS |
| 127 | EVQLLESGGGLVQPGGSLRLSCAASGPTFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 128 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYFDPWGQGTLVTVSS |
| 129 | EVQLLESGGGLVQPGGSLRLSCAASGFTFSDAYMEWVRQAPGKGLEWVAASRNKANDYTTEY |
| | ADSVKGRFTISRDNSKNTLYLQMNSLRAEDTAVYYCARDYYGSHYWYFDVWGQGTLVTVSS |
| 130 | EVQLLESGGGLVQPGGSLRLSCAASGFTFSSYYMEWVRQAPGKGLEWVAASRNKANDYTYEY |
| | ADSVKGRFTISRDNSKNTLYLQMNSLRAEDTAVYYCARDYYESSYWYFDVWGQGTLVTVSS |
| 131 | EVQLLESGGGLVQPGGSLRLSCAASGDTFSDFYMEWVRQAPGKGLEWVAASRNKANDYSTEY |
| | ADSVKGRFTISRDNSKNTLYLQMNSLRAEDTAVYYCARDYYGSSYWYFDVWGQGTLVTVSS |
| 132 | EVQLLESGGGLVQPGGSLRLSCAASGFTFSEFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 133 | EVQLLESGGGLVQPGGSLRLSCAASGFTFSSYYMEWVRQAPGKGLEWVAASRSKANDYTTEY |
| | ADSVKGRFTISRDNSKNTLYLQMNSLRAEDTAVYYCARDYYGSQYWYFDVWGQGTLVTVSS |
| 134 | EVQLLESGGGLVQPGGSLRLSCAASGFHFSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 135 | EVQLLESGGGLVQPGGSLRLSCAASGFTFSDGYMEWVRQAPGKGLEWVAASRNKANDYTTAY |
| | ADSVKGRFTISRDNSKNTLYLQMNSLRAEDTAVYYCARDYYGSSYWYFDVWGQGTLVTVSS |
| 136 | EVQLLESGGGLVQPGGSLRLSCAASGFTFSSYYMEWVRQAPGKGLEWVAASRNTANDYTTEY |
| | ADSVKGRFTISRDNSKNTLYLQMNSLRAEDTAVYYCARDYYGSSYWYFDVWGQGTLVTVSS |
| 137 | EVQLLESGGGLVQPGGSLRLSCAASGITFSDFYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 138 | EVQLLESGGGLVQPGGSLRLSCAASGFGFSDFYMEWVRQAPGKGLEWVAASRNKANDYTTEY |
| | ADSVKGRFTISRDNSKNTLYLQMNSLRAEDTAVYYCARDYYSSSYWYFDVWGQGTLVTVSS |
| 139 | EVQLLESGGGLVQPGGSLRLSCAASGFTFSDYYMEWVRQAPGKGLEWVAASRNKANDYNTEY |
| | ADSVKGRFTISRDNSKNTLYLQMNSLRAEDTAVYYCARDYYGSSYWYFDVWGQGTLVTVSS |
| 140 | EVQLLESGGGLVQPGGSLRLSCAASGFTFSDFYVEWVRQAPGKGLEWVAASRNKANTYTTEY |
| | ADSVKGRFTISRDNSKNTLYLQMNSLRAEDTAVYYCARDYYGSSYWYFDVWGQGTLVTVSS |
| 141 | EVQLLESGGGLVQPGGSLRLSCAASGFQFSDFYMEWVRQAPGKGLEWVAASRWKANDYTTEY |
| | ADSVKGRFTISRDNSKNTLYLQMNSLRAEDTAVYYCARDYYGSSYWYFDVWGQGTLVTVSS |
| 142 | EVQLLESGGGLVQPGGSLRLSCAASGFTFSDFYMEWVRQAPGKGLEWVAASRNKANDYTTEV |
| | ADSVKGRFTISRDNSKNTLYLQMNSLRAEDTAVYYCARDYYGSSNWYFDVWGQGTLVTVSS |
| 143 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDYYGSSYWYNDVWGQGTLVTVSS |
| 144 | EVQLLESGGGLVQPGGSLRLSCAASGSTFSDFYMEWVRQAPGKGLEWVAASRNKANDETTEY |
| | ADSVKGRFTISRDNSKNTLYLQMNSLRAEDTAVYYCARDYYGSSYWYFDVWGQGTLVTVSS |
| 145 | EVQLLESGGGLVQPGGSLRLSCAASGFTFSDYYMEWVRQAPGKGLEWVAASRNKANDYTTEY |
| | ADSVKGRFTISRDNSKNTLYLQMNSLRAEDTAVYYCARDYYESSYWYFDVWGQGTLVTVSS |
| 146 | EVQLLESGGGLVQPGGSLRLSCAASGFTTSDFYMEWVRQAPGKGLEWVAASRNKANGYTTEY |
| | ADSVKGRFTISRDNSKNTLYLQMNSLRAEDTAVYYCARDYYGSSYWYFDVWGQGTLVTVSS |
| 147 | EVQLLESGGGLVQPGGSLRLSCAASGFTNSDFYMEWVRQAPGKGLEWVAASRNKANDYTTEL |
| | ADSVKGRFTISRDNSKNTLYLQMNSLRAEDTAVYYCARDYYGSSYWYFDVWGQGTLVTVSS |
| 148 | EVQLLESGGGLVQPGGSLRLSCAASGETFSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 149 | EVQLLESGGGLVQPGGSLRLSCAASGFTEFSSYYMEWVRQAPGKGLEWVAASRNKANDYTTEY |
| | ADSVKGRFTISRDNSKNTLYLQMNSLRAEDTAVYYCARDYYDSSYWYFDVWGQGTLVTVSS |
| 150 | EVQLLESGGGLVQPGGSLRLSCAASGFTFSDQYMEWVRQAPGKGLEWVAASRNKNNDYTTEY |
| | ADSVKGRFTISRDNSKNTLYLQMNSLRAEDTAVYYCARDYYGASYWYFDVWGQGTLVTVSS |
| 151 | EVQLLESGGGLVQPGGSLRLSCAASGFTFSDYYMEWVRQAPGKGLEWVAASRNKGNDYTTAY |
| | ADSVKGRFTISRDNSKNTLYLQMNSLRAEDTAVYYCARDYFGSSYWYFDVWGQGTLVTVSS |
| 152 | EVQLLESGGGLVQPGGSLRLSCAASGFTQSDFYMEWVRQAPGKGLEWVAASRNKGNSYTTAY |
| | ADSVKGRFTISRDNSKNTLYLQMNSLRAEDTAVYYCARDYYGSSYWYFDVWGQGTLVTVSS |
| 153 | EVQLLESGGGLVQPGGSLRLSCAASGFTFSDYYMEWVRQAPGKGLEWVAASRNKANSYTTAY |
| | ADSVKGRFTISRDNSKNTLYLQMNSLRAEDTAVYYCARDYYGSSYWYFDVWGQGTLVTVSS |
| 154 | EVQLLESGGGLVQPGGSLRLSCAASGFTFSDGYMEWVRQAPGKGLEWVAASRNKANFYTTEY |
| | ADSVKGRFTISRDNSKNTLYLQMNSLRAEDTAVYYCARDHYGSSYWYFDVWGQGTLVTVSS |
| 155 | EVQLLESGGGLVQPGGSLRLSCAASGFSFSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 156 | EVQLLESGGGLVQPGGSLRLSCAASGFTFSDFYMEWVRQAPGKGLEWVAASRNKYNDYTTEY |
| | ADSVKGRFTISRDNSKNTLYLQMNSLRAEDTAVYYCARDYYGSSYWYFDVWGQGTLVTVSS |
| 157 | EVQLLESGGGLVQPGGSLRLSCAASGFTFDDFYMEWVRQAPGKGLEWVAASRNEANDYTTEY |
| | ADSVKGRFTISRDNSKNTLYLQMNSLRAEDTAVYYCARDYYGSSYWYFDVWGQGTLVTVSS |
| 158 | EVQLLESGGGLVQPGGSLRLSCAASGFTFSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 159 | EVQLLESGGGLVQPGGSLRLSCAASGFTFSDFYIEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 160 | EVQLLESGGGLVQPGGSLRLSCAASGFTQSDFYMEWVRQAPGKGLEWVAASRNKANDYTTEY |
| | ADSVKGRFTISRDNSKNTLYLQMNSLRAEDTAVYYCARDYYGESYWYFDVWGQGTLVTVSS |
| 161 | EVQLLESGGGLVQPGGSLRLSCAASGPTFSDFYMEWVRQAPGKGLEWVAASRNKANGYTTEY |
| | ADSVKGRFTISRDNSKNTLYLQMNSLRAEDTAVYYCARDYYGSSYWYFDVWGQGTLVTVSS |
| 162 | EVQLLESGGGLVQPGGSLRLSCAASGFTDSDFYMEWVRQAPGKGLEWVAASRNKANGYTTEY |
| | ADSVKGRFTISRDNSKNTLYLQMNSLRAEDTAVYYCARDYYGSSYWYFDVWGQGTLVTVSS |
| 163 | EVQLLESGGGLVQPGGSLRLSCAASGDTFSDFYMEWVRQAPGKGLEWVAASRWKANDYTTEY |
| | ADSVKGRFTISRDNSKNTLYLQMNSLRAEDTAVYYCARDYYGSSYWYFDVWGQGTLVTVSS |
| 164 | EVQLLESGGGLVQPGGSLRLSCAASGFTFSSYYMEWVRQAPGKGLEWVAASRNKANDYTTEY |
| | ADSVKGRFTISRDNSKNTLYLQMNSLRAEDTAVYYCARDYYLSSYWYFDVWGQGTLVTVSS |
| 165 | EVQLLESGGGLVQPGGSLRLSCAASGFTFSDGYMEWVRQAPGKGLEWVAAIRNKANDYTTEY |
| | ADSVKGRFTISRDNSKNTLYLQMNSLRAEDTAVYYCARDYYGSHYWYFDVWGQGTLVTVSS |
| 166 | EVQLLESGGGLVQPGGSLRLSCAASGFTFSDYYMEWVRQAPGKGLEWVAASRNKGNDYTTAY |
| | ADSVKGRFTISRDNSKNTLYLQMNSLRAEDTAVYYCARDYYGSSYWYFDVWGQGTLVTVSS |
| 167 | EVQLLESGGGLVQPGGSLRLSCAASGFTFSDYYMEWVRQAPGKGLEWVAASRNVANDYTTEY |
| | ADSVKGRFTISRDNSKNTLYLQMNSLRAEDTAVYYCARDYYGSSYWYFDVWGQGTLVTVSS |
| 168 | EVQLLESGGGLVQPGGSLRLSCAASGFTGSDFYMEWVRQAPGKGLEWVAASRWKANDYTTEY |
| | ADSVKGRFTISRDNSKNTLYLQMNSLRAEDTAVYYCARDYYGSSYWYFDVWGQGTLVTVSS |
| 169 | EVQLLESGGGLVQPGGSLRLSCAASGFTFSDQYMEWVRQAPGKGLEWVAASRNKANDWTTEY |
| | ADSVKGRFTISRDNSKNTLYLQMNSLRAEDTAVYYCARDYYGSSYWYFDVWGQGTLVTVSS |
| 170 | EVQLLESGGGLVQPGGSLRLSCAASGNTFSDFYMEWVRQAPGKGLEWVAASRNKANYYTTEY |
| | ADSVKGRFTISRDNSKNTLYLQMNSLRAEDTAVYYCARDYYGSSYWYFDVWGQGTLVTVSS |
| 171 | EVQLLESGGGLVQPGGSLRLSCAASGFTGSDFYMEWVRQAPGKGLEWVAASRNKANDYTTEY |
| | ADSVKGRFTISRDNSKNTLYLQMNSLRAEDTAVYYCARDYYGSPYWYFDVWGQGTLVTVSS |
| 172 | EVQLLESGGGLVQPGGSLRLSCAASGFTSSDFYLEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 173 | EVQLLESGGGLVQPGGSLRLSCAASGFTFSPFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 174 | EVQLLESGGGLVQPGGSLRLSCAASGFTASDFYMEWVRQAPGKGLEWVAASRWKANDYTTEY |
| | ADSVKGRFTISRDNSKNTLYLQMNSLRAEDTAVYYCARDIYGSSYWYFDVWGQGTLVTVSS |
| 175 | EVQLLESGGGLVQPGGSLRLSCAASGFTFSDYYMEWVRQAPGKGLEWVAASRNKWNDYTTEY |
| | ADSVKGRFTISRDNSKNTLYLQMNSLRAEDTAVYYCARDYYLSSYWYFDVWGQGTLVTVSS |
| 176 | EVQLLESGGGLVQPGGSLRLSCAASGFTTSDFYMEWVRQAPGKGLEWVAASRNKANDYTTEY |
| | ADSVKGRFTISRDNSKNTLYLQMNSLRAEDTAVYYCARDYYGSSYWYFDYWGQGTLVTVSS |
| 177 | EVQLLESGGGLVQPGGSLRLSCAASGFTFSDFYMEWVRQAPGKGLEWVAASRNKANDYTTEY |
| | ADSVKGRFTISRDNSKNTLYLQMNSLRAEDTAVYYCARDFYGSSYWYFDVWGQGTLVTVSS |
| 178 | EVQLLESGGGLVQPGGSLRLSCAASGFTGSDFYMEWVRQAPGKGLEWVAASRNKANDYTTEY |
| | ADSVKGRFTISRDNSKNTLYLQMNSLRAEDTAVYYCARDYYGSSYWYFDVWGQGTLVTVSS |
| 179 | EVQLLESGGGLVQPGGSLRLSCAASGNTFSDFYMEWVRQAPGKGLEWVAASRNKWNDYTTEY |
| | ADSVKGRFTISRDNSKNTLYLQMNSLRAEDTAVYYCARDYYGSSYWYFDVWGQGTLVTVSS |
| 180 | EVQLLESGGGLVQPGGSLRLSCAASGFTFSDHYMEWVRQAPGKGLEWVAASRWKANDYTTEY |
| | ADSVKGRFTISRDNSKNTLYLQMNSLRAEDTAVYYCARDYYGSSYWYFDVWGQGTLVTVSS |
| 181 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAEFYSYPLTFGGGTKVEIK |
| 182 | DIQMTQSPSSLSASVGDRVTITCTASESLYSSKHKVHYLAWYQQKPGKAPKLLIYGASNREI |
| | GVPSRFSGSGSGTDFTLTISSLQPEDFATYYCQQFYSYPLTFGGGTKVEIK |
| 183 | DIQMTQSPSSLSASVGDRVTITCTASESLYSSKHKVHYLAWYQQKPGKAPKLLIYGASHRYI |
| | GVPSRFSGSGSGTDFTLTISSLQPEDFATYYCAQFYAYPLTFGGGTKVEIK |
| 184 | DIQMTQSPSSLSASVGDRVTITCTASESLYSSKHKVHYLAWYQQKPGKAPKLLIYGASNRYI |
| | GVPSRESGSGSGTDFTLTISSLQPEDFATYYCAQFYSYPLTFGGGTKVEIK |
| 185 | DIQMTQSPSSLSASVGDRVTITCTASESLYSSKHKVHYLAWYQQKPGKAPKLLIYGASSLQI |
| | GVPSRFSGSGSGTDFTLTISSLQPEDFATYYCAQFYSYPLTFGGGTKVEIK |
| 186 | DIQMTQSPSSLSASVGDRVTITCTASESLYSSKHKVHYLAWYQQKPGKAPKLLIYGASNRYT |
| | GVPSRFSGSGSGTDFTLTISSLQPEDFATYYCAQFYHYPLTFGGGTKVEIK |
| 187 | DIQMTQSPSSLSASVGDRVTITCTASESLYSSKHKVHYLAWYQQKPGKAPKLLIYGASNRYH |
| | GVPSRFSGSGSGTDFTLTISSLQPEDFATYYCAQFYSYPLTFGGGTKVEIK |
| 188 | DIQMTQSPSSLSASVGDRVTITCVASESLYSSKHKVHYLAWYQQKPGKAPKLLIYGASNRYI |
| | GVPSRFSGSGSGTDFTLTISSLQPEDFATYYCAQFYSYPLTFGGGTKVEIK |
| 189 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYQYPLTFGGGTKVEIK |
| 190 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYYYPLTFGGGTKVEIK |
| 191 | DIQMTQSPSSLSASVGDRVTITCTASESLYSSKHKVHYLAWYQQKPGKAPKLLIYGSSNRYI |
| | GVPSRFSGSGSGTDFTLTISSLQPEDFATYYCAQFYAYPLTFGGGTKVEIK |
| 192 | DIQMTQSPSSLSASVGDRVTITCTASESLYSSKHKVHYLAWYQQKPGKAPKLLIYGASNRFI |
| | GVPSRESGSGSGTDFTLTISSLQPEDFATYYCAQFYYYPLTFGGGTKVEIK |
| 193 | DIQMTQSPSSLSASVGDRVTITCRASESISSSKNKVHYLAWYQQKPGKAPKLLIYGASNRYY |
| | GVPSRFSGSGSGTDFTLTISSLQPEDFATYYCAQFYSYPLTFGGGTKVEIK |
| 194 | DIQMTQSPSSLSASVGDRVTITCTASESLYSSKHKVHYLAWYQQKPGKAPKLLIYGAANRYI |
| | GVPSRESGSGSGTDFTLTISSLQPEDFATYYCAQFYVYPLTFGGGTKVEIK |
| 195 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCGQFYSYPLTFGGGTKVEIK |
| 196 | DIQMTQSPSSLSASVGDRVTITCTASESLYSSKHKVHYLAWYQQKPGKAPKLLIYGASNRYV |
| | GVPSRFSGSGSGTDFTLTISSLQPEDFATYYCAQFYSYPLTFGGGTKVEIK |
| 197 | DIQMTQSPSSLSASVGDRVTITCTASESLYSAKHKVHYLAWYQQKPGKAPKLLIYGSSNRYI |
| | GVPSRFSGSGSGTDFTLTISSLQPEDFATYYCAQFYDYPLTFGGGTKVEIK |
| 198 | DIQMTQSPSSLSASVGDRVTITCTASESLYSSKHKVHYLAWYQQKPGKAPKLLIYGASNRYD |
| | GVPSRFSGSGSGTDFTLTISSLQPEDFATYYCAQFYSYPLTFGGGTKVEIK |
| 199 | DIQMTQSPSSLSASVGDRVTITCTASESLYSSKHKVNYLAWYQQKPGKAPKLLIYGASNRYI |
| | GVPSRFSGSGSGTDFTLTISSLQPEDFATYYCAQFYQYPLTFGGGTKVEIK |
| 200 | DIQMTQSPSSLSASVGDRVTITCTASESLYSSKHKVHYVAWYQQKPGKAPKLLIYGASNRYI |
| | GVPSRFSGSGSGTDFTLTISSLQPEDFATYYCAQFYYYPLTFGGGTKVEIK |
| 201 | DIQMTQSPSSLSASVGDRVTITCTASFSLYSSKHKVHYLAWYQQKPGKAPKLLIYGASNRYP |
| | GVPSRFSGSGSGTDFTLTISSLQPEDFATYYCAQFYYYPLTFGGGTKVEIK |
| 202 | DIQMTQSPSSLSASVGDRVTITCTASESLYSSKHKVHYLAWYQQKPGKAPKLLIYGAVNRYI |
| | GVPSRFSGSGSGTDFTLTISSLQPEDFATYYCAQFYRYPLTFGGGTKVEIK |
| 203 | DIQMTQSPSSLSASVGDRVTITCTASESLYSSKHKVHYLAWYQQKPGKAPKLLIYGASNGYI |
| | GVPSRFSGSGSGTDFTLTISSLQPEDFATYYCAQFYTYPLTFGGGTKVEIK |
| 204 | DIQMTQSPSSLSASVGDRVTITCTASESLYGSKHKVHYLAWYQQKPGKAPKLLIYGASNRYS |
| | GVPSRESGSGSGTDFTLTISSLQPEDFATYYCAQFYSYPLTFGGGTKVEIK |
| 205 | DIQMTQSPSSLSASVGDRVTITCTASESLYSSKHKVHYLAWYQQKPGKAPKLLIYGASNLYS |
| | GVPSRFSGSGSGTDFTLTISSLQPEDFATYYCAQFYSYPLTFGGGTKVEIK |
| 206 | DIQMTQSPSSLSASVGDRVTITCTASESLYSSKHKVHYLAWYQQKPGKAPKLLIYGASNRYF |
| | GVPSRFSGSGSGTDFTLTISSLQPEDFATYYCAQFYSYPLTFGGGTKVEIK |
| 207 | DIQMTQSPSSLSASVGDRVTITCTASESLYSSKHKVHYLAWYQQKPGKAPKLLIYGASNLQS |
| | GVPSRFSGSGSGTDFTLTISSLQPEDFATYYCAQFYYYPLTFGGGTKVEIK |
| 208 | DIQMTQSPSSLSASVGDRVTITCRASESISSSKHKHYLAWYQQKPGKAPKLLIYGASNRYIG |
| | VPSRFSGSGSGTDFTLTISSLQPEDFATYYCAQFYVYPLTFGGGTKVEIK |
| 209 | DIQMTQSPSSLSASVGDRVTITCTASESLYSSKHKVHYLAWYQQKPGKAPKLLIYGASNRYF |
| | GVPSRFSGSGSGTDFTLTISSLQPEDFATYYCAQFYQYPLTFGGGTKVEIK |
| 210 | DIQMTQSPSSLSASVGDRVTITCTASESLYSSKHKVHYLAWYQQKPGKAPKLLIYGLSNRYI |
| | GVPSRFSGSGSGTDFTLTISSLQPEDFATYYCAQFYSYPLTFGGGTKVEIK |
| 211 | DIQMTQSPSSLSASVGDRVTITCTASESLYSSKHKVHYLAWYQQKPGKAPKLLIYGASNRQS |
| | GVPSRFSGSGSGTDFTLTISSLQPEDFATYYCAQFYWYPLTFGGGTKVEIK |
| 212 | DIQMTQSPSSLSASVGDRVTITCTASESLYSSKHKVHYLAWYQQKPGKAPKLLIYGASNRQS |
| | GVPSRFSGSGSGTDFTLTISSLQPEDFATYYCAQFYAYPLTFGGGTKVEIK |
| 213 | DIQMTQSPSSLSASVGDRVTITCTASESLYSSKHKVHYLAWYQQKPGKAPKLLIYGASNRYH |
| | GVPSRESGSGSGTDFTLTISSLQPEDFATYYCAQFYYYPLTFGGGTKVEIK |
| 214 | DIQMTQSPSSLSASVGDRVTITCTASESLYSSKHKVHYLAWYQQKPGKAPKLLIYGAFNRYT |
| | GVPSRFSGSGSGTDFTLTISSLQPEDFATYYCAQFYTYPLTFGGGTKVEIK |
| 215 | DIQMTQSPSSLSASVGDRVTITCTASESLYSSKHKVHYLAWYQQKPGKAPKLLIYGASNRLI |
| | GVPSRFSGSGSGTDFTLTISSLQPEDFATYYCAQFYWYPLTFGGGTKVEIK |
| 216 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCTQFYSYPLTFGGGTKVEIK |
| 217 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYVYPLTFGGGTKVEIK |
| 218 | DIQMTQSPSSLSASVGDRVTITCTASESLYSSKHKVHYLAWYQQKPGKAPKLLIYGQSNRYI |
| | GVPSRFSGSGSGTDFTLTISSLQPEDFATYYCAQFYSYPLTFGGGTKVEIK |
| 219 | DIQMTQSPSSLSASVGDRVTITCTASEWLYSSKHKVHYLAWYQQKPGKAPKLLIYGASNRNI |
| | GVPSRFSGSGSGTDFTLTISSLQPEDFATYYCAQFYYYPLTFGGGTKVEIK |
| 220 | DIQMTQSPSSLSASVGDRVTITCTASESLYSSKHKVHYLAWYQQKPGKAPKLLIYGAGNRYI |
| | GVPSRFSGSGSGTDFTLTISSLQPEDFATYYCAQFYFYPLTFGGGTKVEIK |
| 221 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYWYPLTFGGGTKVEIK |
| 222 | DIQMTQSPSSLSASVGDRVTITCTASESLYSSKHKVHYLAWYQQKPGKAPKLLIYGASNRYG |
| | GVPSRFSGSGSGTDFTLTISSLQPEDFATYYCAQFYEYPLTFGGGTKVEIK |
| 223 | DIQMTQSPSSLSASVGDRVTITCTATESLYSSKHKVHYLAWYQQKPGKAPKLLIYGASNRYT |
| | GVPSRFSGSGSGTDFTLTISSLQPEDFATYYCAQFYSYPLTFGGGTKVEIK |
| 224 | DIQMTQSPSSLSASVGDRVTITCTASESLYSSKHKVHYLAWYQQKPGKAPKLLIYGASGRYI |
| | GVPSRFSGSGSGTDFTLTISSLQPEDFATYYCAQFYSYPLTFGGGTKVEIK |
| 225 | DIQMTQSPSSLSASVGDRVTITCTASESLYSGKHKVHYLAWYQQKPGKAPKLLIYGASNLQI |
| | GVPSRESGSGSGTDFTLTISSLQPEDFATYYCAQFYYYPLTFGGGTKVEIK |
| 226 | DIQMTQSPSSLSASVGDRVTITCFASESLYSSKHKVHYLAWYQQKPGKAPKLLIYGASNRGI |
| | GVPSRFSGSGSGTDFTLTISSLQPEDFATYYCAQFYSYPLTFGGGTKVEIK |
| 227 | DIQMTQSPSSLSASVGDRVTITCTASQSLYSSKNKVHYLAWYQQKPGKAPKLLIYGESNRYI |
| | GVPSRFSGSGSGTDFTLTISSLQPEDFATYYCAQFYSYPLTFGGGTKVEIK |
| 228 | DIQMTQSPSSLSASVGDRVTITCEASESLYSSKHKVHYLAWYQQKPGKAPKLLIYGASNRYP |
| | GVPSRFSGSGSGTDFTLTISSLQPEDFATYYCAQFYEYPLTFGGGTKVEIK |
| 229 | DIQMTQSPSSLSASVGDRVTITCTASESLYSSKHKVHYLAWYQQKPGKAPKLLIYGVSNRYI |
| | GVPSRFSGSGSGTDFTLTISSLQPEDFATYYCAQFYSYPLTFGGGTKVEIK |
| 230 | DIQMTQSPSSLSASVGDRVTITCTASESLYSSKHKVHYLAWYQQKPGKAPKLLIYGASNRYG |
| | GVPSRFSGSGSGTDFTLTISSLQPEDFATYYCAQFYSYPLTFGGGTKVEIK |
| 231 | DIQMTQSPSSLSASVGDRVTITCTASESLYSSKHKVHYLAWYQQKPGKAPKLLIYGASNRPI |
| | GVPSRFSGSGSGTDFTLTISSLQPEDFATYYCAQFYSYPLTFGGGTKVEIK |
| 232 | DIQMTQSPSSLSASVGDRVTITCTASESISSSKHKVHYLAWYQQKPGKAPKLLIYGASNLYS |
| | GVPSRFSGSGSGTDFTLTISSLQPEDFATYYCAHFYSYPLTFGGGTKVEIK |
| 233 | DIQMTQSPSSLSASVGDRVTITCTASESLYSSKHKVHYLAWYQQKPGKAPKLLIYGAGNRYI |
| | GVPSRFSGSGSGTDFTLTISSLQPEDFATYYCAQFYEYPLTFGGGTKVEIK |
| 234 | DIQMTQSPSSLSASVGDRVTITCTASESLYSSKHKVHYLAWYQQKPGKAPKLLIYGASFRYI |
| | GVPSRESGSGSGTDFTLTISSLQPEDFATYYCAQFYSYPLTFGGGTKVEIK |
| 235 | DIQMTQSPSSLSASVGDRVTITCTASESLYSSKHKVHYLAWYQQKPGKAPKLLIYGASNRGI |
| | GVPSRFSGSGSGTDFTLTISSLQPEDFATYYCAQFYSYPLTFGGGTKVEIK |
| 236 | DIQMTQSPSSLSASVGDRVTITCTASESLYSSKHKVHYLAWYQQKPGKAPKLLIYGADNRYI |
| | GVPSRESGSGSGTDFTLTISSLQPEDFATYYCAQFYHYPLTFGGGTKVEIK |
| 237 | DIQMTQSPSSLSASVGDRVTITCTASESLYSSKHKVHYLAWYQQKPGKAPKLLIYGASNRYD |
| | GVPSRFSGSGSGTDFTLTISSLQPEDFATYYCAQFYEYPLTFGGGTKVEIK |
| 238 | DIQMTQSPSSLSASVGDRVTITCTASESLYSSKHKVHYLAWYQQKPGKAPKLLIYGASNRQS |
| | GVPSRFSGSGSGTDFTLTISSLQPEDFATYYCAQFYVYPLTFGGGTKVEIK |
| 239 | DIQMTQSPSSLSASVGDRVTITCTASESLYSSKHKVHYLAWYQQKPGKAPKLLIYGASNRYQ |
| | GVPSRFSGSGSGTDFTLTISSLQPEDFATYYCAQFYFYPLTFGGGTKVEIK |
| 240 | DIQMTQSPSSLSASVGDRVTITCTASESLYSSKHKVHYLAWYQQKPGKAPKLLIYGASNRYS |
| | GVPSRFSGSGSGTDFTLTISSLQPEDFATYYCAQFYKYPLTFGGGTKVEIK |
| 241 | DIQMTQSPSSLSASVGDRVTITCTASESLYSSKHKVHYLAWYQQKPGKAPKLLIYGASSRYI |
| | GVPSRFSGSGSGTDFTLTISSLQPEDFATYYCAQFYEYPLTFGGGTKVEIK |
| 242 | DIQMTQSPSSLSASVGDRVTITCTASESLYSSKHKVHYLAWYQQKPGKAPKLLIYGTSNRYI |
| | GVPSRFSGSGSGTDFTLTISSLQPEDFATYYCAQFYEYPLTFGGGTKVEIK |
| 243 | DIQMTQSPSSLSASVGDRVTITCTASESLYSQKHKVHYLAWYQQKPGKAPKLLIYGASNRYI |
| | GVPSRFSGSGSGTDFTLTISSLQPEDFATYYCAQFYYYPLTFGGGTKVEIK |
| 244 | DIQMTQSPSSLSASVGDRVTITCTASESLYSSKHKVHYLAWYQQKPGKAPKLLIYGASNRYI |
| | GVPSRESGSGSGTDFTLTISSLQPEDFATYYCAQFYFYPLTFGGGTKVEIK |
| 245 | DIQMTQSPSSLSASVGDRVTITCTASESLYSSKHKVHYLAWYQQKPGKAPKLLIYGASNRQS |
| | GVPSRFSGSGSGTDFTLTISSLQPEDFATYYCAQFYSYPLTFGGGTKVEIK |
| 246 | DIQMTQSPSSLSASVGDRVTITCEASESLYSSKHKVHYLAWYQQKPGKAPKLLIYGASNRLI |
| | GVPSRFSGSGSGTDFTLTISSLQPEDFATYYCAQFYEYPLTFGGGTKVEIK |
| 247 | DIQMTQSPSSLSASVGDRVTITCTASESLYSSKHSVHYLAWYQQKPGKAPKLLIYGASNRFI |
| | GVPSRFSGSGSGTDFTLTISSLQPEDFATYYCAQFYSYPLTFGGGTKVEIK |
| 248 | DIQMTQSPSSLSASVGDRVTITCTASEELYSSKHKVHYLAWYQQKPGKAPKLLIYGASNRYI |
| | GVPSRFSGSGSGTDFTLTISSLQPEDFATYYCAQFYSYPLTFGGGTKVEIK |
| 249 | DIQMTQSPSSLSASVGDRVTITCTASQSISSSKHKVHYLAWYQQKPGKAPKLLIYGASNHYI |
| | GVPSRFSGSGSGTDFTLTISSLQPEDFATYYCAQFYSYPLTFGGGTKVEIK |
| 250 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYEYPLTFGGGTKVEIK |
| 251 | DIQMTQSPSSLSASVGDRVTITCTASESLYSSKHKVHYLAWYQQKPGKAPKLLIYGASNRQI |
| | GVPSRFSGSGSGTDFTLTISSLQPEDFATYYCAQFYTYPLTFGGGTKVEIK |
| 252 | DIQMTQSPSSLSASVGDRVTITCTASESLSSSKHKHYLAWYQQKPGKAPKLLIYGASSRYSG |
| | VPSRFSGSGSGTDFTLTISSLQPEDFATYYCAQFYSYPLTFGGGTKVEIK |
| 253 | DIQMTQSPSSLSASVGDRVTITCTASESLYSSKHKVHYLAWYQQKPGKAPKLLIYGASNRDI |
| | GVPSRFSGSGSGTDFTLTISSLQPEDFATYYCGQFYSYPLTFGGGTKVEIK |
| 254 | DIQMTQSPSSLSASVGDRVTITCTASESLYSSKHKVHYLAWYQQKPGKAPKLLIYGSSNRYI |
| | GVPSRFSGSGSGTDFTLTISSLQPEDFATYYCAQFYQYPLTFGGGTKVEIK |
| 255 | DIQMTQSPSSLSASVGDRVTITCTASESLYSSKHKVHYLAWYQQKPGKAPKLLIYGISNRYI |
| | GVPSRFSGSGSGTDFTLTISSLQPEDFATYYCAQFYSYPLTFGGGTKVEIK |
| 256 | DIQMTQSPSSLSASVGDRVTITCTAEESLYSSKHKVHYLAWYQQKPGKAPKLLIYGASNLYS |
| | GVPSRFSGSGSGTDFTLTISSLQPEDFATYYCAQFYSYPLTFGGGTKVEIK |
| 257 | DIQMTQSPSSLSASVGDRVTITCTASESLYSSKHKVHYLAWYQQKPGKAPKLLIYGASNRQI |
| | GVPSRFSGSGSGTDFTLTISSLQPEDFATYYCAQFYSYPLTFGGGTKVEIK |
| 258 | DIQMTQSPSSLSASVGDRVTITCTASESLYSSKHKVHYLAWYQQKPGKAPKLLIYGASNRYI |
| | GVPSRESGSGSGTDFTLTISSLQPEDFATYYCAQFYDYPLTFGGGTKVEIK |
| 259 | DIQMTQSPSSLSASVGDRVTITCTASESLYSSKHKVHYLAWYQQKPGKAPKLLIYGLSNRYI |
| | GVPSRFSGSGSGTDFTLTISSLQPEDFATYYCAQFFSYPLTFGGGTKVEIK |
| 260 | DIQMTQSPSSLSASVGDRVTITCTASESLYSSKHKVHYLAWYQQKPGKAPKLLIYGASSLYS |
| | GVPSRFSGSGSGTDFTLTISSLQPEDFATYYCAQFYEYPLTFGGGTKVEIK |
| 261 | DIQMTQSPSSLSASVGDRVTITCTASESLYSSKHGVHYLAWYQQKPGKAPKLLIYGASNRYI |
| | GVPSRFSGSGSGTDFTLTISSLQPEDFATYYCAQFYQYPLTFGGGTKVEIK |
| 262 | DIQMTQSPSSLSASVGDRVTITCTASESLYSSKHKVHYLAWYQQKPGKAPKLLIYGASNRAI |
| | GVPSRFSGSGSGTDFTLTISSLQPEDFATYYCAQFYTYPLTFGGGTKVEIK |
| 263 | DIQMTQSPSSLSASVGDRVTITCTASESLSSSKHKVHYLAWYQQKPGKAPKLLIYGASNRYI |
| | GVPSRFSGSGSGTDFTLTISSLQPEDFATYYCAQFYSYPLTFGGGTKVEIK |
| 264 | DIQMTQSPSSLSASVGDRVTITCTASESLYSSKHKVHYLAWYQQKPGKAPKLLIYGASNRYA |
| | GVPSRFSGSGSGTDFTLTISSLQPEDFATYYCGQFYSYPLTFGGGTKVEIK |
| 265 | DIQMTQSPSSLSASVGDRVTITCTASQSLYSSKHKVHYLAWYQQKPGKAPKLLIYGASNRYI |
| | GVPSRFSGSGSGTDFTLTISSLQPEDFATYYCAQFYVYPLTFGGGTKVEIK |
| 266 | DIQMTQSPSSLSASVGDRVTITCRASESLYSSLHKYLAWYQQKPGKAPKLLIYGASNLQIGV |
| | PSRFSGSGSGTDFTLTISSLQPEDFATYYCAQFYSYPLTFGGGTKVEIK |
| 267 | DIQMTQSPSSLSASVGDRVTITCTASESLYSSKHKVHYLAWYQQKPGKAPKLLIYGASNRVI |
| | GVPSRFSGSGSGTDFTLTISSLQPEDFATYYCAQFYSYPLTFGGGTKVEIK |
| 268 | DIQMTQSPSSLSASVGDRVTITCTASESLYSSKHKVHYLAWYQQKPGKAPKLLIYGPSNRYI |
| | GVPSRFSGSGSGTDFTLTISSLQPEDFATYYCAQFYSYPLTFGGGTKVEIK |
| 269 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYSYPLTFGGGTKVEIK |
| 270 | DIVMTQSPDSLAVSLGERATINCTASESVYSSKNKKHYLAWYQQKPGQPPKLLIYGASNRYI |
| | GVPDRFSGSGSGTDFTLTISSLQAEDVAVYYCAQFYSYPLTFGGGTKVEIK |
| 271 | DIVMTQSPDSLAVSLGERATINCTASESLYSSKGKVHYLAWYQQKPGQPPKLLIYGASNRYI |
| | GVPDRFSGSGSGTDFTLTISSLQAEDVAVYYCVQFYSYPLTFGGGTKVEIK |
| 272 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCGQFYSYPLTFGGGTKVEIK |
| 273 | DIVMTQSPDSLAVSLGERATINCTASESLYSSKHKVHYLAWYQQKPGQPPKLLIYGASNRYD |
| | GVPDRFSGSGSGTDFTLTISSLQAEDVAVYYCAQFYSYPLTFGGGTKVEIK |
| 274 | DIVMTQSPDSLAVSLGERATINCTASQSVYSSKNKVHYLAWYQQKPGQPPKLLIYGASNRYI |
| | GVPDRFSGSGSGTDFTLTISSLQAEDVAVYYCAQFYSYPLTFGGGTKVEIK |
| 275 | DIVMTQSPDSLAVSLGERATINCTSSQSLLSSSHNKNYLAWYQQKPGQPPKLLIYGASNRYI |
| | GVPDRFSGSGSGTDFTLTISSLQAEDVAVYYCAQFYSYPLTFGGGTKVEIK |
| 276 | DIVMTQSPDSLAVSLGERATINCTASESLYSPKHKVHYLAWYQQKPGQPPKLLIYGASNRYI |
| | GVPDRFSGSGSGTDFTLTISSLQAEDVAVYYCAQFYIYPLTFGGGTKVEIK |
| 277 | DIVMTQSPDSLAVSLGERATINCTASESLWSSKHKVHYLAWYQQKPGQPPKLLIYGASNRYI |
| | GVPDRFSGSGSGTDFTLTISSLQAEDVAVYYCAQFYSYPLTFGGGTKVEIK |
| 278 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYIYPLTFGGGTKVEIK |
| 279 | DIVMTQSPDSLAVSLGERATINCKASQSVLSSKNNVNYLAWYQQKPGQPPKLLIYGASNRYS |
| | GVPDRFSGSGSGTDFTLTISSLQAEDVAVYYCAQFYSYPLTFGGGTKVEIK |
| 280 | DIVMTQSPDSLAVSLGERATINCTASESLYSSKWKVHYLAWYQQKPGQPPKLLIYGASNRYI |
| | GVPDRFSGSGSGTDFTLTISSLQAEDVAVYYCAQFYSYPLTFGGGTKVEIK |
| 281 | DIVMTQSPDSLAVSLGERATINCTASESLYSSKHKVHYLAWYQQKPGQPPKLLIYGASNRVI |
| | GVPDRFSGSGSGTDFTLTISSLQAEDVAVYYCANFYSYPLTFGGGTKVEIK |
| 282 | DIVMTQSPDSLAVSLGERATINCKSSESVYSSKHKKHYLAWYQQKPGQPPKLLIYGASNRYI |
| | GVPDRFSGSGSGTDFTLTISSLQAEDVAVYYCAQFYSYPLTFGGGTKVEIK |
| 283 | DIVMTQSPDSLAVSLGERATINCTASESLYSSKHKVDYLAWYQQKPGQPPKLLIYGASNRYI |
| | GVPDRFSGSGSGTDFTLTISSLQAEDVAVYYCAQFYSYPLTFGGGTKVEIK |
| 284 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCANFYSYPLTFGGGTKVEIK |
| 285 | DIVMTQSPDSLAVSLGERATINCTASESLYSPKHKVHYLAWYQQKPGQPPKLLIYGAFNRYI |
| | GVPDRFSGSGSGTDFTLTISSLQAEDVAVYYCAQFYSYPLTFGGGTKVEIK |
| 286 | DIVMTQSPDSLAVSLGERATINCTASESVYSSKNKKHYLAWYQQKPGQPPKLLIYGASNRYI |
| | GVPDRFSGSGSGTDFTLTISSLQAEDVAVYYCAQFYTYPLTFGGGTKVEIK |
| 287 | DIVMTQSPDSLAVSLGERATINCKSSQSLYYSSNKKNYLAWYQQKPGQPPKLLIYGASNRYG |
| | GVPDRFSGSGSGTDFTLTISSLQAEDVAVYYCAQFYRYPLTFGGGTKVEIK |
| 288 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAHFYSYPLTFGGGTKVEIK |
| 289 | DIVMTQSPDSLAVSLGERATINCTASESLYSSKHKVHYLAWYQQKPGQPPKLLIYGASNRYS |
| | GVPDRFSGSGSGTDFTLTISSLQAEDVAVYYCAQFYSYPLTFGGGTKVEIK |
| 290 | DIVMTQSPDSLAVSLGERATINCTASESLYSSKHKVHYLAWYQQKPGQPPKLLIYGASNRYV |
| | GVPDRFSGSGSGTDFTLTISSLQAEDVAVYYCAQFYSYPLTFGGGTKVEIK |
| 291 | DIVMTQSPDSLAVSLGERATINCTASESLYSSKHKVHYLAWYQQKPGQPPKLLIYGASNREI |
| | GVPDRFSGSGSGTDFTLTISSLQAEDVAVYYCAQFYFYPLTFGGGTKVEIK |
| 292 | DIVMTQSPDSLAVSLGERATINCTASTSLYSSKHKVHYLAWYQQKPGQPPKLLIYGASNRYI |
| | GVPDRFSGSGSGTDFTLTISSLQAEDVAVYYCAQFYTYPLTFGGGTKVEIK |
| 293 | DIVMTQSPDSLAVSLGERATINCTASESLYGSKHKVHYLAWYQQKPGQPPKLLIYGASNRYI |
| | GVPDRFSGSGSGTDFTLTISSLQAEDVAVYYCAQFYSYPLTFGGGTKVEIK |
| 294 | DIVMTQSPDSLAVSLGERATINCKASQSLYSSKHKKHYLAWYQQKPGQPPKLLIYGASNRYP |
| | GVPDRFSGSGSGTDFTLTISSLQAEDVAVYYCAQFYRYPLTFGGGTKVEIK |
| 295 | DIVMTQSPDSLAVSLGERATINCTASSSLYSSKHKVHYLAWYQQKPGQPPKLLIYGASNRYI |
| | GVPDRFSGSGSGTDFTLTISSLQAEDVAVYYCAQFYSYPLTFGGGTKVEIK |
| 296 | DIVMTQSPDSLAVSLGERATINCTASESLYSSKHKVHYLAWYQQKPGQPPKLLIYGAGNRYI |
| | GVPDRFSGSGSGTDFTLTISSLQAEDVAVYYCAQFYHYPLTFGGGTKVEIK |
| 297 | DIVMTQSPDSLAVSLGERATINCTASESLYSSKHKVHYLAWYQQKPGQPPKLLIYGASNRYD |
| | GVPDRFSGSGSGTDFTLTISSLQAEDVAVYYCANFYSYPLTFGGGTKVEIK |
| 298 | DIVMTQSPDSLAVSLGERATINCTASESLYSSKHKVHYLAWYQQKPGQPPKLLIYGAGNRYI |
| | GVPDRFSGSGSGTDFTLTISSLQAEDVAVYYCAQFYSYPLTFGGGTKVEIK |
| 299 | DIVMTQSPDSLAVSLGERATINCTASESLYSSKHKVHYLAWYQQKPGQPPKLLIYGASNRQI |
| | GVPDRFSGSGSGTDFTLTISSLQAEDVAVYYCAQFYHYPLTFGGGTKVEIK |
| 300 | DIVMTQSPDSLAVSLGERATINCTASESLYSVKHKVHYLAWYQQKPGQPPKLLIYGASNRYI |
| | GVPDRFSGSGSGTDFTLTISSLQAEDVAVYYCAQFYSYPLTFGGGTKVEIK |
| 301 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYVYPLTFGGGTKVEIK |
| 302 | DIVMTQSPDSLAVSLGERATINCTASESLYSSKHKVHYLAWYQQKPGQPPKLLIYGANNRYI |
| | GVPDRFSGSGSGTDFTLTISSLQAEDVAVYYCAQFYSYPLTFGGGTKVEIK |
| 303 | DIVMTQSPDSLAVSLGERATINCGASESLYSSKSKVHYLAWYQQKPGQPPKLLIYGASNRYI |
| | GVPDRFSGSGSGTDFTLTISSLQAEDVAVYYCAQFYSYPLTFGGGTKVEIK |
| 304 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYEYPLTFGGGTKVEIK |
| 305 | DIVMTQSPDSLAVSLGERATINCTASESLYSSKHKVHYLAWYQQKPGQPPKLLIYGAHNRYI |
| | GVPDRFSGSGSGTDFTLTISSLQAEDVAVYYCANFYSYPLTFGGGTKVEIK |
| 306 | DIVMTQSPDSLAVSLGERATINCTSSESVYSSSHKVHYLAWYQQKPGQPPKLLIYGASNRYI |
| | GVPDRFSGSGSGTDFTLTISSLQAEDVAVYYCAQFYYYPLTFGGGTKVEIK |
| 307 | DIVMTQSPDSLAVSLGERATINCTASESLYSSKHKVHYLAWYQQKPGQPPKLLIYGNSNRYI |
| | GVPDRFSGSGSGTDFTLTISSLQAEDVAVYYCGQFYSYPLTFGGGTKVEIK |
| 308 | DIVMTQSPDSLAVSLGERATINCTASESLYSSKHKKNYLAWYQQKPGQPPKLLIYGASWRYI |
| | GVPDRFSGSGSGTDFTLTISSLQAEDVAVYYCAQFYSYPLTFGGGTKVEIK |
| 309 | DIVMTQSPDSLAVSLGERATINCTASESLYSSKHKVHYLAWYQQKPGQPPKLLIYGASTRYS |
| | GVPDRFSGSGSGTDFTLTISSLQAEDVAVYYCAQFYTYPLTFGGGTKVEIK |
| 310 | DIVMTQSPDSLAVSLGERATINCKSSESLLYSKNNKNYLAWYQQKPGQPPKLLIYGASNRYS |
| | GVPDRFSGSGSGTDFTLTISSLQAEDVAVYYCAQFYSYPLTFGGGTKVEIK |
| 311 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAEFYSYPLTFGGGTKVEIK |
| 312 | DIVMTQSPDSLAVSLGERATINCTASESIYSSKHKVHYLAWYQQKPGQPPKLLIYGASNRYI |
| | GVPDRFSGSGSGTDFTLTISSLQAEDVAVYYCAQFYSYPLTFGGGTKVEIK |
| 313 | DIVMTQSPDSLAVSLGERATINCTSSQSVLSSKHNKNYLAWYQQKPGQPPKLLIYGASNRYW |
| | GVPDRFSGSGSGTDFTLTISSLQAEDVAVYYCAQFYSYPLTFGGGTKVEIK |
| 314 | DIVMTQSPDSLAVSLGERATINCTASESLYSSKHKVHYLAWYQQKPGQPPKLLIYGASNRYP |
| | GVPDRFSGSGSGTDFTLTISSLQAEDVAVYYCAQFYSYPLTFGGGTKVEIK |
| 315 | DIVMTQSPDSLAVSLGERATINCTASEDLYSSKHKVHYLAWYQQKPGQPPKLLIYGTSNRYI |
| | GVPDRFSGSGSGTDFTLTISSLQAEDVAVYYCAQYYSYPLTFGGGTKVEIK |
| 316 | DIVMTQSPDSLAVSLGERATINCKASQSVLYSKNKKHYLAWYQQKPGQPPKLLIYGASNRQI |
| | GVPDRFSGSGSGTDFTLTISSLQAEDVAVYYCAQFYVYPLTFGGGTKVEIK |
| 317 | DIVMTQSPDSLAVSLGERATINCTASESLYSSKHKLHYLAWYQQKPGQPPKLLIYGASNRYI |
| | GVPDRFSGSGSGTDFTLTISSLQAEDVAVYYCGQFYSYPLTFGGGTKVEIK |
| 318 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYDYPLTFGGGTKVEIK |
| 319 | DIVMTQSPDSLAVSLGERATINCTASESLYSSKHKVVYLAWYQQKPGQPPKLLIYGASNRYI |
| | GVPDRFSGSGSGTDFTLTISSLQAEDVAVYYCAQFYFYPLTFGGGTKVEIK |
| 320 | DIVMTQSPDSLAVSLGERATINCTASESLYSSKHKVHYLAWYQQKPGQPPKLLIYGASNRYL |
| | GVPDRFSGSGSGTDFTLTISSLQAEDVAVYYCAQFYSYPLTFGGGTKVEIK |
| 321 | DIVMTQSPDSLAVSLGERATINCTSSESLYSSKNKVNYLAWYQQKPGQPPKLLIYGASNRYI |
| | GVPDRFSGSGSGTDFTLTISSLQAEDVAVYYCAQFYFYPLTFGGGTKVEIK |
| 322 | DIVMTQSPDSLAVSLGERATINCTASESLYSSKHKVHYLAWYQQKPGQPPKLLIYGASNRYN |
| | GVPDRFSGSGSGTDFTLTISSLQAEDVAVYYCAQFYTYPLTFGGGTKVEIK |
| 323 | DIVMTQSPDSLAVSLGERATINCTSSQSLYSSKHNKHYLAWYQQKPGQPPKLLIYGASNRYI |
| | GVPDRFSGSGSGTDFTLTISSLQAEDVAVYYCAQFYSYPLTFGGGTKVEIK |
| 324 | DIVMTQSPDSLAVSLGERATINCEASESLYSSKHKVHYLAWYQQKPGQPPKLLIYGASNRYI |
| | GVPDRFSGSGSGTDFTLTISSLQAEDVAVYYCAQFYSYPLTFGGGTKVEIK |
| 325 | DIVMTQSPDSLAVSLGERATINCTASESLYSSKHKVHYLAWYQQKPGQPPKLLIYGASNRAI |
| | GVPDRFSGSGSGTDFTLTISSLQAEDVAVYYCAQFYFYPLTFGGGTKVEIK |
| 326 | DIVMTQSPDSLAVSLGERATINCTASESLYSNKHKVHYLAWYQQKPGQPPKLLIYGASNRYI |
| | GVPDRFSGSGSGTDFTLTISSLQAEDVAVYYCAQFYSYPLTFGGGTKVEIK |
| 327 | DIVMTQSPDSLAVSLGERATINCTASENLYSSKHKVHYLAWYQQKPGQPPKLLIYGASNRYS |
| | GVPDRFSGSGSGTDFTLTISSLQAEDVAVYYCAQFYSYPLTFGGGTKVEIK |
| 328 | DIVMTQSPDSLAVSLGERATINCTASESLYSSKHKVHYLAWYQQKPGQPPKLLIYGASQRYI |
| | GVPDRFSGSGSGTDFTLTISSLQAEDVAVYYCAQFYLYPLTFGGGTKVEIK |
| 329 | DIVMTQSPDSLAVSLGERATINCTASESLYSSKHKVHYLAWYQQKPGQPPKLLIYGASNREI |
| | GVPDRFSGSGSGTDFTLTISSLQAEDVAVYYCAQFYSYPLTFGGGTKVEIK |
| 330 | DIVMTQSPDSLAVSLGERATINCTASESLYSSKHKVHYLAWYQQKPGQPPKLLIYGASNRYT |
| | GVPDRFSGSGSGTDFTLTISSLQAEDVAVYYCAQFYSYPLTFGGGTKVEIK |
| 331 | DIVMTQSPDSLAVSLGERATINCTASESLYSSKHKVHYLAWYQQKPGQPPKLLIYGAANRYI |
| | GVPDRFSGSGSGTDFTLTISSLQAEDVAVYYCAQFYSYPLTFGGGTKVEIK |
| 332 | DIVMTQSPDSLAVSLGERATINCTASESLYSSKHKVHYLAWYQQKPGQPPKLLIYGAQNRYI |
| | GVPDRFSGSGSGTDFTLTISSLQAEDVAVYYCGQFYSYPLTFGGGTKVEIK |
| 333 | DIVMTQSPDSLAVSLGERATINCTASESLYSSKIKVHYLAWYQQKPGQPPKLLIYGAHNRYI |
| | GVPDRFSGSGSGTDFTLTISSLQAEDVAVYYCAQFYSYPLTFGGGTKVEIK |
| 334 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYTYPLTFGGGTKVEIK |
| 335 | DIVMTQSPDSLAVSLGERATINCTASESLYSSKHKVHYLAWYQQKPGQPPKLLIYGASNRYS |
| | GVPDRFSGSGSGTDFTLTISSLQAEDVAVYYCAQFYHYPLTFGGGTKVEIK |
| 336 | DIVMTQSPDSLAVSLGERATINCTASESLYSSKHKVHYLAWYQQKPGQPPKLLIYGATNRYI |
| | GVPDRFSGSGSGTDFTLTISSLQAEDVAVYYCAEFYSYPLTFGGGTKVEIK |
| 337 | DIVMTQSPDSLAVSLGERATINCTASESLYSSKHKVHYLAWYQQKPGQPPKLLIYGASGRYI |
| | GVPDRFSGSGSGTDFTLTISSLQAEDVAVYYCAQFYSYPLTFGGGTKVEIK |
| 338 | DIVMTQSPDSLAVSLGERATINCKSSQSLLYSKNNVNYLAWYQQKPGQPPKLLIYGESNRYI |
| | GVPDRFSGSGSGTDFTLTISSLQAEDVAVYYCAQFYEYPLTFGGGTKVEIK |
| 339 | DIVMTQSPDSLAVSLGERATINCKSSESLYSSKNKVHYLAWYQQKPGQPPKLLIYGASNRYI |
| | GVPDRFSGSGSGTDFTLTISSLQAEDVAVYYCAQFYHYPLTFGGGTKVEIK |
| 340 | DIVMTQSPDSLAVSLGERATINCTASESLYSSKHKVNYLAWYQQKPGQPPKLLIYGASNRYI |
| | GVPDRFSGSGSGTDFTLTISSLQAEDVAVYYCAQFYSYPLTFGGGTKVEIK |
| 341 | DIVMTQSPDSLAVSLGERATINCTSSESLLSSKHKVHYLAWYQQKPGQPPKLLIYGASNRYT |
| | GVPDRFSGSGSGTDFTLTISSLQAEDVAVYYCAQFYSYPLTFGGGTKVEIK |
| 342 | DIVMTQSPDSLAVSLGERATINCTASQSVYSSKHKKHYLAWYQQKPGQPPKLLIYGANNRYI |
| | GVPDRFSGSGSGTDFTLTISSLQAEDVAVYYCGQFYSYPLTFGGGTKVEIK |
| 343 | DIVMTQSPDSLAVSLGERATINCTASQSVYSSKHNKHYLAWYQQKPGQPPKLLIYGASNPYI |
| | GVPDRFSGSGSGTDFTLTISSLQAEDVAVYYCAQFYSYPLTFGGGTKVEIK |
| 344 | DIVMTQSPDSLAVSLGERATINCTASESLYSGKHKVHYLAWYQQKPGQPPKLLIYGASNRYI |
| | GVPDRFSGSGSGTDFTLTISSLQAEDVAVYYCAQFYSYPLTFGGGTKVEIK |
| 345 | DIVMTQSPDSLAVSLGERATINCTSSQSLLYSSNKKHYLAWYQQKPGQPPKLLIYGASNRYI |
| | GVPDRFSGSGSGTDFTLTISSLQAEDVAVYYCAQFYEYPLTFGGGTKVEIK |
| 346 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAQFYNYPLTFGGGTKVEIK |
| 347 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCASFYSYPLTFGGGTKVEIK |
| 348 | DIVMTQSPDSLAVSLGERATINCKSSQSVLSSKHKKNYLAWYQQKPGQPPKLLIYGASNRYI |
| | GVPDRFSGSGSGTDFTLTISSLQAEDVAVYYCAQFYSYPLTFGGGTKVEIK |
| 349 | DIVMTQSPDSLAVSLGERATINCTASESLYSSKHKVHYLAWYQQKPGQPPKLLIYGAQNRYI |
| | GVPDRFSGSGSGTDFTLTISSLQAEDVAVYYCAQFYSYPLTFGGGTKVEIK |
| 350 | DIVMTQSPDSLAVSLGERATINCTASQSVYSSKNKKHYLAWYQQKPGQPPKLLIYGASNRYI |
| | GVPDRFSGSGSGTDFTLTISSLQAEDVAVYYCAQFYRYPLTFGGGTKVEIK |
| 351 | DIVMTQSPDSLAVSLGERATINCTASESVLSSKHKKNYLAWYQQKPGQPPKLLIYGASTRYI |
| | GVPDRFSGSGSGTDFTLTISSLQAEDVAVYYCAQFYSYPLTFGGGTKVEIK |
| 352 | DIVMTQSPDSLAVSLGERATINCTASVSLYSSKHKVHYLAWYQQKPGQPPKLLIYGPSNRYI |
| | GVPDRFSGSGSGTDFTLTISSLQAEDVAVYYCAQFYSYPLTFGGGTKVEIK |
| 353 | DIVMTQSPDSLAVSLGERATINCTASESLYSSKHKVHYLAWYQQKPGQPPKLLIYGASNRPI |
| | GVPDRFSGSGSGTDFTLTISSLQAEDVAVYYCGQFYSYPLTFGGGTKVEIK |
| 354 | DIVMTQSPDSLAVSLGERATINCTASESLYSSKHKVHYLAWYQQKPGQPPKLLIYGASNRYI |
| | GVPDRFSGSGSGTDFTLTISSLQAEDVAVYYCAAFYSYPLTFGGGTKVEIK |
| 355 | DIVMTQSPDSLAVSLGERATINCTASESLYSPKHKVHYLAWYQQKPGQPPKLLIYGAENRYI |
| | GVPDRFSGSGSGTDFTLTISSLQAEDVAVYYCAQFYSYPLTFGGGTKVEIK |
| 356 | DIVMTQSPDSLAVSLGERATINCTASESLYSSKHKVHYLAWYQQKPGQPPKLLIYGASHRQI |
| | GVPDRFSGSGSGTDFTLTISSLQAEDVAVYYCAQFYSYPLTFGGGTKVEIK |
| 357 | DIVMTQSPDSLAVSLGERATINCTASESLYSSKHKVHYLAWYQQKPGQPPKLLIYGASQRYI |
| | GVPDRFSGSGSGTDFTLTISSLQAEDVAVYYCAQFYSYPLTFGGGTKVEIK |
| 358 | DIVMTQSPDSLAVSLGERATINCTASTSLYSSKHKVHYLAWYQQKPGQPPKLLIYGASNRYI |
| | GVPDRFSGSGSGTDFTLTISSLQAEDVAVYYCAQFYSYPLTFGGGTKVEIK |
| 359 | DIVMTQSPDSLAVSLGERATINCKSSESLYSSKNKVHYLAWYQQKPGQPPKLLIYGASNRYS |
| | GVPDRFSGSGSGTDFTLTISSLQAEDVAVYYCAQFYSYPLTFGGGTKVEIK |
| 360 | DIVMTQSPDSLAVSLGERATINCTASESLYSTKHKVHYLAWYQQKPGQPPKLLIYGASHRYI |
| | GVPDRFSGSGSGTDFTLTISSLQAEDVAVYYCAQFYQYPLTFGGGTKVEIK |
| 361 | DIVMTQSPDSLAVSLGERATINCTASESLYSSKHKVHYLAWYQQKPGQPPKLLIYGVSNRYI |
| | GVPDRFSGSGSGTDFTLTISSLQAEDVAVYYCAQFYSYPLTFGGGTKVEIK |
| 362 | DIVMTQSPDSLAVSLGERATINCTASESLYSSKHKVHYLAWYQQKPGQPPKLLIYGASNRVI |
| | GVPDRFSGSGSGTDFTLTISSLQAEDVAVYYCAQFYIYPLTFGGGTKVEIK |
| 363 | DIVMTQSPDSLAVSLGERATINCTASESLYSSNHKVHYLAWYQQKPGQPPKLLIYGVSNRYI |
| | GVPDRFSGSGSGTDFTLTISSLQAEDVAVYYCAQFYSYPLTFGGGTKVEIK |
| 364 | DIVMTQSPDSLAVSLGERATINCKASQSVLSSKNNVNYLAWYQQKPGQPPKLLIYGASNRYI |
| | GVPDRFSGSGSGTDFTLTISSLQAEDVAVYYCAQFYQYPLTFGGGTKVEIK |
| 365 | DIVMTQSPDSLAVSLGERATINCTASESLYSSKHKVHYLAWYQQKPGQPPKLLIYGASNRNI |
| | GVPDRFSGSGSGTDFTLTISSLQAEDVAVYYCAHFYSYPLTFGGGTKVEIK |
| 366 | DIVMTQSPDSLAVSLGERATINCTASESLYSSKHKVHYLAWYQQKPGQPPKLLIYGASNRYQ |
| | GVPDRFSGSGSGTDFTLTISSLQAEDVAVYYCAQFYWYPLTFGGGTKVEIK |
| 367 | DIVMTQSPDSLAVSLGERATINCTASESLYSSKHKVHYLAWYQQKPGQPPKLLIYGASNTYI |
| | GVPDRFSGSGSGTDFTLTISSLQAEDVAVYYCAQFYSYPLTFGGGTKVEIK |
| 368 | DIVMTQSPDSLAVSLGERATINCTASESLYSSKHKVHYLAWYQQKPGQPPKLLIYGASNRYS |
| | GVPDRFSGSGSGTDFTLTISSLQAEDVAVYYCAQFYNYPLTFGGGTKVEIK |
| 369 | FTFSDYYME |
| 370 | FGFSDFYME |
| 371 | FTFSDQYIE |
| 372 | FTFSDGYME |
| 373 | FTASDFYME |
| 374 | FTFSDSYME |
| 375 | FTFEDFYME |
| 376 | FTFSDFYME |
| 377 | FTFSDQYME |
| 378 | FQFSDFYME |
| 379 | FEFSDNYME |
| 380 | FEFSDFYME |
| 381 | FTFSDAYME |
| 382 | FTFSDFYME |
| 383 | FTFSSYYME |
| 384 | FTGSDFYME |
| 385 | FAFSDFYME |
| 386 | FTHSDFYME |
| 387 | FTFSDTYME |
| 388 | FTFSDDYME |
| 389 | FTFSDNYME |
| 390 | FTSSDFYME |
| 391 | FTFSTFYME |
| 392 | FTFSDEYME |
| 393 | FWFSDFYME |
| 394 | FTFSDVYME |
| 395 | ATFSDFYME |
| 396 | FDFSDFYME |
| 397 | WTFSDFYME |
| 398 | FTFSSFYME |
| 399 | FTFNDFYME |
| 400 | FTFSDHYME |
| 401 | FTFSDGYIE |
| 402 | GTFSDFYME |
| 403 | FTQSDFYME |
| 404 | DTFSDFYME |
| 405 | FTTSDFYME |
| 406 | ETFSDFYME |
| 407 | FPFSDFYME |
| 408 | ATFSDFYVE |
| 409 | FTDSDFYME |
| 410 | NTFSDFYME |
| 411 | DTFSDFYLE |
| 412 | FLFSDFYME |
| 413 | FTNSDFYME |
| 414 | QTFSDFYME |
| 415 | FTFSDPYME |
| 416 | TTFSDFYME |
| 417 | PTFSDFYME |
| 418 | FTFSEFYME |
| 419 | FHFSDFYME |
| 420 | ITFSDFYME |
| 421 | FTFSDFYVE |
| 422 | STFSDFYME |
| 423 | FSFSDFYME |
| 424 | FTFDDFYME |
| 425 | FTFSDFYIE |
| 426 | FTSSDFYLE |
| 427 | FTFSPFYME |
| 428 | AASRNKANDYTTEYA |
| 429 | AASRNKWNDYTTEYA |
| 430 | AASRNKANDYTTSYA |
| 431 | AASRNKANDYTTELA |
| 432 | AASRGKGNSYTTEYA |
| 433 | AASRNKANDYVTEYA |
| 434 | AASRNKYNDYTTEYA |
| 435 | AASRNKGNSYTTEYA |
| 436 | AASRNKANTYTTEYA |
| 437 | AASRNKNNDYTTEYA |
| 438 | AASRNIANDYTTEYA |
| 439 | AASRNSGNDYTTEYA |
| 440 | AASRNKANDYTVEYA |
| 441 | AASRNKANDYYTEYA |
| 442 | AASRHKANDYTTEYA |
| 443 | AASRNKANDYQTEYA |
| 444 | AASRNQANDYTTEYA |
| 445 | AASRNGANDYTTEYA |
| 446 | AASRNKANDYTTDYA |
| 447 | AASRNKANDYTNEYA |
| 448 | AASRNAANDYTTEYA |
| 449 | AASRNKANDQTTEYA |
| 450 | AASRNSGNSYTTEYA |
| 451 | AASRNKANSYTTEYA |
| 452 | AASRWKANDYTTEYA |
| 453 | AAIRNKANSYTTEYA |
| 454 | AASRNKANSYTTAYA |
| 455 | AASRNKANDYTTTYA |
| 456 | AASRNKGNDYTTEYA |
| 457 | GASRNKHNDYTTEYA |
| 458 | AASRNKANAYTTEYA |
| 459 | AASRNKENDYTTEYA |
| 460 | AASRNKANGYTTEYA |
| 461 | AASRNKANEYTTEYA |
| 462 | AASRNKANLYTTEYA |
| 463 | AASRGKGNSYTTAYA |
| 464 | AASRNKGNSYTTAYA |
| 465 | AASRNNANDYTTEYA |
| 466 | AASRNKANGYTTEYT |
| 467 | AASRNSGNSYTTAYA |
| 468 | AATRNKANDYTTEYA |
| 469 | AASRNKANDFTTEYA |
| 470 | AASRNKANDYTYEYA |
| 471 | AASRNKANDYSTEYA |
| 472 | AASRSKANDYTTEYA |
| 473 | AASRNKANDYTTAYA |
| 474 | AASRNTANDYTTEYA |
| 475 | AASRNKANDYNTEYA |
| 476 | AASRNKANDYTTEVA |
| 477 | AASRNKGNDYTTAYA |
| 478 | AASRNKANFYTTEYA |
| 479 | AASRNEANDYTTEYA |
| 480 | AAIRNKANDYTTEYA |
| 481 | AASRNVANDYTTEYA |
| 482 | AASRNKANDWTTEYA |
| 483 | AASRNKANYYTTEYA |
| 484 | CARDYYGSSYWYFDVW |
| 485 | CARDYYGSSYWYYDVW |
| 486 | CARDYLGSSYWYFDVW |
| 487 | CARDYFGSSYWYFDVW |
| 488 | CARDYYASSYWYFDVW |
| 489 | CARDYYGSPYWYFDVW |
| 490 | CARDYYGSSYWYFDSW |
| 491 | CARDYYGSSYWYFDTW |
| 492 | CARDYYGSSYWYFDLW |
| 493 | CARDYYGSHYWYFDVW |
| 494 | CARDYQGSSYWYFDVW |
| 495 | CARDYYGHSYWYFDVW |
| 496 | CARDYYGSSYWYLDVW |
| 497 | CARDYYGNSYWYFDVW |
| 498 | CARDYYGSAYWYFDVW |
| 499 | CARDYYGQSYWYFDVW |
| 500 | CARDYYGSSTWYFDVW |
| 501 | CARDYYESSYWYFDVW |
| 502 | CARDYYGSSHWYFDVW |
| 503 | CARDYYGDSYWYFDVW |
| 504 | CARDYYGSSYWYNDVW |
| 505 | CARDYYHSSYWYFDVW |
| 506 | CARDIYGSSYWYFDVW |
| 507 | CARDYYGSSYWYDDVW |
| 508 | CARDYYGYSYWYFDVW |
| 509 | CARDYYGSSQWYFDVW |
| 510 | CARDFYGSSYWYFDVW |
| 511 | CARDYYGSSYWYHDVW |
| 512 | CARDYYGSSYWYFDPW |
| 513 | CARDYYGSQYWYFDVW |
| 514 | CARDYYSSSYWYFDVW |
| 515 | CARDYYGSSNWYFDVW |
| 516 | CARDYYFSSYWYFDVW |
| 517 | CARDYYDSSYWYFDVW |
| 518 | CARDYYGASYWYFDVW |
| 519 | CARDHYGSSYWYFDVW |
| 520 | CARDYYGESYWYFDVW |
| 521 | CARDYYLSSYWYFDVW |
| 522 | CARDYYGSSYWYFDYW |
| 523 | TASESLYSSKHKVHYLA |
| 524 | VASESLYSSKHKVHYLA |
| 525 | RASESISSSKNKVHYLA |
| 526 | TASESLYSAKHKVHYLA |
| 527 | TASESLYSSKHKVNYLA |
| 528 | TASESLYSSKHKVHYVA |
| 529 | TASFSLYSSKHKVHYLA |
| 530 | TASESLYGSKHKVHYLA |
| 531 | RASESISSSKHKHYLA |
| 532 | TASEWLYSSKHKVHYLA |
| 533 | TATESLYSSKHKVHYLA |
| 534 | TASESLYSGKHKVHYLA |
| 535 | FASESLYSSKHKVHYLA |
| 536 | TASQSLYSSKNKVHYLA |
| 537 | EASESLYSSKHKVHYLA |
| 538 | TASESISSSKHKVHYLA |
| 539 | TASESLYSQKHKVHYLA |
| 540 | TASESLYSSKHSVHYLA |
| 541 | TASEELYSSKHKVHYLA |
| 542 | TASQSISSSKHKVHYLA |
| 543 | TASESLSSSKHKHYLA |
| 544 | TAEESLYSSKHKVHYLA |
| 545 | TASESLYSSKHGVHYLA |
| 546 | TASESLSSSKHKVHYLA |
| 547 | TASQSLYSSKHKVHYLA |
| 548 | RASESLYSSLHKYLA |
| 549 | TASESVYSSKNKKHYLA |
| 550 | TASESLYSSKGKVHYLA |
| 551 | TASQSVYSSKNKVHYLA |
| 552 | TSSQSLLSSSHNKNYLA |
| 553 | TASESLYSPKHKVHYLA |
| 554 | TASESLWSSKHKVHYLA |
| 555 | KASQSVLSSKNNVNYLA |
| 556 | TASESLYSSKWKVHYLA |
| 557 | KSSESVYSSKHKKHYLA |
| 558 | TASESLYSSKHKVDYLA |
| 559 | KSSQSLYYSSNKKNYLA |
| 560 | TASTSLYSSKHKVHYLA |
| 561 | KASQSLYSSKHKKHYLA |
| 562 | TASSSLYSSKHKVHYLA |
| 563 | TASESLYSVKHKVHYLA |
| 564 | GASESLYSSKSKVHYLA |
| 565 | TSSESVYSSSHKVHYLA |
| 566 | TASESLYSSKHKKNYLA |
| 567 | KSSESLLYSKNNKNYLA |
| 568 | TASESIYSSKHKVHYLA |
| 569 | TSSQSVLSSKHNKNYLA |
| 570 | TASEDLYSSKHKVHYLA |
| 571 | KASQSVLYSKNKKHYLA |
| 572 | TASESLYSSKHKLHYLA |
| 573 | TASESLYSSKHKVVYLA |
| 574 | TSSESLYSSKNKVNYLA |
| 575 | TSSQSLYSSKHNKHYLA |
| 576 | TASESLYSNKHKVHYLA |
| 577 | TASENLYSSKHKVHYLA |
| 578 | TASESLYSSKIKVHYLA |
| 579 | KSSQSLLYSKNNVNYLA |
| 580 | KSSESLYSSKNKVHYLA |
| 581 | TSSESLLSSKHKVHYLA |
| 582 | TASQSVYSSKHKKHYLA |
| 583 | TASQSVYSSKHNKHYLA |
| 584 | TSSQSLLYSSNKKHYLA |
| 585 | KSSQSVLSSKHKKNYLA |
| 586 | TASQSVYSSKNKKHYLA |
| 587 | TASESVLSSKHKKNYLA |
| 588 | TASVSLYSSKHKVHYLA |
| 589 | TASESLYSTKHKVHYLA |
| 590 | TASESLYSSNHKVHYLA |
| 591 | GASNRYI |
| 592 | GASNREI |
| 593 | GASHRYI |
| 594 | GASSLQI |
| 595 | GASNRYT |
| 596 | GASNRYH |
| 597 | GSSNRYI |
| 598 | GASNRFI |
| 599 | GASNRYY |
| 600 | GAANRYI |
| 601 | GASNRYV |
| 602 | GASNRYD |
| 603 | GASNRYP |
| 604 | GAVNRYI |
| 605 | GASNGYI |
| 606 | GASNRYS |
| 607 | GASNLYS |
| 608 | GASNRYF |
| 609 | GASNLQS |
| 610 | GLSNRYI |
| 611 | GASNRQS |
| 612 | GAFNRYT |
| 613 | GASNRLI |
| 614 | GQSNRYI |
| 615 | GASNRNI |
| 616 | GAGNRYI |
| 617 | GASNRYG |
| 618 | GASGRYI |
| 619 | GASNLQI |
| 620 | GASNRGI |
| 621 | GESNRYI |
| 622 | GVSNRYI |
| 623 | GASNRPI |
| 624 | GASFRYI |
| 625 | GADNRYI |
| 626 | GASNRYQ |
| 627 | GASSRYI |
| 628 | GTSNRYI |
| 629 | GASNHYI |
| 630 | GASNRQI |
| 631 | GASSRYS |
| 632 | GASNRDI |
| 633 | GISNRYI |
| 634 | GASSLYS |
| 635 | GASNRAI |
| 636 | GASNRYA |
| 637 | GASNRVI |
| 638 | GPSNRYI |
| 639 | GAFNRYI |
| 640 | GANNRYI |
| 641 | GAHNRYI |
| 642 | GNSNRYI |
| 643 | GASWRYI |
| 644 | GASTRYS |
| 645 | GASNRYW |
| 646 | GASNRYL |
| 647 | GASNRYN |
| 648 | GASQRYI |
| 649 | GAQNRYI |
| 650 | GATNRYI |
| 651 | GASNPYI |
| 652 | GASTRYI |
| 653 | GAENRYI |
| 654 | GASHRQI |
| 655 | GASNTYI |
| 656 | CAEFYSYPLTF |
| 657 | CQQFYSYPLTF |
| 658 | CAQFYAYPLTE |
| 659 | CAQFYSYPLTE |
| 660 | CAQFYHYPLTF |
| 661 | CAQFYQYPLTF |
| 662 | CAQFYYYPLTF |
| 663 | CAQFYVYPLTF |
| 664 | CGQFYSYPLTF |
| 665 | CAQFYDYPLTF |
| 666 | CAQFYRYPLTF |
| 667 | CAQFYTYPLTF |
| 668 | CAQFYWYPLTF |
| 669 | CTQFYSYPLTF |
| 670 | CAQFYFYPLTF |
| 671 | CAQFYEYPLTF |
| 672 | CAHFYSYPLTF |
| 673 | CAQFYKYPLTF |
| 674 | CAQFFSYPLTF |
| 675 | CVQFYSYPLTF |
| 676 | CAQFYIYPLTF |
| 677 | CANFYSYPLTF |
| 678 | CAQYYSYPLTF |
| 679 | CAQFYLYPLTF |
| 680 | CAQFYNYPLTF |
| 681 | CASFYSYPLTF |
| 682 | CAAFYSYPLTF |
| 683 | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPELLGGPSVF |
| | LFPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPG |
| 684 | ASTKGPSVFPLAPCSRSTSESTAALGCLVKDYFPEPVTVSWNSGALTSGVHTFPAVLQSSGL |
| | YSLSSVVTVPSSSLGTKTYTCNVDHKPSNTKVDKRVESKYGPPCPPCPAPEFLGGPSVFLFP |
| | PKPKDTLMISRTPEVTCVVVDVSQEDPEVQFNWYVDGVEVHNAKTKPREEQENSTYRVVSVL |
| | TVLHQDWLNGKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEGNVFSCSVMHE |
| | ALHNHYTQKSLSLSLG |
| 685 | DIQMTQSPSSLSASVGDRVTITCTASESLYSSKHKVHYLAWYQQKPGKAPKLLIYGASNRYI |
| | GVPSRFSGSGSGTDFTLTISSLQPEDFATYYCAQFYSYPLTFGQGTKVEIK |
| 686 | AGGGGSGGGGSGGGGSAAA |
| | (Note: peptide can be conjugated with biotin at the N terminus |
| | residue, PC linked Tyr at the C terminus residue) |
| 687 | AEAAAKEAAAKEAAAKEAAAKAAA |
| | (Note: peptide can be conjugated with biotin at the N terminus |
| | residue, PC linked Tyr at the C terminus residue |
| 688 | RTVAAPSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSENRGEC |
| 689 | GQPKANPTVTLFPPSSEELQANKATLVCLISDFYPGAVTVAWKADGSPVKAGVETTKPSKQS |
| | NNKYAASSYLSLTPEQWKSHRSYSCQVTHEGSTVEKTVAPTECS |
| 690 | GQPKAAPSVTLFPPSSEELQANKATLVCLISDFYPGAVTVAWKADSSPVKAGVETTTPSKQS |
| | NNKYAASSYLSLTPEQWKSHRSYSCQVTHEGSTVEKTVAPTECS |
| 691 | GQPKAAPSVTLFPPSSEELQANKATLVCLISDFYPGAVTVAWKADSSPVKAGVETTTPSKQS |
| | NNKYAASSYLSLTPEQWKSHKSYSCQVTHEGSTVEKTVAPTECS |
| 692 | GQPKAAPSVTLFPPSSEELQANKATLVCLISDFYPGAVKVAWKADGSPVNTGVETTTPSKQS |
| | NNKYAASSYLSLTPEQWKSHRSYSCQVTHEGSTVEKTVAPAECS |
| 693 | GQPKAAPSVTLFPPSSEELQANKATLVCLVSDFNPGAVTVAWKADGSPVKVGVETTKPSKQS |
| | NNKYAASSYLSLTPEQWKSHRSYSCRVTHEGSTVEKTVAPAECS |

The invention is not to be limited in scope by the specific embodiments described herein. Indeed, various modifications of the invention in addition to those described will become apparent to those skilled in the art from the foregoing description. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

What is claimed:

1. An antibody that specifically binds phosphocholine (PC), the antibody comprising: a VH comprising the CDRH1, CDRH2, and CDRH3 amino acid sequences of any one of the VH amino acid sequences set forth in SEQ ID NOs: 1-180; and a VL comprising the CDRL1, CDRL2, and CDRL3 amino acid sequences of any one of the VL amino acid sequences set forth in SEQ ID NOs: 181-368.

2. An antibody that specifically binds phosphocholine (PC), the antibody comprising: a VH comprising the CDRH1, CDRH2, and CDRH3 amino acid sequences of the VH amino acid sequence set forth in SEQ ID NO: 27; and a VL comprising the CDRL1, CDRL2, and CDRL3 amino acid sequences of the VL amino acid sequence set forth in SEQ ID NO: 208.

3. The antibody of claim 2, wherein the antibody comprises the CDRH1, CDRH2, and CDRH3 amino acid sequences, respectively, set forth in SEQ ID NOs: 386, 428, and 484.

4. The antibody of claim 2, wherein the antibody comprises the CDRL1, CDRL2, and CDRL3 amino acid sequences, respectively, set forth in SEQ ID NOs: 531, 591, and 663.

5. The antibody of claim 2, wherein the antibody comprises the CDRH1, CDRH2, CDRH3, CDRL1, CDRL2, and CDRL3 amino acid sequences, respectively, set forth in SEQ ID NOs: 386, 428, 484, 531, 591, and 663.

6. The antibody of claim 2, wherein the antibody comprises the VH amino acid sequence of SEQ ID NO: 27.

7. The antibody of claim 2, wherein the antibody comprises the VL amino acid sequence of SEQ ID NO: 208.

8. The antibody of claim 2, wherein the VH and VL comprise the amino acid sequences, respectively, set forth in SEQ ID NOs: 27 and 208.

9. The antibody of claim 2, wherein the antibody is a single-chain variable fragment (scFv).

10. The antibody of claim 2, wherein the antibody comprises a heavy chain constant region, or an Fc region thereof.

11. The antibody of claim 10, wherein the heavy chain constant region is selected from the group consisting of a human IgG₁, IgG₂, IgG₃, IgG₄, IgA₁, and IgA₂.

12. The antibody of claim 10, wherein the heavy chain constant region is a human IgG₁.

13. The antibody of claim 10, wherein the heavy chain constant region is a human IgG₄.

14. The antibody of claim 13, wherein the amino acid sequence of the human IgG₄ heavy chain constant region comprises a P at position 228, numbered according to the EU numbering system.

15. The antibody of claim 10, wherein the heavy chain constant region is a variant of a wild-type heavy chain constant region, wherein the variant heavy chain constant region binds to an Fc gamma receptor (FcγR) with lower affinity than the wild-type heavy chain constant region binds to the FcγR.

16. The antibody of claim 15, wherein the amino acid sequence of the heavy chain constant region comprises: A at position 234; A at position 235; A, Q or G at position 297; or A or G at position 329, in each case numbered according to the EU numbering system.

17. The antibody of claim 15, wherein the amino acid sequence of the heavy chain constant region comprises: A at positions 234 and 235; A at positions 234, 235, and 329; or A at positions 234, 235, and G at position 329, in each case numbered according to the EU numbering system.

18. The antibody of claim 10, wherein the heavy chain constant region is a variant of a wild-type heavy chain constant region, and wherein the variant heavy chain constant region has an increased affinity for human neonatal Fc receptor (FcRn) at pH 6 relative to the affinity of the wild-type heavy chain constant region for human FcRn at pH 6.

19. The antibody of claim 18, wherein the amino acid sequence of the heavy chain constant region comprises: L and S at positions 428 and 434, respectively; K, F, and Y at positions 433, 434, and 436, respectively; or Y, T, and E at positions 252, 254, and 256, respectively, in each case numbered according to the EU numbering system.

20. The antibody of claim 2, wherein the antibody comprises a light chain constant region.

21. The antibody of claim 20, wherein the light chain constant region is a human kappa or lambda constant region.

22. The antibody of claim 2, wherein the antibody is conjugated to a cytotoxic agent, cytostatic agent, toxin, radionuclide, or detectable label.

23. A polynucleotide or plurality of polynucleotides encoding a VH and/or a VL, or a heavy chain and/or a light chain of the antibody of claim 1.

24. A vector or plurality of vectors comprising the polynucleotide or plurality of polynucleotides of claim 23.

25. A recombinant host cell comprising the polynucleotide or plurality of polynucleotides of claim 23.

26. A composition comprising the antibody of claim 1, or a polynucleotide or plurality of polynucleotides encoding the antibody, and a pharmaceutically acceptable carrier or excipient.

27. A method of producing an antibody, the method comprising culturing the recombinant host cell of claim 25 under suitable conditions such that the polynucleotide or plurality of polynucleotides is expressed, and the antibody is produced.

28. A method of inhibiting an activity of PC in a subject, the method comprising administering to the subject an effective amount of the antibody of claim 1.

29. A method of treating an inflammatory disorder or degenerative disease in a subject, the method comprising administering to the subject an effective amount of the antibody of claim 1.

30. The method of claim 29, wherein the inflammatory disorder or degenerative disease is selected from the group consisting of organ reperfusion injury, myocardial infarction induced reperfusion injury, Kawasaki disease, non-alcoholic steatohepatitis (NASH), organ transplant rejection, atherosclerosis, Type I or Type 2 Diabetes, rheumatoid arthritis, osteoporosis, acute lung injury, asthma, chronic obstructive pulmonary disease (COPD), lung fibrosis, pain, Alzheimer's disease, amyotrophic lateral sclerosis (ALS), age-related macular degeneration (AMD), stroke, Huntington's disease, frontotemporal dementia (FTD), multiple sclerosis (MS), isolated demyelinating diseases of the central nervous system, osteoarthritis, Crohn's Disease, and ulcerative colitis.


SEQUENCE LISTING (693 sequences)

SEQ ID NO: 1 - protein, 123 aa, synthetic construct
EVQLLESGGGLVQPGGSLRLSCAASGFTFSDYYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 2 - protein, 123 aa, synthetic construct
EVQLLESGGGLVQPGGSLRLSCAASGFGFSDFYMEWVRQAPGKGLEWVAASRNKWNDYTTEYADSVKGRFTISRDNSKNTLYLQMNSLRAEDTAVYYCARDYYGSSYWYYDVWGQGTLVTVSS

SEQ ID NO: 3 - protein, 123 aa, synthetic construct
EVQLLESGGGLVQPGGSLRLSCAASGFTFSDQYIEWVRQAPGKGLEWVAASRNKANDYTTEYADSVKGRFTISRDNSKNTLYLQMNSLRAEDTAVYYCARDYLGSSYWYFDVWGQGTLVTVSS

SEQ ID NO: 4 - protein, 123 aa, synthetic construct
EVQLLESGGGLVQPGGSLRLSCAASGFTFSDGYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 5 - protein, 123 aa, synthetic construct
EVQLLESGGGLVQPGGSLRLSCAASGFTASDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 6 - protein, 123 aa, synthetic construct
EVQLLESGGGLVQPGGSLRLSCAASGFTFSDSYMEWVRQAPGKGLEWVAASRNKANDYTTSYADSVKGRFTISRDNSKNTLYLQMNSLRAEDTAVYYCARDYYGSSYWYFDVWGQGTLVTVSS

SEQ ID NO: 7 - protein, 123 aa, synthetic construct
EVQLLESGGGLVQPGGSLRLSCAASGFTFEDFYMEWVRQAPGKGLEWVAASRNKANDYTTEYADSVKGRFTISRDNSKNTLYLQMNSLRAEDTAVYYCARDYFGSSYWYFDVWGQGTLVTVSS

SEQ ID NO: 8 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 9 - protein, 123 aa, synthetic construct
EVQLLESGGGLVQPGGSLRLSCAASGFTFSDQYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 10 - protein, 123 aa, synthetic construct
EVQLLESGGGLVQPGGSLRLSCAASGFQFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 11 - protein, 123 aa, synthetic construct
EVQLLESGGGLVQPGGSLRLSCAASGFEFSDNYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 12 - protein, 123 aa, synthetic construct
EVQLLESGGGLVQPGGSLRLSCAASGFTFSDGYMEWVRQAPGKGLEWVAASRNKANDYTTEYADSVKGRFTISRDNSKNTLYLQMNSLRAEDTAVYYCARDYYASSYWYFDVWGQGTLVTVSS

SEQ ID NO: 13 - protein, 123 aa, synthetic construct
EVQLLESGGGLVQPGGSLRLSCAASGFTFSDFYMEWVRQAPGKGLEWVAASRNKANDYTTELADSVKGRFTISRDNSKNTLYLQMNSLRAEDTAVYYCARDYYGSSYWYFDVWGQGTLVTVSS

SEQ ID NO: 14 - protein, 123 aa, synthetic construct
EVQLLESGGGLVQPGGSLRLSCAASGFEFSDFYMEWVRQAPGKGLEWVAASRGKGNSYTTEYADSVKGRFTISRDNSKNTLYLQMNSLRAEDTAVYYCARDYYGSSYWYFDVWGQGTLVTVSS

SEQ ID NO: 15 - protein, 123 aa, synthetic construct
EVQLLESGGGLVQPGGSLRLSCAASGFTFSDGYMEWVRQAPGKGLEWVAASRNKANDYTTEYADSVKGRFTISRDNSKNTLYLQMNSLRAEDTAVYYCARDYYGSPYWYFDVWGQGTLVTVSS

SEQ ID NO: 16 - protein, 123 aa, synthetic construct
EVQLLESGGGLVQPGGSLRLSCAASGFTFSDAYMEWVRQAPGKGLEWVAASRNKANDYTTEYADSVKGRFTISRDNSKNTLYLQMNSLRAEDTAVYYCARDYYGSSYWYFDSWGQGTLVTVSS

SEQ ID NO: 17 - protein, 123 aa, synthetic construct
EVQLLESGGGLVQPGGSLRLSCAASGFTESDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 18 - protein, 123 aa, synthetic construct
EVQLLESGGGLVQPGGSLRLSCAASGFTFSDAYMEWVRQAPGKGLEWVAASRNKANDYTTEYADSVKGRFTISRDNSKNTLYLQMNSLRAEDTAVYYCARDYYGSSYWYFDTWGQGTLVTVSS

SEQ ID NO: 19 - protein, 123 aa, synthetic construct
EVQLLESGGGLVQPGGSLRLSCAASGFTFSDQYMEWVRQAPGKGLEWVAASRNKANDYTTEYADSVKGRFTISRDNSKNTLYLQMNSLRAEDTAVYYCARDYYGSSYWYFDLWGQGTLVTVSS

SEQ ID NO: 20 - protein, 123 aa, synthetic construct
EVQLLESGGGLVQPGGSLRLSCAASGFTFSDFYMEWVRQAPGKGLEWVAASRNKANDYVTEYADSVKGRFTISRDNSKNTLYLQMNSLRAEDTAVYYCARDYYGSSYWYFDVWGQGTLVTVSS

SEQ ID NO: 21 - protein, 123 aa, synthetic construct
EVQLLESGGGLVQPGGSLRLSCAASGFTFSSYYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 22 - protein, 123 aa, synthetic construct
EVQLLESGGGLVQPGGSLRLSCAASGFTFSSYYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 23 - protein, 123 aa, synthetic construct
EVQLLESGGGLVQPGGSLRLSCAASGFTGSDFYMEWVRQAPGKGLEWVAASRNKGNSYTTEYADSVKGRFTISRDNSKNTLYLQMNSLRAEDTAVYYCARDYYGSSYWYFDVWGQGTLVTVSS

SEQ ID NO: 24 - protein, 123 aa, synthetic construct
EVQLLESGGGLVQPGGSLRLSCAASGFTFSDYYMEWVRQAPGKGLEWVAASRNKANTYTTEYADSVKGRFTISRDNSKNTLYLQMNSLRAEDTAVYYCARDYYGSSYWYFDVWGQGTLVTVSS

SEQ ID NO: 25 - protein, 123 aa, synthetic construct
EVQLLESGGGLVQPGGSLRLSCAASGFTFSDGYMEWVRQAPGKGLEWVAASRNKNNDYTTEYADSVKGRFTISRDNSKNTLYLQMNSLRAEDTAVYYCARDYFGSSYWYFDVWGQGTLVTVSS

SEQ ID NO: 26 - protein, 123 aa, synthetic construct
EVQLLESGGGLVQPGGSLRLSCAASGFAFSDFYMEWVRQAPGKGLEWVAASRNKANDYTTEYADSVKGRFTISRDNSKNTLYLQMNSLRAEDTAVYYCARDYYGSHYWYFDVWGQGTLVTVSS

SEQ ID NO: 27 - protein, 123 aa, synthetic construct
EVQLLESGGGLVQPGGSLRLSCAASGFTHSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 28 - protein, 123 aa, synthetic construct
EVQLLESGGGLVQPGGSLRLSCAASGFTFSDGYMEWVRQAPGKGLEWVAASRNKNNDYTTEYADSVKGRFTISRDNSKNTLYLQMNSLRAEDTAVYYCARDYYGSSYWYFDVWGQGTLVTVSS

SEQ ID NO: 29 - protein, 123 aa, synthetic construct
EVQLLESGGGLVQPGGSLRLSCAASGFTFSDTYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 30 - protein, 123 aa, synthetic construct
EVQLLESGGGLVQPGGSLRLSCAASGFAFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 31 - protein, 123 aa, synthetic construct
EVQLLESGGGLVQPGGSLRLSCAASGFTFSDGYMEWVRQAPGKGLEWVAASRNIANDYTTEYADSVKGRFTISRDNSKNTLYLQMNSLRAEDTAVYYCARDYYGSSYWYFDVWGQGTLVTVSS

SEQ ID NO: 32 - protein, 123 aa, synthetic construct
EVQLLESGGGLVQPGGSLRLSCAASGFTFSDDYMEWVRQAPGKGLEWVAASRNKANDYTTEYADSVKGRFTISRDNSKNTLYLQMNSLRAEDTAVYYCARDYQGSSYWYFDVWGQGTLVTVSS

SEQ ID NO: 33 - protein, 123 aa, synthetic construct
EVQLLESGGGLVQPGGSLRLSCAASGFTFSDYYMEWVRQAPGKGLEWVAASRNSGNDYTTEYADSVKGRFTISRDNSKNTLYLQMNSLRAEDTAVYYCARDYYGSSYWYFDVWGQGTLVTVSS

SEQ ID NO: 34 - protein, 123 aa, synthetic construct
EVQLLESGGGLVQPGGSLRLSCAASGFTFSDNYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 35 - protein, 123 aa, synthetic construct
EVQLLESGGGLVQPGGSLRLSCAASGFTFSDAYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 36 - protein, 123 aa, synthetic construct
EVQLLESGGGLVQPGGSLRLSCAASGFTFSDFYMEWVRQAPGKGLEWVAASRNKANDYTVEYADSVKGRFTISRDNSKNTLYLQMNSLRAEDTAVYYCARDYYGHSYWYFDVWGQGTLVTVSS

SEQ ID NO: 37 - protein, 123 aa, synthetic construct
EVQLLESGGGLVQPGGSLRLSCAASGFTSSDFYMEWVRQAPGKGLEWVAASRNKANDYYTEYADSVKGRFTISRDNSKNTLYLQMNSLRAEDTAVYYCARDYYGSSYWYFDVWGQGTLVTVSS

SEQ ID NO: 38 - protein, 123 aa, synthetic construct
EVQLLESGGGLVQPGGSLRLSCAASGFGFSDFYMEWVRQAPGKGLEWVAASRHKANDYTTEYADSVKGRFTISRDNSKNTLYLQMNSLRAEDTAVYYCARDYYGSSYWYFDVWGQGTLVTVSS

SEQ ID NO: 39 - protein, 123 aa, synthetic construct
EVQLLESGGGLVQPGGSLRLSCAASGFTFSTFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 40 - protein, 123 aa, synthetic construct
EVQLLESGGGLVQPGGSLRLSCAASGFTFSDEYMEWVRQAPGKGLEWVAASRGKGNSYTTEYADSVKGRFTISRDNSKNTLYLQMNSLRAEDTAVYYCARDYYGSSYWYFDVWGQGTLVTVSS

SEQ ID NO: 41 - protein, 123 aa, synthetic construct
EVQLLESGGGLVQPGGSLRLSCAASGFTFSDQYMEWVRQAPGKGLEWVAASRNKANDYQTEYADSVKGRFTISRDNSKNTLYLQMNSLRAEDTAVYYCARDYYGSSYWYFDVWGQGTLVTVSS

SEQ ID NO: 42 - protein, 123 aa, synthetic construct
EVQLLESGGGLVQPGGSLRLSCAASGFWFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 43 - protein, 123 aa, synthetic construct
EVQLLESGGGLVQPGGSLRLSCAASGFTFSSYYMEWVRQAPGKGLEWVAASRNQANDYTTEYADSVKGRFTISRDNSKNTLYLQMNSLRAEDTAVYYCARDYYGSPYWYFDVWGQGTLVTVSS

SEQ ID NO: 44 - protein, 123 aa, synthetic construct
EVQLLESGGGLVQPGGSLRLSCAASGFTFSDVYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 45 - protein, 123 aa, synthetic construct
EVQLLESGGGLVQPGGSLRLSCAASGFTFSSYYMEWVRQAPGKGLEWVAASRNQANDYTTEYADSVKGRFTISRDNSKNTLYLQMNSLRAEDTAVYYCARDYYGSSYWYLDVWGQGTLVTVSS

SEQ ID NO: 46 - protein, 123 aa, synthetic construct
EVQLLESGGGLVQPGGSLRLSCAASGFTFSDFYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 47 - protein, 123 aa, synthetic construct
EVQLLESGGGLVQPGGSLRLSCAASGATFSDFYMEWVRQAPGKGLEWVAASRNGANDYTTEYADSVKGRFTISRDNSKNTLYLQMNSLRAEDTAVYYCARDYYGNSYWYFDVWGQGTLVTVSS

SEQ ID NO: 48 - protein, 123 aa, synthetic construct
EVQLLESGGGLVQPGGSLRLSCAASGFDFSDFYMEWVRQAPGKGLEWVAASRNKANDYTTDYADSVKGRFTISRDNSKNTLYLQMNSLRAEDTAVYYCARDYFGSSYWYFDVWGQGTLVTVSS

SEQ ID NO: 49 - protein, 123 aa, synthetic construct
EVQLLESGGGLVQPGGSLRLSCAASGFTFSDEYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 50 - protein, 123 aa, synthetic construct
EVQLLESGGGLVQPGGSLRLSCAASGFWFSDFYMEWVRQAPGKGLEWVAASRNKANDYTTEYADSVKGRFTISRDNSKNTLYLQMNSLRAEDTAVYYCARDYYGSAYWYFDVWGQGTLVTVSS

SEQ ID NO: 51 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGHSYWYFDVWGQGTLVTVSS

SEQ ID NO: 52 - protein, 123 aa, synthetic construct
EVQLLESGGGLVQPGGSLRLSCAASGFTFSDYYMEWVRQAPGKGLEWVAASRNKANDYTVEYADSVKGRFTISRDNSKNTLYLQMNSLRAEDTAVYYCARDYYGSSYWYFDVWGQGTLVTVSS

SEQ ID NO: 53 - protein, 123 aa, synthetic construct
EVQLLESGGGLVQPGGSLRLSCAASGWTFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 54 - protein, 123 aa, synthetic construct
EVQLLESGGGLVQPGGSLRLSCAASGFTFSDNYMEWVRQAPGKGLEWVAASRNKANDYTNEYADSVKGRFTISRDNSKNTLYLQMNSLRAEDTAVYYCARDYYGSSYWYFDVWGQGTLVTVSS

SEQ ID NO: 55 - protein, 123 aa, synthetic construct
EVQLLESGGGLVQPGGSLRLSCAASGFTFSSFYMEWVRQAPGKGLEWVAASRNAANDYTTEYADSVKGRFTISRDNSKNTLYLQMNSLRAEDTAVYYCARDYYGSSYWYFDLWGQGTLVTVSS

SEQ ID NO: 56 - protein, 123 aa, synthetic construct
EVQLLESGGGLVQPGGSLRLSCAASGFTASDFYMEWVRQAPGKGLEWVAASRNKANDQTTEYADSVKGRFTISRDNSKNTLYLQMNSLRAEDTAVYYCARDYYGSSYWYFDSWGQGTLVTVSS

SEQ ID NO: 57 - protein, 123 aa, synthetic construct
EVQLLESGGGLVQPGGSLRLSCAASGFTFNDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 58 - protein, 123 aa, synthetic construct
EVQLLESGGGLVQPGGSLRLSCAASGFTFSDHYMEWVRQAPGKGLEWVAASRNSGNSYTTEYADSVKGRFTISRDNSKNTLYLQMNSLRAEDTAVYYCARDYYGSSYWYFDVWGQGTLVTVSS

SEQ ID NO: 59 - protein, 123 aa, synthetic construct
EVQLLESGGGLVQPGGSLRLSCAASGFTFSDGYIEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 60 - protein, 123 aa, synthetic construct
EVQLLESGGGLVQPGGSLRLSCAASGFTSSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 61 - protein, 123 aa, synthetic construct
EVQLLESGGGLVQPGGSLRLSCAASGFTFSDHYMEWVRQAPGKGLEWVAASRNKANDYTTEYADSVKGRFTISRDNSKNTLYLQMNSLRAEDTAVYYCARDYYGQSYWYFDVWGQGTLVTVSS

SEQ ID NO: 62 - protein, 123 aa, synthetic construct
EVQLLESGGGLVQPGGSLRLSCAASGFTGSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 63 - protein, 123 aa, synthetic construct
EVQLLESGGGLVQPGGSLRLSCAASGGTFSDFYMEWVRQAPGKGLEWVAASRNKANTYTTEYADSVKGRFTISRDNSKNTLYLQMNSLRAEDTAVYYCARDYYGSSYWYFDVWGQGTLVTVSS

SEQ ID NO: 64 - protein, 123 aa, synthetic construct
EVQLLESGGGLVQPGGSLRLSCAASGFTFSDAYMEWVRQAPGKGLEWVAASRNKANSYTTEYADSVKGRFTISRDNSKNTLYLQMNSLRAEDTAVYYCARDYYGSSYWYFDVWGQGTLVTVSS

SEQ ID NO: 65 - protein, 123 aa, synthetic construct
EVQLLESGGGLVQPGGSLRLSCAASGFTQSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 66 - protein, 123 aa, synthetic construct
EVQLLESGGGLVQPGGSLRLSCAASGFTFSDYYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 67 - protein, 123 aa, synthetic construct
EVQLLESGGGLVQPGGSLRLSCAASGDTFSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 68 - protein, 123 aa, synthetic construct
EVQLLESGGGLVQPGGSLRLSCAASGFTFSDGYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 69 - protein, 123 aa, synthetic construct
EVQLLESGGGLVQPGGSLRLSCAASGFTFSDDYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 70 - protein, 123 aa, synthetic construct
EVQLLESGGGLVQPGGSLRLSCAASGFTTSDFYMEWVRQAPGKGLEWVAASRNKANSYTTEYADSVKGRFTISRDNSKNTLYLQMNSLRAEDTAVYYCARDYYGSSYWYFDVWGQGTLVTVSS

SEQ ID NO: 71 - protein, 123 aa, synthetic construct
EVQLLESGGGLVQPGGSLRLSCAASGETFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 72 - protein, 123 aa, synthetic construct
EVQLLESGGGLVQPGGSLRLSCAASGFTFSDHYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 73 - protein, 123 aa, synthetic construct
EVQLLESGGGLVQPGGSLRLSCAASGDTFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 74 - protein, 123 aa, synthetic construct
EVQLLESGGGLVQPGGSLRLSCAASGFTGSDFYMEWVRQAPGKGLEWVAASRGKGNSYTTEYADSVKGRFTISRDNSKNTLYLQMNSLRAEDTAVYYCARDYYGSSTWYFDVWGQGTLVTVSS

SEQ ID NO: 75 - protein, 123 aa, synthetic construct
EVQLLESGGGLVQPGGSLRLSCAASGFTFSDFYMEWVRQAPGKGLEWVAASRWKANDYTTEYADSVKGRFTISRDNSKNTLYLQMNSLRAEDTAVYYCARDYYESSYWYFDVWGQGTLVTVSS

SEQ ID NO: 76 - protein, 123 aa, synthetic construct
EVQLLESGGGLVQPGGSLRLSCAASGFPFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 77 - protein, 123 aa, synthetic construct
EVQLLESGGGLVQPGGSLRLSCAASGFTFEDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 78 - protein, 123 aa, synthetic construct
EVQLLESGGGLVQPGGSLRLSCAASGATFSDFYVEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 79 - protein, 123 aa, synthetic construct
EVQLLESGGGLVQPGGSLRLSCAASGFTFSDYYMEWVRQAPGKGLEWVAASRNKANDYTTEYADSVKGRFTISRDNSKNTLYLQMNSLRAEDTAVYYCARDYYGNSYWYFDVWGQGTLVTVSS

SEQ ID NO: 80 - protein, 123 aa, synthetic construct
EVQLLESGGGLVQPGGSLRLSCAASGFTQSDFYMEWVRQAPGKGLEWVAAIRNKANSYTTEYADSVKGRFTISRDNSKNTLYLQMNSLRAEDTAVYYCARDYYGSSYWYFDVWGQGTLVTVSS

SEQ ID NO: 81 - protein, 123 aa, synthetic construct
EVQLLESGGGLVQPGGSLRLSCAASGGTFSDFYMEWVRQAPGKGLEWVAASRNKANSYTTAYADSVKGRFTISRDNSKNTLYLQMNSLRAEDTAVYYCARDYYGSSYWYFDVWGQGTLVTVSS

SEQ ID NO: 82 - protein, 123 aa, synthetic construct
EVQLLESGGGLVQPGGSLRLSCAASGFTFSSYYMEWVRQAPGKGLEWVAASRNKYNDYTTEYADSVKGRFTISRDNSKNTLYLQMNSLRAEDTAVYYCARDYYGSSTWYFDVWGQGTLVTVSS

SEQ ID NO: 83 - protein, 123 aa, synthetic construct
EVQLLESGGGLVQPGGSLRLSCAASGFTFSDFYMEWVRQAPGKGLEWVAASRNKANDYTTTYADSVKGRFTISRDNSKNTLYLQMNSLRAEDTAVYYCARDYYGSSHWYFDVWGQGTLVTVSS

SEQ ID NO: 84 - protein, 123 aa, synthetic construct
EVQLLESGGGLVQPGGSLRLSCAASGFTFSSYYMEWVRQAPGKGLEWVAASRNKGNSYTTEYADSVKGRFTISRDNSKNTLYLQMNSLRAEDTAVYYCARDYYGSSYWYFDVWGQGTLVTVSS

SEQ ID NO: 85 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGSSYWYFDSWGQGTLVTVSS

SEQ ID NO: 86 - protein, 123 aa, synthetic construct
EVQLLESGGGLVQPGGSLRLSCAASGFTFSDYYMEWVRQAPGKGLEWVAASRNKANDYTTEYADSVKGRFTISRDNSKNTLYLQMNSLRAEDTAVYYCARDYYGDSYWYFDVWGQGTLVTVSS

SEQ ID NO: 87 - protein, 123 aa, synthetic construct
EVQLLESGGGLVQPGGSLRLSCAASGFTDSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 88 - protein, 123 aa, synthetic construct
EVQLLESGGGLVQPGGSLRLSCAASGFTFSSYYMEWVRQAPGKGLEWVAASRNKANDYTTEYADSVKGRFTISRDNSKNTLYLQMNSLRAEDTAVYYCARDYYGSSYWYNDVWGQGTLVTVSS

SEQ ID NO: 89 - protein, 123 aa, synthetic construct
EVQLLESGGGLVQPGGSLRLSCAASGFTFSDYYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 90 - protein, 123 aa, synthetic construct
EVQLLESGGGLVQPGGSLRLSCAASGFTFSDYYMEWVRQAPGKGLEWVAASRNKGNDYTTEYADSVKGRFTISRDNSKNTLYLQMNSLRAEDTAVYYCARDYYGSSYWYFDVWGQGTLVTVSS

SEQ ID NO: 91 - protein, 123 aa, synthetic construct
EVQLLESGGGLVQPGGSLRLSCAASGFGFSDFYMEWVRQAPGKGLEWVGASRNKHNDYTTEYADSVKGRFTISRDNSKNTLYLQMNSLRAEDTAVYYCARDYYGSSYWYFDVWGQGTLVTVSS

SEQ ID NO: 92 - protein, 123 aa, synthetic construct
EVQLLESGGGLVQPGGSLRLSCAASGDTFSDFYMEWVRQAPGKGLEWVAASRNKANAYTTEYADSVKGRFTISRDNSKNTLYLQMNSLRAEDTAVYYCARDYYGQSYWYFDVWGQGTLVTVSS

SEQ ID NO: 93 - protein, 123 aa, synthetic construct
EVQLLESGGGLVQPGGSLRLSCAASGNTFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 94 - protein, 123 aa, synthetic construct
EVQLLESGGGLVQPGGSLRLSCAASGFTFSSYYMEWVRQAPGKGLEWVAASRNKFNDYTTEYADSVKGRFTISRDNSKNTLYLQMNSLRAEDTAVYYCARDYYGSSYWYFDVWGQGTLVTVSS

SEQ ID NO: 95 - protein, 123 aa, synthetic construct
EVQLLESGGGLVQPGGSLRLSCAASGFPFSDFYMEWVRQAPGKGLEWVAASRNKANDYTTEYADSVKGRFTISRDNSKNTLYLQMNSLRAEDTAVYYCARDYYGSPYWYFDVWGQGTLVTVSS

SEQ ID NO: 96 - protein, 123 aa, synthetic construct
EVQLLESGGGLVQPGGSLRLSCAASGFTFSDSYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 97 - protein, 123 aa, synthetic construct
EVQLLESGGGLVQPGGSLRLSCAASGDTFSDFYLEWVRQAPGKGLEWVAASRNKANGYTTEYADSVKGRFTISRDNSKNTLYLQMNSLRAEDTAVYYCARDYYHSSYWYFDVWGQGTLVTVSS

SEQ ID NO: 98 - protein, 123 aa, synthetic construct
EVQLLESGGGLVQPGGSLRLSCAASGFTFSSYYMEWVRQAPGKGLEWVAASRNKANDYTTEYADSVKGRFTISRDNSKNTLYLQMNSLRAEDTAVYYCARDYYGSSYWYYDVWGQGTLVTVSS

SEQ ID NO: 99 - protein, 123 aa, synthetic construct
EVQLLESGGGLVQPGGSLRLSCAASGFTHSDFYMEWVRQAPGKGLEWVAASRNKANGYTTEYADSVKGRFTISRDNSKNTLYLQMNSLRAEDTAVYYCARDYYGSSYWYFDSWGQGTLVTVSS

SEQ ID NO: 100 - protein, 123 aa, synthetic construct
EVQLLESGGGLVQPGGSLRLSCAASGFLFSDFYMEWVRQAPGKGLEWVAASRNKANDYTTTYADSVKGRFTISRDNSKNTLYLQMNSLRAEDTAVYYCARDYYGSHYWYFDVWGQGTLVTVSS

SEQ ID NO: 101 - protein, 123 aa, synthetic construct
EVQLLESGGGLVQPGGSLRLSCAASGFTFSSYYMEWVRQAPGKGLEWVAASRNKANEYTTEYADSVKGRFTISRDNSKNTLYLQMNSLRAEDTAVYYCARDYYGSSYWYFDVWGQGTLVTVSS

SEQ ID NO: 102 - protein, 123 aa, synthetic construct
EVQLLESGGGLVQPGGSLRLSCAASGFTFSDFYMEWVRQAPGKGLEWVAASRNKNNDYTTEYADSVKGRFTISRDNSKNTLYLQMNSLRAEDTAVYYCARDYYGSSYWYFDVWGQGTLVTVSS

SEQ ID NO: 103 - protein, 123 aa, synthetic construct
EVQLLESGGGLVQPGGSLRLSCAASGFTFSSYYMEWVRQAPGKGLEWVAASRNKANLYTTEYADSVKGRFTISRDNSKNTLYLQMNSLRAEDTAVYYCARDYYGSSTWYFDVWGQGTLVTVSS

SEQ ID NO: 104 - protein, 123 aa, synthetic construct
EVQLLESGGGLVQPGGSLRLSCAASGFTFSDHYMEWVRQAPGKGLEWVAASRGKGNSYTTAYADSVKGRFTISRDNSKNTLYLQMNSLRAEDTAVYYCARDYYGSSYWYFDVWGQGTLVTVSS

SEQ ID NO: 105 - protein, 123 aa, synthetic construct
EVQLLESGGGLVQPGGSLRLSCAASGFTFSDFYMEWVRQAPGKGLEWVAASRNKGNSYTTAYADSVKGRFTISRDNSKNTLYLQMNSLRAEDTAVYYCARDYYGSSYWYFDVWGQGTLVTVSS

SEQ ID NO: 106 - protein, 123 aa, synthetic construct
EVQLLESGGGLVQPGGSLRLSCAASGFTFSDHYMEWVRQAPGKGLEWVAASRNKYNDYTTEYADSVKGRFTISRDNSKNTLYLQMNSLRAEDTAVYYCARDIYGSSYWYFDVWGQGTLVTVSS

SEQ ID NO: 107 - protein, 123 aa, synthetic construct
EVQLLESGGGLVQPGGSLRLSCAASGFTFSDYYMEWVRQAPGKGLEWVAASRNKANTYTTEYADSVKGRFTISRDNSKNTLYLQMNSLRAEDTAVYYCARDYYGSSYWYDDVWGQGTLVTVSS

SEQ ID NO: 108 - protein, 123 aa, synthetic construct
EVQLLESGGGLVQPGGSLRLSCAASGFTFSDQYMEWVRQAPGKGLEWVAASRNNANDYTTEYADSVKGRFTISRDNSKNTLYLQMNSLRAEDTAVYYCARDYYGSSYWYFDVWGQGTLVTVSS

SEQ ID NO: 109 - protein, 123 aa, synthetic construct
EVQLLESGGGLVQPGGSLRLSCAASGFTNSDFYMEWVRQAPGKGLEWVAASRNKFNDYTTEYADSVKGRFTISRDNSKNTLYLQMNSLRAEDTAVYYCARDYYGSSYWYFDVWGQGTLVTVSS

SEQ ID NO: 110 - protein, 123 aa, synthetic construct
EVQLLESGGGLVQPGGSLRLSCAASGFTFSDYYMEWVRQAPGKGLEWVAASRWKANDYTTEYADSVKGRFTISRDNSKNTLYLQMNSLRAEDTAVYYCARDYYGSSYWYFDVWGQGTLVTVSS

SEQ ID NO: 111 - protein, 123 aa, synthetic construct
EVQLLESGGGLVQPGGSLRLSCAASGQTFSDFYMEWVRQAPGKGLEWVAASRNKANGYTTEYTDSVKGRFTISRDNSKNTLYLQMNSLRAEDTAVYYCARDYYGSSYWYFDVWGQGTLVTVSS

SEQ ID NO: 112 - protein, 123 aa, synthetic construct
EVQLLESGGGLVQPGGSLRLSCAASGFTASDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 113 - protein, 123 aa, synthetic construct
EVQLLESGGGLVQPGGSLRLSCAASGFTFSDPYMEWVRQAPGKGLEWVAASRNKANDYTTEYADSVKGRFTISRDNSKNTLYLQMNSLRAEDTAVYYCARDYYGSPYWYFDVWGQGTLVTVSS

SEQ ID NO: 114 - protein, 123 aa, synthetic construct
EVQLLESGGGLVQPGGSLRLSCAASGFTFSDYYMEWVRQAPGKGLEWVAASRNKANDYTTEYADSVKGRFTISRDNSKNTLYLQMNSLRAEDTAVYYCARDYYGSSYWYFDTWGQGTLVTVSS

SEQ ID NO: 115 - protein, 123 aa, synthetic construct
EVQLLESGGGLVQPGGSLRLSCAASGFTQSDFYMEWVRQAPGKGLEWVAASRNKANAYTTEYADSVKGRFTISRDNSKNTLYLQMNSLRAEDTAVYYCARDYYGYSYWYFDVWGQGTLVTVSS

SEQ ID NO: 116 - protein, 123 aa, synthetic construct
EVQLLESGGGLVQPGGSLRLSCAASGFTFSDEYMEWVRQAPGKGLEWVAASRNSGNSYTTAYADSVKGRFTISRDNSKNTLYLQMNSLRAEDTAVYYCARDYYGSSYWYFDVWGQGTLVTVSS

SEQ ID NO: 117 - protein, 123 aa, synthetic construct
EVQLLESGGGLVQPGGSLRLSCAASGFTFSDYYMEWVRQAPGKGLEWVAASRWKANDYTTEYADSVKGRFTISRDNSKNTLYLQMNSLRAEDTAVYYCARDYYGSSQWYFDVWGQGTLVTVSS

SEQ ID NO: 118 - protein, 123 aa, synthetic construct
EVQLLESGGGLVQPGGSLRLSCAASGFTFSDDYMEWVRQAPGKGLEWVAATRNKANDYTTEYADSVKGRFTISRDNSKNTLYLQMNSLRAEDTAVYYCARDYYGSSYWYFDSWGQGTLVTVSS

SEQ ID NO: 119 - protein, 123 aa, synthetic construct
EVQLLESGGGLVQPGGSLRLSCAASGFTFSDQYMEWVRQAPGKGLEWVAASRNKANDYTVEYADSVKGRFTISRDNSKNTLYLQMNSLRAEDTAVYYCARDYYGSSYWYFDVWGQGTLVTVSS

SEQ ID NO: 120 - protein, 123 aa, synthetic construct
EVQLLESGGGLVQPGGSLRLSCAASGFGFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 121 - protein, 123 aa, synthetic construct
EVQLLESGGGLVQPGGSLRLSCAASGFTGSDFYMEWVRQAPGKGLEWVAASRNSGNSYTTAYADSVKGRFTISRDNSKNTLYLQMNSLRAEDTAVYYCARDYFGSSYWYFDVWGQGTLVTVSS

SEQ ID NO: 122 - protein, 123 aa, synthetic construct
EVQLLESGGGLVQPGGSLRLSCAASGFTFSDEYMEWVRQAPGKGLEWVAASRNKANDFTTEYADSVKGRFTISRDNSKNTLYLQMNSLRAEDTAVYYCARDFYGSSYWYFDVWGQGTLVTVSS

SEQ ID NO: 123 - protein, 123 aa, synthetic construct
EVQLLESGGGLVQPGGSLRLSCAASGFQFSDFYMEWVRQAPGKGLEWVAASRNKGNSYTTAYADSVKGRFTISRDNSKNTLYLQMNSLRAEDTAVYYCARDYYGSSYWYFDVWGQGTLVTVSS

SEQ ID NO: 124 - protein, 123 aa, synthetic construct
EVQLLESGGGLVQPGGSLRLSCAASGTTFSDFYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 125 - protein, 123 aa, synthetic construct
EVQLLESGGGLVQPGGSLRLSCAASGFGFSDFYMEWVRQAPGKGLEWVAASRNKANDYTTEYADSVKGRFTISRDNSKNTLYLQMNSLRAEDTAVYYCARDYYGSSYWYHDVWGQGTLVTVSS

SEQ ID NO: 126 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGSSHWYFDVWGQGTLVTVSS

SEQ ID NO: 127 - protein, 123 aa, synthetic construct
EVQLLESGGGLVQPGGSLRLSCAASGPTFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 128 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGSSYWYFDPWGQGTLVTVSS

SEQ ID NO: 129 - protein, 123 aa, synthetic construct
EVQLLESGGGLVQPGGSLRLSCAASGFTFSDAYMEWVRQAPGKGLEWVAASRNKANDYTTEYADSVKGRFTISRDNSKNTLYLQMNSLRAEDTAVYYCARDYYGSHYWYFDVWGQGTLVTVSS

SEQ ID NO: 130 - protein, 123 aa, synthetic construct
EVQLLESGGGLVQPGGSLRLSCAASGFTFSSYYMEWVRQAPGKGLEWVAASRNKANDYTYEYADSVKGRFTISRDNSKNTLYLQMNSLRAEDTAVYYCARDYYESSYWYFDVWGQGTLVTVSS

SEQ ID NO: 131 - protein, 123 aa, synthetic construct
EVQLLESGGGLVQPGGSLRLSCAASGDTFSDFYMEWVRQAPGKGLEWVAASRNKANDYSTEYADSVKGRFTISRDNSKNTLYLQMNSLRAEDTAVYYCARDYYGSSYWYFDVWGQGTLVTVSS

SEQ ID NO: 132 - protein, 123 aa, synthetic construct
EVQLLESGGGLVQPGGSLRLSCAASGFTFSEFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 133 - protein, 123 aa, synthetic construct
EVQLLESGGGLVQPGGSLRLSCAASGFTFSSYYMEWVRQAPGKGLEWVAASRSKANDYTTEYADSVKGRFTISRDNSKNTLYLQMNSLRAEDTAVYYCARDYYGSQYWYFDVWGQGTLVTVSS

SEQ ID NO: 134 - protein, 123 aa, synthetic construct
EVQLLESGGGLVQPGGSLRLSCAASGFHFSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 135 - protein, 123 aa, synthetic construct
EVQLLESGGGLVQPGGSLRLSCAASGFTFSDGYMEWVRQAPGKGLEWVAASRNKANDYTTAYADSVKGRFTISRDNSKNTLYLQMNSLRAEDTAVYYCARDYYGSSYWYFDVWGQGTLVTVSS

SEQ ID NO: 136 - protein, 123 aa, synthetic construct
EVQLLESGGGLVQPGGSLRLSCAASGFTFSSYYMEWVRQAPGKGLEWVAASRNTANDYTTEYADSVKGRFTISRDNSKNTLYLQMNSLRAEDTAVYYCARDYYGSSYWYFDVWGQGTLVTVSS

SEQ ID NO: 137 - protein, 123 aa, synthetic construct
EVQLLESGGGLVQPGGSLRLSCAASGITFSDFYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 138 - protein, 123 aa, synthetic construct
EVQLLESGGGLVQPGGSLRLSCAASGFGFSDFYMEWVRQAPGKGLEWVAASRNKANDYTTEYADSVKGRFTISRDNSKNTLYLQMNSLRAEDTAVYYCARDYYSSSYWYFDVWGQGTLVTVSS

SEQ ID NO: 139 - protein, 123 aa, synthetic construct
EVQLLESGGGLVQPGGSLRLSCAASGFTFSDYYMEWVRQAPGKGLEWVAASRNKANDYNTEYADSVKGRFTISRDNSKNTLYLQMNSLRAEDTAVYYCARDYYGSSYWYFDVWGQGTLVTVSS

SEQ ID NO: 140 - protein, 123 aa, synthetic construct
EVQLLESGGGLVQPGGSLRLSCAASGFTFSDFYVEWVRQAPGKGLEWVAASRNKANTYTTEYADSVKGRFTISRDNSKNTLYLQMNSLRAEDTAVYYCARDYYGSSYWYFDVWGQGTLVTVSS

SEQ ID NO: 141 - protein, 123 aa, synthetic construct
EVQLLESGGGLVQPGGSLRLSCAASGFQFSDFYMEWVRQAPGKGLEWVAASRWKANDYTTEYADSVKGRFTISRDNSKNTLYLQMNSLRAEDTAVYYCARDYYGSSYWYFDVWGQGTLVTVSS

SEQ ID NO: 142 - protein, 123 aa, synthetic construct
EVQLLESGGGLVQPGGSLRLSCAASGFTFSDFYMEWVRQAPGKGLEWVAASRNKANDYTTEVADSVKGRFTISRDNSKNTLYLQMNSLRAEDTAVYYCARDYYGSSNWYFDVWGQGTLVTVSS

SEQ ID NO: 143 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDYYGSSYWYNDVWGQGTLVTVSS

SEQ ID NO: 144 - protein, 123 aa, synthetic construct
EVQLLESGGGLVQPGGSLRLSCAASGSTFSDFYMEWVRQAPGKGLEWVAASRNKANDFTTEYADSVKGRFTISRDNSKNTLYLQMNSLRAEDTAVYYCARDYYGSSYWYFDVWGQGTLVTVSS

SEQ ID NO: 145 - protein, 123 aa, synthetic construct
EVQLLESGGGLVQPGGSLRLSCAASGFTFSDYYMEWVRQAPGKGLEWVAASRNKANDYTTEYADSVKGRFTISRDNSKNTLYLQMNSLRAEDTAVYYCARDYYFSSYWYFDVWGQGTLVTVSS

SEQ ID NO: 146 - protein, 123 aa, synthetic construct
EVQLLESGGGLVQPGGSLRLSCAASGFTTSDFYMEWVRQAPGKGLEWVAASRNKANGYTTEYADSVKGRFTISRDNSKNTLYLQMNSLRAEDTAVYYCARDYYGSSYWYFDVWGQGTLVTVSS

SEQ ID NO: 147 - protein, 123 aa, synthetic construct
EVQLLESGGGLVQPGGSLRLSCAASGFTNSDFYMEWVRQAPGKGLEWVAASRNKANDYTTELADSVKGRFTISRDNSKNTLYLQMNSLRAEDTAVYYCARDYYGSSYWYFDVWGQGTLVTVSS

SEQ ID NO: 148 - protein, 123 aa, synthetic construct
EVQLLESGGGLVQPGGSLRLSCAASGETFSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 149 - protein, 123 aa, synthetic construct
EVQLLESGGGLVQPGGSLRLSCAASGFTFSSYYMEWVRQAPGKGLEWVAASRNKANDYTTEYADSVKGRFTISRDNSKNTLYLQMNSLRAEDTAVYYCARDYYDSSYWYFDVWGQGTLVTVSS

SEQ ID NO: 150 - protein, 123 aa, synthetic construct
EVQLLESGGGLVQPGGSLRLSCAASGFTFSDQYMEWVRQAPGKGLEWVAASRNKNNDYTTEYADSVKGRFTISRDNSKNTLYLQMNSLRAEDTAVYYCARDYYGASYWYFDVWGQGTLVTVSS

SEQ ID NO: 151 - protein, 123 aa, synthetic construct
EVQLLESGGGLVQPGGSLRLSCAASGFTFSDYYMEWVRQAPGKGLEWVAASRNKGNDYTTAYADSVKGRFTISRDNSKNTLYLQMNSLRAEDTAVYYCARDYFGSSYWYFDVWGQGTLVTVSS

SEQ ID NO: 152 - protein, 123 aa, synthetic construct
EVQLLESGGGLVQPGGSLRLSCAASGFTQSDFYMEWVRQAPGKGLEWVAASRNKGNSYTTAYADSVKGRFTISRDNSKNTLYLQMNSLRAEDTAVYYCARDYYGSSYWYFDVWGQGTLVTVSS

SEQ ID NO: 153 - protein, 123 aa, synthetic construct
EVQLLESGGGLVQPGGSLRLSCAASGFTFSDYYMEWVRQAPGKGLEWVAASRNKANSYTTAYADSVKGRFTISRDNSKNTLYLQMNSLRAEDTAVYYCARDYYGSSYWYFDVWGQGTLVTVSS

SEQ ID NO: 154 - protein, 123 aa, synthetic construct
EVQLLESGGGLVQPGGSLRLSCAASGFTFSDGYMEWVRQAPGKGLEWVAASRNKANFYTTEYADSVKGRFTISRDNSKNTLYLQMNSLRAEDTAVYYCARDHYGSSYWYFDVWGQGTLVTVSS

SEQ ID NO: 155 - protein, 123 aa, synthetic construct
EVQLLESGGGLVQPGGSLRLSCAASGFSFSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 156 - protein, 123 aa, synthetic construct
EVQLLESGGGLVQPGGSLRLSCAASGFTESDFYMEWVRQAPGKGLEWVAASRNKYNDYTTEYADSVKGRFTISRDNSKNTLYLQMNSLRAEDTAVYYCARDYYGSSYWYFDVWGQGTLVTVSS

SEQ ID NO: 157 - protein, 123 aa, synthetic construct
EVQLLESGGGLVQPGGSLRLSCAASGFTFDDFYMEWVRQAPGKGLEWVAASRNEANDYTTEYADSVKGRFTISRDNSKNTLYLQMNSLRAEDTAVYYCARDYYGSSYWYFDVWGQGTLVTVSS

SEQ ID NO: 158 - protein, 123 aa, synthetic construct
EVQLLESGGGLVQPGGSLRLSCAASGFTFSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 159 - protein, 123 aa, synthetic construct
EVQLLESGGGLVQPGGSLRLSCAASGFTFSDFYIEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 160 - protein, 123 aa, synthetic construct
EVQLLESGGGLVQPGGSLRLSCAASGFTQSDFYMEWVRQAPGKGLEWVAASRNKANDYTTEYADSVKGRFTISRDNSKNTLYLQMNSLRAEDTAVYYCARDYYGESYWYFDVWGQGTLVTVSS

SEQ ID NO: 161 - protein, 123 aa, synthetic construct
EVQLLESGGGLVQPGGSLRLSCAASGPTFSDFYMEWVRQAPGKGLEWVAASRNKANGYTTEYADSVKGRFTISRDNSKNTLYLQMNSLRAEDTAVYYCARDYYGSSYWYFDVWGQGTLVTVSS

SEQ ID NO: 162 - protein, 123 aa, synthetic construct
EVQLLESGGGLVQPGGSLRLSCAASGFTDSDFYMEWVRQAPGKGLEWVAASRNKANGYTTEYADSVKGRFTISRDNSKNTLYLQMNSLRAEDTAVYYCARDYYGSSYWYFDVWGQGTLVTVSS

SEQ ID NO: 163 - protein, 123 aa, synthetic construct
EVQLLESGGGLVQPGGSLRLSCAASGDTFSDFYMEWVRQAPGKGLEWVAASRWKANDYTTEYADSVKGRFTISRDNSKNTLYLQMNSLRAEDTAVYYCARDYYGSSYWYFDVWGQGTLVTVSS

SEQ ID NO: 164 - protein, 123 aa, synthetic construct
EVQLLESGGGLVQPGGSLRLSCAASGFTFSSYYMEWVRQAPGKGLEWVAASRNKANDYTTEYADSVKGRFTISRDNSKNTLYLQMNSLRAEDTAVYYCARDYYLSSYWYFDVWGQGTLVTVSS

SEQ ID NO: 165 - protein, 123 aa, synthetic construct
EVQLLESGGGLVQPGGSLRLSCAASGFTFSDGYMEWVRQAPGKGLEWVAAIRNKANDYTTEYADSVKGRFTISRDNSKNTLYLQMNSLRAEDTAVYYCARDYYGSHYWYFDVWGQGTLVTVSS

SEQ ID NO: 166 - protein, 123 aa, synthetic construct
EVQLLESGGGLVQPGGSLRLSCAASGFTFSDYYMEWVRQAPGKGLEWVAASRNKGNDYTTAYADSVKGRFTISRDNSKNTLYLQMNSLRAEDTAVYYCARDYYGSSYWYFDVWGQGTLVTVSS

SEQ ID NO: 167 - protein, 123 aa, synthetic construct
EVQLLESGGGLVQPGGSLRLSCAASGFTFSDYYMEWVRQAPGKGLEWVAASRNVANDYTTEYADSVKGRFTISRDNSKNTLYLQMNSLRAEDTAVYYCARDYYGSSYWYFDVWGQGTLVTVSS

SEQ ID NO: 168 - protein, 123 aa, synthetic construct
EVQLLESGGGLVQPGGSLRLSCAASGFTGSDFYMEWVRQAPGKGLEWVAASRWKANDYTTEYADSVKGRFTISRDNSKNTLYLQMNSLRAEDTAVYYCARDYYGSSYWYFDVWGQGTLVTVSS

SEQ ID NO: 169 - protein, 123 aa, synthetic construct
EVQLLESGGGLVQPGGSLRLSCAASGFTFSDQYMEWVRQAPGKGLEWVAASRNKANDWTTEYADSVKGRFTISRDNSKNTLYLQMNSLRAEDTAVYYCARDYYGSSYWYFDVWGQGTLVTVSS

SEQ ID NO: 170 - protein, 123 aa, synthetic construct
EVQLLESGGGLVQPGGSLRLSCAASGNTFSDFYMEWVRQAPGKGLEWVAASRNKANYYTTEYADSVKGRFTISRDNSKNTLYLQMNSLRAEDTAVYYCARDYYGSSYWYFDVWGQGTLVTVSS

SEQ ID NO: 171 - protein, 123 aa, synthetic construct
EVQLLESGGGLVQPGGSLRLSCAASGFTGSDFYMEWVRQAPGKGLEWVAASRNKANDYTTEYADSVKGRFTISRDNSKNTLYLQMNSLRAEDTAVYYCARDYYGSPYWYFDVWGQGTLVTVSS

SEQ ID NO: 172 - protein, 123 aa, synthetic construct
EVQLLESGGGLVQPGGSLRLSCAASGFTSSDFYLEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 173 - protein, 123 aa, synthetic construct
EVQLLESGGGLVQPGGSLRLSCAASGFTFSPFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 174 - protein, 123 aa, synthetic construct
EVQLLESGGGLVQPGGSLRLSCAASGFTASDFYMEWVRQAPGKGLEWVAASRWKANDYTTEYADSVKGRFTISRDNSKNTLYLQMNSLRAEDTAVYYCARDIYGSSYWYFDVWGQGTLVTVSS

SEQ ID NO: 175 - protein, 123 aa, synthetic construct
EVQLLESGGGLVQPGGSLRLSCAASGFTFSDYYMEWVRQAPGKGLEWVAASRNKWNDYTTEYADSVKGRFTISRDNSKNTLYLQMNSLRAEDTAVYYCARDYYLSSYWYFDVWGQGTLVTVSS

SEQ ID NO: 176 - protein, 123 aa, synthetic construct
EVQLLESGGGLVQPGGSLRLSCAASGFTTSDFYMEWVRQAPGKGLEWVAASRNKANDYTTEYADSVKGRFTISRDNSKNTLYLQMNSLRAEDTAVYYCARDYYGSSYWYFDYWGQGTLVTVSS

SEQ ID NO: 177 - protein, 123 aa, synthetic construct
EVQLLESGGGLVQPGGSLRLSCAASGFTFSDFYMEWVRQAPGKGLEWVAASRNKANDYTTEYADSVKGRFTISRDNSKNTLYLQMNSLRAEDTAVYYCARDFYGSSYWYFDVWGQGTLVTVSS

SEQ ID NO: 178 - protein, 123 aa, synthetic construct
EVQLLESGGGLVQPGGSLRLSCAASGFTGSDFYMEWVRQAPGKGLEWVAASRNKANDYTTEYADSVKGRFTISRDNSKNTLYLQMNSLRAEDTAVYYCARDYYGSSYWYFDVWGQGTLVTVSS

SEQ ID NO: 179 - protein, 123 aa, synthetic construct
EVQLLESGGGLVQPGGSLRLSCAASGNTFSDFYMEWVRQAPGKGLEWVAASRNKWNDYTTEYADSVKGRFTISRDNSKNTLYLQMNSLRAEDTAVYYCARDYYGSSYWYFDVWGQGTLVTVSS

SEQ ID NO: 180 - protein, 123 aa, synthetic construct
EVQLLESGGGLVQPGGSLRLSCAASGFTFSDHYMEWVRQAPGKGLEWVAASRWKANDYTTEYADSVKGRFTISRDNSKNTLYLQMNSLRAEDTAVYYCARDYYGSSYWYFDVWGQGTLVTVSS

SEQ ID NO: 181 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAEFYSYPLTFGGGTKVEIK

SEQ ID NO: 182 - protein, 113 aa, synthetic construct
DIQMTQSPSSLSASVGDRVTITCTASESLYSSKHKVHYLAWYQQKPGKAPKLLIYGASNREIGVPSRFSGSGSGTDFTLTISSLQPEDFATYYCQQFYSYPLTFGGGTKVEIK

SEQ ID NO: 183 - protein, 113 aa, synthetic construct
DIQMTQSPSSLSASVGDRVTITCTASESLYSSKHKVHYLAWYQQKPGKAPKLLIYGASHRYIGVPSRFSGSGSGTDFTLTISSLQPEDFATYYCAQFYAYPLTFGGGTKVEIK

SEQ ID NO: 184 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYSYPLTFGGGTKVEIK

SEQ ID NO: 185 - protein, 113 aa, synthetic construct
DIQMTQSPSSLSASVGDRVTITCTASESLYSSKHKVHYLAWYQQKPGKAPKLLIYGASSLQIGVPSRFSGSGSGTDFTLTISSLQPEDFATYYCAQFYSYPLTFGGGTKVEIK

SEQ ID NO: 186 - protein, 113 aa, synthetic construct
DIQMTQSPSSLSASVGDRVTITCTASESLYSSKHKVHYLAWYQQKPGKAPKLLIYGASNRYTGVPSRFSGSGSGTDFTLTISSLQPEDFATYYCAQFYHYPLTFGGGTKVEIK

SEQ ID NO: 187 - protein, 113 aa, synthetic construct
DIQMTQSPSSLSASVGDRVTITCTASESLYSSKHKVHYLAWYQQKPGKAPKLLIYGASNRYHGVPSRFSGSGSGTDFTLTISSLQPEDFATYYCAQFYSYPLTFGGGTKVEIK

SEQ ID NO: 188 - protein, 113 aa, synthetic construct
DIQMTQSPSSLSASVGDRVTITCVASESLYSSKHKVHYLAWYQQKPGKAPKLLIYGASNRYIGVPSRFSGSGSGTDFTLTISSLQPEDFATYYCAQFYSYPLTFGGGTKVEIK

SEQ ID NO: 189 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYQYPLTFGGGTKVEIK

SEQ ID NO: 190 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYYYPLTFGGGTKVEIK

SEQ ID NO: 191 - protein, 113 aa, synthetic construct
DIQMTQSPSSLSASVGDRVTITCTASESLYSSKHKVHYLAWYQQKPGKAPKLLIYGSSNRYIGVPSRFSGSGSGTDFTLTISSLQPEDFATYYCAQFYAYPLTFGGGTKVEIK

SEQ ID NO: 192 - protein, 113 aa, synthetic construct
DIQMTQSPSSLSASVGDRVTITCTASESLYSSKHKVHYLAWYQQKPGKAPKLLIYGASNRFIGVPSRFSGSGSGTDFTLTISSLQPEDFATYYCAQFYYYPLTFGGGTKVEIK

SEQ ID NO: 193 - protein, 113 aa, synthetic construct
DIQMTQSPSSLSASVGDRVTITCRASESISSSKNKVHYLAWYQQKPGKAPKLLIYGASNRYYGVPSRFSGSGSGTDFTLTISSLQPEDFATYYCAQFYSYPLTFGGGTKVEIK

SEQ ID NO: 194 - protein, 113 aa, synthetic construct
DIQMTQSPSSLSASVGDRVTITCTASESLYSSKHKVHYLAWYQQKPGKAPKLLIYGAANRYIGVPSRFSGSGSGTDFTLTISSLQPEDFATYYCAQFYVYPLTFGGGTKVEIK

SEQ ID NO: 195 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCGQFYSYPLTFGGGTKVEIK

SEQ ID NO: 196 - protein, 113 aa, synthetic construct
DIQMTQSPSSLSASVGDRVTITCTASESLYSSKHKVHYLAWYQQKPGKAPKLLIYGASNRYVGVPSRFSGSGSGTDFTLTISSLQPEDFATYYCAQFYSYPLTFGGGTKVEIK

SEQ ID NO: 197 - protein, 113 aa, synthetic construct
DIQMTQSPSSLSASVGDRVTITCTASESLYSAKHKVHYLAWYQQKPGKAPKLLIYGSSNRYIGVPSRFSGSGSGTDFTLTISSLQPEDFATYYCAQFYDYPLTFGGGTKVEIK

SEQ ID NO: 198 - protein, 113 aa, synthetic construct
DIQMTQSPSSLSASVGDRVTITCTASESLYSSKHKVHYLAWYQQKPGKAPKLLIYGASNRYDGVPSRFSGSGSGTDFTLTISSLQPEDFATYYCAQFYSYPLTFGGGTKVEIK

SEQ ID NO: 199 - protein, 113 aa, synthetic construct
DIQMTQSPSSLSASVGDRVTITCTASESLYSSKHKVNYLAWYQQKPGKAPKLLIYGASNRYIGVPSRFSGSGSGTDFTLTISSLQPEDFATYYCAQFYQYPLTFGGGTKVEIK

SEQ ID NO: 200 - protein, 113 aa, synthetic construct
DIQMTQSPSSLSASVGDRVTITCTASESLYSSKHKVHYVAWYQQKPGKAPKLLIYGASNRYIGVPSRFSGSGSGTDFTLTISSLQPEDFATYYCAQFYYYPLTFGGGTKVEIK

SEQ ID NO: 201 - protein, 113 aa, synthetic construct
DIQMTQSPSSLSASVGDRVTITCTASFSLYSSKHKVHYLAWYQQKPGKAPKLLIYGASNRYPGVPSRFSGSGSGTDFTLTISSLQPEDFATYYCAQFYYYPLTFGGGTKVEIK

SEQ ID NO: 202 - protein, 113 aa, synthetic construct
DIQMTQSPSSLSASVGDRVTITCTASESLYSSKHKVHYLAWYQQKPGKAPKLLIYGAVNRYIGVPSRFSGSGSGTDFTLTISSLQPEDFATYYCAQFYRYPLTFGGGTKVEIK

SEQ ID NO: 203 - protein, 113 aa, synthetic construct
DIQMTQSPSSLSASVGDRVTITCTASESLYSSKHKVHYLAWYQQKPGKAPKLLIYGASNGYIGVPSRFSGSGSGTDFTLTISSLQPEDFATYYCAQFYTYPLTFGGGTKVEIK

SEQ ID NO: 204 - protein, 113 aa, synthetic construct
DIQMTQSPSSLSASVGDRVTITCTASESLYGSKHKVHYLAWYQQKPGKAPKLLIYGASNRYSGVPSRFSGSGSGTDFTLTISSLQPEDFATYYCAQFYSYPLTFGGGTKVEIK

SEQ ID NO: 205 - protein, 113 aa, synthetic construct
DIQMTQSPSSLSASVGDRVTITCTASESLYSSKHKVHYLAWYQQKPGKAPKLLIYGASNLYSGVPSRFSGSGSGTDFTLTISSLQPEDFATYYCAQFYSYPLTFGGGTKVEIK

SEQ ID NO: 206 - protein, 113 aa, synthetic construct
DIQMTQSPSSLSASVGDRVTITCTASESLYSSKHKVHYLAWYQQKPGKAPKLLIYGASNRYFGVPSRFSGSGSGTDFTLTISSLQPEDFATYYCAQFYSYPLTFGGGTKVEIK

SEQ ID NO: 207 - protein, 113 aa, synthetic construct
DIQMTQSPSSLSASVGDRVTITCTASESLYSSKHKVHYLAWYQQKPGKAPKLLIYGASNLQSGVPSRFSGSGSGTDFTLTISSLQPEDFATYYCAQFYYYPLTFGGGTKVEIK

SEQ ID NO: 208 - protein, 112 aa, synthetic construct
DIQMTQSPSSLSASVGDRVTITCRASESISSSKHKHYLAWYQQKPGKAPKLLIYGASNRYIGVPSRFSGSGSGTDFTLTISSLQPEDFATYYCAQFYVYPLTFGGGTKVEIK

SEQ ID NO: 209 - protein, 113 aa, synthetic construct
DIQMTQSPSSLSASVGDRVTITCTASESLYSSKHKVHYLAWYQQKPGKAPKLLIYGASNRYFGVPSRFSGSGSGTDFTLTISSLQPEDFATYYCAQFYQYPLTFGGGTKVEIK

SEQ ID NO: 210 - protein, 113 aa, synthetic construct
DIQMTQSPSSLSASVGDRVTITCTASESLYSSKHKVHYLAWYQQKPGKAPKLLIYGLSNRYIGVPSRFSGSGSGTDFTLTISSLQPEDFATYYCAQFYSYPLTFGGGTKVEIK

SEQ ID NO: 211 - protein, 113 aa, synthetic construct
DIQMTQSPSSLSASVGDRVTITCTASESLYSSKHKVHYLAWYQQKPGKAPKLLIYGASNRQSGVPSRFSGSGSGTDFTLTISSLQPEDFATYYCAQFYWYPLTFGGGTKVEIK

SEQ ID NO: 212 - protein, 113 aa, synthetic construct
DIQMTQSPSSLSASVGDRVTITCTASESLYSSKHKVHYLAWYQQKPGKAPKLLIYGASNRQSGVPSRFSGSGSGTDFTLTISSLQPEDFATYYCAQFYAYPLTFGGGTKVEIK

SEQ ID NO: 213 - protein, 113 aa, synthetic construct
DIQMTQSPSSLSASVGDRVTITCTASESLYSSKHKVHYLAWYQQKPGKAPKLLIYGASNRYHGVPSRFSGSGSGTDFTLTISSLQPEDFATYYCAQFYYYPLTFGGGTKVEIK

SEQ ID NO: 214 - protein, 113 aa, synthetic construct
DIQMTQSPSSLSASVGDRVTITCTASESLYSSKHKVHYLAWYQQKPGKAPKLLIYGAFNRYTGVPSRFSGSGSGTDFTLTISSLQPEDFATYYCAQFYTYPLTFGGGTKVEIK

SEQ ID NO: 215 - protein, 113 aa, synthetic construct
DIQMTQSPSSLSASVGDRVTITCTASESLYSSKHKVHYLAWYQQKPGKAPKLLIYGASNRLIGVPSRFSGSGSGTDFTLTISSLQPEDFATYYCAQFYWYPLTFGGGTKVEIK

SEQ ID NO: 216 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCTQFYSYPLTFGGGTKVEIK

SEQ ID NO: 217 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYVYPLTFGGGTKVEIK

SEQ ID NO: 218 - protein, 113 aa, synthetic construct
DIQMTQSPSSLSASVGDRVTITCTASESLYSSKHKVHYLAWYQQKPGKAPKLLIYGQSNRYIGVPSRFSGSGSGTDFTLTISSLQPEDFATYYCAQFYSYPLTFGGGTKVEIK

SEQ ID NO: 219 - protein, 113 aa, synthetic construct
DIQMTQSPSSLSASVGDRVTITCTASEWLYSSKHKVHYLAWYQQKPGKAPKLLIYGASNRNIGVPSRFSGSGSGTDFTLTISSLQPEDFATYYCAQFYYYPLTFGGGTKVEIK

SEQ ID NO: 220 - protein, 113 aa, synthetic construct
DIQMTQSPSSLSASVGDRVTITCTASESLYSSKHKVHYLAWYQQKPGKAPKLLIYGAGNRYIGVPSRFSGSGSGTDFTLTISSLQPEDFATYYCAQFYFYPLTFGGGTKVEIK

SEQ ID NO: 221 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYWYPLTFGGGTKVEIK

SEQ ID NO: 222 - protein, 113 aa, synthetic construct
DIQMTQSPSSLSASVGDRVTITCTASESLYSSKHKVHYLAWYQQKPGKAPKLLIYGASNRYGGVPSRFSGSGSGTDFTLTISSLQPEDFATYYCAQFYEYPLTFGGGTKVEIK

SEQ ID NO: 223 - protein, 113 aa, synthetic construct
DIQMTQSPSSLSASVGDRVTITCTATESLYSSKHKVHYLAWYQQKPGKAPKLLIYGASNRYTGVPSRFSGSGSGTDFTLTISSLQPEDFATYYCAQFYSYPLTFGGGTKVEIK

SEQ ID NO: 224 - protein, 113 aa, synthetic construct
DIQMTQSPSSLSASVGDRVTITCTASESLYSSKHKVHYLAWYQQKPGKAPKLLIYGASGRYIGVPSRFSGSGSGTDFTLTISSLQPEDFATYYCAQFYSYPLTFGGGTKVEIK

SEQ ID NO: 225 - protein, 113 aa, synthetic construct
DIQMTQSPSSLSASVGDRVTITCTASESLYSGKHKVHYLAWYQQKPGKAPKLLIYGASNLQIGVPSRFSGSGSGTDFTLTISSLQPEDFATYYCAQFYYYPLTFGGGTKVEIK

SEQ ID NO: 226 - protein, 113 aa, synthetic construct
DIQMTQSPSSLSASVGDRVTITCFASESLYSSKHKVHYLAWYQQKPGKAPKLLIYGASNRGIGVPSRFSGSGSGTDFTLTISSLQPEDFATYYCAQFYSYPLTFGGGTKVEIK

SEQ ID NO: 227 - protein, 113 aa, synthetic construct
DIQMTQSPSSLSASVGDRVTITCTASQSLYSSKNKVHYLAWYQQKPGKAPKLLIYGESNRYIGVPSRFSGSGSGTDFTLTISSLQPEDFATYYCAQFYSYPLTFGGGTKVEIK

SEQ ID NO: 228 - protein, 113 aa, synthetic construct
DIQMTQSPSSLSASVGDRVTITCEASESLYSSKHKVHYLAWYQQKPGKAPKLLIYGASNRYPGVPSRFSGSGSGTDFTLTISSLQPEDFATYYCAQFYEYPLTFGGGTKVEIK

SEQ ID NO: 229 - protein, 113 aa, synthetic construct
DIQMTQSPSSLSASVGDRVTITCTASESLYSSKHKVHYLAWYQQKPGKAPKLLIYGVSNRYIGVPSRFSGSGSGTDFTLTISSLQPEDFATYYCAQFYSYPLTFGGGTKVEIK

SEQ ID NO: 230 - protein, 113 aa, synthetic construct
DIQMTQSPSSLSASVGDRVTITCTASESLYSSKHKVHYLAWYQQKPGKAPKLLIYGASNRYGGVPSRFSGSGSGTDFTLTISSLQPEDFATYYCAQFYSYPLTFGGGTKVEIK

SEQ ID NO: 231 - protein, 113 aa, synthetic construct
DIQMTQSPSSLSASVGDRVTITCTASESLYSSKHKVHYLAWYQQKPGKAPKLLIYGASNRPIGVPSRFSGSGSGTDFTLTISSLQPEDFATYYCAQFYSYPLTFGGGTKVEIK

SEQ ID NO: 232 - protein, 113 aa, synthetic construct
DIQMTQSPSSLSASVGDRVTITCTASESISSSKHKVHYLAWYQQKPGKAPKLLIYGASNLYSGVPSRFSGSGSGTDFTLTISSLQPEDFATYYCAHFYSYPLTFGGGTKVEIK

SEQ ID NO: 233 - protein, 113 aa, synthetic construct
DIQMTQSPSSLSASVGDRVTITCTASESLYSSKHKVHYLAWYQQKPGKAPKLLIYGAGNRYIGVPSRFSGSGSGTDFTLTISSLQPEDFATYYCAQFYEYPLTFGGGTKVEIK

SEQ ID NO: 234 - protein, 113 aa, synthetic construct
DIQMTQSPSSLSASVGDRVTITCTASESLYSSKHKVHYLAWYQQKPGKAPKLLIYGASFRYIGVPSRFSGSGSGTDFTLTISSLQPEDFATYYCAQFYSYPLTFGGGTKVEIK

SEQ ID NO: 235 - protein, 113 aa, synthetic construct
DIQMTQSPSSLSASVGDRVTITCTASESLYSSKHKVHYLAWYQQKPGKAPKLLIYGASNRGIGVPSRFSGSGSGTDFTLTISSLQPEDFATYYCAQFYSYPLTFGGGTKVEIK

SEQ ID NO: 236 - protein, 113 aa, synthetic construct
DIQMTQSPSSLSASVGDRVTITCTASESLYSSKHKVHYLAWYQQKPGKAPKLLIYGADNRYIGVPSRFSGSGSGTDFTLTISSLQPEDFATYYCAQFYHYPLTFGGGTKVEIK

SEQ ID NO: 237 - protein, 113 aa, synthetic construct
DIQMTQSPSSLSASVGDRVTITCTASESLYSSKHKVHYLAWYQQKPGKAPKLLIYGASNRYDGVPSRFSGSGSGTDFTLTISSLQPEDFATYYCAQFYEYPLTFGGGTKVEIK

SEQ ID NO: 238 - protein, 113 aa, synthetic construct
DIQMTQSPSSLSASVGDRVTITCTASESLYSSKHKVHYLAWYQQKPGKAPKLLIYGASNRQSGVPSRFSGSGSGTDFTLTISSLQPEDFATYYCAQFYVYPLTFGGGTKVEIK

SEQ ID NO: 239 - protein, 113 aa, synthetic construct
DIQMTQSPSSLSASVGDRVTITCTASESLYSSKHKVHYLAWYQQKPGKAPKLLIYGASNRYQGVPSRFSGSGSGTDFTLTISSLQPEDFATYYCAQFYFYPLTFGGGTKVEIK

SEQ ID NO: 240 - protein, 113 aa, synthetic construct
DIQMTQSPSSLSASVGDRVTITCTASESLYSSKHKVHYLAWYQQKPGKAPKLLIYGASNRYSGVPSRFSGSGSGTDFTLTISSLQPEDFATYYCAQFYKYPLTFGGGTKVEIK

SEQ ID NO: 241 - protein, 113 aa, synthetic construct
DIQMTQSPSSLSASVGDRVTITCTASESLYSSKHKVHYLAWYQQKPGKAPKLLIYGASSRYIGVPSRFSGSGSGTDFTLTISSLQPEDFATYYCAQFYEYPLTFGGGTKVEIK

SEQ ID NO: 242 - protein, 113 aa, synthetic construct
DIQMTQSPSSLSASVGDRVTITCTASESLYSSKHKVHYLAWYQQKPGKAPKLLIYGTSNRYIGVPSRFSGSGSGTDFTLTISSLQPEDFATYYCAQFYEYPLTFGGGTKVEIK

SEQ ID NO: 243 - protein, 113 aa, synthetic construct
DIQMTQSPSSLSASVGDRVTITCTASESLYSQKHKVHYLAWYQQKPGKAPKLLIYGASNRYIGVPSRFSGSGSGTDFTLTISSLQPEDFATYYCAQFYYYPLTFGGGTKVEIK

SEQ ID NO: 244 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYFYPLTFGGGTKVEIK

SEQ ID NO: 245 - protein, 113 aa, synthetic construct
DIQMTQSPSSLSASVGDRVTITCTASESLYSSKHKVHYLAWYQQKPGKAPKLLIYGASNRQSGVPSRFSGSGSGTDFTLTISSLQPEDFATYYCAQFYSYPLTFGGGTKVEIK

SEQ ID NO: 246 - protein, 113 aa, synthetic construct
DIQMTQSPSSLSASVGDRVTITCEASESLYSSKHKVHYLAWYQQKPGKAPKLLIYGASNRLIGVPSRFSGSGSGTDFTLTISSLQPEDFATYYCAQFYEYPLTFGGGTKVEIK

SEQ ID NO: 247 - protein, 113 aa, synthetic construct
DIQMTQSPSSLSASVGDRVTITCTASESLYSSKHSVHYLAWYQQKPGKAPKLLIYGASNRFIGVPSRFSGSGSGTDFTLTISSLQPEDFATYYCAQFYSYPLTFGGGTKVEIK

SEQ ID NO: 248 - protein, 113 aa, synthetic construct
DIQMTQSPSSLSASVGDRVTITCTASEELYSSKHKVHYLAWYQQKPGKAPKLLIYGASNRYIGVPSRFSGSGSGTDFTLTISSLQPEDFATYYCAQFYSYPLTFGGGTKVEIK

SEQ ID NO: 249 - protein, 113 aa, synthetic construct
DIQMTQSPSSLSASVGDRVTITCTASQSISSSKHKVHYLAWYQQKPGKAPKLLIYGASNHYIGVPSRFSGSGSGTDFTLTISSLQPEDFATYYCAQFYSYPLTFGGGTKVEIK

SEQ ID NO: 250 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYEYPLTFGGGTKVEIK

SEQ ID NO: 251 - protein, 113 aa, synthetic construct
DIQMTQSPSSLSASVGDRVTITCTASESLYSSKHKVHYLAWYQQKPGKAPKLLIYGASNRQIGVPSRFSGSGSGTDFTLTISSLQPEDFATYYCAQFYTYPLTFGGGTKVEIK

SEQ ID NO: 252 - protein, 112 aa, synthetic construct
DIQMTQSPSSLSASVGDRVTITCTASESLSSSKHKHYLAWYQQKPGKAPKLLIYGASSRYSGVPSRFSGSGSGTDFTLTISSLQPEDFATYYCAQFYSYPLTFGGGTKVEIK

SEQ ID NO: 253 - protein, 113 aa, synthetic construct
DIQMTQSPSSLSASVGDRVTITCTASESLYSSKHKVHYLAWYQQKPGKAPKLLIYGASNRDIGVPSRFSGSGSGTDFTLTISSLQPEDFATYYCGQFYSYPLTFGGGTKVEIK

SEQ ID NO: 254 - protein, 113 aa, synthetic construct
DIQMTQSPSSLSASVGDRVTITCTASESLYSSKHKVHYLAWYQQKPGKAPKLLIYGSSNRYIGVPSRFSGSGSGTDFTLTISSLQPEDFATYYCAQFYQYPLTFGGGTKVEIK

SEQ ID NO: 255 - protein, 113 aa, synthetic construct
DIQMTQSPSSLSASVGDRVTITCTASESLYSSKHKVHYLAWYQQKPGKAPKLLIYGISNRYIGVPSRFSGSGSGTDFTLTISSLQPEDFATYYCAQFYSYPLTFGGGTKVEIK

SEQ ID NO: 256 - protein, 113 aa, synthetic construct
DIQMTQSPSSLSASVGDRVTITCTAEESLYSSKHKVHYLAWYQQKPGKAPKLLIYGASNLYSGVPSRFSGSGSGTDFTLTISSLQPEDFATYYCAQFYSYPLTFGGGTKVEIK

SEQ ID NO: 257 - protein, 113 aa, synthetic construct
DIQMTQSPSSLSASVGDRVTITCTASESLYSSKHKVHYLAWYQQKPGKAPKLLIYGASNRQIGVPSRFSGSGSGTDFTLTISSLQPEDFATYYCAQFYSYPLTFGGGTKVEIK

SEQ ID NO: 258 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYDYPLTFGGGTKVEIK

SEQ ID NO: 259 - protein, 113 aa, synthetic construct
DIQMTQSPSSLSASVGDRVTITCTASESLYSSKHKVHYLAWYQQKPGKAPKLLIYGLSNRYIGVPSRFSGSGSGTDFTLTISSLQPEDFATYYCAQFFSYPLTFGGGTKVEIK

SEQ ID NO: 260 - protein, 113 aa, synthetic construct
DIQMTQSPSSLSASVGDRVTITCTASESLYSSKHKVHYLAWYQQKPGKAPKLLIYGASSLYSGVPSRFSGSGSGTDFTLTISSLQPEDFATYYCAQFYEYPLTFGGGTKVEIK

SEQ ID NO: 261 - protein, 113 aa, synthetic construct
DIQMTQSPSSLSASVGDRVTITCTASESLYSSKHGVHYLAWYQQKPGKAPKLLIYGASNRYIGVPSRFSGSGSGTDFTLTISSLQPEDFATYYCAQFYQYPLTFGGGTKVEIK

SEQ ID NO: 262 - protein, 113 aa, synthetic construct
DIQMTQSPSSLSASVGDRVTITCTASESLYSSKHKVHYLAWYQQKPGKAPKLLIYGASNRAIGVPSRFSGSGSGTDFTLTISSLQPEDFATYYCAQFYTYPLTFGGGTKVEIK

SEQ ID NO: 263 - protein, 113 aa, synthetic construct
DIQMTQSPSSLSASVGDRVTITCTASESLSSSKHKVHYLAWYQQKPGKAPKLLIYGASNRYIGVPSRFSGSGSGTDFTLTISSLQPEDFATYYCAQFYSYPLTFGGGTKVEIK

SEQ ID NO: 264 - protein, 113 aa, synthetic construct
DIQMTQSPSSLSASVGDRVTITCTASESLYSSKHKVHYLAWYQQKPGKAPKLLIYGASNRYAGVPSRFSGSGSGTDFTLTISSLQPEDFATYYCGQFYSYPLTFGGGTKVEIK

SEQ ID NO: 265 - protein, 113 aa, synthetic construct
DIQMTQSPSSLSASVGDRVTITCTASQSLYSSKHKVHYLAWYQQKPGKAPKLLIYGASNRYIGVPSRFSGSGSGTDFTLTISSLQPEDFATYYCAQFYVYPLTFGGGTKVEIK

SEQ ID NO: 266 - protein, 111 aa, synthetic construct
DIQMTQSPSSLSASVGDRVTITCRASESLYSSLHKYLAWYQQKPGKAPKLLIYGASNLQIGVPSRFSGSGSGTDFTLTISSLQPEDFATYYCAQFYSYPLTFGGGTKVEIK

SEQ ID NO: 267 - protein, 113 aa, synthetic construct
DIQMTQSPSSLSASVGDRVTITCTASESLYSSKHKVHYLAWYQQKPGKAPKLLIYGASNRVIGVPSRFSGSGSGTDFTLTISSLQPEDFATYYCAQFYSYPLTFGGGTKVEIK

SEQ ID NO: 268 - protein, 113 aa, synthetic construct
DIQMTQSPSSLSASVGDRVTITCTASESLYSSKHKVHYLAWYQQKPGKAPKLLIYGPSNRYIGVPSRFSGSGSGTDFTLTISSLQPEDFATYYCAQFYSYPLTFGGGTKVEIK

SEQ ID NO: 269 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 270 - protein, 113 aa, synthetic construct
DIVMTQSPDSLAVSLGERATINCTASESVYSSKNKKHYLAWYQQKPGQPPKLLIYGASNRYIGVPDRFSGSGSGTDFTLTISSLQAEDVAVYYCAQFYSYPLTFGGGTKVEIK

SEQ ID NO: 271 - protein, 113 aa, synthetic construct
DIVMTQSPDSLAVSLGERATINCTASESLYSSKGKVHYLAWYQQKPGQPPKLLIYGASNRYIGVPDRFSGSGSGTDFTLTISSLQAEDVAVYYCVQFYSYPLTFGGGTKVEIK

SEQ ID NO: 272 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCGQFYSYPLTFGGGTKVEIK

SEQ ID NO: 273 - protein, 113 aa, synthetic construct
DIVMTQSPDSLAVSLGERATINCTASESLYSSKHKVHYLAWYQQKPGQPPKLLIYGASNRYDGVPDRFSGSGSGTDFTLTISSLQAEDVAVYYCAQFYSYPLTFGGGTKVEIK

SEQ ID NO: 274 - protein, 113 aa, synthetic construct
DIVMTQSPDSLAVSLGERATINCTASQSVYSSKNKVHYLAWYQQKPGQPPKLLIYGASNRYIGVPDRFSGSGSGTDFTLTISSLQAEDVAVYYCAQFYSYPLTFGGGTKVEIK

SEQ ID NO: 275 - protein, 113 aa, synthetic construct
DIVMTQSPDSLAVSLGERATINCTSSQSLLSSSHNKNYLAWYQQKPGQPPKLLIYGASNRYIGVPDRFSGSGSGTDFTLTISSLQAEDVAVYYCAQFYSYPLTFGGGTKVEIK

SEQ ID NO: 276 - protein, 113 aa, synthetic construct
DIVMTQSPDSLAVSLGERATINCTASESLYSPKHKVHYLAWYQQKPGQPPKLLIYGASNRYIGVPDRFSGSGSGTDFTLTISSLQAEDVAVYYCAQFYIYPLTFGGGTKVEIK

SEQ ID NO: 277 - protein, 113 aa, synthetic construct
DIVMTQSPDSLAVSLGERATINCTASESLWSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 278 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYIYPLTFGGGTKVEIK

SEQ ID NO: 279 - protein, 113 aa, synthetic construct
DIVMTQSPDSLAVSLGERATINCKASQSVLSSKNNVNYLAWYQQKPGQPPKLLIYGASNRYSGVPDRFSGSGSGTDFTLTISSLQAEDVAVYYCAQFYSYPLTFGGGTKVEIK

SEQ ID NO: 280 - protein, 113 aa, synthetic construct
DIVMTQSPDSLAVSLGERATINCTASESLYSSKWKVHYLAWYQQKPGQPPKLLIYGASNRYIGVPDRFSGSGSGTDFTLTISSLQAEDVAVYYCAQFYSYPLTFGGGTKVEIK

SEQ ID NO: 281 - protein, 113 aa, synthetic construct
DIVMTQSPDSLAVSLGERATINCTASESLYSSKHKVHYLAWYQQKPGQPPKLLIYGASNRVIGVPDRFSGSGSGTDFTLTISSLQAEDVAVYYCANFYSYPLTFGGGTKVEIK

SEQ ID NO: 282 - protein, 113 aa, synthetic construct
DIVMTQSPDSLAVSLGERATINCKSSESVYSSKHKKHYLAWYQQKPGQPPKLLIYGASNRYIGVPDRFSGSGSGTDFTLTISSLQAEDVAVYYCAQFYSYPLTFGGGTKVEIK

SEQ ID NO: 283 - protein, 113 aa, synthetic construct
DIVMTQSPDSLAVSLGERATINCTASESLYSSKHKVDYLAWYQQKPGQPPKLLIYGASNRYIGVPDRFSGSGSGTDFTLTISSLQAEDVAVYYCAQFYSYPLTFGGGTKVEIK

SEQ ID NO: 284 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCANFYSYPLTFGGGTKVEIK

SEQ ID NO: 285 - protein, 113 aa, synthetic construct
DIVMTQSPDSLAVSLGERATINCTASESLYSPKHKVHYLAWYQQKPGQPPKLLIYGAFNRYIGVPDRFSGSGSGTDFTLTISSLQAEDVAVYYCAQFYSYPLTFGGGTKVEIK

SEQ ID NO: 286 - protein, 113 aa, synthetic construct
DIVMTQSPDSLAVSLGERATINCTASESVYSSKNKKHYLAWYQQKPGQPPKLLIYGASNRYIGVPDRFSGSGSGTDFTLTISSLQAEDVAVYYCAQFYTYPLTFGGGTKVEIK

SEQ ID NO: 287 - protein, 113 aa, synthetic construct
DIVMTQSPDSLAVSLGERATINCKSSQSLYYSSNKKNYLAWYQQKPGQPPKLLIYGASNRYGGVPDRFSGSGSGTDFTLTISSLQAEDVAVYYCAQFYRYPLTFGGGTKVEIK

SEQ ID NO: 288 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAHFYSYPLTFGGGTKVEIK

SEQ ID NO: 289 - protein, 113 aa, synthetic construct
DIVMTQSPDSLAVSLGERATINCTASESLYSSKHKVHYLAWYQQKPGQPPKLLIYGASNRYSGVPDRFSGSGSGTDFTLTISSLQAEDVAVYYCAQFYSYPLTFGGGTKVEIK

SEQ ID NO: 290 - protein, 113 aa, synthetic construct
DIVMTQSPDSLAVSLGERATINCTASESLYSSKHKVHYLAWYQQKPGQPPKLLIYGASNRYVGVPDRFSGSGSGTDFTLTISSLQAEDVAVYYCAQFYSYPLTFGGGTKVEIK

SEQ ID NO: 291 - protein, 113 aa, synthetic construct
DIVMTQSPDSLAVSLGERATINCTASESLYSSKHKVHYLAWYQQKPGQPPKLLIYGASNREIGVPDRFSGSGSGTDFTLTISSLQAEDVAVYYCAQFYFYPLTFGGGTKVEIK

SEQ ID NO: 292 - protein, 113 aa, synthetic construct
DIVMTQSPDSLAVSLGERATINCTASTSLYSSKHKVHYLAWYQQKPGQPPKLLIYGASNRYIGVPDRFSGSGSGTDFTLTISSLQAEDVAVYYCAQFYTYPLTFGGGTKVEIK

SEQ ID NO: 293 - protein, 113 aa, synthetic construct
DIVMTQSPDSLAVSLGERATINCTASESLYGSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 294 - protein, 113 aa, synthetic construct
DIVMTQSPDSLAVSLGERATINCKASQSLYSSKHKKHYLAWYQQKPGQPPKLLIYGASNRYPGVPDRFSGSGSGTDFTLTISSLQAEDVAVYYCAQFYRYPLTFGGGTKVEIK

SEQ ID NO: 295 - protein, 113 aa, synthetic construct
DIVMTQSPDSLAVSLGERATINCTASSSLYSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 296 - protein, 113 aa, synthetic construct
DIVMTQSPDSLAVSLGERATINCTASESLYSSKHKVHYLAWYQQKPGQPPKLLIYGAGNRYIGVPDRFSGSGSGTDFTLTISSLQAEDVAVYYCAQFYHYPLTFGGGTKVEIK

SEQ ID NO: 297 - protein, 113 aa, synthetic construct
DIVMTQSPDSLAVSLGERATINCTASESLYSSKHKVHYLAWYQQKPGQPPKLLIYGASNRYDGVPDRFSGSGSGTDFTLTISSLQAEDVAVYYCANFYSYPLTFGGGTKVEIK

SEQ ID NO: 298 - protein, 113 aa, synthetic construct
DIVMTQSPDSLAVSLGERATINCTASESLYSSKHKVHYLAWYQQKPGQPPKLLIYGAGNRYIGVPDRFSGSGSGTDFTLTISSLQAEDVAVYYCAQFYSYPLTFGGGTKVEIK

SEQ ID NO: 299 - protein, 113 aa, synthetic construct
DIVMTQSPDSLAVSLGERATINCTASESLYSSKHKVHYLAWYQQKPGQPPKLLIYGASNRQIGVPDRFSGSGSGTDFTLTISSLQAEDVAVYYCAQFYHYPLTFGGGTKVEIK

SEQ ID NO: 300 - protein, 113 aa, synthetic construct
DIVMTQSPDSLAVSLGERATINCTASESLYSVKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 301 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYVYPLTFGGGTKVEIK

SEQ ID NO: 302 - protein, 113 aa, synthetic construct
DIVMTQSPDSLAVSLGERATINCTASESLYSSKHKVHYLAWYQQKPGQPPKLLIYGANNRYIGVPDRFSGSGSGTDFTLTISSLQAEDVAVYYCAQFYSYPLTFGGGTKVEIK

SEQ ID NO: 303 - protein, 113 aa, synthetic construct
DIVMTQSPDSLAVSLGERATINCGASESLYSSKSKVHYLAWYQQKPGQPPKLLIYGASNRYIGVPDRFSGSGSGTDFTLTISSLQAEDVAVYYCAQFYSYPLTFGGGTKVEIK

SEQ ID NO: 304 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYEYPLTFGGGTKVEIK

SEQ ID NO: 305 - protein, 113 aa, synthetic construct
DIVMTQSPDSLAVSLGERATINCTASESLYSSKHKVHYLAWYQQKPGQPPKLLIYGAHNRYIGVPDRFSGSGSGTDFTLTISSLQAEDVAVYYCANFYSYPLTFGGGTKVEIK

SEQ ID NO: 306 - protein, 113 aa, synthetic construct
DIVMTQSPDSLAVSLGERATINCTSSESVYSSSHKVHYLAWYQQKPGQPPKLLIYGASNRYIGVPDRFSGSGSGTDFTLTISSLQAEDVAVYYCAQFYYYPLTFGGGTKVEIK

SEQ ID NO: 307 - protein, 113 aa, synthetic construct
DIVMTQSPDSLAVSLGERATINCTASESLYSSKHKVHYLAWYQQKPGQPPKLLIYGNSNRYIGVPDRFSGSGSGTDFTLTISSLQAEDVAVYYCGQFYSYPLTFGGGTKVEIK

SEQ ID NO: 308 - protein, 113 aa, synthetic construct
DIVMTQSPDSLAVSLGERATINCTASESLYSSKHKKNYLAWYQQKPGQPPKLLIYGASWRYIGVPDRFSGSGSGTDFTLTISSLQAEDVAVYYCAQFYSYPLTFGGGTKVEIK

SEQ ID NO: 309 - protein, 113 aa, synthetic construct
DIVMTQSPDSLAVSLGERATINCTASESLYSSKHKVHYLAWYQQKPGQPPKLLIYGASTRYSGVPDRFSGSGSGTDFTLTISSLQAEDVAVYYCAQFYTYPLTFGGGTKVEIK

SEQ ID NO: 310 - protein, 113 aa, synthetic construct
DIVMTQSPDSLAVSLGERATINCKSSESLLYSKNNKNYLAWYQQKPGQPPKLLIYGASNRYSGVPDRFSGSGSGTDFTLTISSLQAEDVAVYYCAQFYSYPLTFGGGTKVEIK

SEQ ID NO: 311 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAEFYSYPLTFGGGTKVEIK

SEQ ID NO: 312 - protein, 113 aa, synthetic construct
DIVMTQSPDSLAVSLGERATINCTASESIYSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 313 - protein, 113 aa, synthetic construct
DIVMTQSPDSLAVSLGERATINCTSSQSVLSSKHNKNYLAWYQQKPGQPPKLLIYGASNRYWGVPDRFSGSGSGTDFTLTISSLQAEDVAVYYCAQFYSYPLTFGGGTKVEIK

SEQ ID NO: 314 - protein, 113 aa, synthetic construct
DIVMTQSPDSLAVSLGERATINCTASESLYSSKHKVHYLAWYQQKPGQPPKLLIYGASNRYPGVPDRFSGSGSGTDFTLTISSLQAEDVAVYYCAQFYSYPLTFGGGTKVEIK

SEQ ID NO: 315 - protein, 113 aa, synthetic construct
DIVMTQSPDSLAVSLGERATINCTASEDLYSSKHKVHYLAWYQQKPGQPPKLLIYGTSNRYIGVPDRFSGSGSGTDFTLTISSLQAEDVAVYYCAQYYSYPLTFGGGTKVEIK

SEQ ID NO: 316 - protein, 113 aa, synthetic construct
DIVMTQSPDSLAVSLGERATINCKASQSVLYSKNKKHYLAWYQQKPGQPPKLLIYGASNRQIGVPDRFSGSGSGTDFTLTISSLQAEDVAVYYCAQFYVYPLTFGGGTKVEIK

SEQ ID NO: 317 - protein, 113 aa, synthetic construct
DIVMTQSPDSLAVSLGERATINCTASESLYSSKHKLHYLAWYQQKPGQPPKLLIYGASNRYIGVPDRFSGSGSGTDFTLTISSLQAEDVAVYYCGQFYSYPLTFGGGTKVEIK

SEQ ID NO: 318 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYDYPLTFGGGTKVEIK

SEQ ID NO: 319 - protein, 113 aa, synthetic construct
DIVMTQSPDSLAVSLGERATINCTASESLYSSKHKVVYLAWYQQKPGQPPKLLIYGASNRYIGVPDRFSGSGSGTDFTLTISSLQAEDVAVYYCAQFYFYPLTFGGGTKVEIK

SEQ ID NO: 320 - protein, 113 aa, synthetic construct
DIVMTQSPDSLAVSLGERATINCTASESLYSSKHKVHYLAWYQQKPGQPPKLLIYGASNRYLGVPDRFSGSGSGTDFTLTISSLQAEDVAVYYCAQFYSYPLTFGGGTKVEIK

SEQ ID NO: 321 - protein, 113 aa, synthetic construct
DIVMTQSPDSLAVSLGERATINCTSSESLYSSKNKVNYLAWYQQKPGQPPKLLIYGASNRYIGVPDRFSGSGSGTDFTLTISSLQAEDVAVYYCAQFYFYPLTFGGGTKVEIK

SEQ ID NO: 322 - protein, 113 aa, synthetic construct
DIVMTQSPDSLAVSLGERATINCTASESLYSSKHKVHYLAWYQQKPGQPPKLLIYGASNRYNGVPDRFSGSGSGTDFTLTISSLQAEDVAVYYCAQFYTYPLTFGGGTKVEIK

SEQ ID NO: 323 - protein, 113 aa, synthetic construct
DIVMTQSPDSLAVSLGERATINCTSSQSLYSSKHNKHYLAWYQQKPGQPPKLLIYGASNRYIGVPDRFSGSGSGTDFTLTISSLQAEDVAVYYCAQFYSYPLTFGGGTKVEIK

SEQ ID NO: 324 - protein, 113 aa, synthetic construct
DIVMTQSPDSLAVSLGERATINCEASESLYSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 325 - protein, 113 aa, synthetic construct
DIVMTQSPDSLAVSLGERATINCTASESLYSSKHKVHYLAWYQQKPGQPPKLLIYGASNRAIGVPDRFSGSGSGTDFTLTISSLQAEDVAVYYCAQFYFYPLTFGGGTKVEIK

SEQ ID NO: 326 - protein, 113 aa, synthetic construct
DIVMTQSPDSLAVSLGERATINCTASESLYSNKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 327 - protein, 113 aa, synthetic construct
DIVMTQSPDSLAVSLGERATINCTASENLYSSKHKVHYLAWYQQKPGQPPKLLIYGASNRYSGVPDRFSGSGSGTDFTLTISSLQAEDVAVYYCAQFYSYPLTFGGGTKVEIK

SEQ ID NO: 328 - protein, 113 aa, synthetic construct
DIVMTQSPDSLAVSLGERATINCTASESLYSSKHKVHYLAWYQQKPGQPPKLLIYGASQRYIGVPDRFSGSGSGTDFTLTISSLQAEDVAVYYCAQFYLYPLTFGGGTKVEIK

SEQ ID NO: 329 - protein, 113 aa, synthetic construct
DIVMTQSPDSLAVSLGERATINCTASESLYSSKHKVHYLAWYQQKPGQPPKLLIYGASNREIGVPDRFSGSGSGTDFTLTISSLQAEDVAVYYCAQFYSYPLTFGGGTKVEIK

SEQ ID NO: 330 - protein, 113 aa, synthetic construct
DIVMTQSPDSLAVSLGERATINCTASESLYSSKHKVHYLAWYQQKPGQPPKLLIYGASNRYTGVPDRFSGSGSGTDFTLTISSLQAEDVAVYYCAQFYSYPLTFGGGTKVEIK

SEQ ID NO: 331 - protein, 113 aa, synthetic construct
DIVMTQSPDSLAVSLGERATINCTASESLYSSKHKVHYLAWYQQKPGQPPKLLIYGAANRYIGVPDRFSGSGSGTDFTLTISSLQAEDVAVYYCAQFYSYPLTFGGGTKVEIK

SEQ ID NO: 332 - protein, 113 aa, synthetic construct
DIVMTQSPDSLAVSLGERATINCTASESLYSSKHKVHYLAWYQQKPGQPPKLLIYGAQNRYIGVPDRFSGSGSGTDFTLTISSLQAEDVAVYYCGQFYSYPLTFGGGTKVEIK

SEQ ID NO: 333 - protein, 113 aa, synthetic construct
DIVMTQSPDSLAVSLGERATINCTASESLYSSKIKVHYLAWYQQKPGQPPKLLIYGAHNRYIGVPDRFSGSGSGTDFTLTISSLQAEDVAVYYCAQFYSYPLTFGGGTKVEIK

SEQ ID NO: 334 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYTYPLTFGGGTKVEIK

SEQ ID NO: 335 - protein, 113 aa, synthetic construct
DIVMTQSPDSLAVSLGERATINCTASESLYSSKHKVHYLAWYQQKPGQPPKLLIYGASNRYSGVPDRFSGSGSGTDFTLTISSLQAEDVAVYYCAQFYHYPLTFGGGTKVEIK

SEQ ID NO: 336 - protein, 113 aa, synthetic construct
DIVMTQSPDSLAVSLGERATINCTASESLYSSKHKVHYLAWYQQKPGQPPKLLIYGATNRYIGVPDRFSGSGSGTDFTLTISSLQAEDVAVYYCAEFYSYPLTFGGGTKVEIK

SEQ ID NO: 337 - protein, 113 aa, synthetic construct
DIVMTQSPDSLAVSLGERATINCTASESLYSSKHKVHYLAWYQQKPGQPPKLLIYGASGRYIGVPDRFSGSGSGTDFTLTISSLQAEDVAVYYCAQFYSYPLTFGGGTKVEIK

SEQ ID NO: 338 - protein, 113 aa, synthetic construct
DIVMTQSPDSLAVSLGERATINCKSSQSLLYSKNNVNYLAWYQQKPGQPPKLLIYGESNRYIGVPDRFSGSGSGTDFTLTISSLQAEDVAVYYCAQFYEYPLTFGGGTKVEIK

SEQ ID NO: 339 - protein, 113 aa, synthetic construct
DIVMTQSPDSLAVSLGERATINCKSSESLYSSKNKVHYLAWYQQKPGQPPKLLIYGASNRYIGVPDRFSGSGSGTDFTLTISSLQAEDVAVYYCAQFYHYPLTFGGGTKVEIK

SEQ ID NO: 340 - protein, 113 aa, synthetic construct
DIVMTQSPDSLAVSLGERATINCTASESLYSSKHKVNYLAWYQQKPGQPPKLLIYGASNRYIGVPDRFSGSGSGTDFTLTISSLQAEDVAVYYCAQFYSYPLTFGGGTKVEIK

SEQ ID NO: 341 - protein, 113 aa, synthetic construct
DIVMTQSPDSLAVSLGERATINCTSSESLLSSKHKVHYLAWYQQKPGQPPKLLIYGASNRYTGVPDRFSGSGSGTDFTLTISSLQAEDVAVYYCAQFYSYPLTFGGGTKVEIK

SEQ ID NO: 342 - protein, 113 aa, synthetic construct
DIVMTQSPDSLAVSLGERATINCTASQSVYSSKHKKHYLAWYQQKPGQPPKLLIYGANNRYIGVPDRFSGSGSGTDFTLTISSLQAEDVAVYYCGQFYSYPLTFGGGTKVEIK

SEQ ID NO: 343 - protein, 113 aa, synthetic construct
DIVMTQSPDSLAVSLGERATINCTASQSVYSSKHNKHYLAWYQQKPGQPPKLLIYGASNPYIGVPDRFSGSGSGTDFTLTISSLQAEDVAVYYCAQFYSYPLTFGGGTKVEIK

SEQ ID NO: 344 - protein, 113 aa, synthetic construct
DIVMTQSPDSLAVSLGERATINCTASESLYSGKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 345 - protein, 113 aa, synthetic construct
DIVMTQSPDSLAVSLGERATINCTSSQSLLYSSNKKHYLAWYQQKPGQPPKLLIYGASNRYIGVPDRFSGSGSGTDFTLTISSLQAEDVAVYYCAQFYEYPLTFGGGTKVEIK

SEQ ID NO: 346 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAQFYNYPLTFGGGTKVEIK

SEQ ID NO: 347 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCASFYSYPLTFGGGTKVEIK

SEQ ID NO: 348 - protein, 113 aa, synthetic construct
DIVMTQSPDSLAVSLGERATINCKSSQSVLSSKHKKNYLAWYQQKPGQPPKLLIYGASNRYIGVPDRFSGSGSGTDFTLTISSLQAEDVAVYYCAQFYSYPLTFGGGTKVEIK

SEQ ID NO: 349 - protein, 113 aa, synthetic construct
DIVMTQSPDSLAVSLGERATINCTASESLYSSKHKVHYLAWYQQKPGQPPKLLIYGAQNRYIGVPDRFSGSGSGTDFTLTISSLQAEDVAVYYCAQFYSYPLTFGGGTKVEIK

SEQ ID NO: 350 - protein, 113 aa, synthetic construct
DIVMTQSPDSLAVSLGERATINCTASQSVYSSKNKKHYLAWYQQKPGQPPKLLIYGASNRYIGVPDRFSGSGSGTDFTLTISSLQAEDVAVYYCAQFYRYPLTFGGGTKVEIK

SEQ ID NO: 351 - protein, 113 aa, synthetic construct
DIVMTQSPDSLAVSLGERATINCTASESVLSSKHKKNYLAWYQQKPGQPPKLLIYGASTRYIGVPDRFSGSGSGTDFTLTISSLQAEDVAVYYCAQFYSYPLTFGGGTKVEIK

SEQ ID NO: 352 - protein, 113 aa, synthetic construct
DIVMTQSPDSLAVSLGERATINCTASVSLYSSKHKVHYLAWYQQKPGQPPKLLIYGPSNRYIGVPDRFSGSGSGTDFTLTISSLQAEDVAVYYCAQFYSYPLTFGGGTKVEIK

SEQ ID NO: 353 - protein, 113 aa, synthetic construct
DIVMTQSPDSLAVSLGERATINCTASESLYSSKHKVHYLAWYQQKPGQPPKLLIYGASNRPIGVPDRFSGSGSGTDFTLTISSLQAEDVAVYYCGQFYSYPLTFGGGTKVEIK

SEQ ID NO: 354 - protein, 113 aa, synthetic construct
DIVMTQSPDSLAVSLGERATINCTASESLYSSKHKVHYLAWYQQKPGQPPKLLIYGASNRYIGVPDRFSGSGSGTDFTLTISSLQAEDVAVYYCAAFYSYPLTFGGGTKVEIK

SEQ ID NO: 355 - protein, 113 aa, synthetic construct
DIVMTQSPDSLAVSLGERATINCTASESLYSPKHKVHYLAWYQQKPGQPPKLLIYGAENRYIGVPDRFSGSGSGTDFTLTISSLQAEDVAVYYCAQFYSYPLTFGGGTKVEIK

SEQ ID NO: 356 - protein, 113 aa, synthetic construct
DIVMTQSPDSLAVSLGERATINCTASESLYSSKHKVHYLAWYQQKPGQPPKLLIYGASHRQIGVPDRFSGSGSGTDFTLTISSLQAEDVAVYYCAQFYSYPLTFGGGTKVEIK

SEQ ID NO: 357 - protein, 113 aa, synthetic construct
DIVMTQSPDSLAVSLGERATINCTASESLYSSKHKVHYLAWYQQKPGQPPKLLIYGASQRYIGVPDRFSGSGSGTDFTLTISSLQAEDVAVYYCAQFYSYPLTFGGGTKVEIK

SEQ ID NO: 358 - protein, 113 aa, synthetic construct
DIVMTQSPDSLAVSLGERATINCTASTSLYSSKHKVHYLAWYQQKPGQPPKLLIYGASNRYIGVPDRFSGSGSGTDFTLTISSLQAEDVAVYYCAQFYSYPLTFGGGTKVEIK

SEQ ID NO: 359 - protein, 113 aa, synthetic construct
DIVMTQSPDSLAVSLGERATINCKSSESLYSSKNKVHYLAWYQQKPGQPPKLLIYGASNRYSGVPDRFSGSGSGTDFTLTISSLQAEDVAVYYCAQFYSYPLTFGGGTKVEIK

SEQ ID NO: 360 - protein, 113 aa, synthetic construct
DIVMTQSPDSLAVSLGERATINCTASESLYSTKHKVHYLAWYQQKPGQPPKLLIYGASHRYIGVPDRFSGSGSGTDFTLTISSLQAEDVAVYYCAQFYQYPLTFGGGTKVEIK

SEQ ID NO: 361 - protein, 113 aa, synthetic construct
DIVMTQSPDSLAVSLGERATINCTASESLYSSKHKVHYLAWYQQKPGQPPKLLIYGVSNRYIGVPDRFSGSGSGTDFTLTISSLQAEDVAVYYCAQFYSYPLTFGGGTKVEIK

SEQ ID NO: 362 - protein, 113 aa, synthetic construct
DIVMTQSPDSLAVSLGERATINCTASESLYSSKHKVHYLAWYQQKPGQPPKLLIYGASNRVIGVPDRFSGSGSGTDFTLTISSLQAEDVAVYYCAQFYIYPLTFGGGTKVEIK

SEQ ID NO: 363 - protein, 113 aa, synthetic construct
DIVMTQSPDSLAVSLGERATINCTASESLYSSNHKVHYLAWYQQKPGQPPKLLIYGVSNRYIGVPDRFSGSGSGTDFTLTISSLQAEDVAVYYCAQFYSYPLTFGGGTKVEIK

SEQ ID NO: 364 - protein, 113 aa, synthetic construct
DIVMTQSPDSLAVSLGERATINCKASQSVLSSKNNVNYLAWYQQKPGQPPKLLIYGASNRYIGVPDRFSGSGSGTDFTLTISSLQAEDVAVYYCAQFYQYPLTFGGGTKVEIK

SEQ ID NO: 365 - protein, 113 aa, synthetic construct
DIVMTQSPDSLAVSLGERATINCTASESLYSSKHKVHYLAWYQQKPGQPPKLLIYGASNRNIGVPDRFSGSGSGTDFTLTISSLQAEDVAVYYCAHFYSYPLTFGGGTKVEIK

SEQ ID NO: 366 - protein, 113 aa, synthetic construct
DIVMTQSPDSLAVSLGERATINCTASESLYSSKHKVHYLAWYQQKPGQPPKLLIYGASNRYQGVPDRFSGSGSGTDFTLTISSLQAEDVAVYYCAQFYWYPLTFGGGTKVEIK

SEQ ID NO: 367 - protein, 113 aa, synthetic construct
DIVMTQSPDSLAVSLGERATINCTASESLYSSKHKVHYLAWYQQKPGQPPKLLIYGASNTYIGVPDRFSGSGSGTDFTLTISSLQAEDVAVYYCAQFYSYPLTFGGGTKVEIK

SEQ ID NO: 368 - protein, 113 aa, synthetic construct
DIVMTQSPDSLAVSLGERATINCTASESLYSSKHKVHYLAWYQQKPGQPPKLLIYGASNRYSGVPDRFSGSGSGTDFTLTISSLQAEDVAVYYCAQFYNYPLTFGGGTKVEIK

SEQ ID NO: 369 - protein, 9 aa, synthetic construct
FTFSDYYME

SEQ ID NO: 370 - protein, 9 aa, synthetic construct
FGFSDFYME

SEQ ID NO: 371 - protein, 9 aa, synthetic construct
FTFSDQYIE

SEQ ID NO: 372 - protein, 9 aa, synthetic construct
FTFSDGYME

SEQ ID NO: 373 - protein, 9 aa, synthetic construct
FTASDFYME

SEQ ID NO: 374 - protein, 9 aa, synthetic construct
FTFSDSYME

SEQ ID NO: 375 - protein, 9 aa, synthetic construct
FTFEDFYME

SEQ ID NO: 376 - protein, 9 aa, synthetic construct
FTFSDFYME

SEQ ID NO: 377 - protein, 9 aa, synthetic construct
FTFSDQYME

SEQ ID NO: 378 - protein, 9 aa, synthetic construct
FQFSDFYME

SEQ ID NO: 379 - protein, 9 aa, synthetic construct
FEFSDNYME

SEQ ID NO: 380 - protein, 9 aa, synthetic construct
FEFSDFYME

SEQ ID NO: 381 - protein, 9 aa, synthetic construct
FTFSDAYME

SEQ ID NO: 382 - protein, 9 aa, synthetic construct
FTESDFYME

SEQ ID NO: 383 - protein, 9 aa, synthetic construct
FTFSSYYME

SEQ ID NO: 384 - protein, 9 aa, synthetic construct
FTGSDFYME

SEQ ID NO: 385 - protein, 9 aa, synthetic construct
FAFSDFYME

SEQ ID NO: 386 - protein, 9 aa, synthetic construct
FTHSDFYME

SEQ ID NO: 387 - protein, 9 aa, synthetic construct
FTFSDTYME

SEQ ID NO: 388 - protein, 9 aa, synthetic construct
FTFSDDYME

SEQ ID NO: 389 - protein, 9 aa, synthetic construct
FTFSDNYME

SEQ ID NO: 390 - protein, 9 aa, synthetic construct
FTSSDFYME

SEQ ID NO: 391 - protein, 9 aa, synthetic construct
FTFSTFYME

SEQ ID NO: 392 - protein, 9 aa, synthetic construct
FTFSDEYME

SEQ ID NO: 393 - protein, 9 aa, synthetic construct
FWFSDFYME

SEQ ID NO: 394 - protein, 9 aa, synthetic construct
FTFSDVYME

SEQ ID NO: 395 - protein, 9 aa, synthetic construct
ATFSDFYME

SEQ ID NO: 396 - protein, 9 aa, synthetic construct
FDFSDFYME

SEQ ID NO: 397 - protein, 9 aa, synthetic construct
WTFSDFYME

SEQ ID NO: 398 - protein, 9 aa, synthetic construct
FTFSSFYME

SEQ ID NO: 399 - protein, 9 aa, synthetic construct
FTFNDFYME

SEQ ID NO: 400 - protein, 9 aa, synthetic construct
FTFSDHYME

SEQ ID NO: 401 - protein, 9 aa, synthetic construct
FTFSDGYIE

SEQ ID NO: 402 - protein, 9 aa, synthetic construct
GTFSDFYME

SEQ ID NO: 403 - protein, 9 aa, synthetic construct
FTQSDFYME

SEQ ID NO: 404 - protein, 9 aa, synthetic construct
DTFSDFYME

SEQ ID NO: 405 - protein, 9 aa, synthetic construct
FTTSDFYME

SEQ ID NO: 406 - protein, 9 aa, synthetic construct
ETFSDFYME

SEQ ID NO: 407 - protein, 9 aa, synthetic construct
FPFSDFYME

SEQ ID NO: 408 - protein, 9 aa, synthetic construct
ATFSDFYVE

SEQ ID NO: 409 - protein, 9 aa, synthetic construct
FTDSDFYME

SEQ ID NO: 410 - protein, 9 aa, synthetic construct
NTFSDFYME

SEQ ID NO: 411 - protein, 9 aa, synthetic construct
DTFSDFYLE

SEQ ID NO: 412 - protein, 9 aa, synthetic construct
FLFSDFYME

SEQ ID NO: 413 - protein, 9 aa, synthetic construct
FTNSDFYME

SEQ ID NO: 414 - protein, 9 aa, synthetic construct
QTFSDFYME

SEQ ID NO: 415 - protein, 9 aa, synthetic construct
FTFSDPYME

SEQ ID NO: 416 - protein, 9 aa, synthetic construct
TTFSDFYME

SEQ ID NO: 417 - protein, 9 aa, synthetic construct
PTFSDFYME

SEQ ID NO: 418 - protein, 9 aa, synthetic construct
FTFSEFYME

SEQ ID NO: 419 - protein, 9 aa, synthetic construct
FHFSDFYME

SEQ ID NO: 420 - protein, 9 aa, synthetic construct
ITFSDFYME

SEQ ID NO: 421 - protein, 9 aa, synthetic construct
FTFSDFYVE

SEQ ID NO: 422 - protein, 9 aa, synthetic construct
STFSDFYME

SEQ ID NO: 423 - protein, 9 aa, synthetic construct
FSFSDFYME

SEQ ID NO: 424 - protein, 9 aa, synthetic construct
FTFDDFYME

SEQ ID NO: 425 - protein, 9 aa, synthetic construct
FTFSDFYIE

SEQ ID NO: 426 - protein, 9 aa, synthetic construct
FTSSDFYLE

SEQ ID NO: 427 - protein, 9 aa, synthetic construct
FTFSPFYME

SEQ ID NO: 428 - protein, 15 aa, synthetic construct
AASRNKANDYTTEYA

SEQ ID NO: 429 - protein, 15 aa, synthetic construct
AASRNKWNDYTTEYA

SEQ ID NO: 430 - protein, 15 aa, synthetic construct
AASRNKANDYTTSYA

SEQ ID NO: 431 - protein, 15 aa, synthetic construct
AASRNKANDYTTELA

SEQ ID NO: 432 - protein, 15 aa, synthetic construct
AASRGKGNSYTTEYA

SEQ ID NO: 433 - protein, 15 aa, synthetic construct
AASRNKANDYVTEYA

SEQ ID NO: 434 - protein, 15 aa, synthetic construct
AASRNKYNDYTTEYA

SEQ ID NO: 435 - protein, 15 aa, synthetic construct
AASRNKGNSYTTEYA

SEQ ID NO: 436 - protein, 15 aa, synthetic construct
AASRNKANTYTTEYA

SEQ ID NO: 437 - protein, 15 aa, synthetic construct
AASRNKNNDYTTEYA

SEQ ID NO: 438 - protein, 15 aa, synthetic construct
AASRNIANDYTTEYA

SEQ ID NO: 439 - protein, 15 aa, synthetic construct
AASRNSGNDYTTEYA

SEQ ID NO: 440 - protein, 15 aa, synthetic construct
AASRNKANDYTVEYA

SEQ ID NO: 441 - protein, 15 aa, synthetic construct
AASRNKANDYYTEYA

SEQ ID NO: 442 - protein, 15 aa, synthetic construct
AASRHKANDYTTEYA

SEQ ID NO: 443 - protein, 15 aa, synthetic construct
AASRNKANDYQTEYA

SEQ ID NO: 444 - protein, 15 aa, synthetic construct
AASRNQANDYTTEYA

SEQ ID NO: 445 - protein, 15 aa, synthetic construct
AASRNGANDYTTEYA

SEQ ID NO: 446 - protein, 15 aa, synthetic construct
AASRNKANDYTTDYA

SEQ ID NO: 447 - protein, 15 aa, synthetic construct
AASRNKANDYTNEYA

SEQ ID NO: 448 - protein, 15 aa, synthetic construct
AASRNAANDYTTEYA

SEQ ID NO: 449 - protein, 15 aa, synthetic construct
AASRNKANDQTTEYA

SEQ ID NO: 450 - protein, 15 aa, synthetic construct
AASRNSGNSYTTEYA

SEQ ID NO: 451 - protein, 15 aa, synthetic construct
AASRNKANSYTTEYA

SEQ ID NO: 452 - protein, 15 aa, synthetic construct
AASRWKANDYTTEYA

SEQ ID NO: 453 - protein, 15 aa, synthetic construct
AAIRNKANSYTTEYA

SEQ ID NO: 454 - protein, 15 aa, synthetic construct
AASRNKANSYTTAYA

SEQ ID NO: 455 - protein, 15 aa, synthetic construct
AASRNKANDYTTTYA

SEQ ID NO: 456 - protein, 15 aa, synthetic construct
AASRNKGNDYTTEYA

SEQ ID NO: 457 - protein, 15 aa, synthetic construct
GASRNKHNDYTTEYA

SEQ ID NO: 458 - protein, 15 aa, synthetic construct
AASRNKANAYTTEYA

SEQ ID NO: 459 - protein, 15 aa, synthetic construct
AASRNKFNDYTTEYA

SEQ ID NO: 460 - protein, 15 aa, synthetic construct
AASRNKANGYTTEYA

SEQ ID NO: 461 - protein, 15 aa, synthetic construct
AASRNKANEYTTEYA

SEQ ID NO: 462 - protein, 15 aa, synthetic construct
AASRNKANLYTTEYA

SEQ ID NO: 463 - protein, 15 aa, synthetic construct
AASRGKGNSYTTAYA

SEQ ID NO: 464 - protein, 15 aa, synthetic construct
AASRNKGNSYTTAYA

SEQ ID NO: 465 - protein, 15 aa, synthetic construct
AASRNNANDYTTEYA

SEQ ID NO: 466 - protein, 15 aa, synthetic construct
AASRNKANGYTTEYT

SEQ ID NO: 467 - protein, 15 aa, synthetic construct
AASRNSGNSYTTAYA

SEQ ID NO: 468 - protein, 15 aa, synthetic construct
AATRNKANDYTTEYA

SEQ ID NO: 469 - protein, 15 aa, synthetic construct
AASRNKANDFTTEYA

SEQ ID NO: 470 - protein, 15 aa, synthetic construct
AASRNKANDYTYEYA

SEQ ID NO: 471 - protein, 15 aa, synthetic construct
AASRNKANDYSTEYA

SEQ ID NO: 472 - protein, 15 aa, synthetic construct
AASRSKANDYTTEYA

SEQ ID NO: 473 - protein, 15 aa, synthetic construct
AASRNKANDYTTAYA

SEQ ID NO: 474 - protein, 15 aa, synthetic construct
AASRNTANDYTTEYA

SEQ ID NO: 475 - protein, 15 aa, synthetic construct
AASRNKANDYNTEYA

SEQ ID NO: 476 - protein, 15 aa, synthetic construct
AASRNKANDYTTEVA

SEQ ID NO: 477 - protein, 15 aa, synthetic construct
AASRNKGNDYTTAYA

SEQ ID NO: 478 - protein, 15 aa, synthetic construct
AASRNKANFYTTEYA

SEQ ID NO: 479 - protein, 15 aa, synthetic construct
AASRNEANDYTTEYA

SEQ ID NO: 480 - protein, 15 aa, synthetic construct
AAIRNKANDYTTEYA

SEQ ID NO: 481 - protein, 15 aa, synthetic construct
AASRNVANDYTTEYA

SEQ ID NO: 482 - protein, 15 aa, synthetic construct
AASRNKANDWTTEYA

SEQ ID NO: 483 - protein, 15 aa, synthetic construct
AASRNKANYYTTEYA

SEQ ID NO: 484 - protein, 16 aa, synthetic construct
CARDYYGSSYWYFDVW

SEQ ID NO: 485 - protein, 16 aa, synthetic construct
CARDYYGSSYWYYDVW

SEQ ID NO: 486 - protein, 16 aa, synthetic construct
CARDYLGSSYWYFDVW

SEQ ID NO: 487 - protein, 16 aa, synthetic construct
CARDYFGSSYWYFDVW

SEQ ID NO: 488 - protein, 16 aa, synthetic construct
CARDYYASSYWYFDVW

SEQ ID NO: 489 - protein, 16 aa, synthetic construct
CARDYYGSPYWYFDVW

SEQ ID NO: 490 - protein, 16 aa, synthetic construct
CARDYYGSSYWYFDSW

SEQ ID NO: 491 - protein, 16 aa, synthetic construct
CARDYYGSSYWYFDTW

SEQ ID NO: 492 - protein, 16 aa, synthetic construct
CARDYYGSSYWYFDLW

SEQ ID NO: 493 - protein, 16 aa, synthetic construct
CARDYYGSHYWYFDVW

SEQ ID NO: 494 - protein, 16 aa, synthetic construct
CARDYQGSSYWYFDVW

SEQ ID NO: 495 - protein, 16 aa, synthetic construct
CARDYYGHSYWYFDVW

SEQ ID NO: 496 - protein, 16 aa, synthetic construct
CARDYYGSSYWYLDVW

SEQ ID NO: 497 - protein, 16 aa, synthetic construct
CARDYYGNSYWYFDVW

SEQ ID NO: 498 - protein, 16 aa, synthetic construct
CARDYYGSAYWYFDVW

SEQ ID NO: 499 - protein, 16 aa, synthetic construct
CARDYYGQSYWYFDVW

SEQ ID NO: 500 - protein, 16 aa, synthetic construct
CARDYYGSSTWYFDVW

SEQ ID NO: 501 - protein, 16 aa, synthetic construct
CARDYYESSYWYFDVW

SEQ ID NO: 502 - protein, 16 aa, synthetic construct
CARDYYGSSHWYFDVW

SEQ ID NO: 503 - protein, 16 aa, synthetic construct
CARDYYGDSYWYFDVW

SEQ ID NO: 504 - protein, 16 aa, synthetic construct
CARDYYGSSYWYNDVW

SEQ ID NO: 505 - protein, 16 aa, synthetic construct
CARDYYHSSYWYFDVW

SEQ ID NO: 506 - protein, 16 aa, synthetic construct
CARDIYGSSYWYFDVW

SEQ ID NO: 507 - protein, 16 aa, synthetic construct
CARDYYGSSYWYDDVW

SEQ ID NO: 508 - protein, 16 aa, synthetic construct
CARDYYGYSYWYFDVW

SEQ ID NO: 509 - protein, 16 aa, synthetic construct
CARDYYGSSQWYFDVW

SEQ ID NO: 510 - protein, 16 aa, synthetic construct
CARDFYGSSYWYFDVW

SEQ ID NO: 511 - protein, 16 aa, synthetic construct
CARDYYGSSYWYHDVW

SEQ ID NO: 512 - protein, 16 aa, synthetic construct
CARDYYGSSYWYFDPW

SEQ ID NO: 513 - protein, 16 aa, synthetic construct
CARDYYGSQYWYFDVW

SEQ ID NO: 514 - protein, 16 aa, synthetic construct
CARDYYSSSYWYFDVW

SEQ ID NO: 515 - protein, 16 aa, synthetic construct
CARDYYGSSNWYFDVW

SEQ ID NO: 516 - protein, 16 aa, synthetic construct
CARDYYFSSYWYFDVW

SEQ ID NO: 517 - protein, 16 aa, synthetic construct
CARDYYDSSYWYFDVW

SEQ ID NO: 518 - protein, 16 aa, synthetic construct
CARDYYGASYWYFDVW

SEQ ID NO: 519 - protein, 16 aa, synthetic construct
CARDHYGSSYWYFDVW

SEQ ID NO: 520 - protein, 16 aa, synthetic construct
CARDYYGESYWYFDVW

SEQ ID NO: 521 - protein, 16 aa, synthetic construct
CARDYYLSSYWYFDVW

SEQ ID NO: 522 - protein, 16 aa, synthetic construct
CARDYYGSSYWYFDYW

SEQ ID NO: 523 - protein, 17 aa, synthetic construct
TASESLYSSKHKVHYLA

SEQ ID NO: 524 - protein, 17 aa, synthetic construct
VASESLYSSKHKVHYLA

SEQ ID NO: 525 - protein, 17 aa, synthetic construct
RASESISSSKNKVHYLA

SEQ ID NO: 526 - protein, 17 aa, synthetic construct
TASESLYSAKHKVHYLA

SEQ ID NO: 527 - protein, 17 aa, synthetic construct
TASESLYSSKHKVNYLA

SEQ ID NO: 528 - protein, 17 aa, synthetic construct
TASESLYSSKHKVHYVA

SEQ ID NO: 529 - protein, 17 aa, synthetic construct
TASFSLYSSKHKVHYLA

SEQ ID NO: 530 - protein, 17 aa, synthetic construct
TASESLYGSKHKVHYLA

SEQ ID NO: 531 - protein, 16 aa, synthetic construct
RASESISSSKHKHYLA

SEQ ID NO: 532 - protein, 17 aa, synthetic construct
TASEWLYSSKHKVHYLA

SEQ ID NO: 533 - protein, 17 aa, synthetic construct
TATESLYSSKHKVHYLA

SEQ ID NO: 534 - protein, 17 aa, synthetic construct
TASESLYSGKHKVHYLA

SEQ ID NO: 535 - protein, 17 aa, synthetic construct
FASESLYSSKHKVHYLA

SEQ ID NO: 536 - protein, 17 aa, synthetic construct
TASQSLYSSKNKVHYLA

SEQ ID NO: 537 - protein, 17 aa, synthetic construct
EASESLYSSKHKVHYLA

SEQ ID NO: 538 - protein, 17 aa, synthetic construct
TASESISSSKHKVHYLA

SEQ ID NO: 539 - protein, 17 aa, synthetic construct
TASESLYSQKHKVHYLA

SEQ ID NO: 540 - protein, 17 aa, synthetic construct
TASESLYSSKHSVHYLA

SEQ ID NO: 541 - protein, 17 aa, synthetic construct
TASEELYSSKHKVHYLA

SEQ ID NO: 542 - protein, 17 aa, synthetic construct
TASQSISSSKHKVHYLA

SEQ ID NO: 543 - protein, 16 aa, synthetic construct
TASESLSSSKHKHYLA

SEQ ID NO: 544 - protein, 17 aa, synthetic construct
TAEESLYSSKHKVHYLA

SEQ ID NO: 545 - protein, 17 aa, synthetic construct
TASESLYSSKHGVHYLA

SEQ ID NO: 546 - protein, 17 aa, synthetic construct
TASESLSSSKHKVHYLA

SEQ ID NO: 547 - protein, 17 aa, synthetic construct
TASQSLYSSKHKVHYLA

SEQ ID NO: 548 - protein, 15 aa, synthetic construct
RASESLYSSLHKYLA

SEQ ID NO: 549 - protein, 17 aa, synthetic construct
TASESVYSSKNKKHYLA

SEQ ID NO: 550 - protein, 17 aa, synthetic construct
TASESLYSSKGKVHYLA

SEQ ID NO: 551 - protein, 17 aa, synthetic construct
TASQSVYSSKNKVHYLA

SEQ ID NO: 552 - protein, 17 aa, synthetic construct
TSSQSLLSSSHNKNYLA

SEQ ID NO: 553 - protein, 17 aa, synthetic construct
TASESLYSPKHKVHYLA

SEQ ID NO: 554 - protein, 17 aa, synthetic construct
TASESLWSSKHKVHYLA

SEQ ID NO: 555 - protein, 17 aa, synthetic construct
KASQSVLSSKNNVNYLA

SEQ ID NO: 556 - protein, 17 aa, synthetic construct
TASESLYSSKWKVHYLA

SEQ ID NO: 557 - protein, 17 aa, synthetic construct
KSSESVYSSKHKKHYLA

SEQ ID NO: 558 - protein, 17 aa, synthetic construct
TASESLYSSKHKVDYLA

SEQ ID NO: 559 - protein, 17 aa, synthetic construct
KSSQSLYYSSNKKNYLA

SEQ ID NO: 560 - protein, 17 aa, synthetic construct
TASTSLYSSKHKVHYLA

SEQ ID NO: 561 - protein, 17 aa, synthetic construct
KASQSLYSSKHKKHYLA

SEQ ID NO: 562 - protein, 17 aa, synthetic construct
TASSSLYSSKHKVHYLA

SEQ ID NO: 563 - protein, 17 aa, synthetic construct
TASESLYSVKHKVHYLA

SEQ ID NO: 564 - protein, 17 aa, synthetic construct
GASESLYSSKSKVHYLA

SEQ ID NO: 565 - protein, 17 aa, synthetic construct
TSSESVYSSSHKVHYLA

SEQ ID NO: 566 - protein, 17 aa, synthetic construct
TASESLYSSKHKKNYLA

SEQ ID NO: 567 - protein, 17 aa, synthetic construct
KSSESLLYSKNNKNYLA

SEQ ID NO: 568 - protein, 17 aa, synthetic construct
TASESIYSSKHKVHYLA

SEQ ID NO: 569 - protein, 17 aa, synthetic construct
TSSQSVLSSKHNKNYLA

SEQ ID NO: 570 - protein, 17 aa, synthetic construct
TASEDLYSSKHKVHYLA

SEQ ID NO: 571 - protein, 17 aa, synthetic construct
KASQSVLYSKNKKHYLA

SEQ ID NO: 572 - protein, 17 aa, synthetic construct
TASESLYSSKHKLHYLA

SEQ ID NO: 573 - protein, 17 aa, synthetic construct
TASESLYSSKHKVVYLA

SEQ ID NO: 574 - protein, 17 aa, synthetic construct
TSSESLYSSKNKVNYLA

SEQ ID NO: 575 - protein, 17 aa, synthetic construct
TSSQSLYSSKHNKHYLA

SEQ ID NO: 576 - protein, 17 aa, synthetic construct
TASESLYSNKHKVHYLA

SEQ ID NO: 577 - protein, 17 aa, synthetic construct
TASENLYSSKHKVHYLA

SEQ ID NO: 578 - protein, 17 aa, synthetic construct
TASESLYSSKIKVHYLA

SEQ ID NO: 579 - protein, 17 aa, synthetic construct
KSSQSLLYSKNNVNYLA

SEQ ID NO: 580 - protein, 17 aa, synthetic construct
KSSESLYSSKNKVHYLA

SEQ ID NO: 581 - protein, 17 aa, synthetic construct
TSSESLLSSKHKVHYLA

SEQ ID NO: 582 - protein, 17 aa, synthetic construct
TASQSVYSSKHKKHYLA

SEQ ID NO: 583 - protein, 17 aa, synthetic construct
TASQSVYSSKHNKHYLA

SEQ ID NO: 584 - protein, 17 aa, synthetic construct
TSSQSLLYSSNKKHYLA

SEQ ID NO: 585 - protein, 17 aa, synthetic construct
KSSQSVLSSKHKKNYLA

SEQ ID NO: 586 - protein, 17 aa, synthetic construct
TASQSVYSSKNKKHYLA

SEQ ID NO: 587 - protein, 17 aa, synthetic construct
TASESVLSSKHKKNYLA

SEQ ID NO: 588 - protein, 17 aa, synthetic construct
TASVSLYSSKHKVHYLA

SEQ ID NO: 589 - protein, 17 aa, synthetic construct
TASESLYSTKHKVHYLA

SEQ ID NO: 590 - protein, 17 aa, synthetic construct
TASESLYSSNHKVHYLA

SEQ ID NO: 591 - protein, 7 aa, synthetic construct
GASNRYI

SEQ ID NO: 592 - protein, 7 aa, synthetic construct
GASNREI

SEQ ID NO: 593 - protein, 7 aa, synthetic construct
GASHRYI

SEQ ID NO: 594 - protein, 7 aa, synthetic construct
GASSLQI

SEQ ID NO: 595 - protein, 7 aa, synthetic construct
GASNRYT

SEQ ID NO: 596 - protein, 7 aa, synthetic construct
GASNRYH

SEQ ID NO: 597 - protein, 7 aa, synthetic construct
GSSNRYI

SEQ ID NO: 598 - protein, 7 aa, synthetic construct
GASNRFI

SEQ ID NO: 599 - protein, 7 aa, synthetic construct
GASNRYY

SEQ ID NO: 600 - protein, 7 aa, synthetic construct
GAANRYI

SEQ ID NO: 601 - protein, 7 aa, synthetic construct
GASNRYV

SEQ ID NO: 602 - protein, 7 aa, synthetic construct
GASNRYD

SEQ ID NO: 603 - protein, 7 aa, synthetic construct
GASNRYP

SEQ ID NO: 604 - protein, 7 aa, synthetic construct
GAVNRYI

SEQ ID NO: 605 - protein, 7 aa, synthetic construct
GASNGYI

SEQ ID NO: 606 - protein, 7 aa, synthetic construct
GASNRYS

SEQ ID NO: 607 - protein, 7 aa, synthetic construct
GASNLYS

SEQ ID NO: 608 - protein, 7 aa, synthetic construct
GASNRYF

SEQ ID NO: 609 - protein, 7 aa, synthetic construct
GASNLQS

SEQ ID NO: 610 - protein, 7 aa, synthetic construct
GLSNRYI

SEQ ID NO: 611 - protein, 7 aa, synthetic construct
GASNRQS

SEQ ID NO: 612 - protein, 7 aa, synthetic construct
GAFNRYT

SEQ ID NO: 613 - protein, 7 aa, synthetic construct
GASNRLI

SEQ ID NO: 614 - protein, 7 aa, synthetic construct
GQSNRYI

SEQ ID NO: 615 - protein, 7 aa, synthetic construct
GASNRNI

SEQ ID NO: 616 - protein, 7 aa, synthetic construct
GAGNRYI

SEQ ID NO: 617 - protein, 7 aa, synthetic construct
GASNRYG

SEQ ID NO: 618 - protein, 7 aa, synthetic construct
GASGRYI

SEQ ID NO: 619 - protein, 7 aa, synthetic construct
GASNLQI

SEQ ID NO: 620 - protein, 7 aa, synthetic construct
GASNRGI

SEQ ID NO: 621 - protein, 7 aa, synthetic construct
GESNRYI

SEQ ID NO: 622 - protein, 7 aa, synthetic construct
GVSNRYI

SEQ ID NO: 623 - protein, 7 aa, synthetic construct
GASNRPI

SEQ ID NO: 624 - protein, 7 aa, synthetic construct
GASFRYI

SEQ ID NO: 625 - protein, 7 aa, synthetic construct
GADNRYI

SEQ ID NO: 626 - protein, 7 aa, synthetic construct
GASNRYQ

SEQ ID NO: 627 - protein, 7 aa, synthetic construct
GASSRYI

SEQ ID NO: 628 - protein, 7 aa, synthetic construct
GTSNRYI

SEQ ID NO: 629 - protein, 7 aa, synthetic construct
GASNHYI

SEQ ID NO: 630 - protein, 7 aa, synthetic construct
GASNRQI

SEQ ID NO: 631 - protein, 7 aa, synthetic construct
GASSRYS

SEQ ID NO: 632 - protein, 7 aa, synthetic construct
GASNRDI

SEQ ID NO: 633 - protein, 7 aa, synthetic construct
GISNRYI

SEQ ID NO: 634 - protein, 7 aa, synthetic construct
GASSLYS

SEQ ID NO: 635 - protein, 7 aa, synthetic construct
GASNRAI

SEQ ID NO: 636 - protein, 7 aa, synthetic construct
GASNRYA

SEQ ID NO: 637 - protein, 7 aa, synthetic construct
GASNRVI

SEQ ID NO: 638 - protein, 7 aa, synthetic construct
GPSNRYI

SEQ ID NO: 639 - protein, 7 aa, synthetic construct
GAFNRYI

SEQ ID NO: 640 - protein, 7 aa, synthetic construct
GANNRYI

SEQ ID NO: 641 - protein, 7 aa, synthetic construct
GAHNRYI

SEQ ID NO: 642 - protein, 7 aa, synthetic construct
GNSNRYI

SEQ ID NO: 643 - protein, 7 aa, synthetic construct
GASWRYI

SEQ ID NO: 644 - protein, 7 aa, synthetic construct
GASTRYS

SEQ ID NO: 645 - protein, 7 aa, synthetic construct
GASNRYW

SEQ ID NO: 646 - protein, 7 aa, synthetic construct
GASNRYL

SEQ ID NO: 647 - protein, 7 aa, synthetic construct
GASNRYN

SEQ ID NO: 648 - protein, 7 aa, synthetic construct
GASQRYI

SEQ ID NO: 649 - protein, 7 aa, synthetic construct
GAQNRYI

SEQ ID NO: 650 - protein, 7 aa, synthetic construct
GATNRYI

SEQ ID NO: 651 - protein, 7 aa, synthetic construct
GASNPYI

SEQ ID NO: 652 - protein, 7 aa, synthetic construct
GASTRYI

SEQ ID NO: 653 - protein, 7 aa, synthetic construct
GAENRYI

SEQ ID NO: 654 - protein, 7 aa, synthetic construct
GASHRQI

SEQ ID NO: 655 - protein, 7 aa, synthetic construct
GASNTYI

SEQ ID NO: 656 - protein, 11 aa, synthetic construct
CAEFYSYPLTF

SEQ ID NO: 657 - protein, 11 aa, synthetic construct [sequence omitted]

SEQ ID NO: 658 - protein, 11 aa, synthetic construct
CAQFYAYPLTF

SEQ ID NO: 659 - protein, 11 aa, synthetic construct
CAQFYSYPLTF

SEQ ID NO: 660 - protein, 11 aa, synthetic construct
CAQFYHYPLTF

SEQ ID NO: 661 - protein, 11 aa, synthetic construct
CAQFYQYPLTF

SEQ ID NO: 662 - protein, 11 aa, synthetic construct
CAQFYYYPLTF

SEQ ID NO: 663 - protein, 11 aa, synthetic construct
CAQFYVYPLTF

SEQ ID NO: 664 - protein, 11 aa, synthetic construct
CGQFYSYPLTF

SEQ ID NO: 665 - protein, 11 aa, synthetic construct
CAQFYDYPLTF

SEQ ID NO: 666 - protein, 11 aa, synthetic construct
CAQFYRYPLTF

SEQ ID NO: 667 - protein, 11 aa, synthetic construct
CAQFYTYPLTF

SEQ ID NO: 668 - protein, 11 aa, synthetic construct
CAQFYWYPLTF

SEQ ID NO: 669 - protein, 11 aa, synthetic construct
CTQFYSYPLTF

SEQ ID NO: 670 - protein, 11 aa, synthetic construct
CAQFYFYPLTF

SEQ ID NO: 671 - protein, 11 aa, synthetic construct
CAQFYEYPLTF

SEQ ID NO: 672 - protein, 11 aa, synthetic construct
CAHFYSYPLTF

SEQ ID NO: 673 - protein, 11 aa, synthetic construct
CAQFYKYPLTF

SEQ ID NO: 674 - protein, 11 aa, synthetic construct
CAQFFSYPLTF

SEQ ID NO: 675 - protein, 11 aa, synthetic construct
CVQFYSYPLTF

SEQ ID NO: 676 - protein, 11 aa, synthetic construct
CAQFYIYPLTF

SEQ ID NO: 677 - protein, 11 aa, synthetic construct
CANFYSYPLTF

SEQ ID NO: 678 - protein, 11 aa, synthetic construct
CAQYYSYPLTF

SEQ ID NO: 679 - protein, 11 aa, synthetic construct
CAQFYLYPLTF

SEQ ID NO: 680 - protein, 11 aa, synthetic construct
CAQFYNYPLTF

SEQ ID NO: 681 - protein, 11 aa, synthetic construct
CASFYSYPLTF

SEQ ID NO: 682 - protein, 11 aa, synthetic construct
CAAFYSYPLTF

SEQ ID NO: 683 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 684 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 685 - protein, 113 aa, synthetic construct
DIQMTQSPSSLSASVGDRVTITCTASESLYSSKHKVHYLAWYQQKPGKAPKLLIYGASNRYIGVPSRFSGSGSGTDFTLTISSLQPEDFATYYCAQFYSYPLTFGQGTKVEIK

SEQ ID NO: 686 - protein, 19 aa, synthetic construct
AGGGGSGGGGSGGGGSAAA

SEQ ID NO: 687 - protein, 24 aa, synthetic construct
AEAAAKEAAAKEAAAKEAAAKAAA

SEQ ID NO: 688 - protein, 107 aa, synthetic construct [sequence omitted]

SEQ ID NO: 689 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 690 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 691 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 692 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 693 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS